(19) United States
(12) Patent Application Publication
(10) Pub. No.: US 2019/0198775 A1
(43) Pub. Date: Jun. 27, 2019

(54) COMPOSITION AND ORGANIC OPTOELECTRONIC DEVICE AND DISPLAY DEVICE

(71) Applicant: Samsung SDI Co., Ltd., Yongin-si (KR)

(72) Inventors: Jinhyun Lui, Suwon-si (KR); Byoungkwan Lee, Suwon-si (KR); Ho Kuk Jung, Suwon-si (KR); Youngkyoung Jo, Suwon-si (KR); Changwoo Kim, Suwon-si (KR); Min Seok Seo, Suwon-si (KR); Sangshin Lee, Suwon-si (KR); Seungjae Lee, Suwon-si (KR); Sung-Hyun Jung, Suwon-si (KR); Pyeongseok Cho, Suwon-si (KR)

(21) Appl. No.: 16/224,906

(22) Filed: Dec. 19, 2018

(30) Foreign Application Priority Data
Dec. 27, 2017 (KR) 10-2017-0181466

(51) Int. Cl. H01L 51/00; H01L 51/50

(52) U.S. Cl. CPC H01L 51/0072; H01L 51/0067; H01L 51/0073; C07D 405/14; H01L 51/5016; H01L 51/5036; H01L 51/0074

(57) ABSTRACT

A composition, an organic optoelectronic device, and a display device, the composition including a first compound represented by Chemical Formula 1 and a second compound represented by Chemical Formula 2 or Chemical Formula 3:

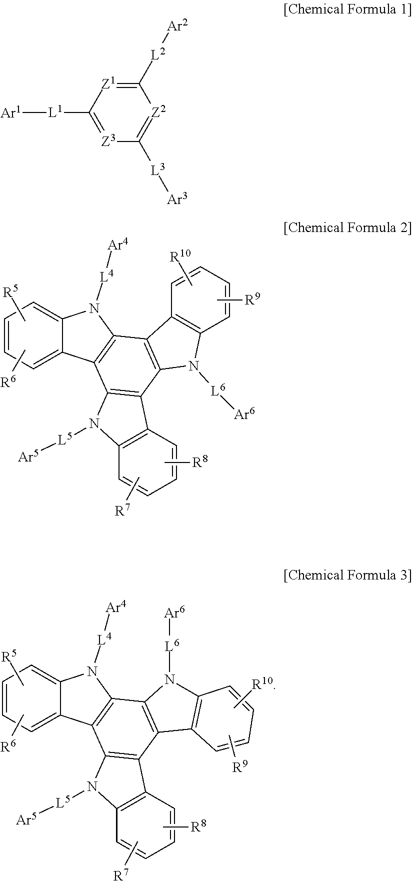

### CROSS-REFERENCE TO RELATED APPLICATION

Korean Patent Application No. 10-2017-0181466 filed in the Korean Intellectual Property Office on Dec. 27, 2017, and entitled: “Composition and Organic Optoelectronic Device and Display Device,” is incorporated by reference herein in its entirety.

### BACKGROUND

### 1. Field

Embodiments relate to a composition, an organic optoelectronic device, and a display device are disclosed.

### 2. Description of the Related Art

An organic optoelectronic device (organic optoelectronic diode) is a device that converts electrical energy into photoenergy, and vice versa.

An organic optoelectronic device may be classified as follows in accordance with its driving principles. One is a photoelectric device where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of the organic optoelectronic device may include an organic photoelectric device, an organic light emitting diode, an organic solar cell, and an organic photo conductor drum.

Of these, an organic light emitting diode (OLED) has recently drawn attention due to an increase in demand for flat panel displays. The organic light emitting diode converts electrical energy into light by applying current to an organic light emitting material and performance of an organic light emitting diode may be affected by organic materials disposed between electrodes.

### SUMMARY

The embodiments may be realized by providing a composition including a first compound represented by Chemical Formula 1, and a second compound represented by Chemical Formula 2 or Chemical Formula 3:

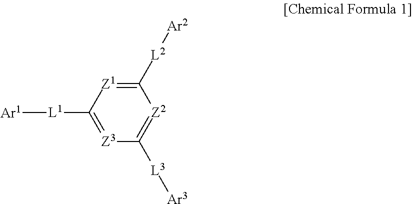

wherein, in Chemical Formula 1, Z¹ to Z³ are independently N or CR^{a}, in which R^{a} is hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, at least two of Z′ to Z³ are N, L′ to L³ are independently a single bond, a substituted or unsubstituted C6 to C20 arylene group, a substituted or unsubstituted divalent C3 to C20 heterocyclic group, or a combination thereof, Ar¹ to Ar³ are independently a substituted or unsubstituted C6 to C30 aryl group, a substituted or unsubstituted C3 to C30 heterocyclic group, or a combination thereof, and at least one of Ar¹ to Ar³ is a group represented by Chemical Formula A,

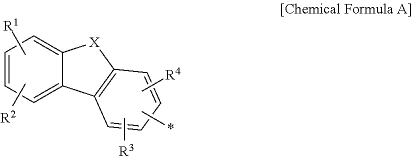

wherein, in Chemical Formula A, X is O or S, R¹ to R⁴ are independently hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, R¹ and R² are separate or are linked with each other to form a ring, R³ and R⁴ are separate or are linked with each other to form a ring, and * is a linking point with Chemical Formula 1,

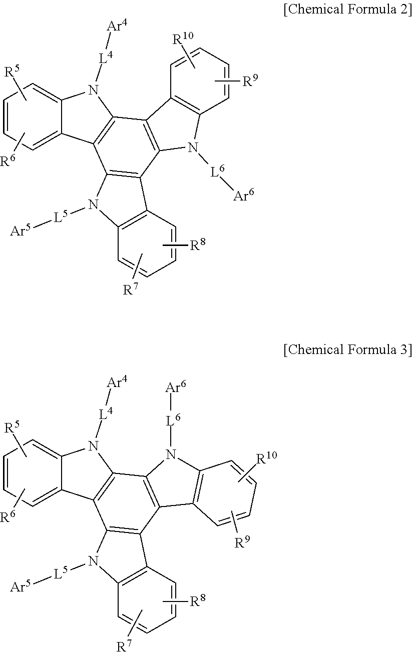

wherein, in Chemical Formula 2 and Chemical Formula 3, Ar⁴ to Ar⁶ are independently hydrogen, deuterium, a substituted or unsubstituted C1 to C30 alkyl group, a substituted or unsubstituted C6 to C30 aryl group, a substituted or unsubstituted C3 to C30 heterocyclic group, or a combination thereof, L⁴ to L⁶ are independently a single bond, a substituted or unsubstituted C6 to C20 arylene group, a substituted or unsubstituted divalent C3 to C20 heterocyclic group, or a combination thereof, R⁵ to R¹⁰ are independently hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, R⁵ and R⁶ are separate or are linked with each other to form a ring, R⁷ and R⁸ are separate or are linked with each other to form a ring, and R⁹ and R¹⁰ are separate or are linked with each other to form a ring.

The first compound may be represented by one of Chemical Formulae 1A to 1C:

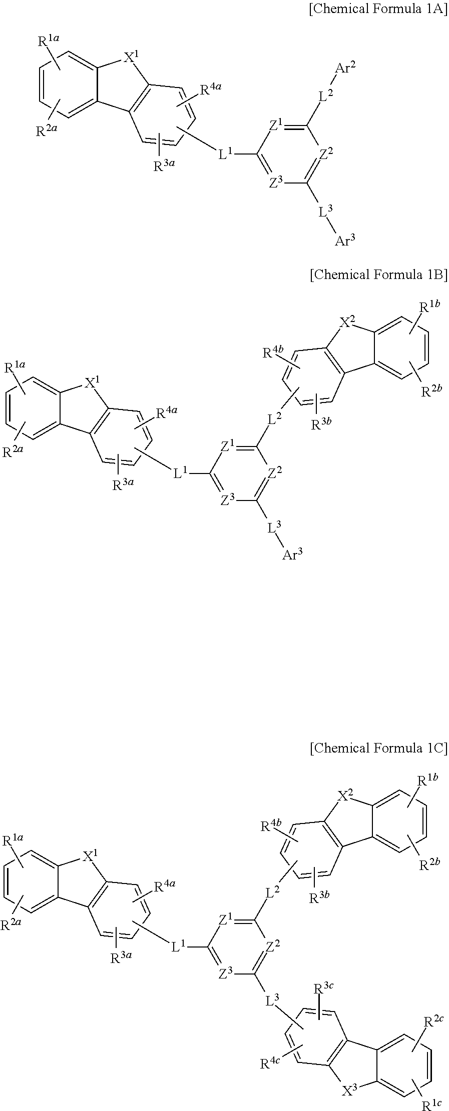

wherein, in Chemical Formulae 1A to 1C, Z¹ to Z³ are independently N or CR^{a}, in which R^{a} is hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, at least two of Z¹ to Z³ are N, L¹ to L³ are independently a single bond, a substituted or unsubstituted C6 to C20 arylene group, a substituted or unsubstituted divalent C3 to C20 heterocyclic group, or a combination thereof, Ar² and Ar³ are independently a substituted or unsubstituted C6 to C30 aryl group, a substituted or unsubstituted C3 to C30 heterocyclic group, or a combination thereof, X¹ to X³ are independently O or S, and R^{1a} to R^{4a}, R^{1b} to R^{4b}, and R^{1c} to R^{4c} are independently hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof.

Ar² and Ar³ may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted pyridinyl group, a substituted or unsubstituted pyrimidinyl group, a substituted or unsubstituted triazinyl group, or a combination thereof.

The group represented by Chemical Formula A may be a group represented by one of Chemical Formulae A-1 to A-4:

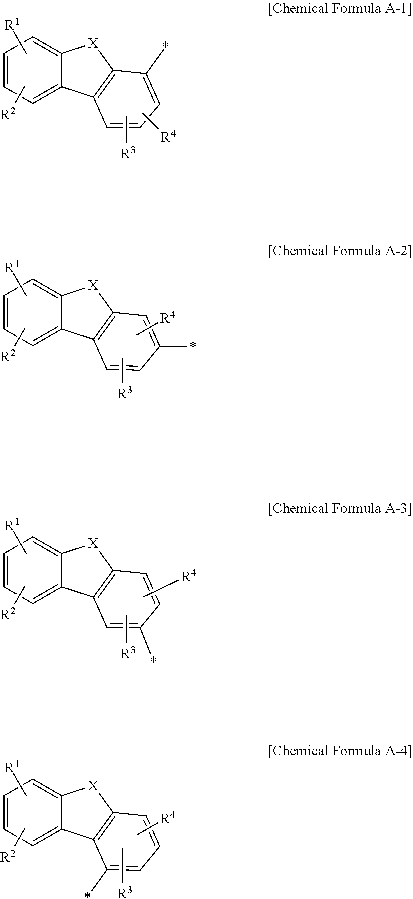

wherein, in Chemical Formulae A-1 to A-4, X is O or S, R¹ to R⁴ are independently hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, R¹ and R² are separate or are linked with each other to form a ring, R³ and R⁴ are separate or are linked with each other to form a ring, and * is a linking point with Chemical Formula 1.

Ar¹ to Ar³ may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted pyridinyl group, a substituted or unsubstituted pyrimidinyl group, a substituted or unsubstituted triazinyl group, a group represented by Chemical Formula A, or a combination thereof.

The first compound may be one of the compounds listed in Group 1:

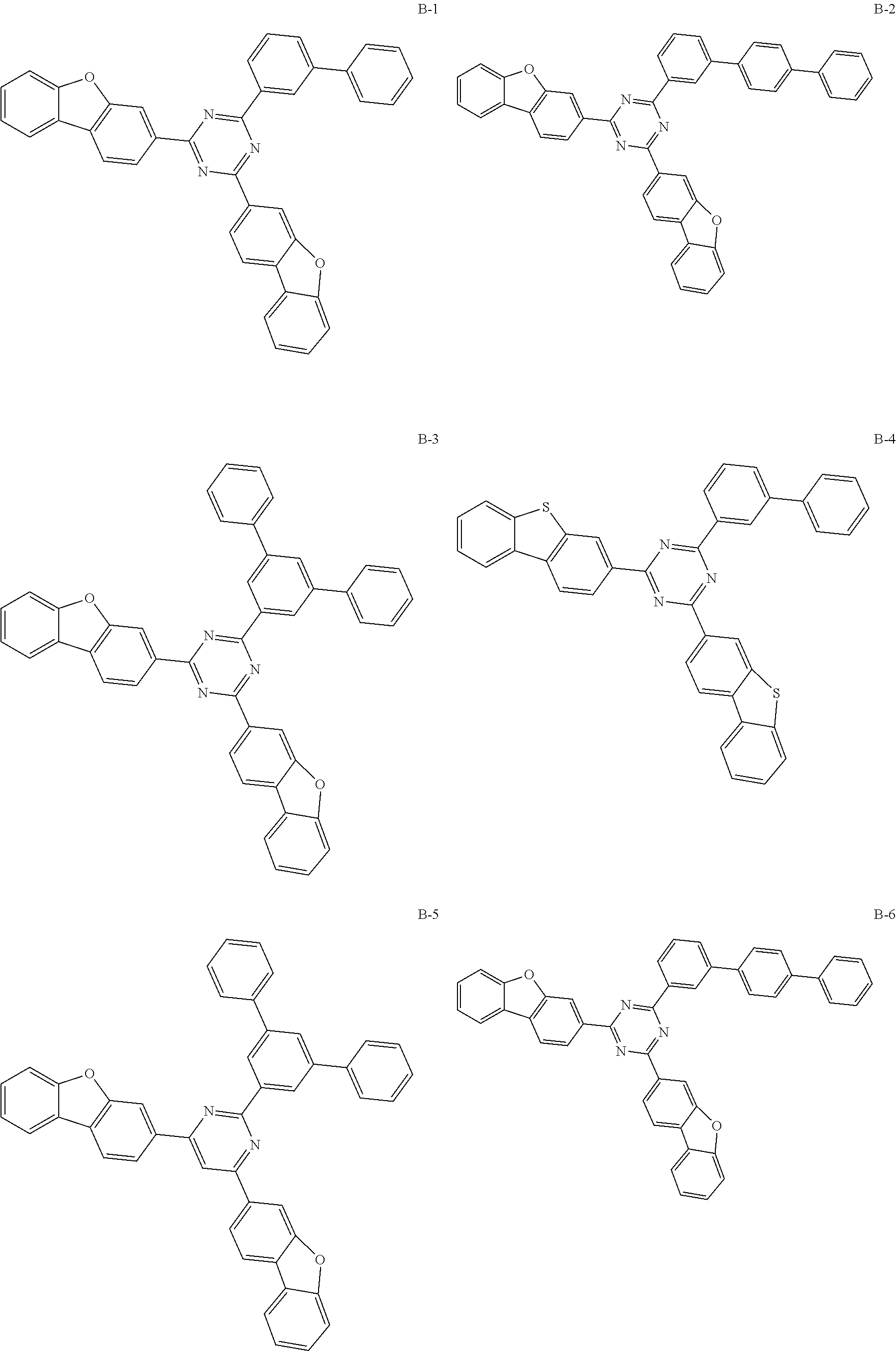

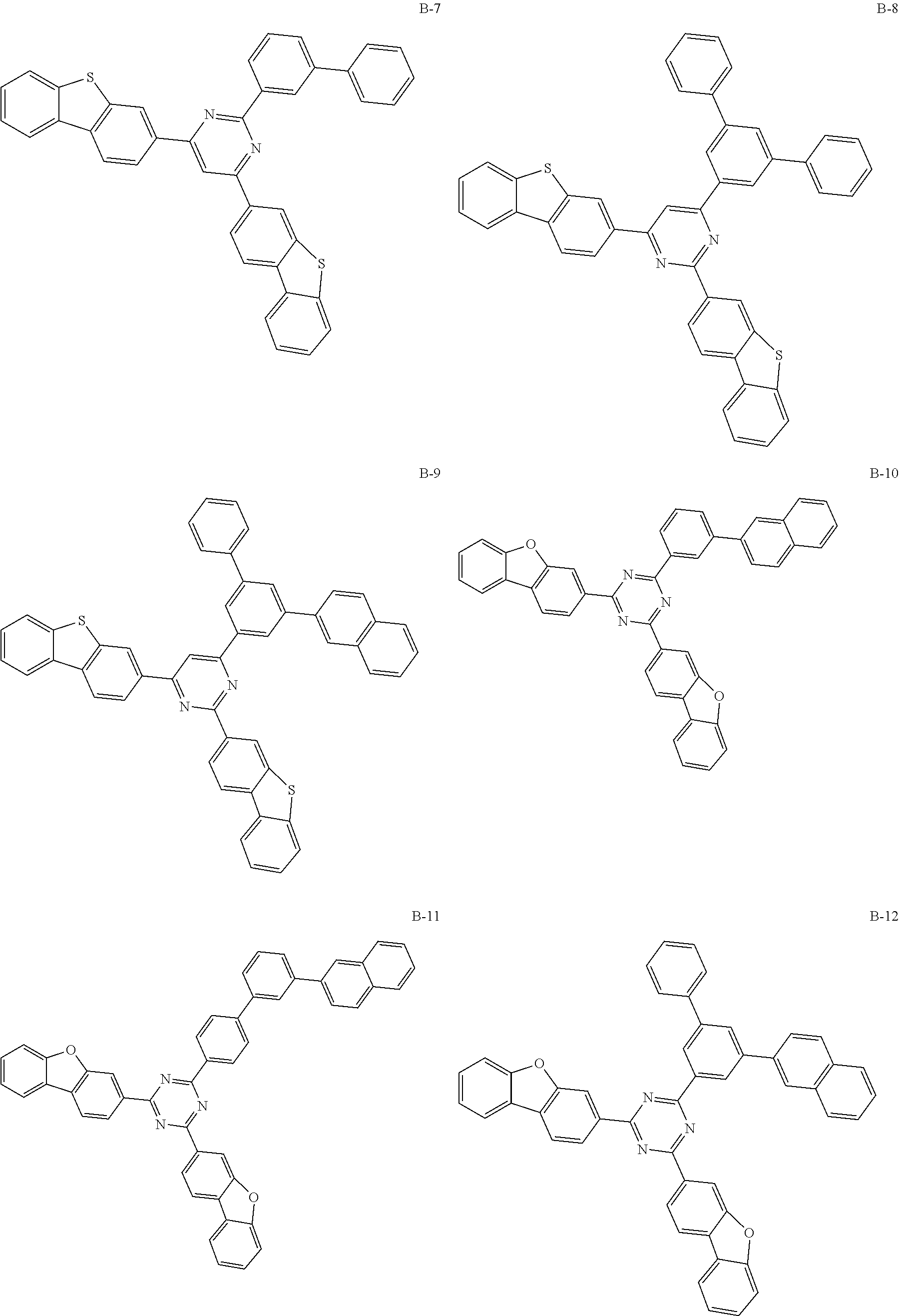

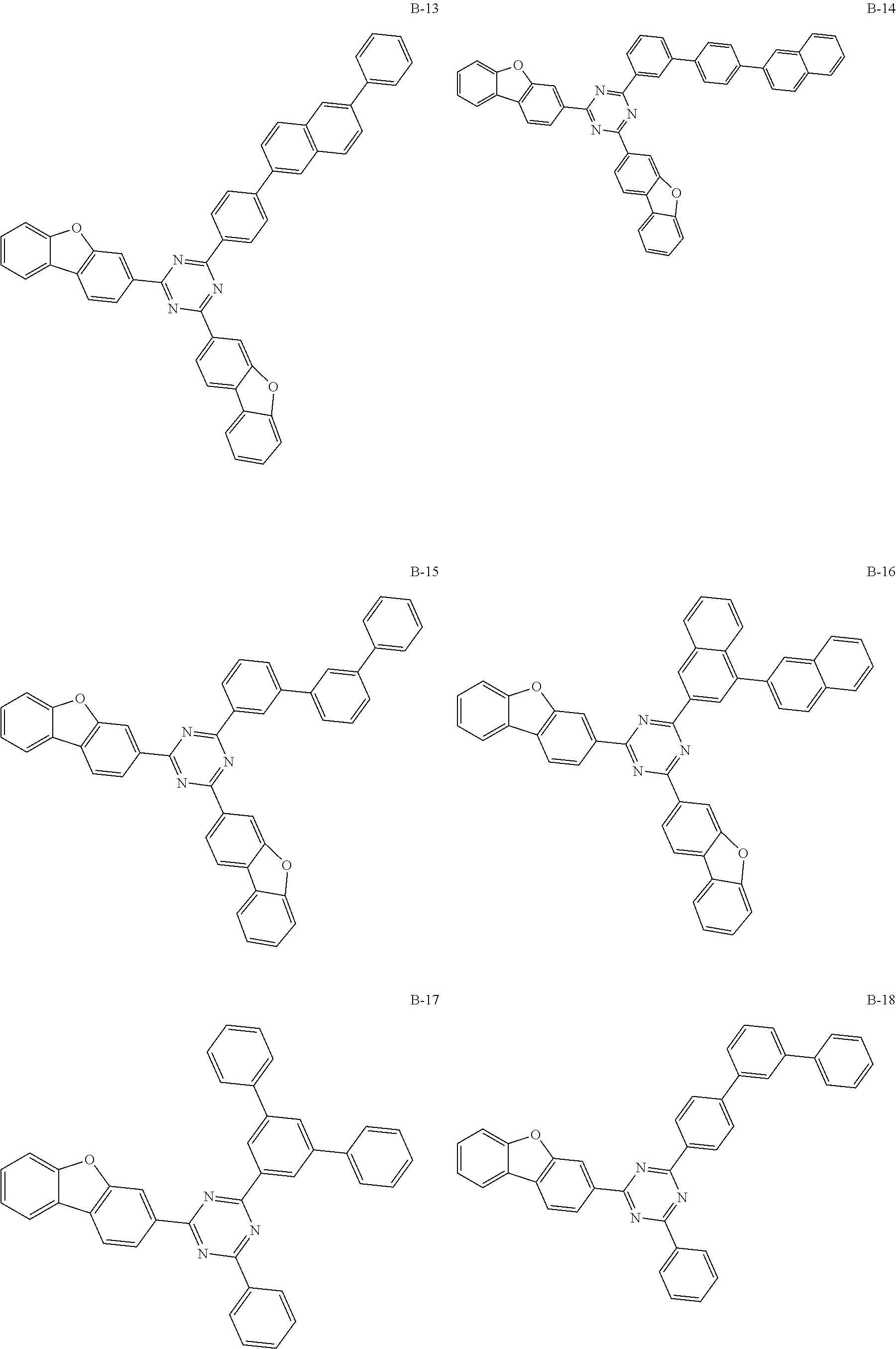

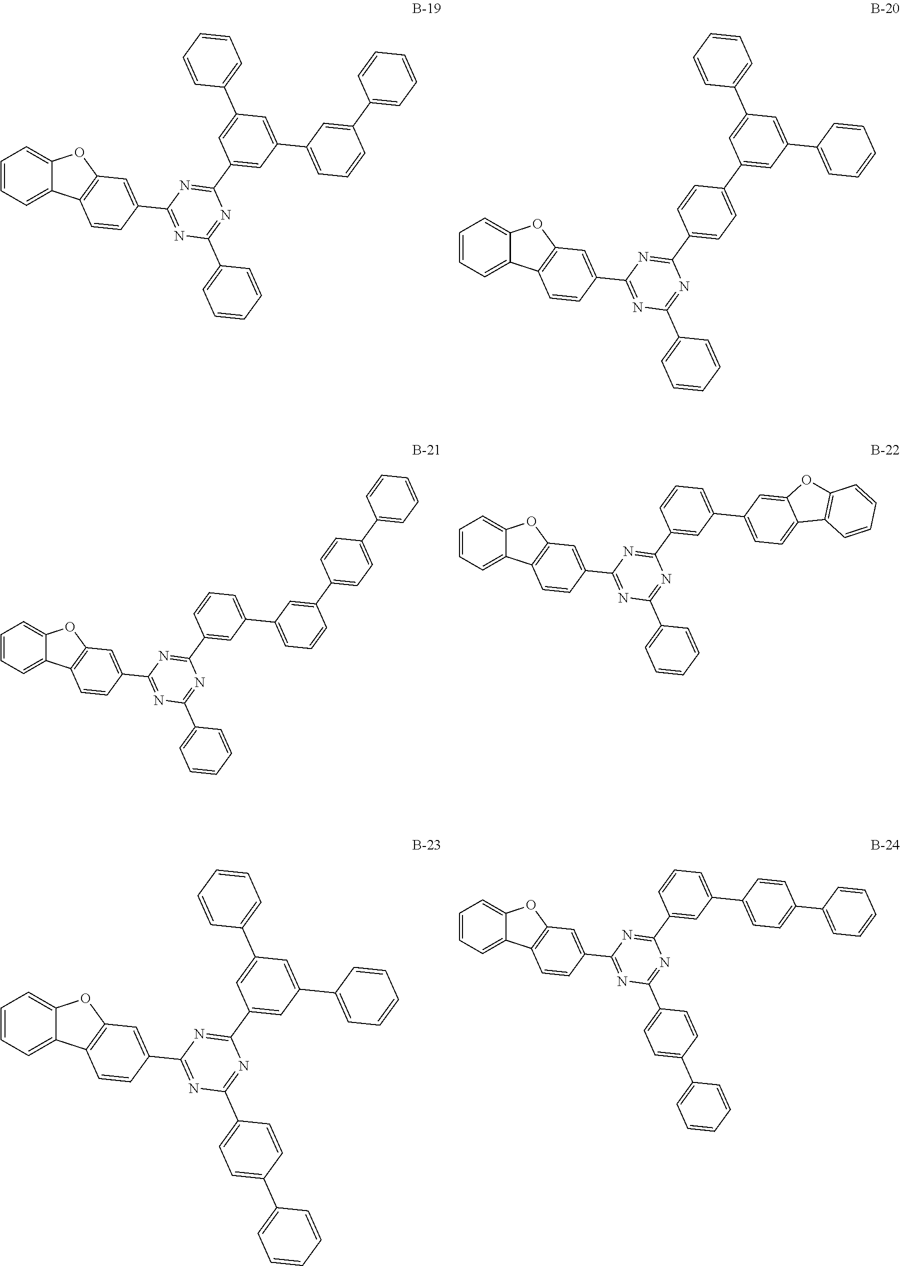

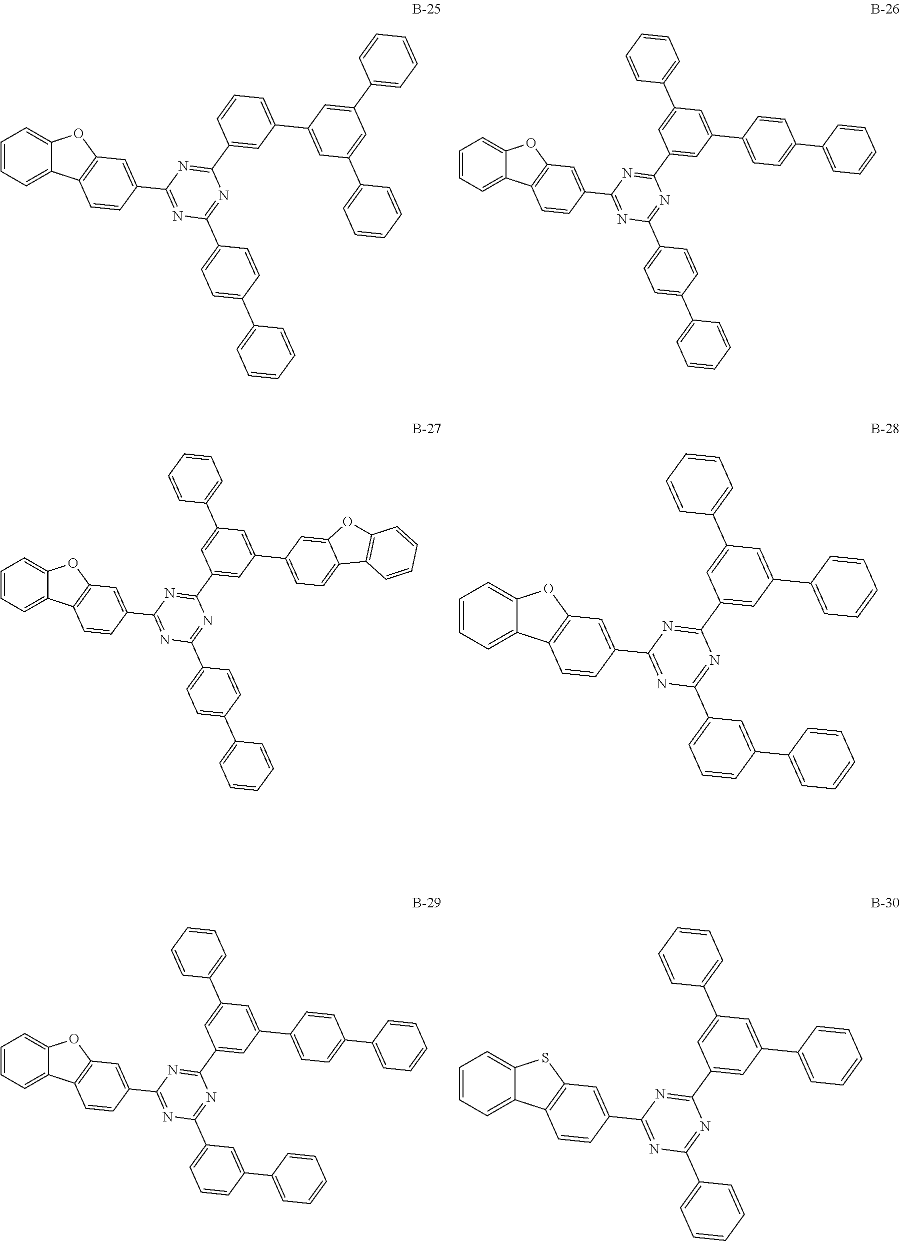

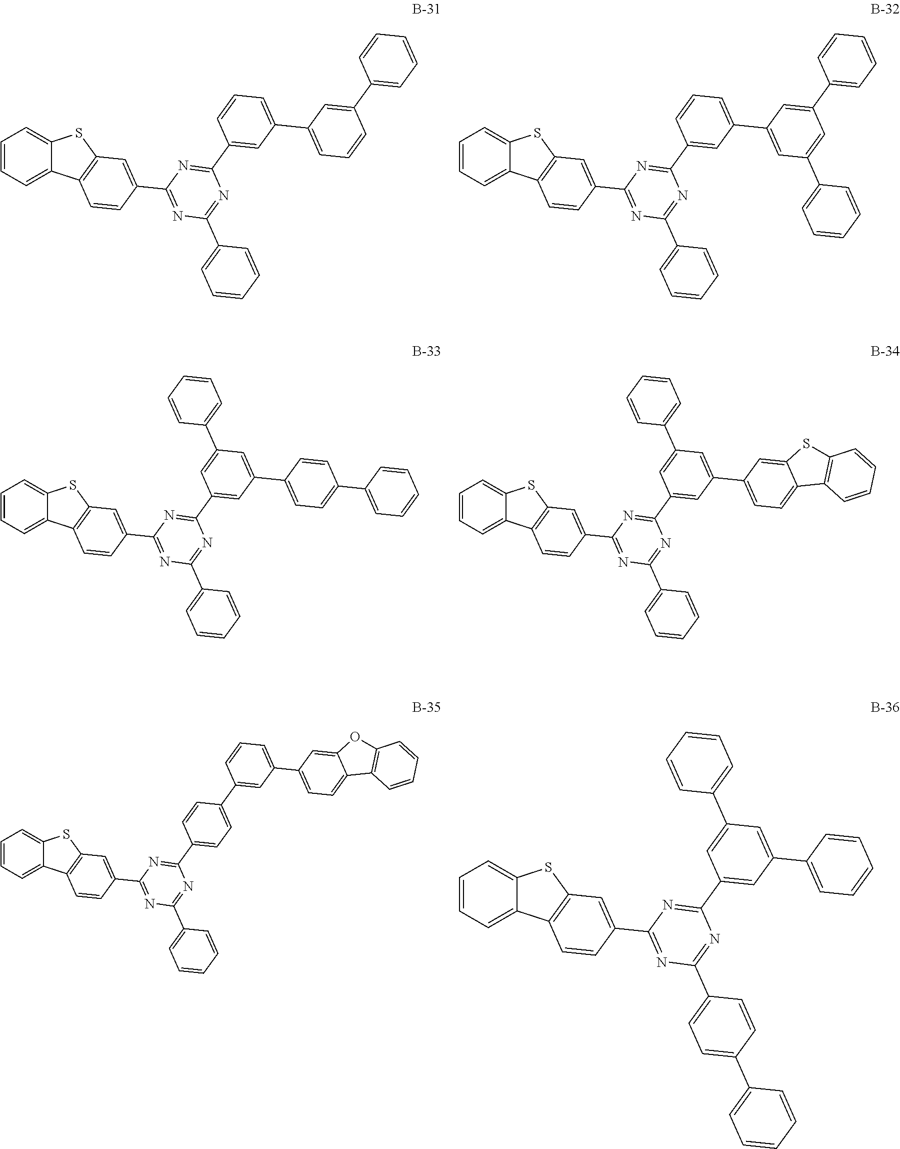

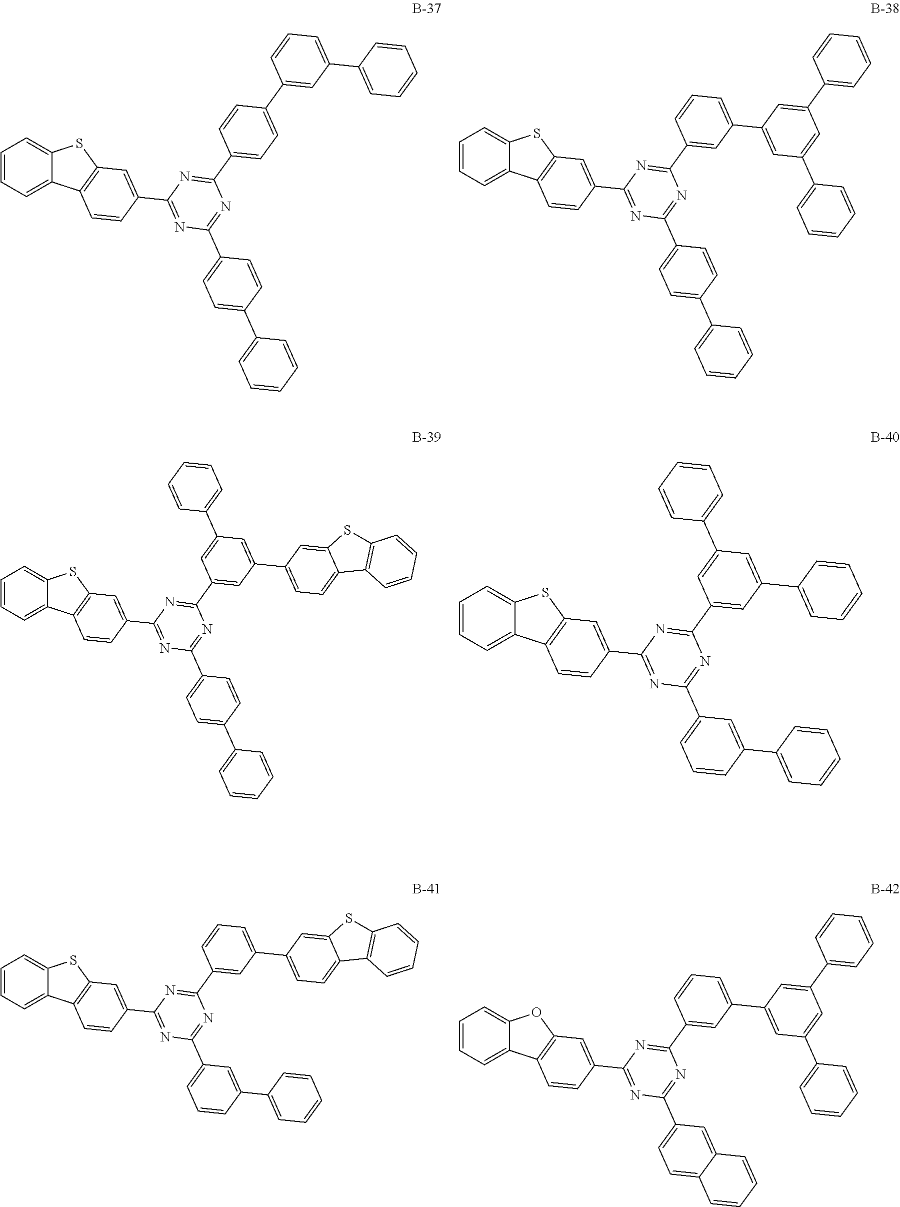

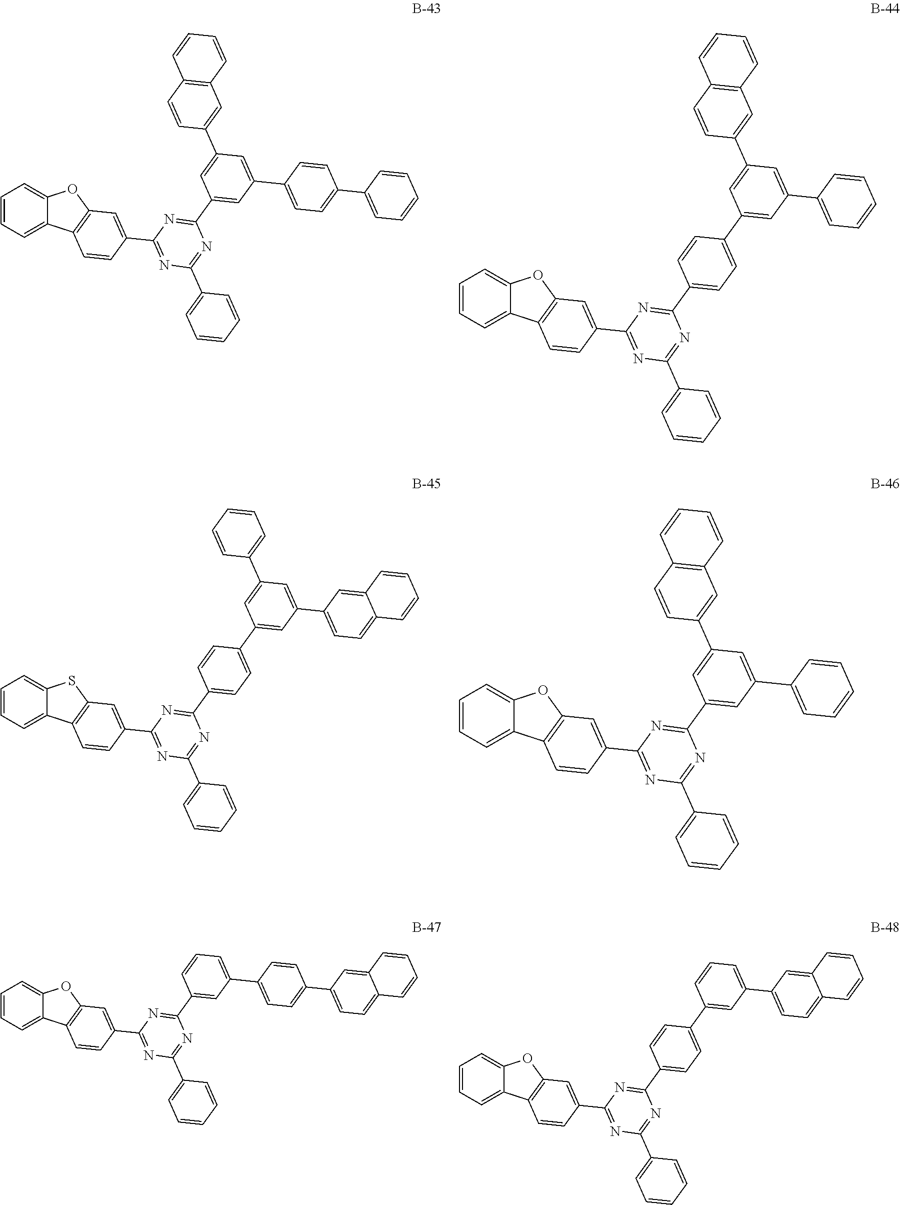

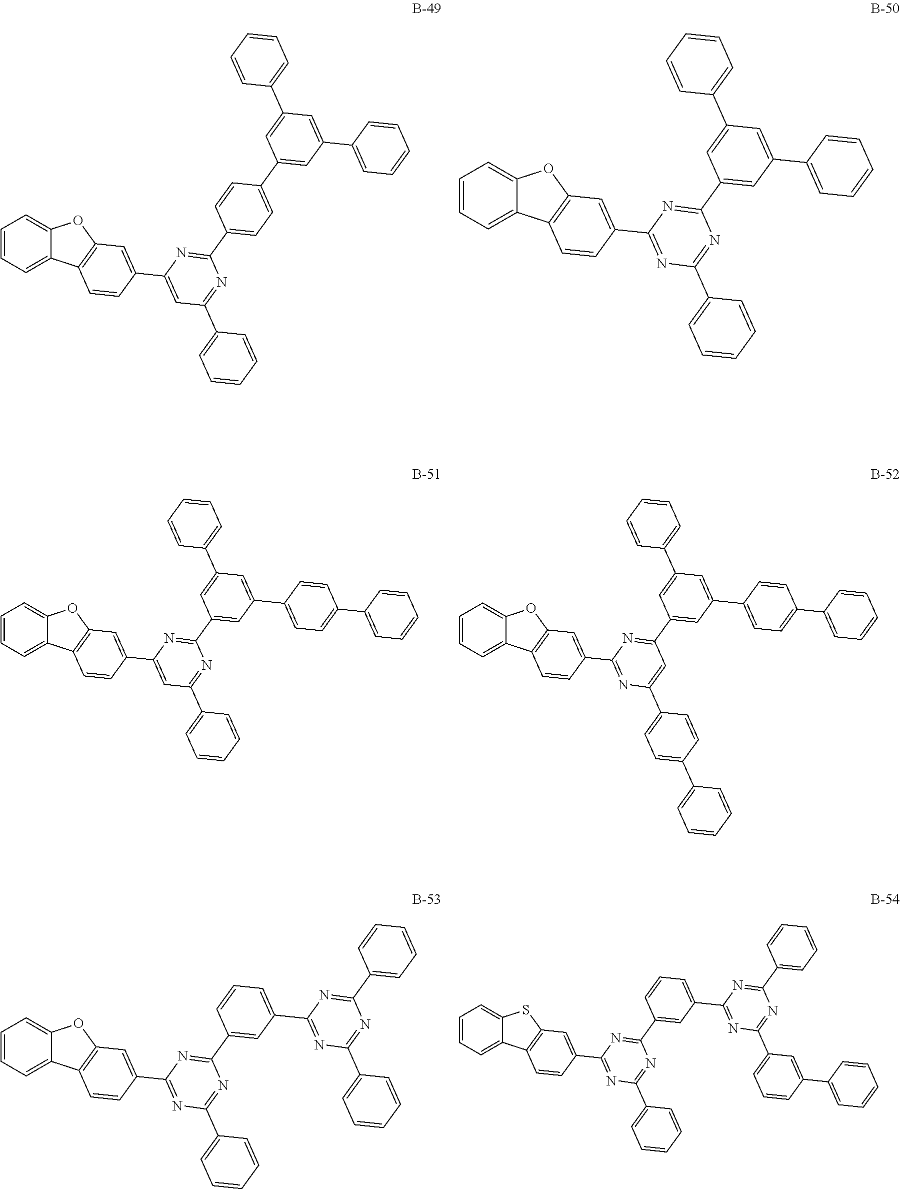

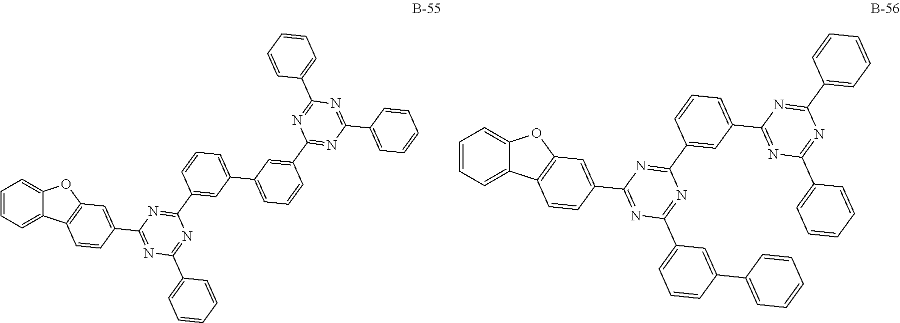 [SMILES: c1ccc(-c2nc(-c3ccccc3)nc(-c3cccc(-c4cccc(-c5nc(-c6ccccc6)nc(-c6ccc7c(c6)oc6ccccc67)n5)c4)c3)n2)cc1 ; c1ccc(-c2cccc(-c3nc(-c4cccc(-c5nc(-c6ccccc6)nc(-c6ccccc6)n5)c4)nc(-c4ccc5c(c4)oc4ccccc45)n3)c2)cc1]

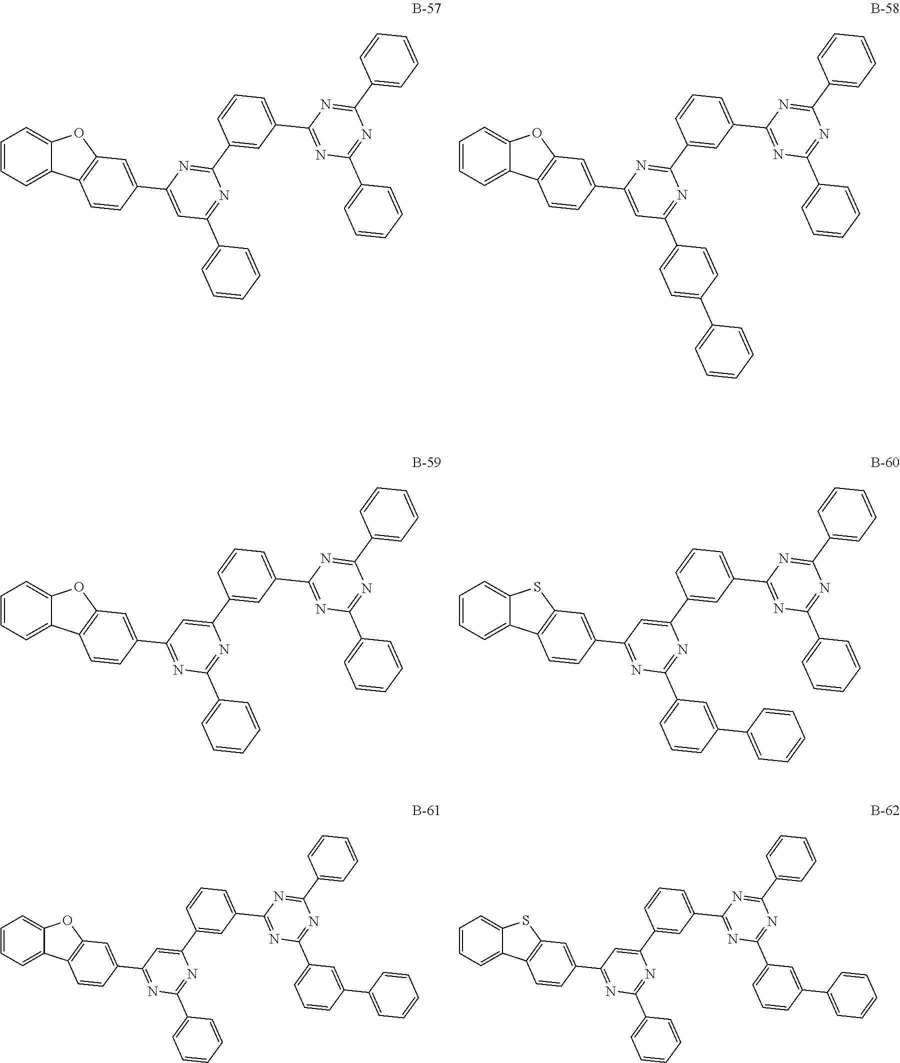

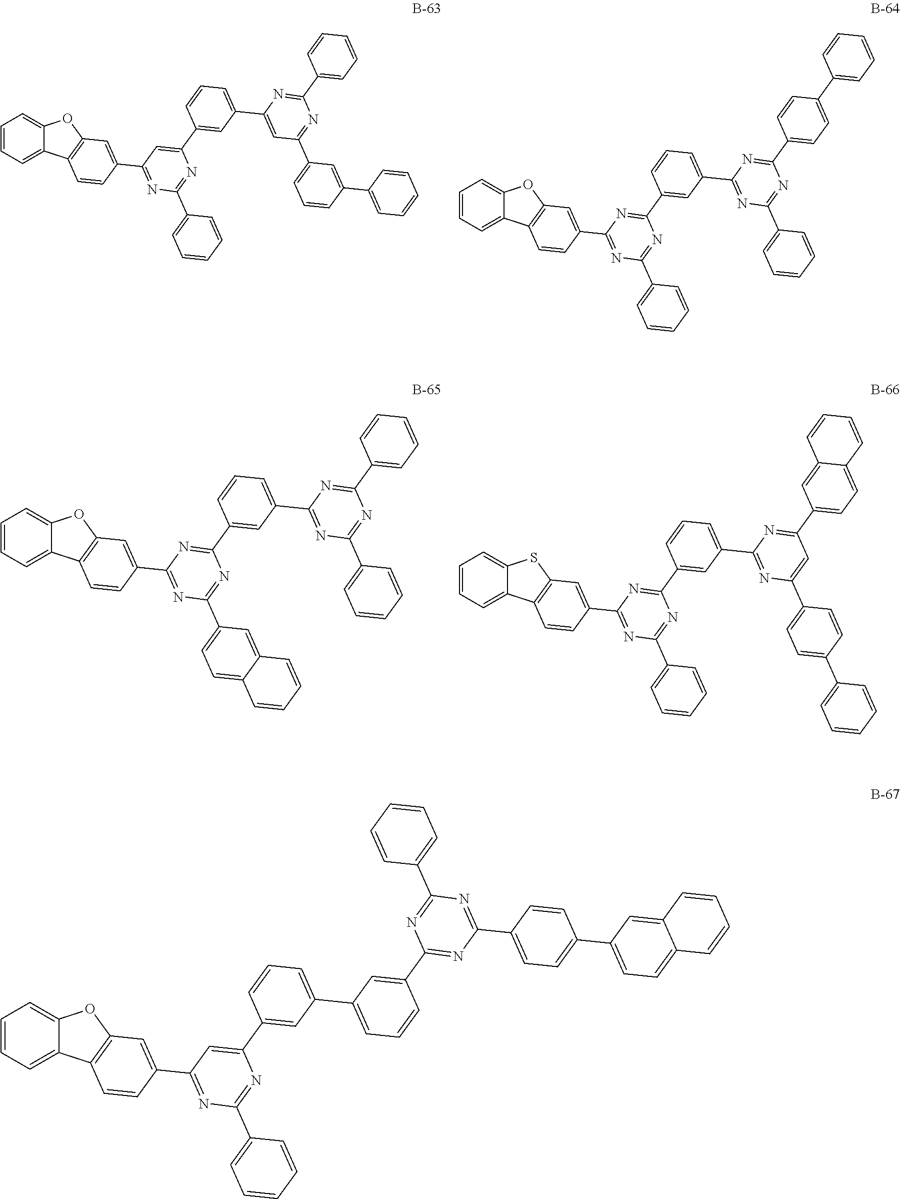

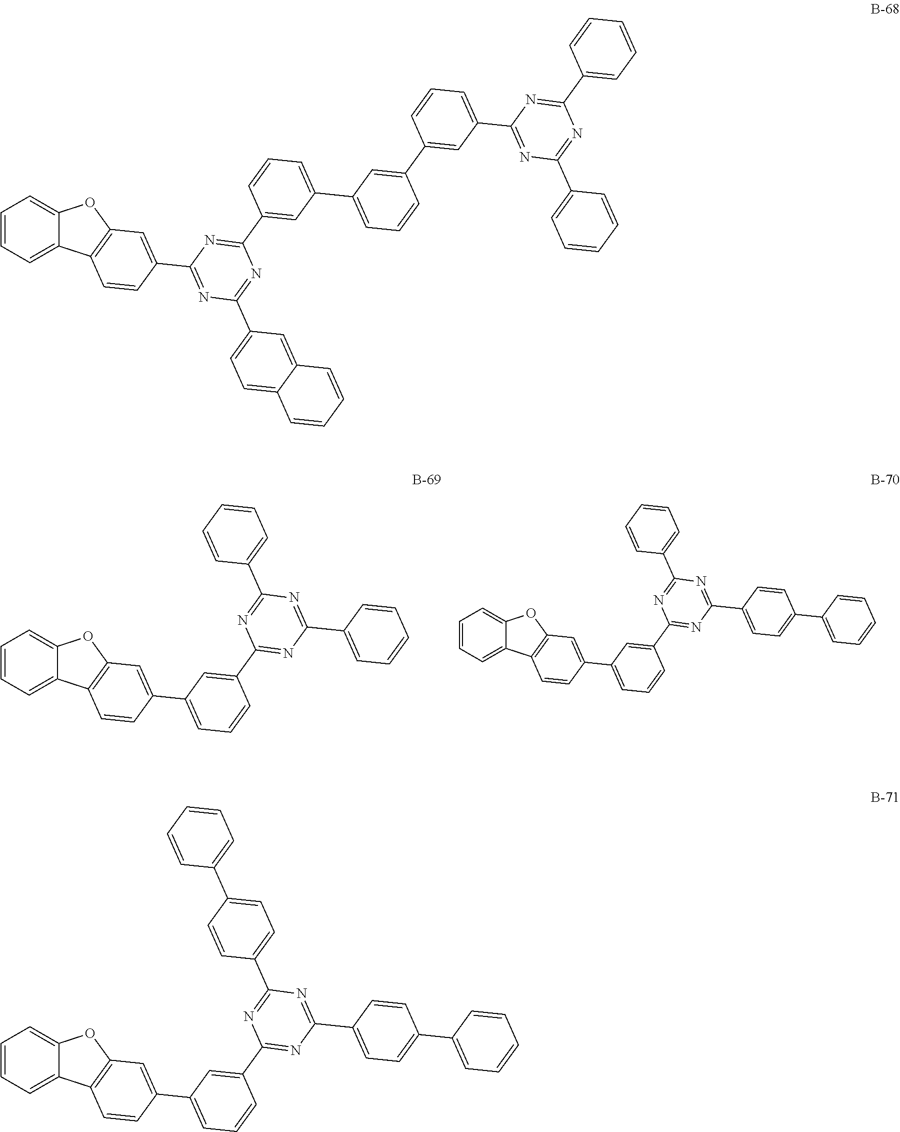 [SMILES: c1ccc(-c2nc(-c3ccccc3)nc(-c3cccc(-c4cccc(-c5cccc(-c6nc(-c7ccc8ccccc8c7)nc(-c7ccc8c(c7)oc7ccccc78)n6)c5)c4)c3)n2)cc1 ; c1ccc(-c2nc(-c3ccccc3)nc(-c3cccc(-c4ccc5c(c4)oc4ccccc45)c3)n2)cc1 ; c1ccc(-c2ccc(-c3nc(-c4ccccc4)nc(-c4cccc(-c5ccc6c(c5)oc5ccccc56)c4)n3)cc2)cc1 ; c1ccc(-c2ccc(-c3nc(-c4ccc(-c5ccccc5)cc4)nc(-c4cccc(-c5ccc6c(c5)oc5ccccc56)c4)n3)cc2)cc1]

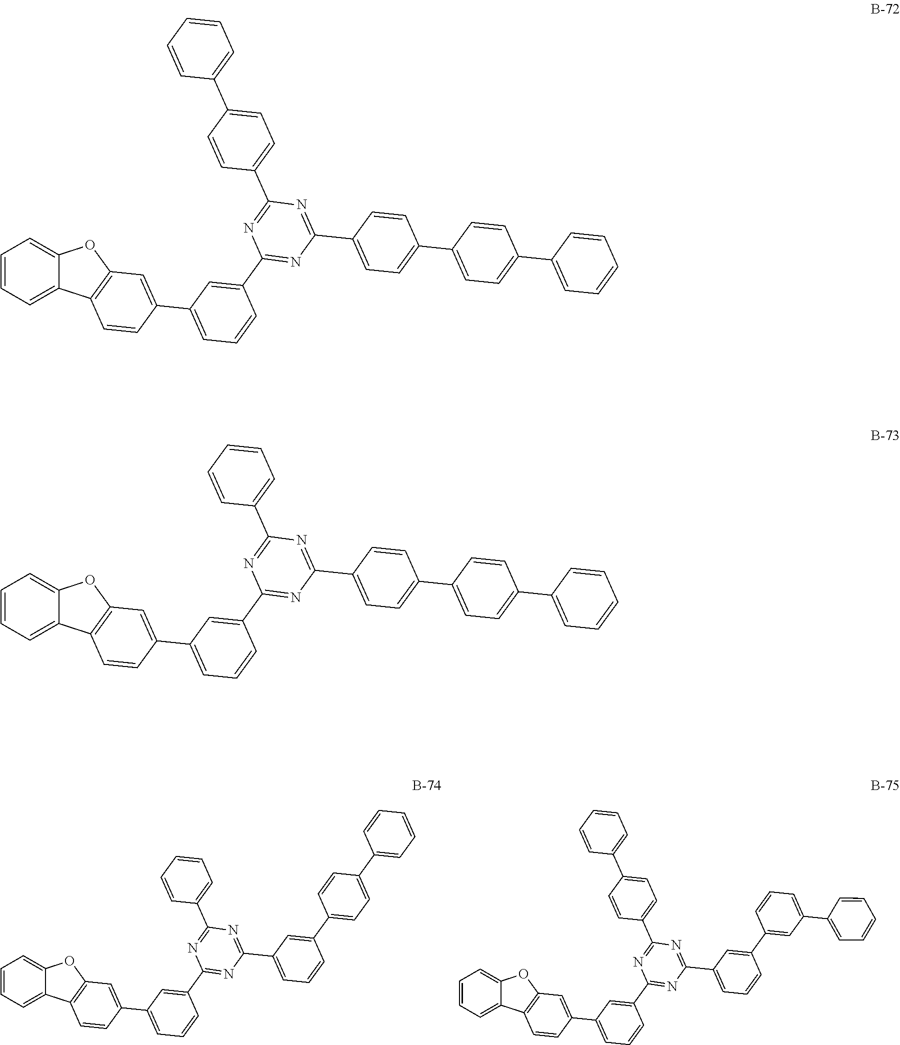 [SMILES: c1ccc(-c2ccc(-c3ccc(-c4nc(-c5ccc(-c6ccccc6)cc5)nc(-c5cccc(-c6ccc7c(c6)oc6ccccc67)c5)n4)cc3)cc2)cc1 ; c1ccc(-c2ccc(-c3ccc(-c4nc(-c5ccccc5)nc(-c5cccc(-c6ccc7c(c6)oc6ccccc67)c5)n4)cc3)cc2)cc1 ; c1ccc(-c2ccc(-c3cccc(-c4nc(-c5ccccc5)nc(-c5cccc(-c6ccc7c(c6)oc6ccccc67)c5)n4)c3)cc2)cc1 ; c1ccc(-c2ccc(-c3nc(-c4cccc(-c5cccc(-c6ccccc6)c5)c4)nc(-c4cccc(-c5ccc6c(c5)oc5ccccc56)c4)n3)cc2)cc1]

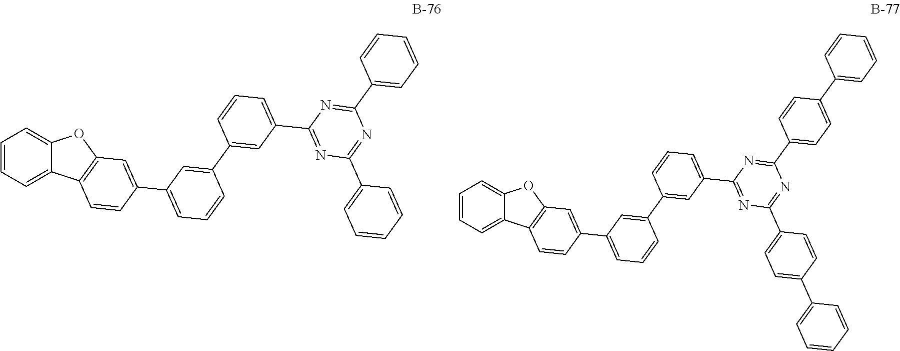 [SMILES: c1ccc(-c2nc(-c3ccccc3)nc(-c3cccc(-c4cccc(-c5ccc6c(c5)oc5ccccc56)c4)c3)n2)cc1 ; c1ccc(-c2ccc(-c3nc(-c4ccc(-c5ccccc5)cc4)nc(-c4cccc(-c5cccc(-c6ccc7c(c6)oc6ccccc67)c5)c4)n3)cc2)cc1]

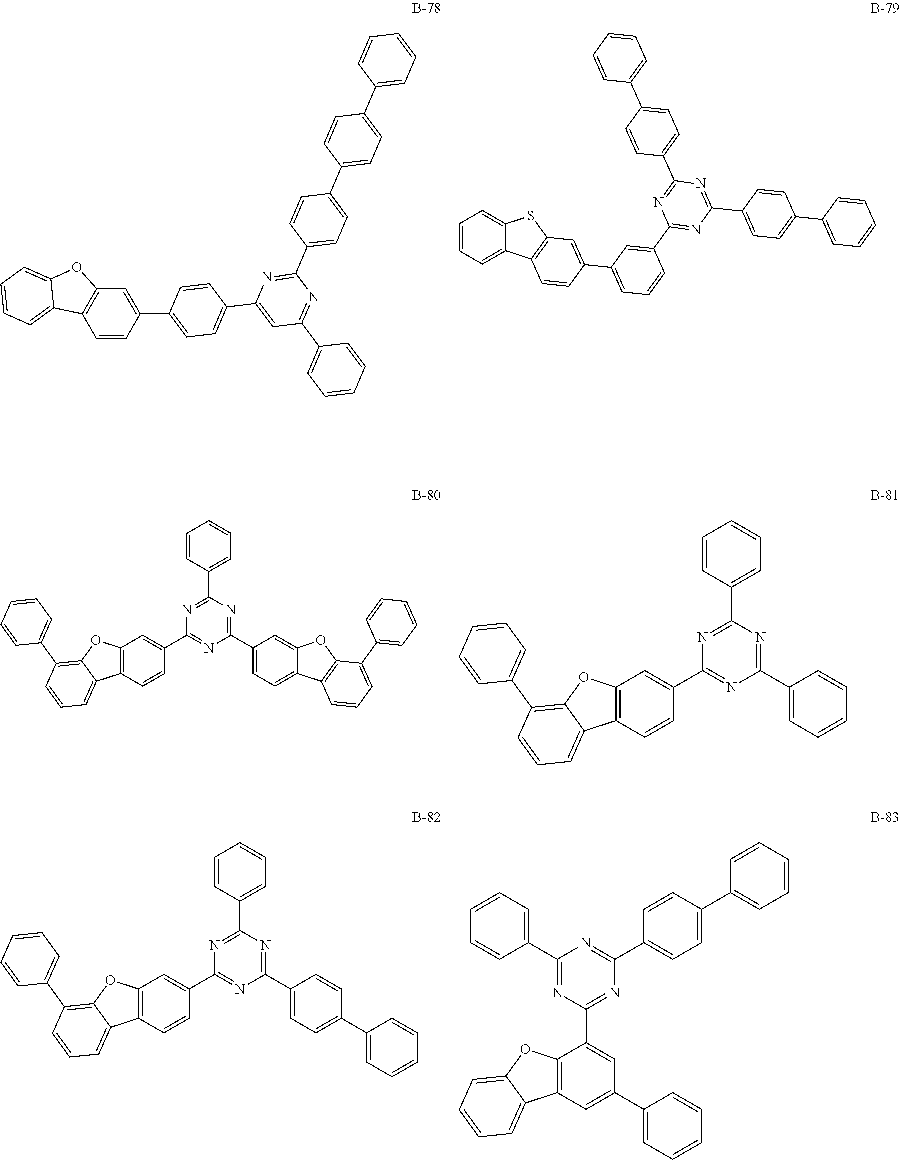 [SMILES: c1ccc(-c2ccc(-c3ccc(-c4nc(-c5ccccc5)cc(-c5ccc(-c6ccc7c(c6)oc6ccccc67)cc5)n4)cc3)cc2)cc1 ; c1ccc(-c2ccc(-c3nc(-c4ccc(-c5ccccc5)cc4)nc(-c4cccc(-c5ccc6c(c5)sc5ccccc56)c4)n3)cc2)cc1 ; c1ccc(-c2nc(-c3ccc4c(c3)oc3c(-c5ccccc5)cccc34)nc(-c3ccc4c(c3)oc3c(-c5ccccc5)cccc34)n2)cc1 ; c1ccc(-c2nc(-c3ccccc3)nc(-c3ccc4c(c3)oc3c(-c5ccccc5)cccc34)n2)cc1 ; c1ccc(-c2ccc(-c3nc(-c4ccccc4)nc(-c4ccc5c(c4)oc4c(-c6ccccc6)cccc45)n3)cc2)cc1 ; c1ccc(-c2ccc(-c3nc(-c4ccccc4)nc(-c4cc(-c5ccccc5)cc5c4oc4ccccc45)n3)cc2)cc1]

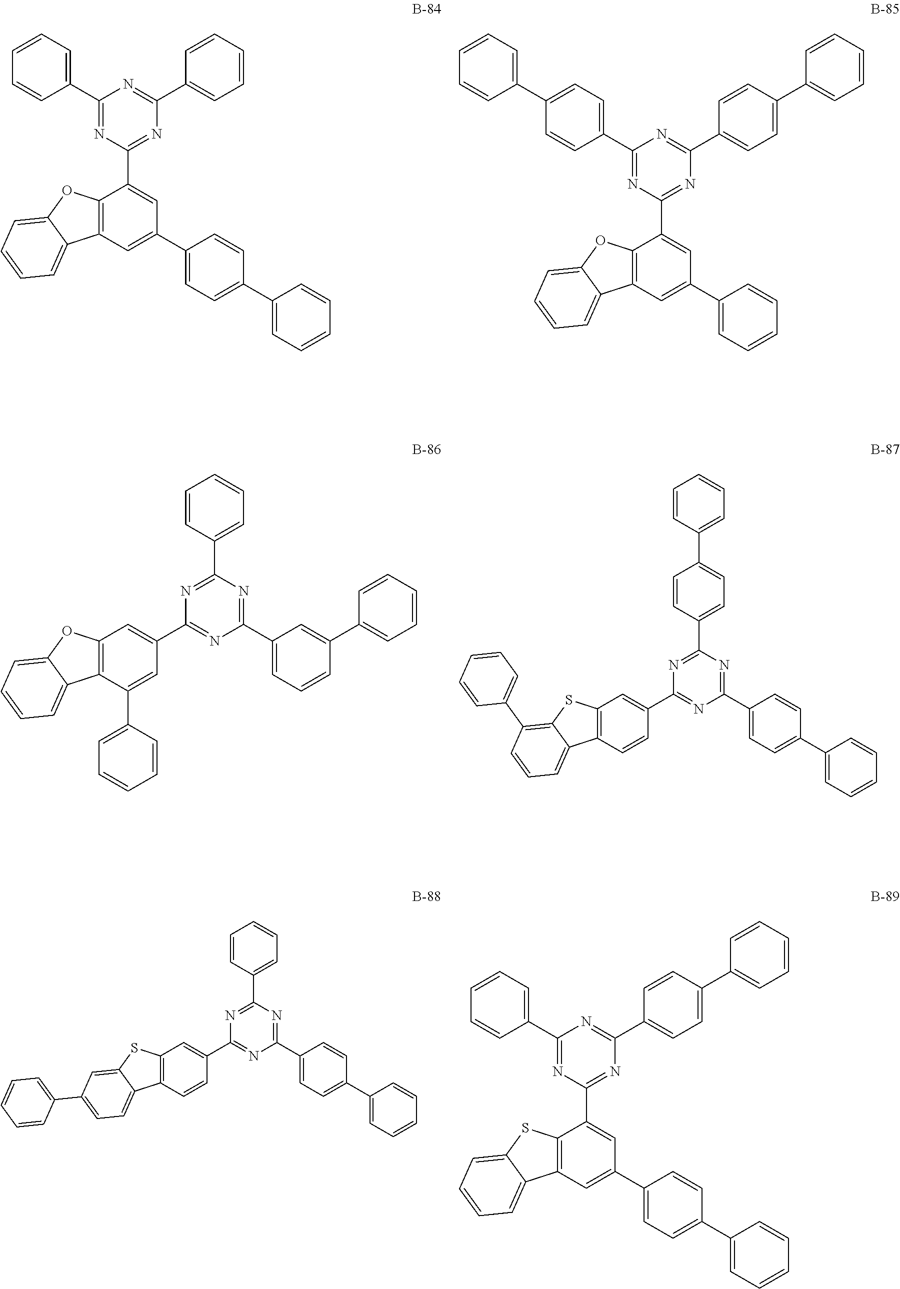 [SMILES: c1ccc(-c2ccc(-c3cc(-c4nc(-c5ccccc5)nc(-c5ccccc5)n4)c4oc5ccccc5c4c3)cc2)cc1 ; c1ccc(-c2ccc(-c3nc(-c4ccc(-c5ccccc5)cc4)nc(-c4cc(-c5ccccc5)cc5c4oc4ccccc45)n3)cc2)cc1 ; c1ccc(-c2cccc(-c3nc(-c4ccccc4)nc(-c4cc(-c5ccccc5)c5c(c4)oc4ccccc45)n3)c2)cc1 ; c1ccc(-c2ccc(-c3nc(-c4ccc(-c5ccccc5)cc4)nc(-c4ccc5c(c4)sc4c(-c6ccccc6)cccc45)n3)cc2)cc1 ; c1ccc(-c2ccc(-c3nc(-c4ccccc4)nc(-c4ccc5c(c4)sc4cc(-c6ccccc6)ccc45)n3)cc2)cc1 ; c1ccc(-c2ccc(-c3cc(-c4nc(-c5ccccc5)nc(-c5ccc(-c6ccccc6)cc5)n4)c4sc5ccccc5c4c3)cc2)cc1]

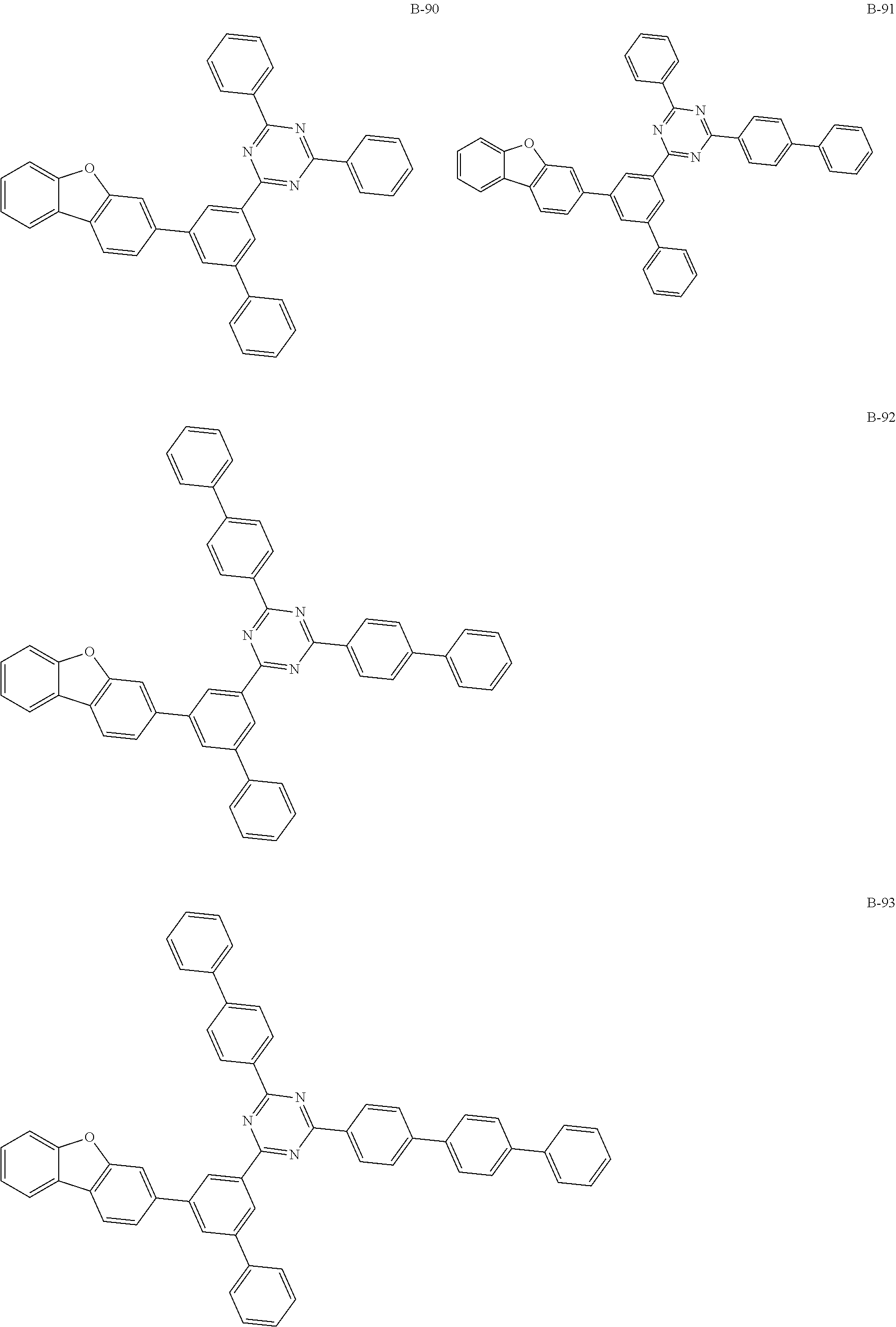

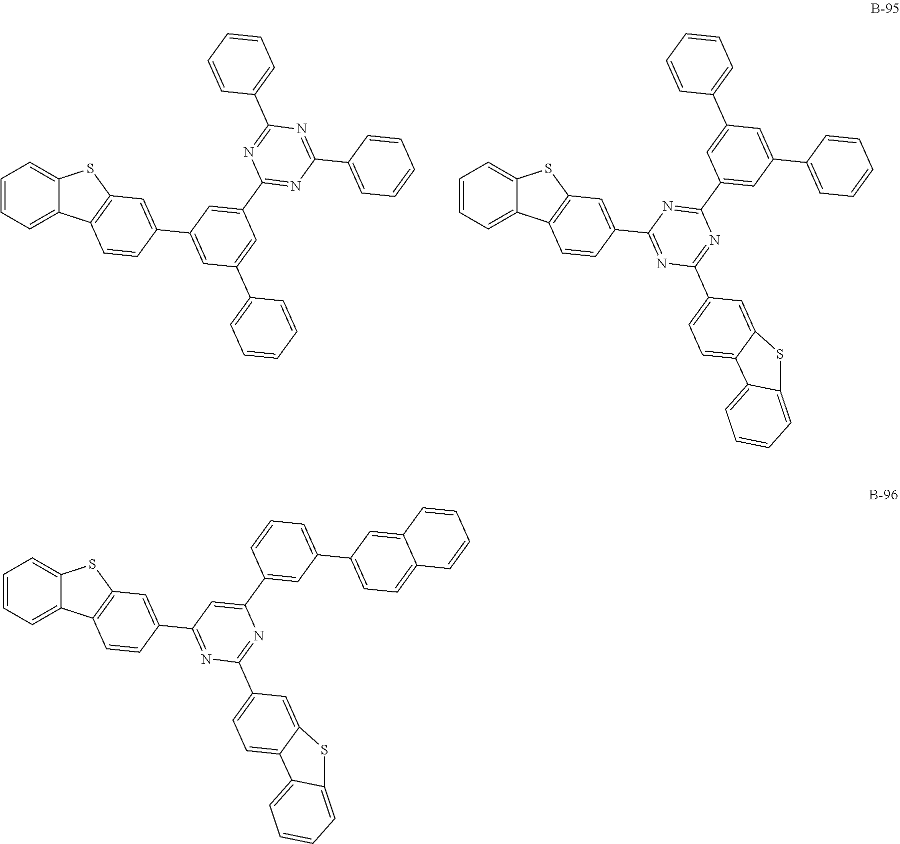

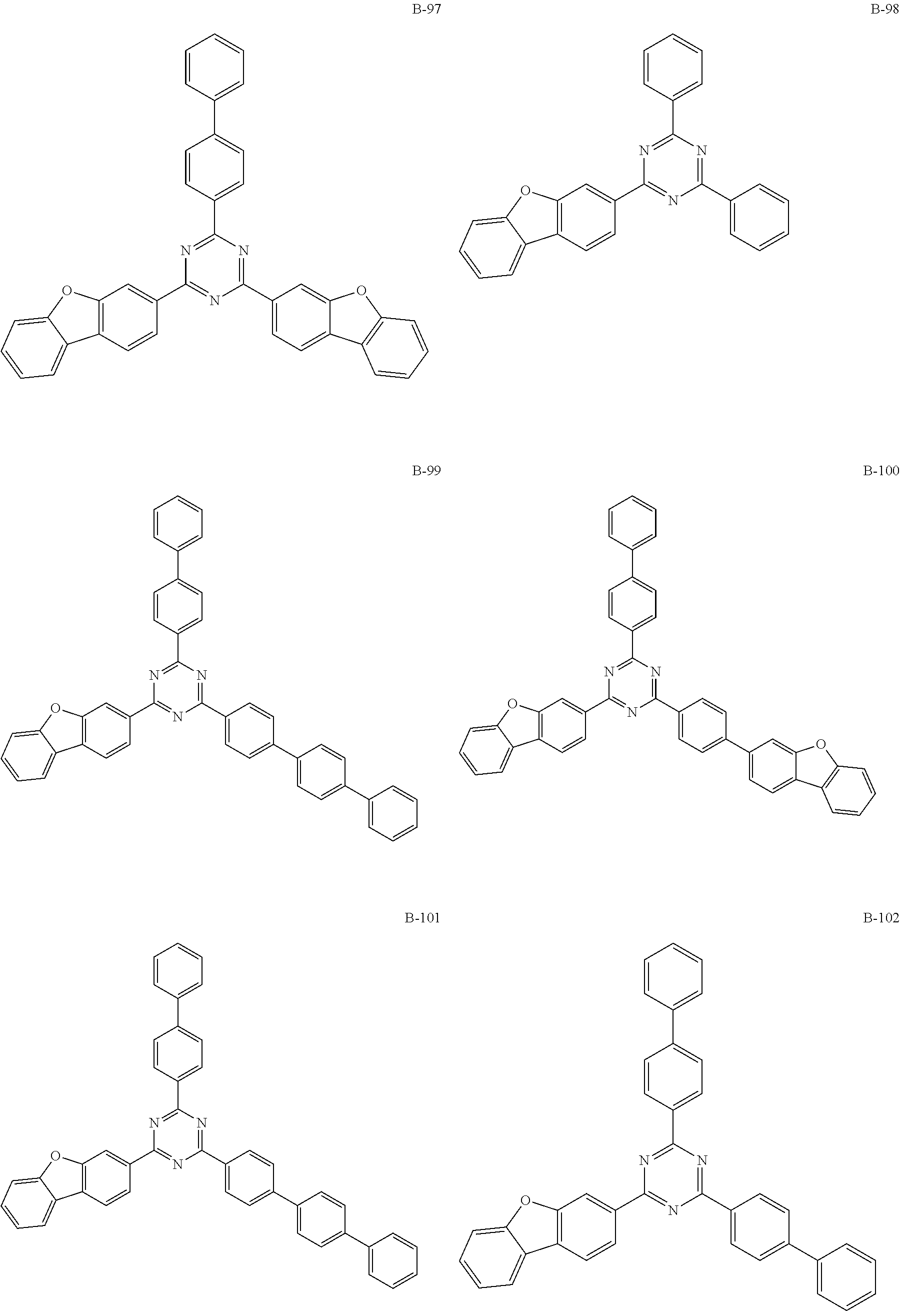 [SMILES: c1ccc(-c2ccc(-c3nc(-c4ccc5c(c4)oc4ccccc45)nc(-c4ccc5c(c4)oc4ccccc45)n3)cc2)cc1 ; c1ccc(-c2nc(-c3ccccc3)nc(-c3ccc4c(c3)oc3ccccc34)n2)cc1 ; c1ccc(-c2ccc(-c3ccc(-c4nc(-c5ccc(-c6ccccc6)cc5)nc(-c5ccc6c(c5)oc5ccccc56)n4)cc3)cc2)cc1 ; c1ccc(-c2ccc(-c3nc(-c4ccc(-c5ccc6c(c5)oc5ccccc56)cc4)nc(-c4ccc5c(c4)oc4ccccc45)n3)cc2)cc1 ; c1ccc(-c2ccc(-c3ccc(-c4nc(-c5ccc(-c6ccccc6)cc5)nc(-c5ccc6c(c5)oc5ccccc56)n4)cc3)cc2)cc1 ; c1ccc(-c2ccc(-c3nc(-c4ccc(-c5ccccc5)cc4)nc(-c4ccc5c(c4)oc4ccccc45)n3)cc2)cc1]

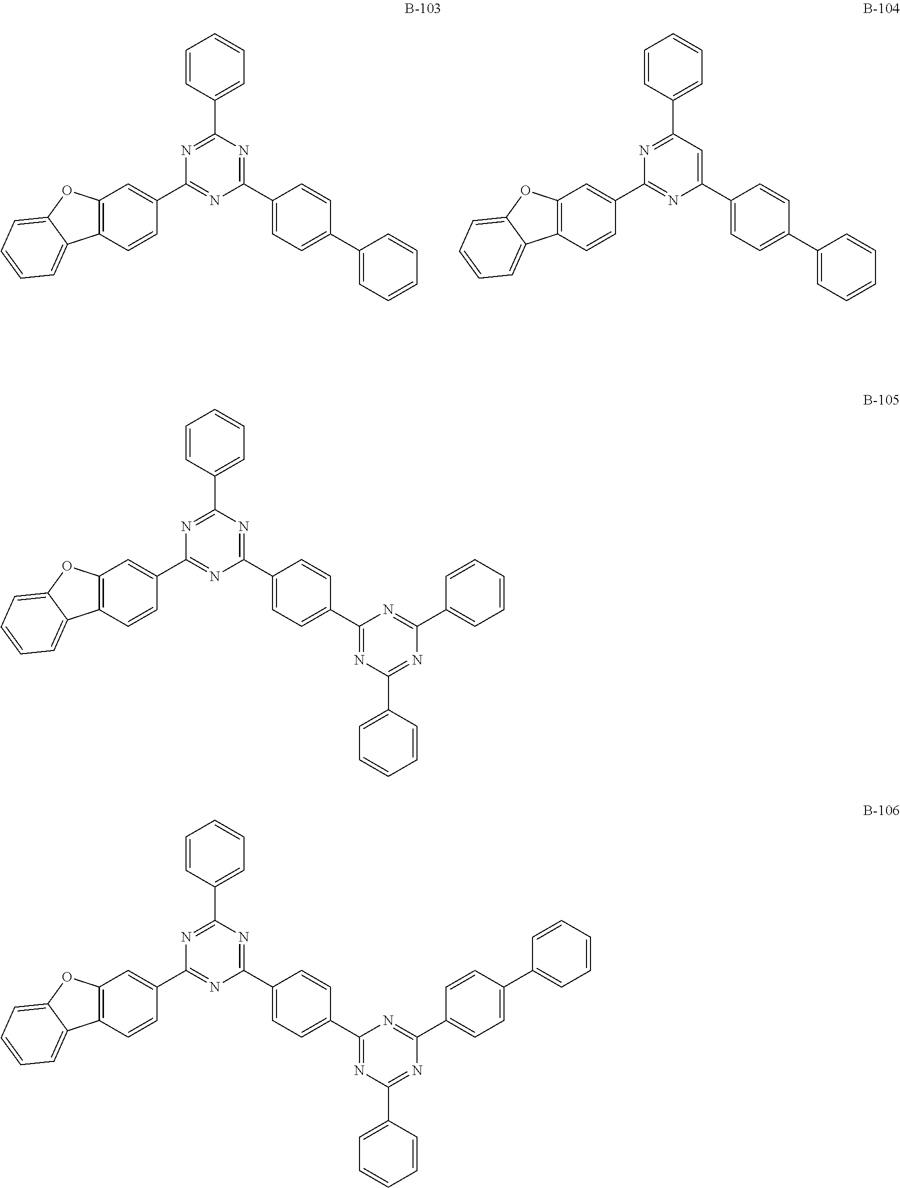

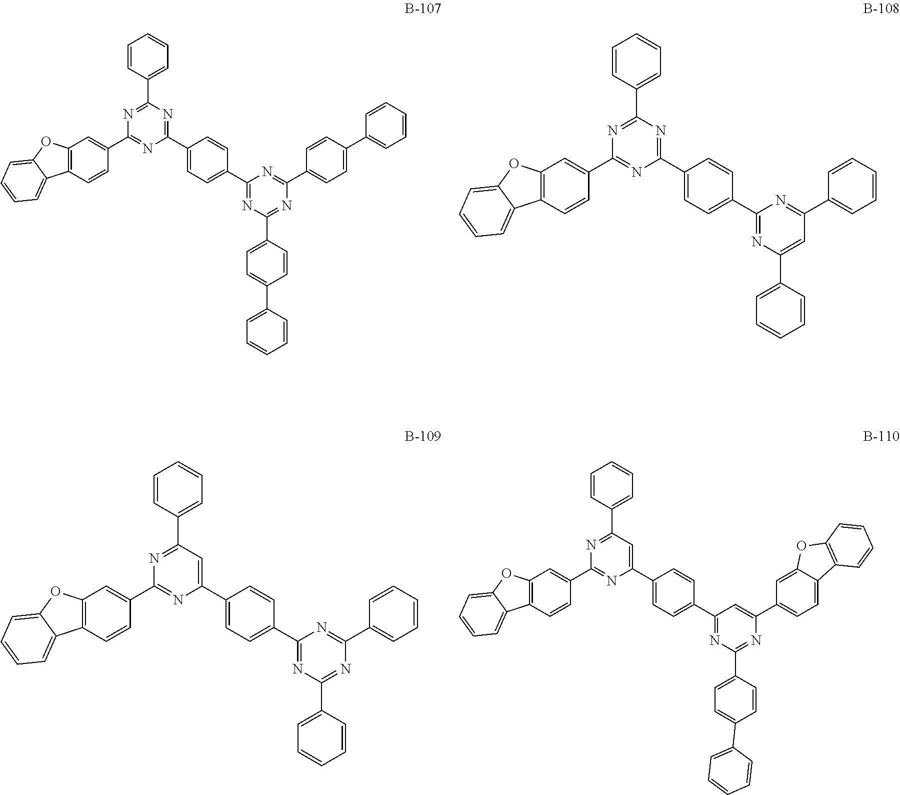 [SMILES: c1ccc(-c2ccc(-c3nc(-c4ccc(-c5ccccc5)cc4)nc(-c4ccc(-c5nc(-c6ccccc6)nc(-c6ccc7c(c6)oc6ccccc67)n5)cc4)n3)cc2)cc1 ; c1ccc(-c2cc(-c3ccccc3)nc(-c3ccc(-c4nc(-c5ccccc5)nc(-c5ccc6c(c5)oc5ccccc56)n4)cc3)n2)cc1 ; c1ccc(-c2cc(-c3ccc(-c4nc(-c5ccccc5)nc(-c5ccccc5)n4)cc3)nc(-c3ccc4c(c3)oc3ccccc34)n2)cc1 ; c1ccc(-c2ccc(-c3nc(-c4ccc(-c5cc(-c6ccccc6)nc(-c6ccc7c(c6)oc6ccccc67)n5)cc4)cc(-c4ccc5c(c4)oc4ccccc45)n3)cc2)cc1]

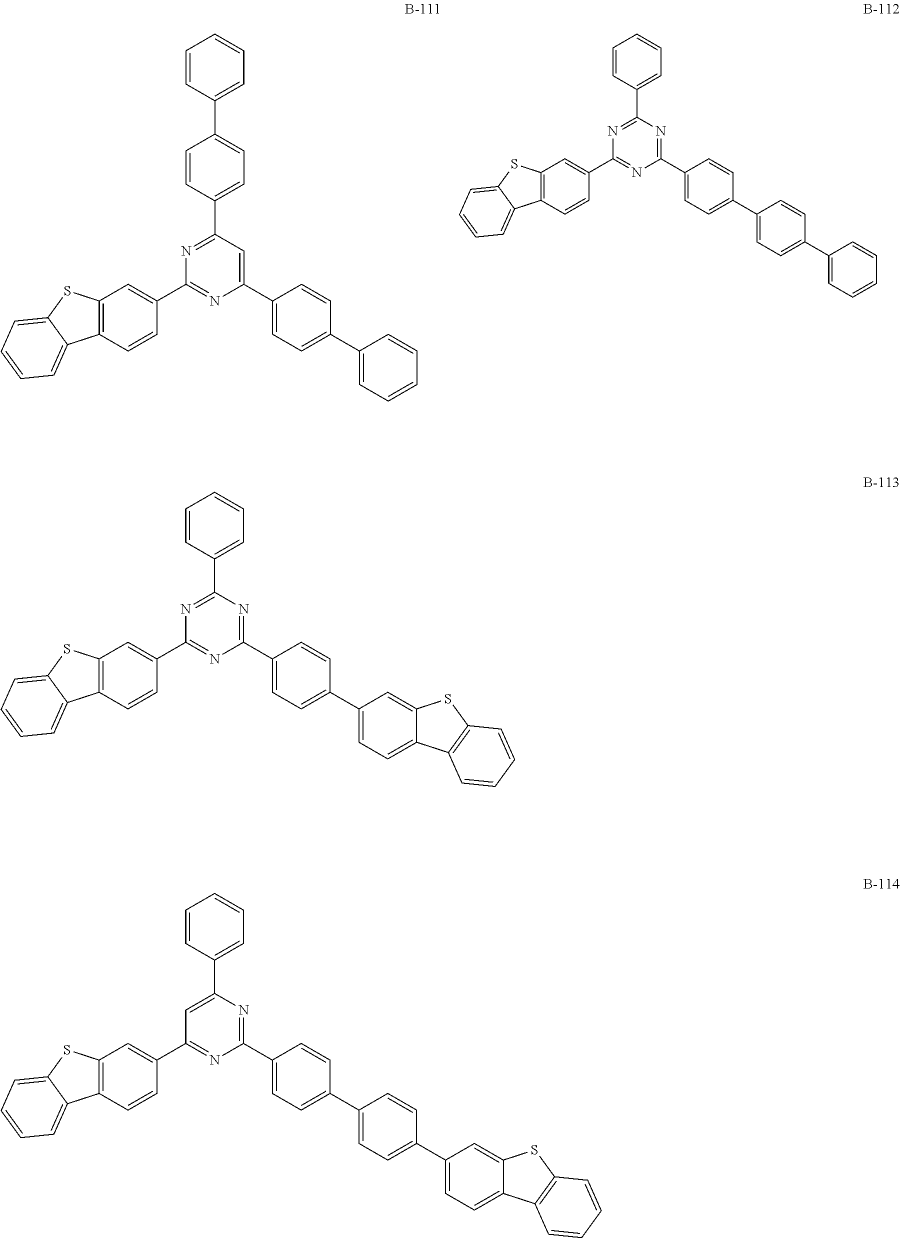 [SMILES: c1ccc(-c2ccc(-c3cc(-c4ccc(-c5ccccc5)cc4)nc(-c4ccc5c(c4)sc4ccccc45)n3)cc2)cc1 ; c1ccc(-c2ccc(-c3ccc(-c4nc(-c5ccccc5)nc(-c5ccc6c(c5)sc5ccccc56)n4)cc3)cc2)cc1 ; c1ccc(-c2nc(-c3ccc(-c4ccc5c(c4)sc4ccccc45)cc3)nc(-c3ccc4c(c3)sc3ccccc34)n2)cc1 ; c1ccc(-c2cc(-c3ccc4c(c3)sc3ccccc34)nc(-c3ccc(-c4ccc(-c5ccc6c(c5)sc5ccccc56)cc4)cc3)n2)cc1]

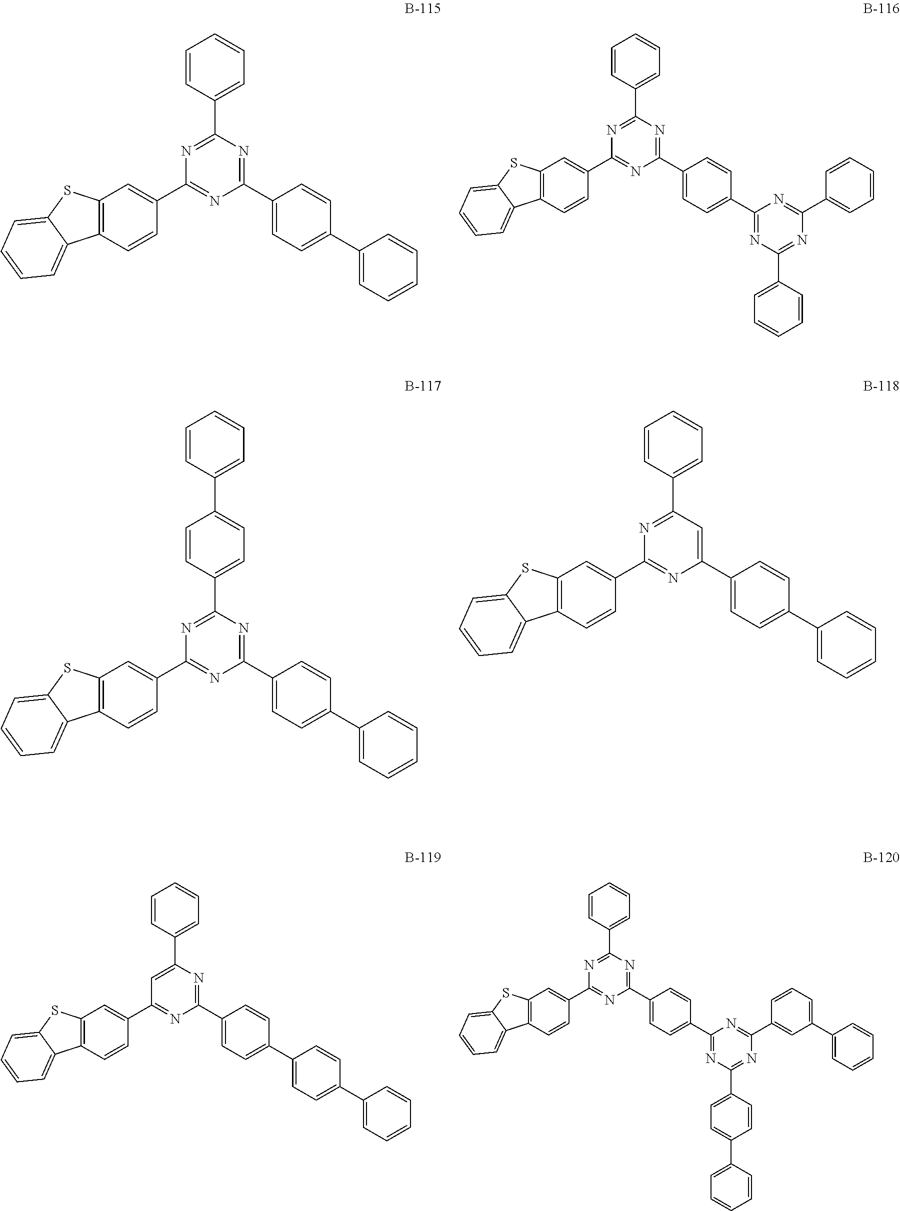 [SMILES: c1ccc(-c2ccc(-c3nc(-c4ccccc4)nc(-c4ccc5c(c4)sc4ccccc45)n3)cc2)cc1 ; c1ccc(-c2nc(-c3ccccc3)nc(-c3ccc(-c4nc(-c5ccccc5)nc(-c5ccc6c(c5)sc5ccccc56)n4)cc3)n2)cc1 ; c1ccc(-c2ccc(-c3nc(-c4ccc(-c5ccccc5)cc4)nc(-c4ccc5c(c4)sc4ccccc45)n3)cc2)cc1 ; c1ccc(-c2ccc(-c3cc(-c4ccccc4)nc(-c4ccc5c(c4)sc4ccccc45)n3)cc2)cc1 ; c1ccc(-c2ccc(-c3ccc(-c4nc(-c5ccccc5)cc(-c5ccc6c(c5)sc5ccccc56)n4)cc3)cc2)cc1 ; c1ccc(-c2ccc(-c3nc(-c4ccc(-c5nc(-c6ccccc6)nc(-c6ccc7c(c6)sc6ccccc67)n5)cc4)nc(-c4cccc(-c5ccccc5)c4)n3)cc2)cc1]

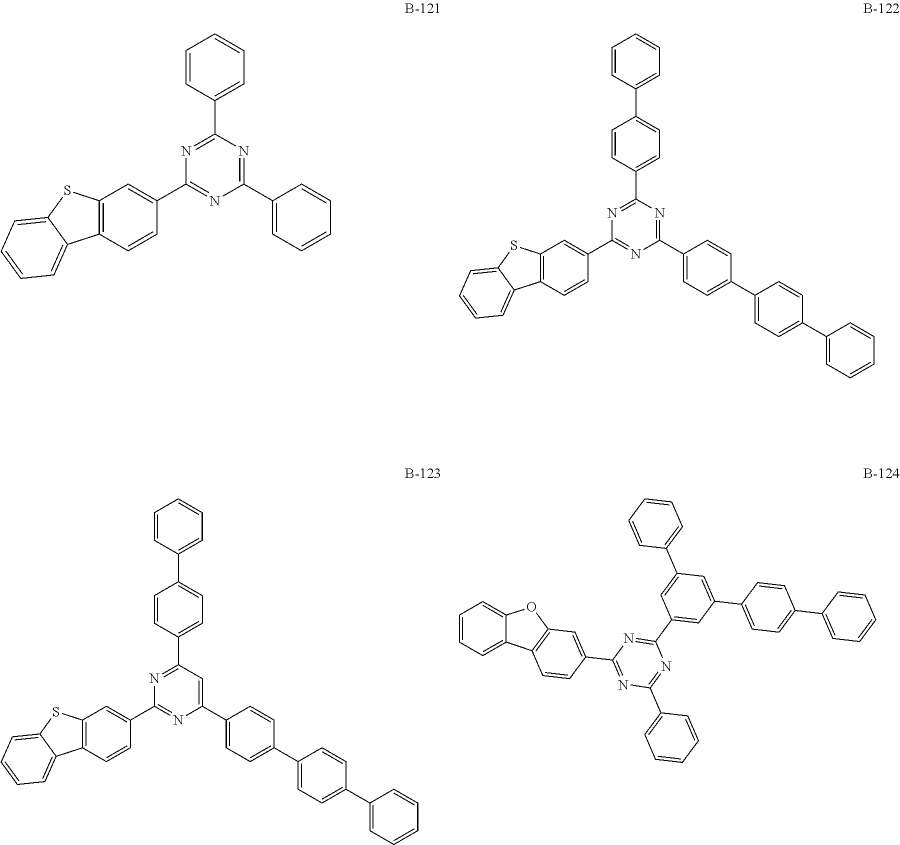 [SMILES: c1ccc(-c2nc(-c3ccccc3)nc(-c3ccc4c(c3)sc3ccccc34)n2)cc1 ; c1ccc(-c2ccc(-c3ccc(-c4nc(-c5ccc(-c6ccccc6)cc5)nc(-c5ccc6c(c5)sc5ccccc56)n4)cc3)cc2)cc1 ; c1ccc(-c2ccc(-c3ccc(-c4cc(-c5ccc(-c6ccccc6)cc5)nc(-c5ccc6c(c5)sc5ccccc56)n4)cc3)cc2)cc1 ; c1ccc(-c2ccc(-c3cc(-c4ccccc4)cc(-c4nc(-c5ccccc5)nc(-c5ccc6c(c5)oc5ccccc56)n4)c3)cc2)cc1]

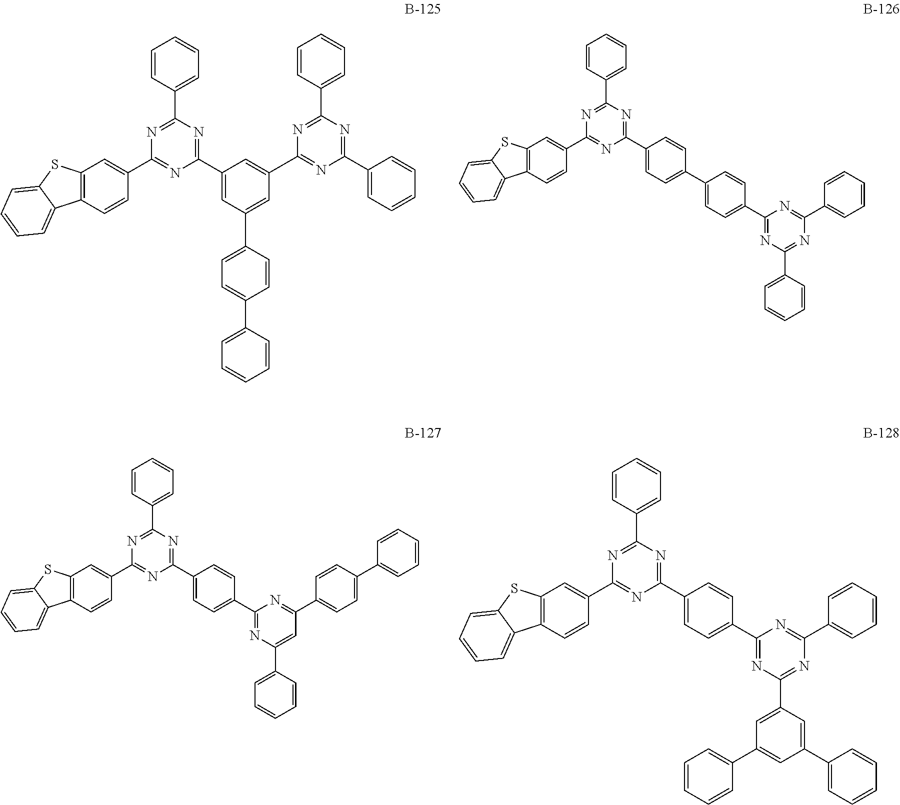 [SMILES: c1ccc(-c2ccc(-c3cc(-c4nc(-c5ccccc5)nc(-c5ccccc5)n4)cc(-c4nc(-c5ccccc5)nc(-c5ccc6c(c5)sc5ccccc56)n4)c3)cc2)cc1 ; c1ccc(-c2nc(-c3ccccc3)nc(-c3ccc(-c4ccc(-c5nc(-c6ccccc6)nc(-c6ccc7c(c6)sc6ccccc67)n5)cc4)cc3)n2)cc1 ; c1ccc(-c2ccc(-c3cc(-c4ccccc4)nc(-c4ccc(-c5nc(-c6ccccc6)nc(-c6ccc7c(c6)sc6ccccc67)n5)cc4)n3)cc2)cc1 ; c1ccc(-c2cc(-c3ccccc3)cc(-c3nc(-c4ccccc4)nc(-c4ccc(-c5nc(-c6ccccc6)nc(-c6ccc7c(c6)sc6ccccc67)n5)cc4)n3)c2)cc1]

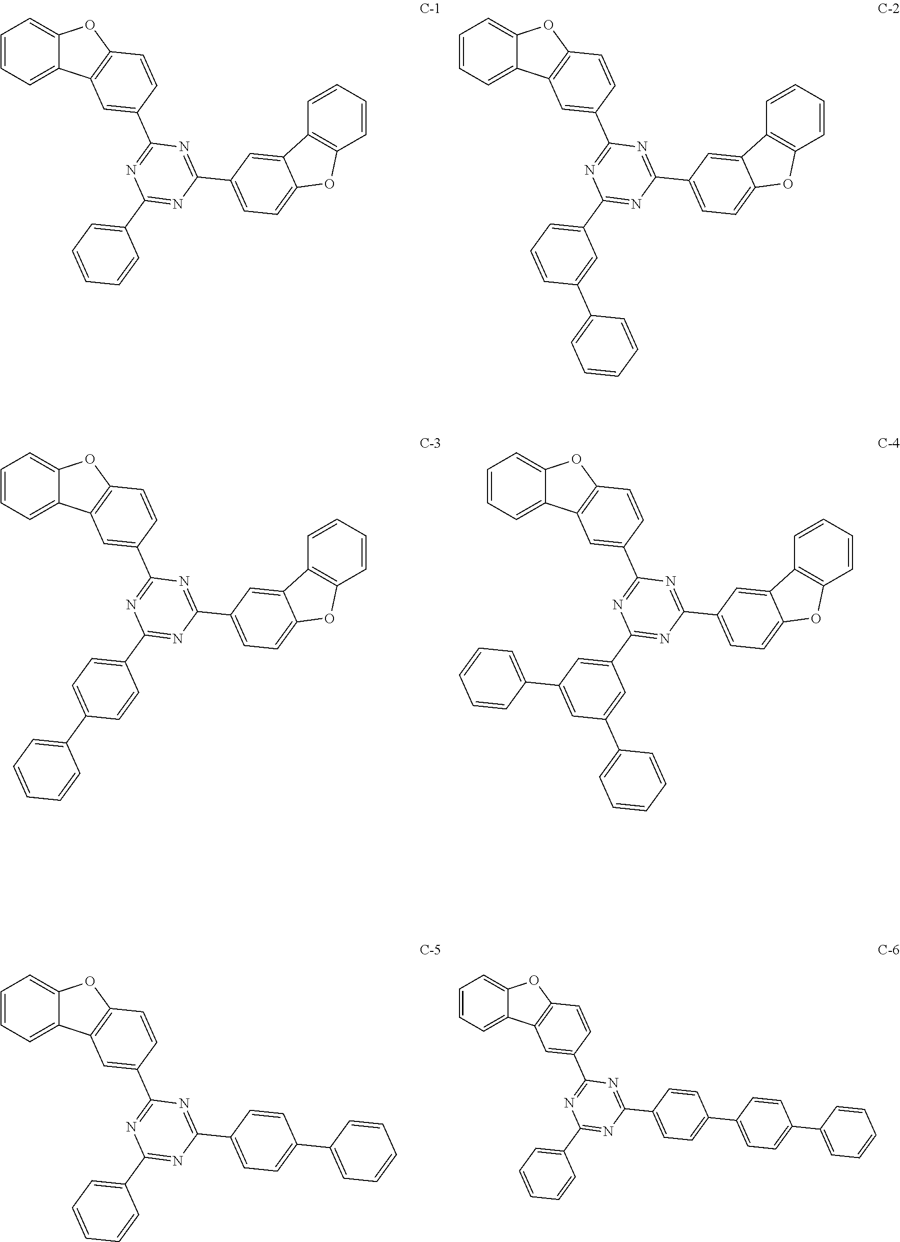 [SMILES: c1ccc(-c2nc(-c3ccc4oc5ccccc5c4c3)nc(-c3ccc4oc5ccccc5c4c3)n2)cc1 ; c1ccc(-c2cccc(-c3nc(-c4ccc5oc6ccccc6c5c4)nc(-c4ccc5oc6ccccc6c5c4)n3)c2)cc1 ; c1ccc(-c2ccc(-c3nc(-c4ccc5oc6ccccc6c5c4)nc(-c4ccc5oc6ccccc6c5c4)n3)cc2)cc1 ; c1ccc(-c2cc(-c3ccccc3)cc(-c3nc(-c4ccc5oc6ccccc6c5c4)nc(-c4ccc5oc6ccccc6c5c4)n3)c2)cc1 ; c1ccc(-c2ccc(-c3nc(-c4ccccc4)nc(-c4ccc5oc6ccccc6c5c4)n3)cc2)cc1 ; c1ccc(-c2ccc(-c3ccc(-c4nc(-c5ccccc5)nc(-c5ccc6oc7ccccc7c6c5)n4)cc3)cc2)cc1]

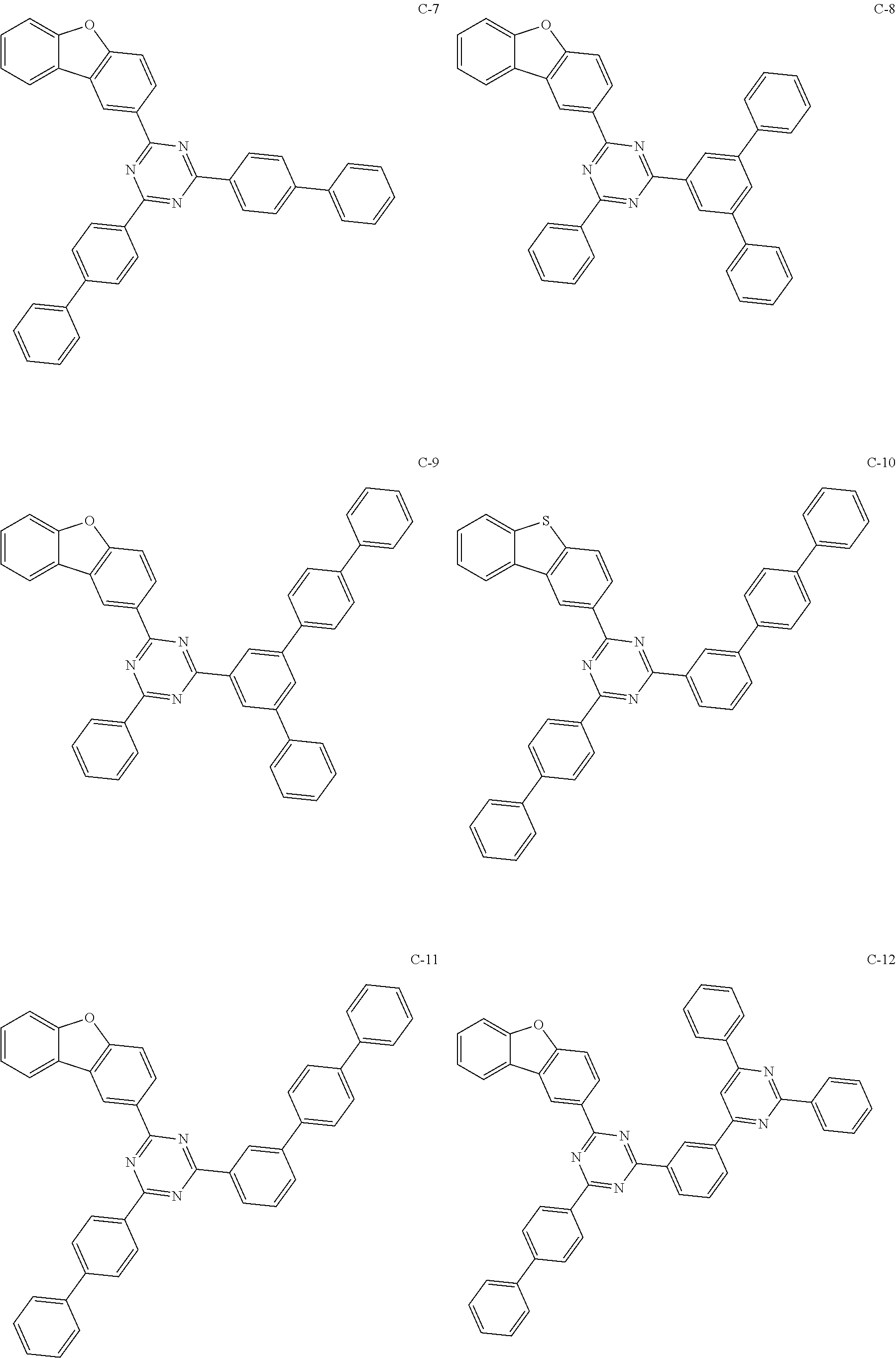

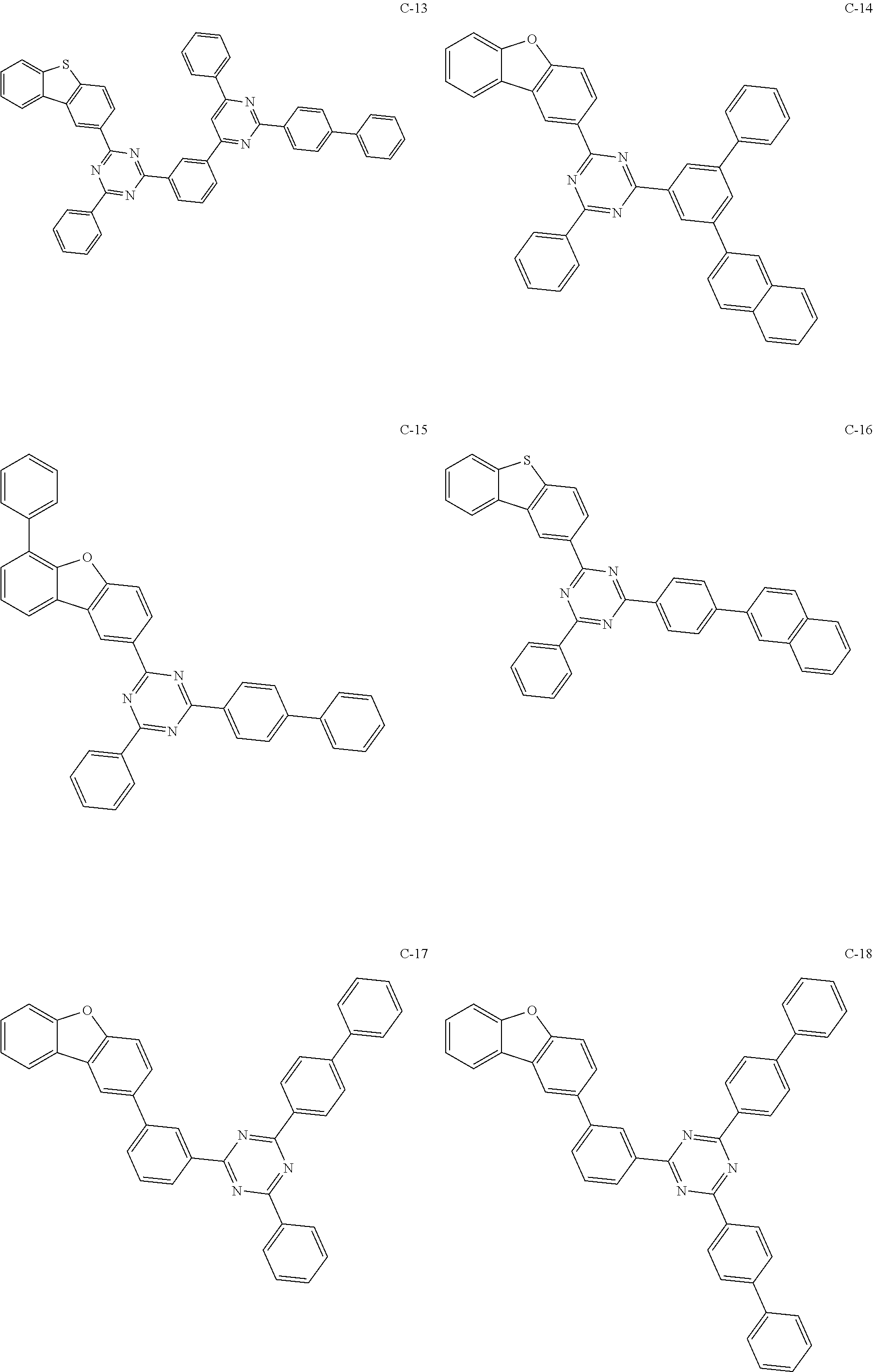

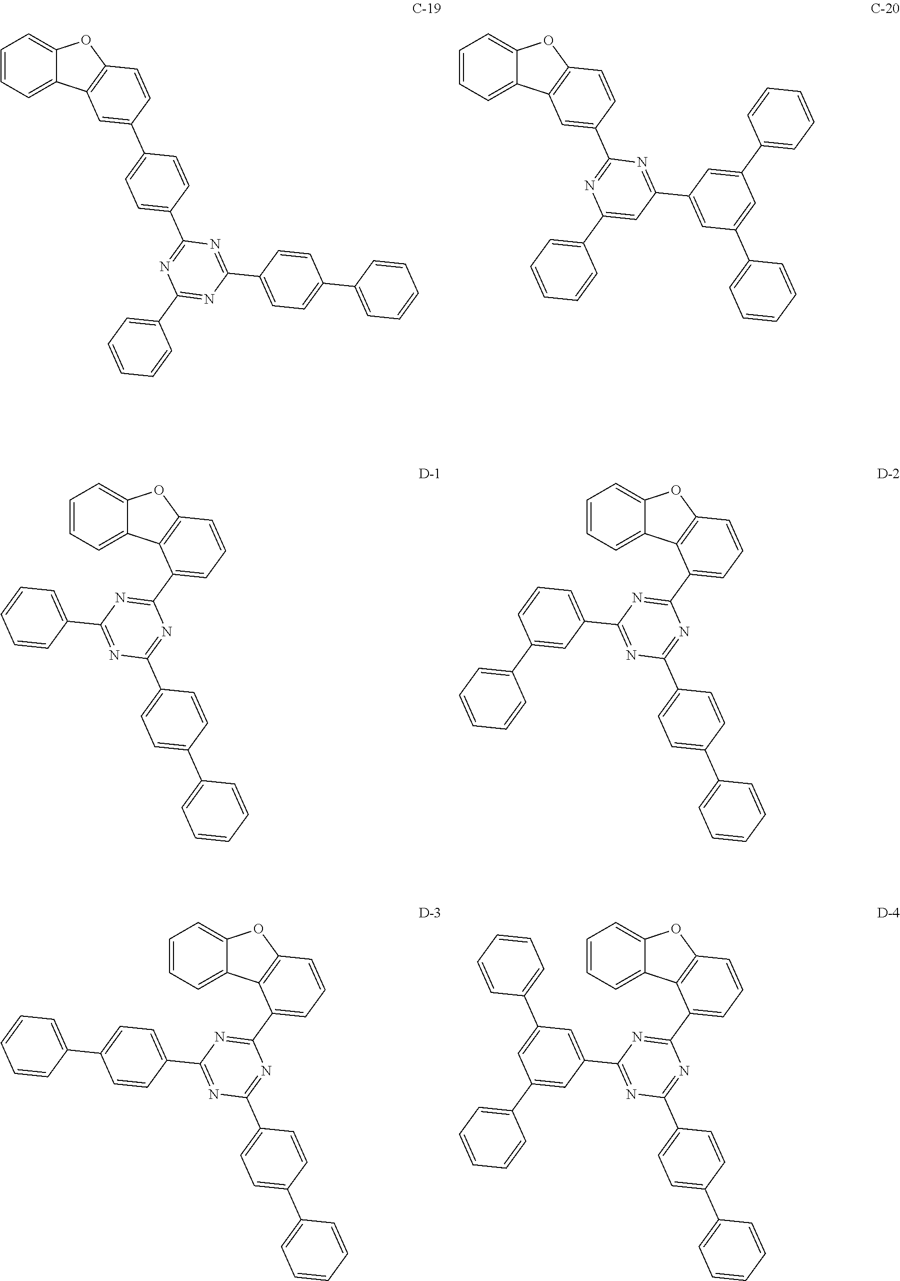 [SMILES: c1ccc(-c2ccc(-c3nc(-c4ccccc4)nc(-c4ccc(-c5ccc6oc7ccccc7c6c5)cc4)n3)cc2)cc1 ; c1ccc(-c2cc(-c3ccccc3)cc(-c3cc(-c4ccccc4)nc(-c4ccc5oc6ccccc6c5c4)n3)c2)cc1 ; c1ccc(-c2ccc(-c3nc(-c4ccccc4)nc(-c4cccc5oc6ccccc6c45)n3)cc2)cc1 ; c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cc(-c5ccccc5)cc(-c5ccccc5)c4)nc(-c4cccc5oc6ccccc6c45)n3)cc2)cc1]

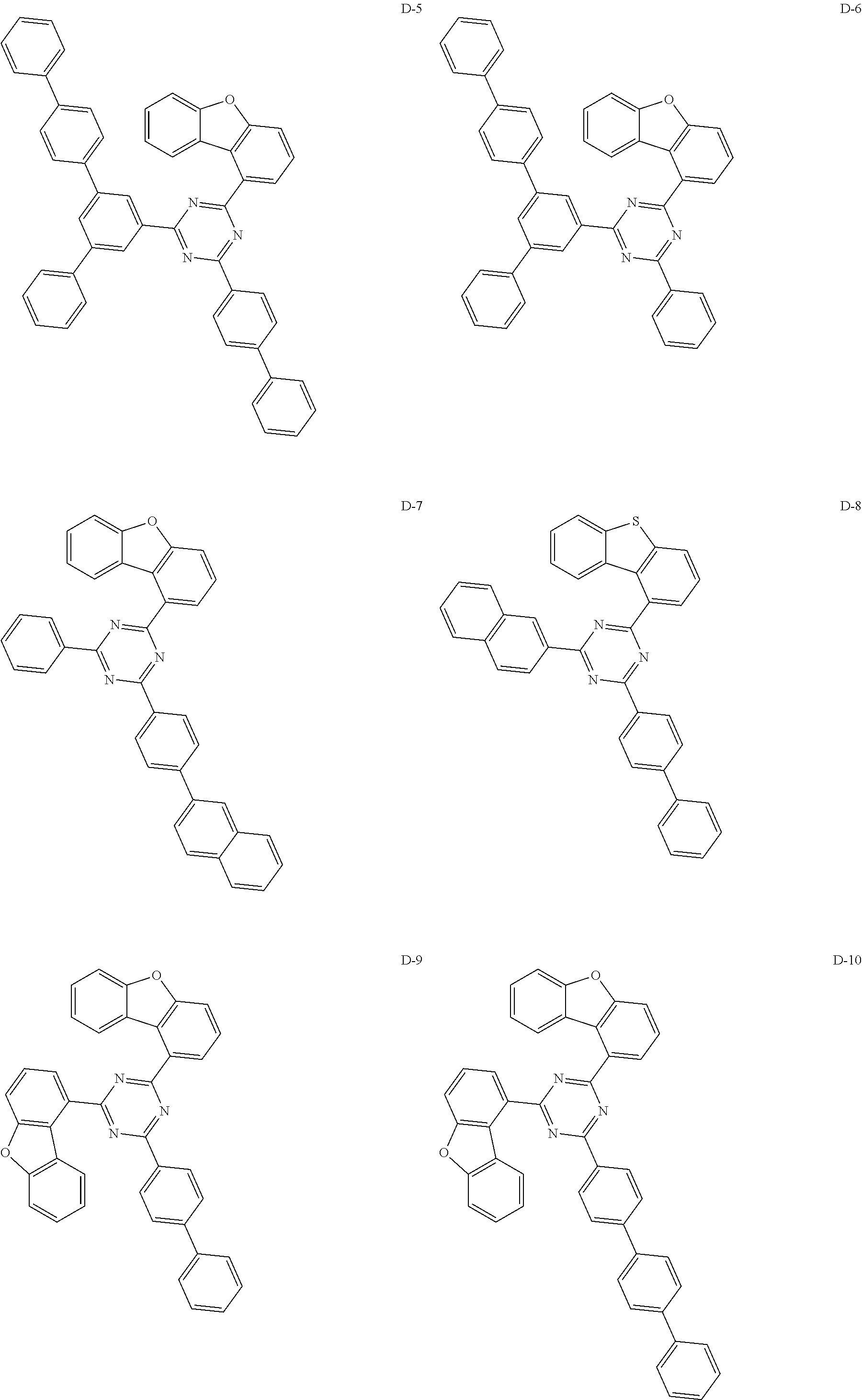 [SMILES: c1ccc(-c2ccc(-c3cc(-c4ccccc4)cc(-c4nc(-c5ccc(-c6ccccc6)cc5)nc(-c5cccc6oc7ccccc7c56)n4)c3)cc2)cc1 ; c1ccc(-c2ccc(-c3cc(-c4ccccc4)cc(-c4nc(-c5ccccc5)nc(-c5cccc6oc7ccccc7c56)n4)c3)cc2)cc1 ; c1ccc(-c2nc(-c3ccc(-c4ccc5ccccc5c4)cc3)nc(-c3cccc4oc5ccccc5c34)n2)cc1 ; c1ccc(-c2ccc(-c3nc(-c4ccc5ccccc5c4)nc(-c4cccc5sc6ccccc6c45)n3)cc2)cc1 ; c1ccc(-c2ccc(-c3nc(-c4cccc5oc6ccccc6c45)nc(-c4cccc5oc6ccccc6c45)n3)cc2)cc1 ; c1ccc(-c2ccc(-c3ccc(-c4nc(-c5cccc6oc7ccccc7c56)nc(-c5cccc6oc7ccccc7c56)n4)cc3)cc2)cc1]

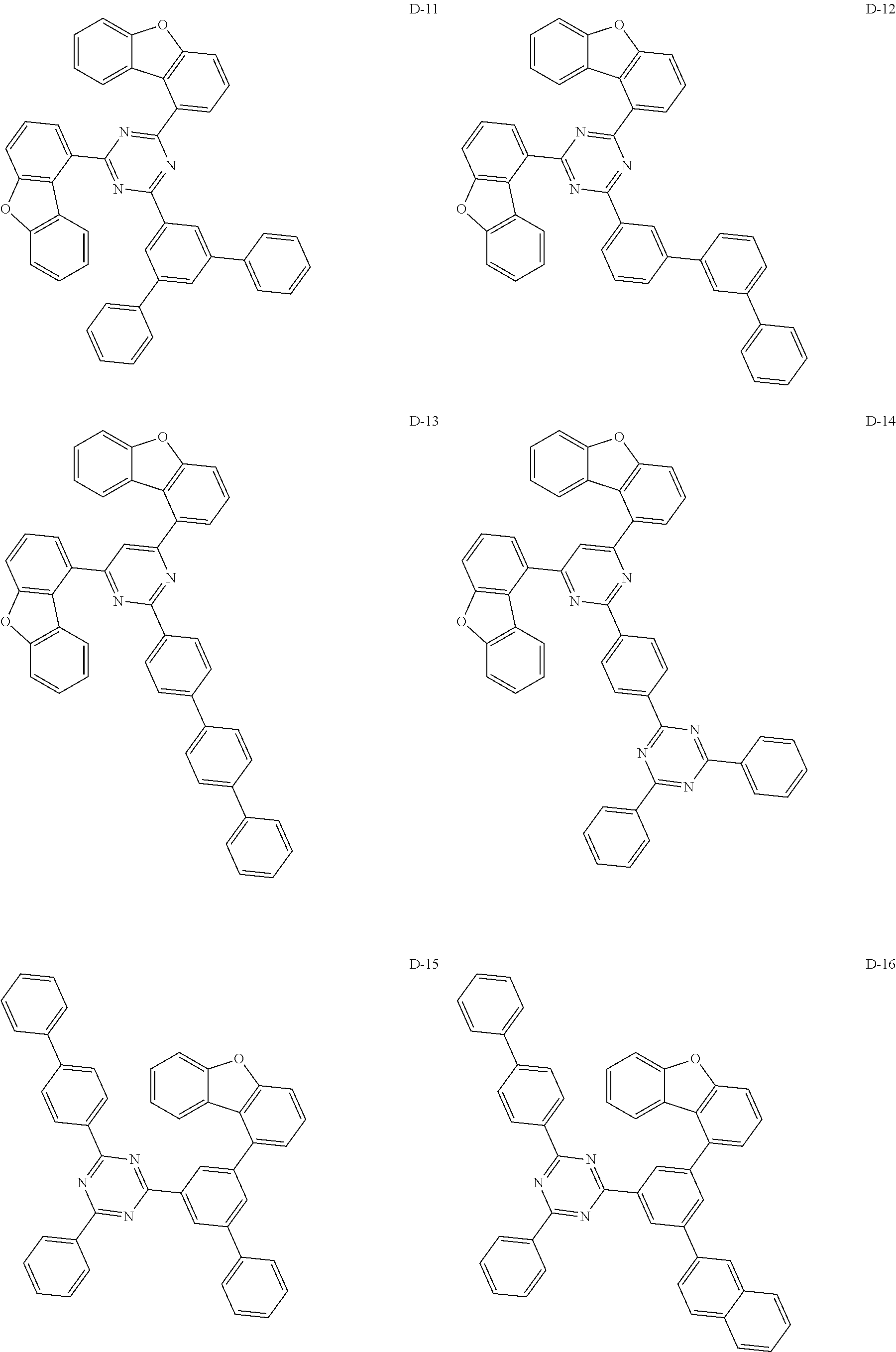

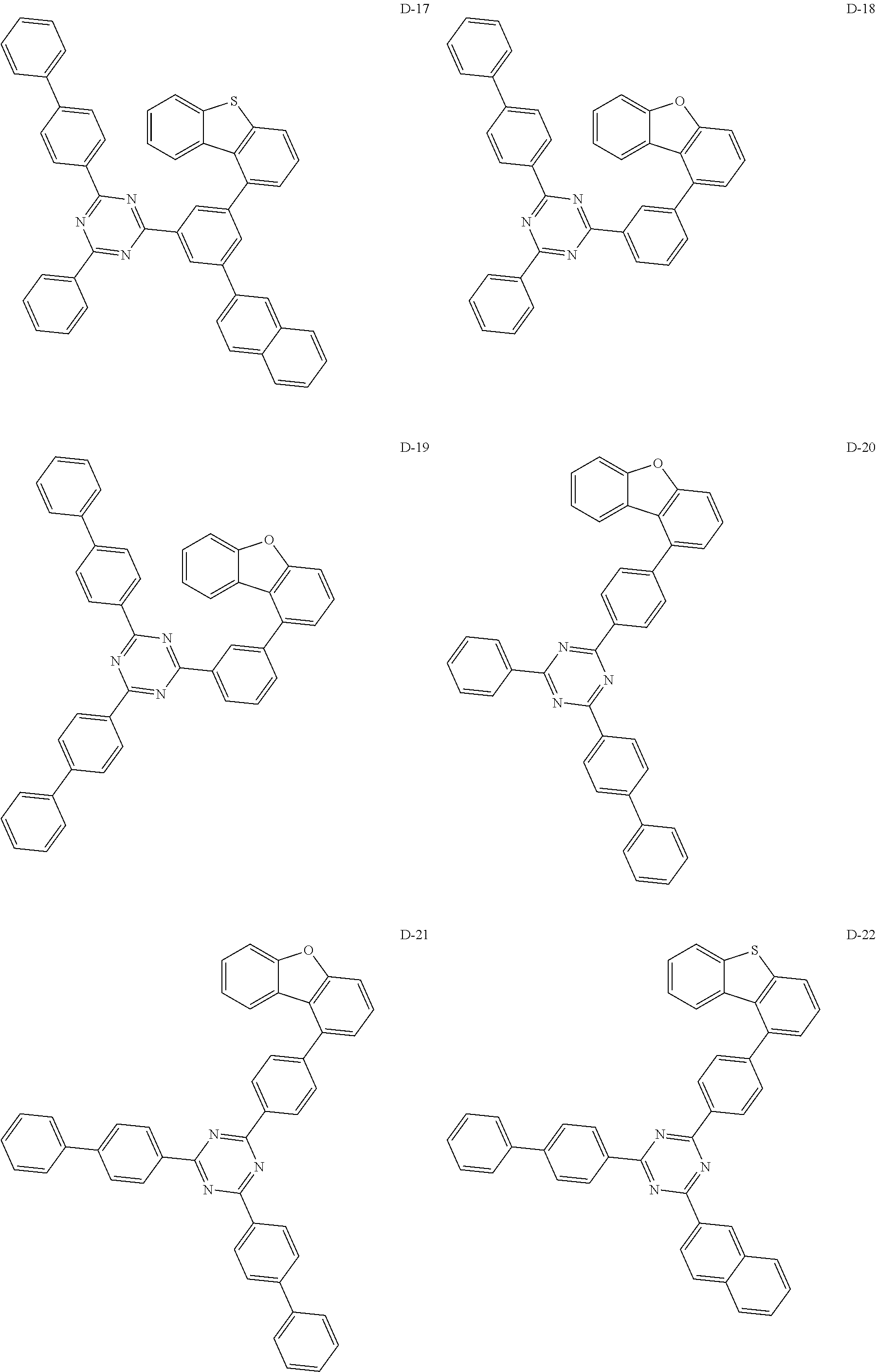

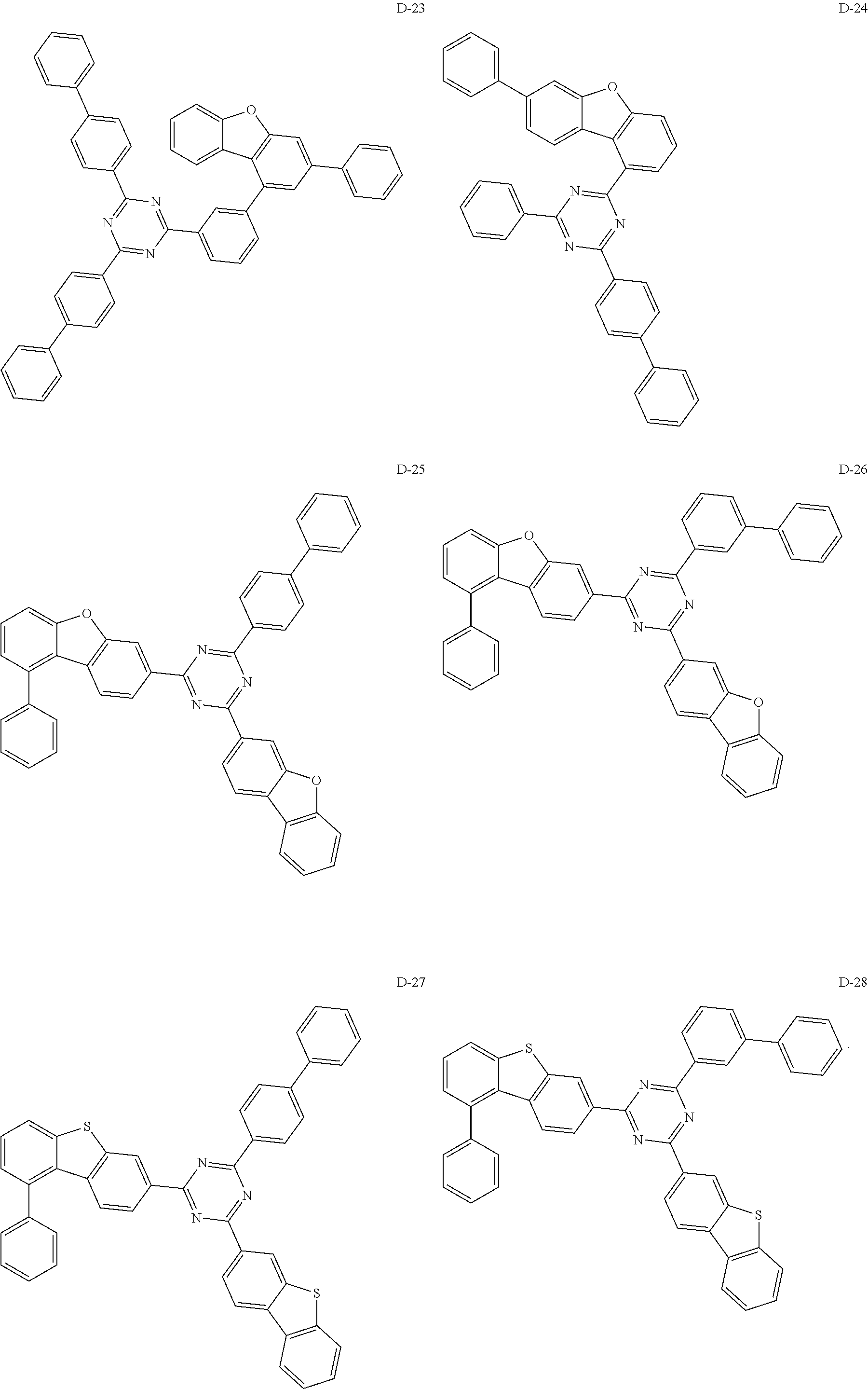

Ar⁴ to Ar⁶ of Chemical Formula 2 and Chemical Formula 3 may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a combination thereof.

The second compound may be one of the compounds listed in Group 2:

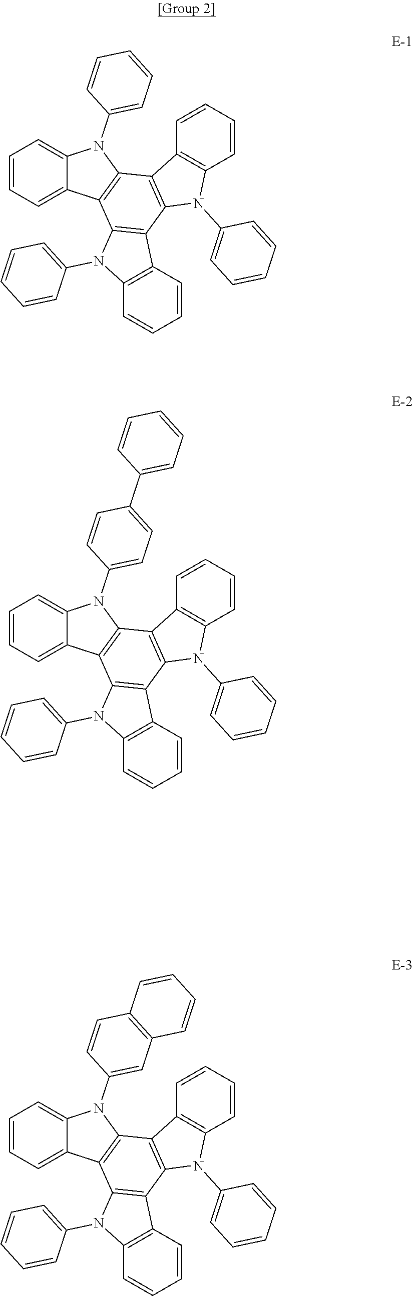 [SMILES: c1ccc(-n2c3ccccc3c3c2c2c4ccccc4n(-c4ccccc4)c2c2c4ccccc4n(-c4ccccc4)c32)cc1 ; c1ccc(-c2ccc(-n3c4ccccc4c4c5c(c6ccccc6n5-c5ccccc5)c5c(c6ccccc6n5-c5ccccc5)c43)cc2)cc1 ; c1ccc(-n2c3ccccc3c3c2c2c4ccccc4n(-c4ccc5ccccc5c4)c2c2c4ccccc4n(-c4ccccc4)c32)cc1]

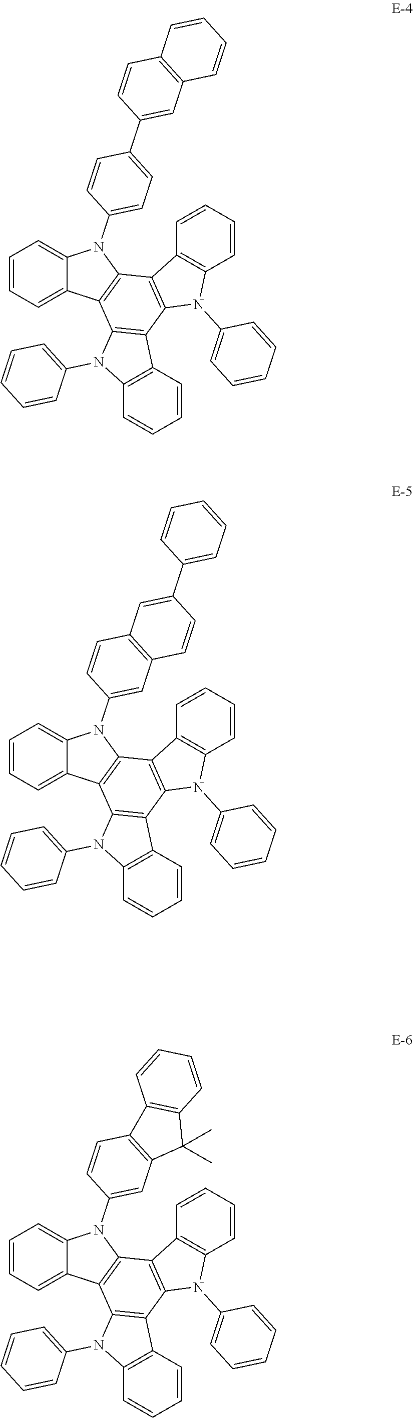 [SMILES: c1ccc(-n2c3ccccc3c3c2c2c4ccccc4n(-c4ccc(-c5ccc6ccccc6c5)cc4)c2c2c4ccccc4n(-c4ccccc4)c32)cc1 ; c1ccc(-c2ccc3cc(-n4c5ccccc5c5c6c(c7ccccc7n6-c6ccccc6)c6c(c7ccccc7n6-c6ccccc6)c54)ccc3c2)cc1 ; CC1(C)c2ccccc2-c2ccc(-n3c4ccccc4c4c5c(c6ccccc6n5-c5ccccc5)c5c(c6ccccc6n5-c5ccccc5)c43)cc21]

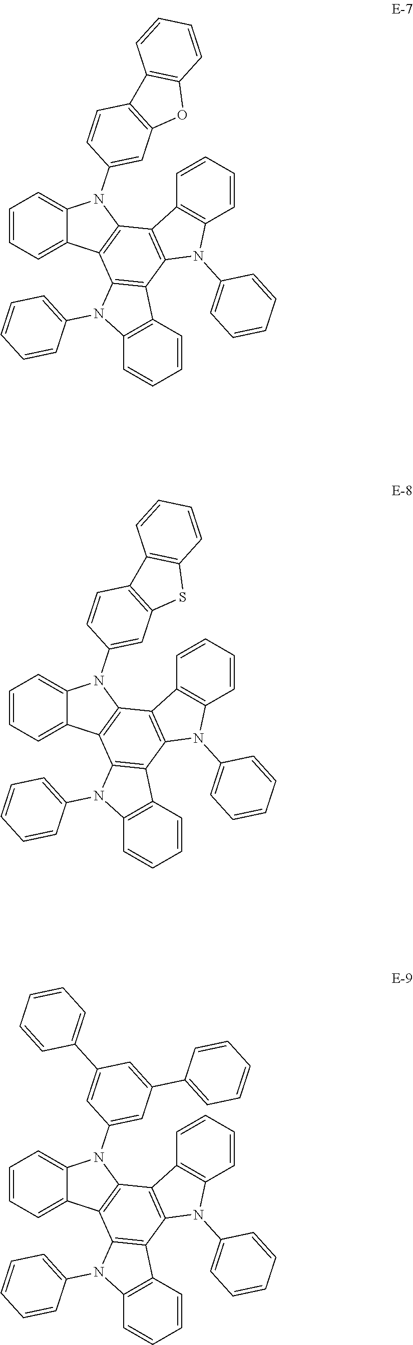 [SMILES: c1ccc(-n2c3ccccc3c3c2c2c4ccccc4n(-c4ccc5c(c4)oc4ccccc45)c2c2c4ccccc4n(-c4ccccc4)c32)cc1 ; c1ccc(-n2c3ccccc3c3c2c2c4ccccc4n(-c4ccc5c(c4)sc4ccccc45)c2c2c4ccccc4n(-c4ccccc4)c32)cc1 ; c1ccc(-c2cc(-c3ccccc3)cc(-n3c4ccccc4c4c5c(c6ccccc6n5-c5ccccc5)c5c(c6ccccc6n5-c5ccccc5)c43)c2)cc1]

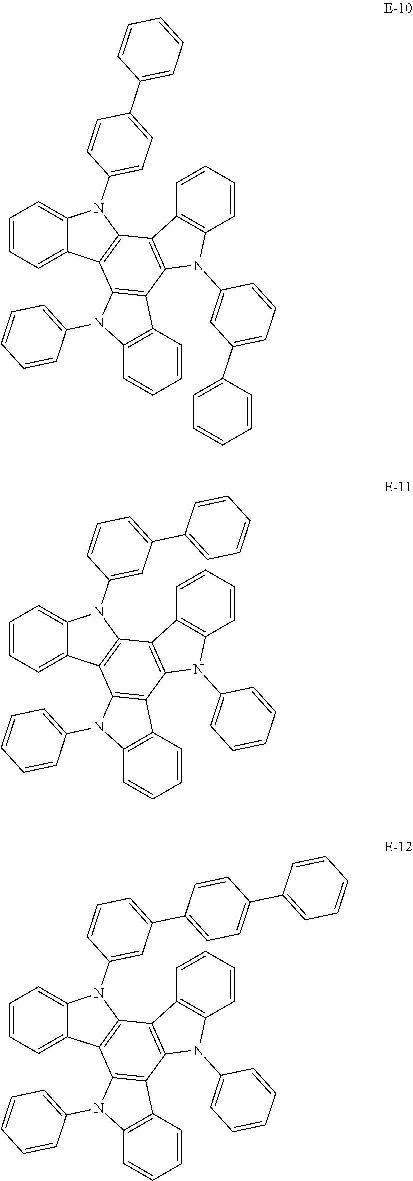 [SMILES: c1ccc(-c2ccc(-n3c4ccccc4c4c3c3c5ccccc5n(-c5cccc(-c6ccccc6)c5)c3c3c5ccccc5n(-c5ccccc5)c43)cc2)cc1 ; c1ccc(-c2cccc(-n3c4ccccc4c4c5c(c6ccccc6n5-c5ccccc5)c5c(c6ccccc6n5-c5ccccc5)c43)c2)cc1 ; c1ccc(-c2ccc(-c3cccc(-n4c5ccccc5c5c6c(c7ccccc7n6-c6ccccc6)c6c(c7ccccc7n6-c6ccccc6)c54)c3)cc2)cc1]

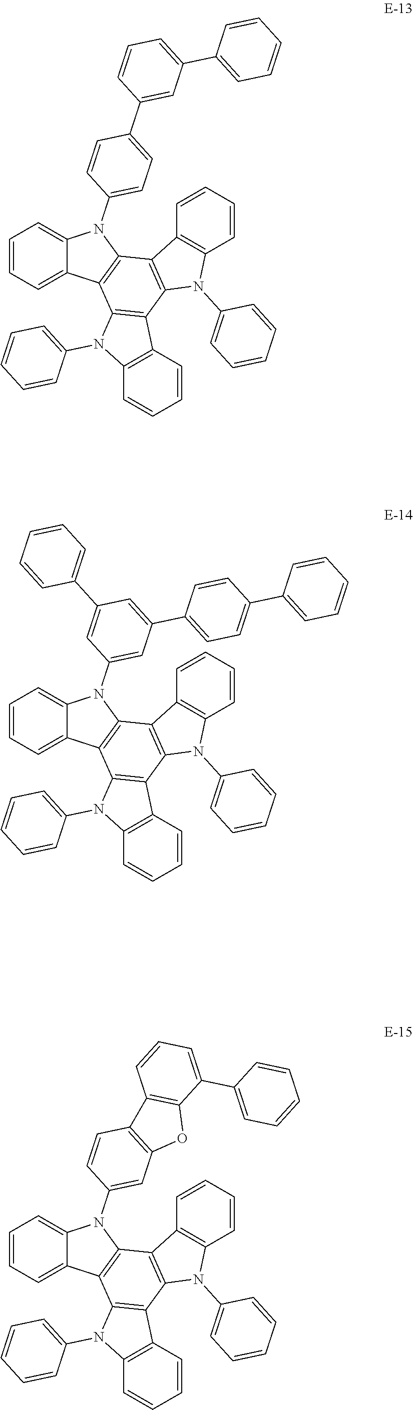 [SMILES: c1ccc(-c2cccc(-c3ccc(-n4c5ccccc5c5c6c(c7ccccc7n6-c6ccccc6)c6c(c7ccccc7n6-c6ccccc6)c54)cc3)c2)cc1 ; c1ccc(-c2ccc(-c3cc(-c4ccccc4)cc(-n4c5ccccc5c5c6c(c7ccccc7n6-c6ccccc6)c6c(c7ccccc7n6-c6ccccc6)c54)c3)cc2)cc1 ; c1ccc(-c2cccc3c2oc2cc(-n4c5ccccc5c5c6c(c7ccccc7n6-c6ccccc6)c6c(c7ccccc7n6-c6ccccc6)c54)ccc23)cc1]

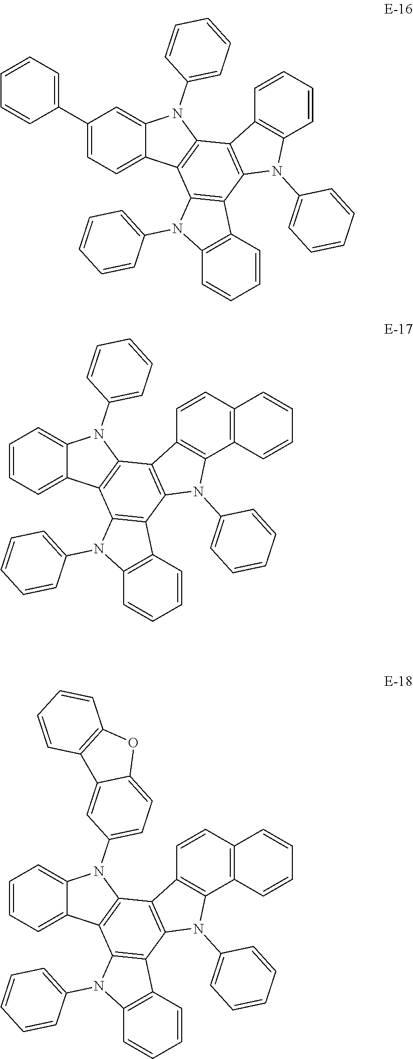 [SMILES: c1ccc(-c2ccc3c4c5c(c6ccccc6n5-c5ccccc5)c5c(c6ccccc6n5-c5ccccc5)c4n(-c4ccccc4)c3c2)cc1 ; c1ccc(-n2c3ccccc3c3c2c2c4ccc5ccccc5c4n(-c4ccccc4)c2c2c4ccccc4n(-c4ccccc4)c32)cc1 ; c1ccc(-n2c3ccccc3c3c2c2c4ccccc4n(-c4ccc5oc6ccccc6c5c4)c2c2c4ccc5ccccc5c4n(-c4ccccc4)c23)cc1]

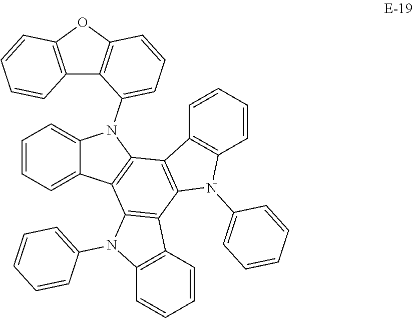 [SMILES: c1ccc(-n2c3ccccc3c3c2c2c4ccccc4n(-c4cccc5oc6ccccc6c45)c2c2c4ccccc4n(-c4ccccc4)c32)cc1]

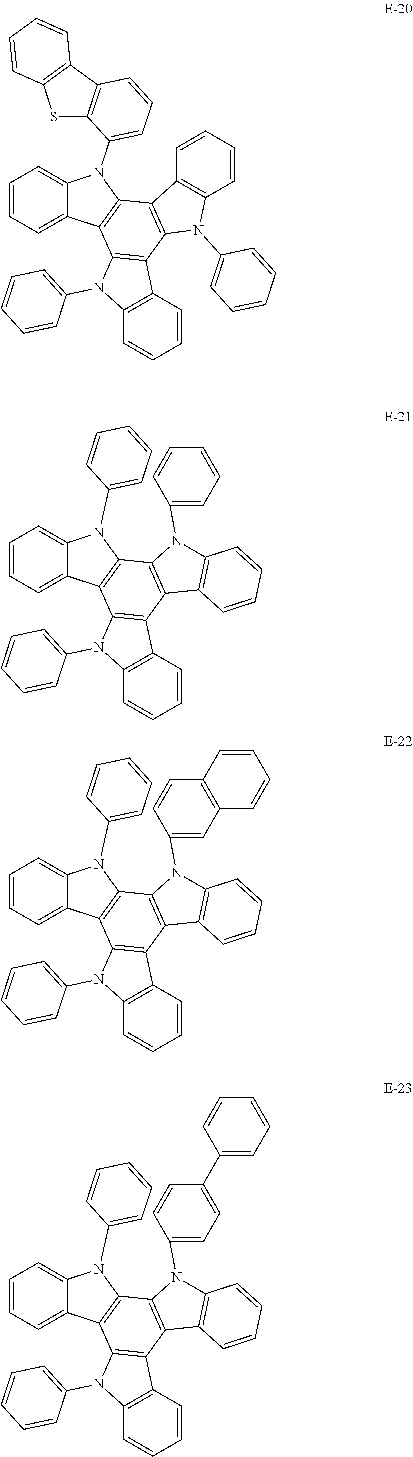

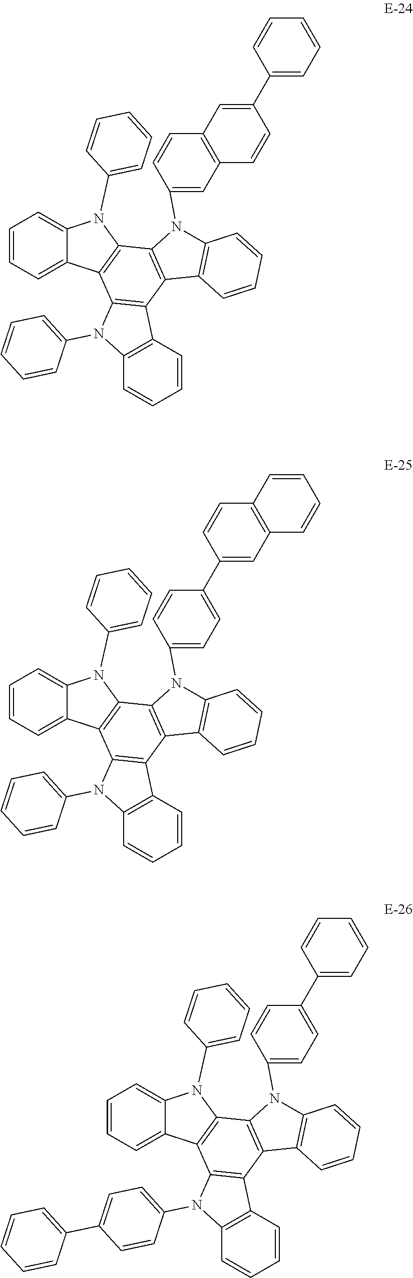 [SMILES: c1ccc(-c2ccc3cc(-n4c5ccccc5c5c6c7ccccc7n(-c7ccccc7)c6c6c7ccccc7n(-c7ccccc7)c6c54)ccc3c2)cc1 ; c1ccc(-n2c3ccccc3c3c4c5ccccc5n(-c5ccc(-c6ccc7ccccc7c6)cc5)c4c4c(c5ccccc5n4-c4ccccc4)c32)cc1 ; c1ccc(-c2ccc(-n3c4ccccc4c4c5c6ccccc6n(-c6ccc(-c7ccccc7)cc6)c5c5c(c6ccccc6n5-c5ccccc5)c43)cc2)cc1]

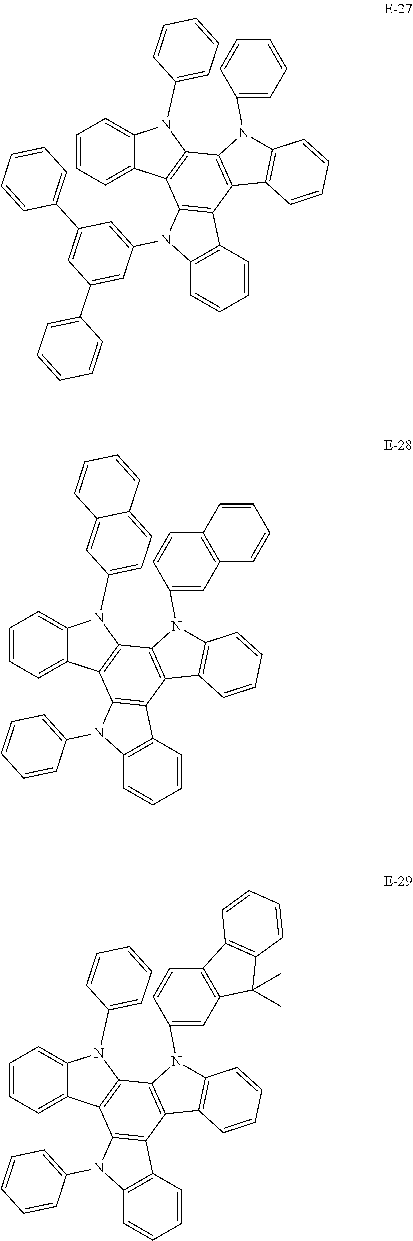 [SMILES: c1ccc(-c2cc(-c3ccccc3)cc(-n3c4ccccc4c4c5c6ccccc6n(-c6ccccc6)c5c5c(c6ccccc6n5-c5ccccc5)c43)c2)cc1 ; c1ccc(-n2c3ccccc3c3c4c5ccccc5n(-c5ccc6ccccc6c5)c4c4c(c5ccccc5n4-c4ccc5ccccc5c4)c32)cc1 ; CC1(C)c2ccccc2-c2ccc(-n3c4ccccc4c4c5c6ccccc6n(-c6ccccc6)c5c5c6ccccc6n(-c6ccccc6)c5c43)cc21]

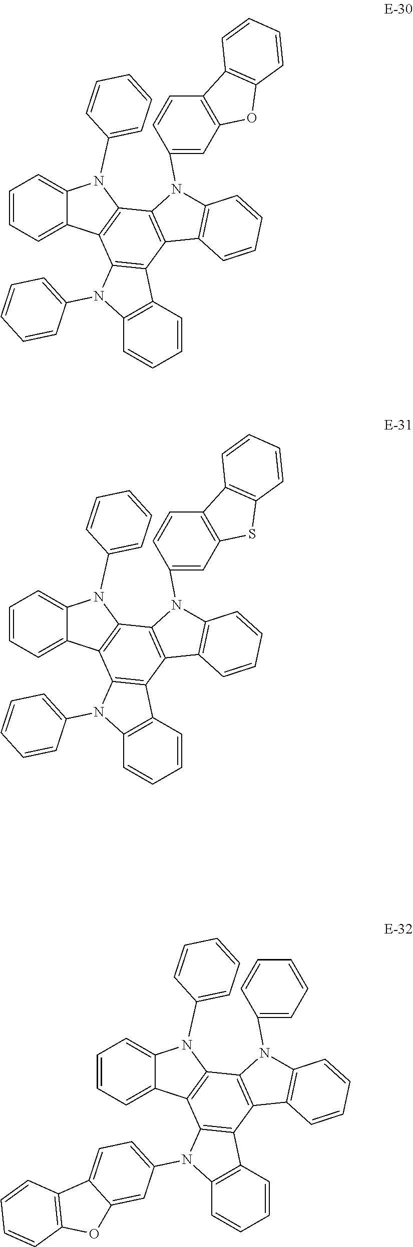 [SMILES: c1ccc(-n2c3ccccc3c3c4c5ccccc5n(-c5ccc6c(c5)oc5ccccc56)c4c4c(c5ccccc5n4-c4ccccc4)c32)cc1 ; c1ccc(-n2c3ccccc3c3c4c5ccccc5n(-c5ccc6c(c5)sc5ccccc56)c4c4c(c5ccccc5n4-c4ccccc4)c32)cc1 ; c1ccc(-n2c3ccccc3c3c4c5ccccc5n(-c5ccc6c(c5)oc5ccccc56)c4c4c5ccccc5n(-c5ccccc5)c4c32)cc1]

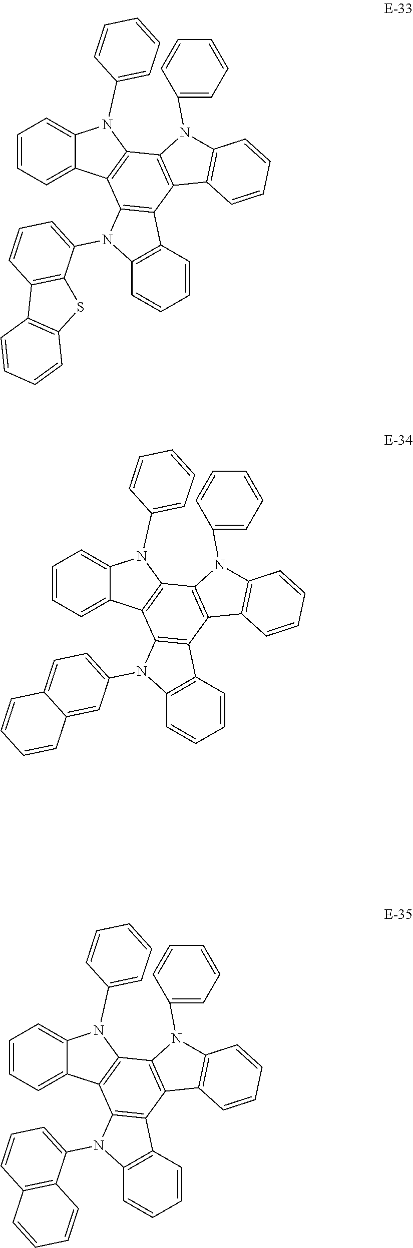 [SMILES: c1ccc(-n2c3ccccc3c3c4c5ccccc5n(-c5cccc6c5sc5ccccc56)c4c4c5ccccc5n(-c5ccccc5)c4c32)cc1 ; c1ccc(-n2c3ccccc3c3c4c5ccccc5n(-c5ccc6ccccc6c5)c4c4c5ccccc5n(-c5ccccc5)c4c32)cc1 ; c1ccc(-n2c3ccccc3c3c4c5ccccc5n(-c5cccc6ccccc56)c4c4c5ccccc5n(-c5ccccc5)c4c32)cc1]

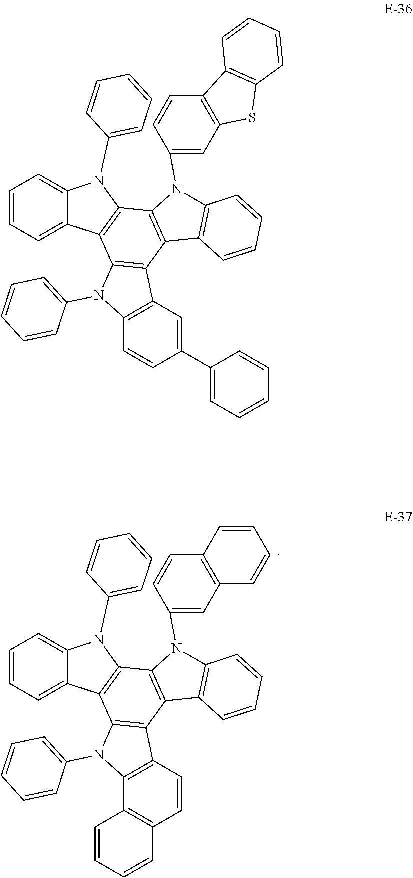 [SMILES: c1ccc(-c2ccc3c(c2)c2c4c5ccccc5n(-c5ccc6c(c5)sc5ccccc56)c4c4c(c5ccccc5n4-c4ccccc4)c2n3-c2ccccc2)cc1 ; c1ccc(-n2c3ccccc3c3c2c2c(c4ccccc4n2-c2ccc4ccccc4c2)c2c4ccc5ccccc5c4n(-c4ccccc4)c23)cc1]

The composition may further include a dopant.

The embodiments may be realized by providing an organic optoelectronic device including an anode and a cathode facing each other, and at least one organic layer between the anode and the cathode, wherein the organic layer includes the composition according to an embodiment.

The organic layer may include a light emitting layer, and the light emitting layer may include the composition.

The first compound and the second compound may be a phosphorescent host of the light emitting layer.

The composition may be a red light emitting composition.

The composition may be a green light emitting composition.

The embodiments may be realized by providing a display device including the organic optoelectronic device according to an embodiment.

### BRIEF DESCRIPTION OF THE DRAWINGS

Features will be apparent to those of skill in the art by describing in detail exemplary embodiments with reference to the attached drawings in which:

FIGS. 1 and 2 illustrate cross-sectional views showing organic light emitting diodes according to embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or element, it can be directly on the other layer or element, or intervening layers may also be present. Like reference numerals refer to like elements throughout.

As used herein, when a definition is not otherwise provided, the term “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heterocyclic group, a C1 to C20 alkoxy group, a C1 to C10 trifluoroalkyl group, a cyano group, or a combination thereof.

In one example of the present disclosure, the term “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, or a C2 to C30 heteroaryl group. In addition, in specific examples of the present disclosure, the “substituted” refers to replacement of at least one hydrogen of a substituent or a compound by deuterium, a C1 to C20 alkyl group, a C6 to C30 aryl group, or a C2 to C30 heteroaryl group. In addition, in specific examples of the present disclosure, the term “substituted” refers to replacement of at least one hydrogen of a substituent or a compound by deuterium, a C1 to C5 alkyl group, a C6 to C18 aryl group, a pyridinyl group, a quinolinyl group, an isoquinolinyl group, a dibenzofuranyl group, a dibenzothiophenyl group, or a carbazolyl group. In addition, in specific examples of the present disclosure, the “substituted” refers to replacement of at least one hydrogen of a substituent or a compound by deuterium, a C1 to C5 alkyl group, a C6 to C18 aryl group, a dibenzofuranyl group, or a dibenzothiophenyl group. In addition, in specific examples of the present disclosure, the “substituted” refers to replacement of at least one hydrogen of a substituent or a compound by deuterium, a methyl group, an ethyl group, a propanyl group, a butyl group, a phenyl group, a biphenyl group, a terphenyl group, a naphthyl group, a triphenyl group, a dibenzofuranyl group, or a dibenzothiophenyl group.

As used herein, when a definition is not otherwise provided, “hetero” refers to one including one to three heteroatoms selected from N, O, S, P, and Si, and remaining carbons in one functional group.

As used herein, the “aryl group” refers to a group including at least one hydrocarbon aromatic moiety, and all the elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic or fused ring polycyclic (i.e., rings sharing adjacent pairs of carbon atoms) functional group.

As used herein, the “heterocyclic group” is a generic concept including a heteroaryl group and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, the “heteroaryl group” may refer to an aryl group including at least one heteroatom selected from N, O, S, P, and Si. Two or more heteroaryl groups are linked by a sigma bond directly, or when the C2 to C60 heteroaryl group includes two or more rings, the two or more rings may be fused. When the heteroaryl group is a fused ring, each ring may include 1 to 3 heteroatoms.

Specific examples of the heterocyclic group may be a pyridinyl group, a pyrimidinyl group, a pyrazinyl group, a pyridazinyl group, a triazinyl group, a quinolinyl group, an isoquinolinyl group, and the like.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but are not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof, but are not limited thereto.

In the present specification, hole characteristics refer to an ability to donate an electron to form a hole when an electric field is applied, and that a hole formed in the anode may be easily injected into a light emitting layer, and a hole formed in a light emitting layer may be easily transported into an anode and transported in the light emitting layer due to conductive characteristics according to a highest occupied molecular orbital (HOMO) level.

In addition, electron characteristics refer to an ability to accept an electron when an electric field is applied, and that an electron formed in a cathode may be easily injected into a light emitting layer, and an electron formed in a light emitting layer may be easily transported into a cathode and transported in the light emitting layer due to conductive characteristics according to a lowest unoccupied molecular orbital (LUMO) level.

Hereinafter, a composition for an organic optoelectronic device according to an embodiment is described.

A composition for an organic optoelectronic device according to an embodiment may include a first compound having electron characteristics and a second compound having hole characteristics.

The first compound may be represented by Chemical Formula 1.

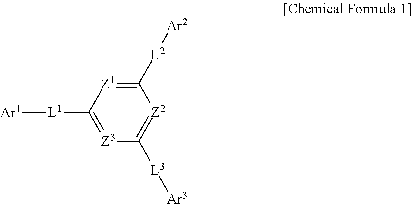

In Chemical Formula 1,

Z¹ to Z³ may independently be, e.g., N or CR^{a}. R^{a} may be or may include, e.g., hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof.

In an implementation, at least two of Z¹ to Z³ may be N.

L¹ to L³ may independently be or include, e.g., a single bond, a substituted or unsubstituted C6 to C20 arylene group, a substituted or unsubstituted divalent C3 to C20 heterocyclic group, or a combination thereof.

Ar¹ to Ar³ may independently be or include, e.g., a substituted or unsubstituted C6 to C30 aryl group, a substituted or unsubstituted C3 to C30 heterocyclic group, or a combination thereof.

In an implementation, at least one of Ar¹ to Ar³ may be a group represented by Chemical Formula A. For example, the group represented by Chemical Formula A may be a subset of the substituted or unsubstituted C3 to C30 heterocyclic group.

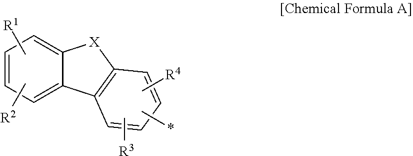

In Chemical Formula A,

X may be, e.g., O or S, and

R¹ to R⁴ may independently be or include, e.g., hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof.

In an implementation, R¹ and R² may be separate or linked with each other to form a ring,

In an implementation, R³ and R⁴ may be separate or linked with each other to form a ring, and

* is a linking point with Chemical Formula 1.

The first compound may be a compound capable of accepting electron, when an electric field is applied, e.g., a compound having electron characteristics. For example, the first compound may have structure where a fused ring represented by Chemical Formula A is linked with a nitrogen-containing ring, e.g., a pyrimidine or a triazine ring, to easily accept electrons when an electric field is applied. Thus, a driving voltage of an organic optoelectronic device including the first compound may be lowered.

In an implementation, at least two of Z¹ to Z³ may be nitrogen (N) and the remaining one may be CR^{a}.

For example, Z¹ and Z² may be nitrogen and Z³ may be CR^{a}.

For example, Z² and Z³ may be nitrogen and Z¹ may be CR^{a}.

For example, Z¹ and Z³ may be nitrogen and Z² may be CR^{a}.

For example, Z¹ to Z³ may be nitrogen (N).

In an implementation, L¹ to L³ may independently be, e.g., a single bond or a substituted or unsubstituted C6 to C20 arylene group.

In an implementation, L¹ to L³ may independently be, e.g., a single bond, a substituted or unsubstituted phenylene group, a substituted or unsubstituted biphenylene group, a substituted or unsubstituted terphenylene group, or a substituted or unsubstituted naphthylene group.

In an implementation, L¹ to L³ may independently be, e.g., a single bond, a substituted or unsubstituted m-phenylene group, a substituted or unsubstituted p-phenylene group, a substituted or unsubstituted o-phenylene group, a substituted or unsubstituted m-biphenylene group, a substituted or unsubstituted p-biphenylene group, a substituted or unsubstituted o-biphenylene group, a substituted or unsubstituted m-terphenylene group, a substituted or unsubstituted p-terphenylene group, or a substituted or unsubstituted o-terphenylene group. Herein, “substituted” may for example refer to replacement of at least one hydrogen by deuterium, a C1 to C20 alkyl group, a C6 to C20 aryl group, a halogen, a cyano group, or a combination thereof.

In an implementation, Ar¹ to Ar³ may independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted pyridinyl group, a substituted or unsubstituted pyrimidinyl group, a substituted or unsubstituted triazinyl group, the group represented by Chemical Formula A, or a combination thereof.

In an implementation, the group represented by Chemical Formula A may be, e.g., a group represented by one of Chemical Formula A-1 to A-4 according to a binding position.

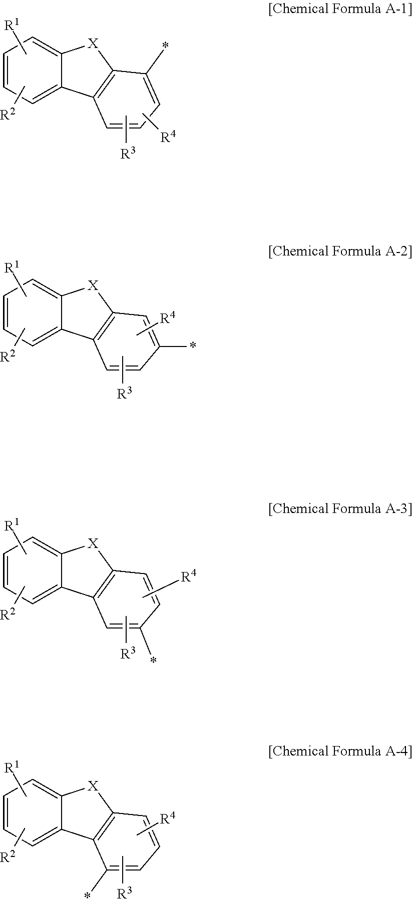

In Chemical Formulae A-1 to A-4, X and R¹ to R⁴ may be the same as described above.

In an implementation, the group represented by Chemical Formula A may be represented by Chemical Formula A-1 or A-2.

In an implementation first compound may be, e.g., represented by one of

Chemical Formulae 1A to 1C according the number of the groups represented by Chemical Formula A.

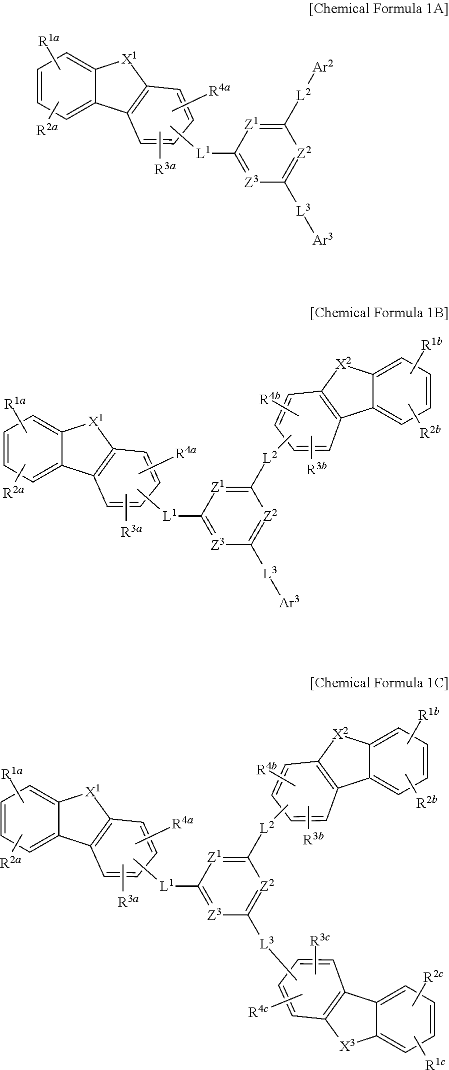

In Chemical Formulae 1A to 1C,

Z¹ to Z³, L¹ to L³, Ar², and Ar³ may be the same as described above,

X¹ to X³ may independently be O or S, and

R^{1a} to R^{4a}, R^{1b} to R^{4b}, and R^{1c} to R^{4c} may independently be, e.g., hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof.

For example, in Chemical Formula 1B, X¹ and X² may be the same or different.

For example, in Chemical Formula 1B, X¹ and X² may be the same and X¹ and X² may be O respectively.

For example, in Chemical Formula 1B, X¹ and X² may be the same and X¹ and X² may be S respectively.

For example, in Chemical Formula 1B, X¹ and X² may be different and X¹may be S and X² may be O or X¹ may be O and X² may be S.

For example, in Chemical Formula 1C, X¹ to X³ may be the same or different.

For example, in Chemical Formula 1C, X¹ to X³ may be the same and X¹ to X³ may be O respectively.

For example, in Chemical Formula 1C, X¹ to X³ may be the same and X¹ to X³ may be S respectively.

For example, in Chemical Formula 1C, one of X¹ to X³ may be different, two of X¹ to X³ may be S and one of X¹ to X³ may be O or two of X¹ to X³ may be O and one of X¹ to X³ may be S.

For example, the first compound may be represented by Chemical Formula 1B.

For example, in Chemical Formula 1B L¹ and L² may be a single bond, respectively.

In an implementation, the compound represented by Chemical Formula 1B may be represented by Chemical Formula 1B-1.

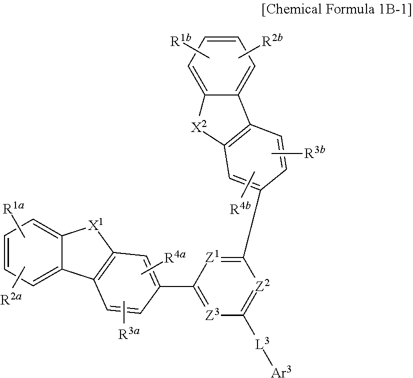

In Chemical Formula 1B-1,

Z¹ to Z³, Ar³, L³, R^{1a} to R^{4a}, and R^{1b} to R^{4b} may be the same as described above.

The first compound represented by Chemical Formula 1B-1 may have an effectively expanded LUMO energy band and increased planarity of a molecular structure and thus may have a structure easily accepting electrons, when an electric field is applied, and accordingly, much lower a driving voltage of an organic optoelectronic device manufactured by applying the first compound. In addition, this expansion of LUMO and the fusion of rings increases stability regarding electrons of the pyrimidine or triazine ring and thus effectively may help improve a life-span of the organic optoelectronic device manufactured by applying the first compound.

For example, in Chemical Formula 1B-1, X¹ and X² may be O respectively.

In an implementation first compound may be, e.g., one of the compounds of Group 1.

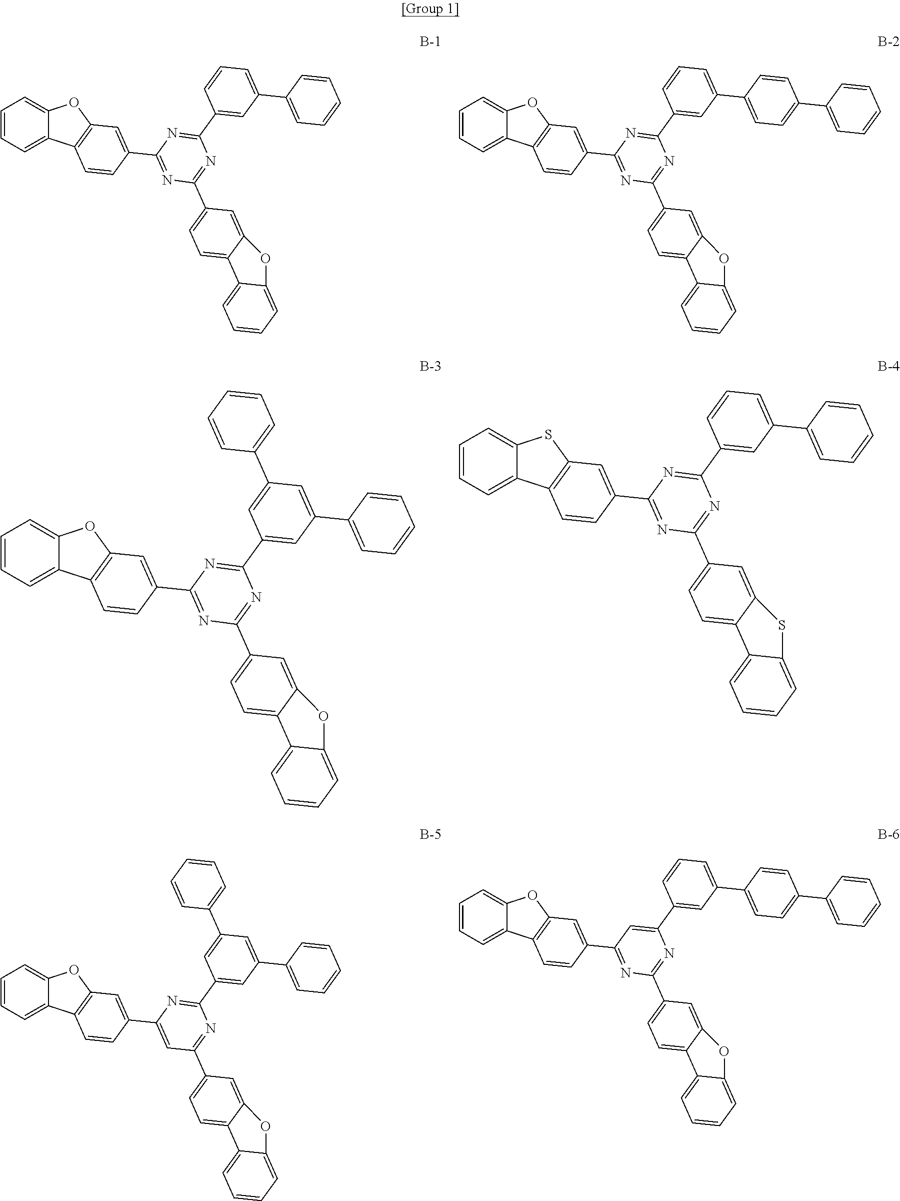

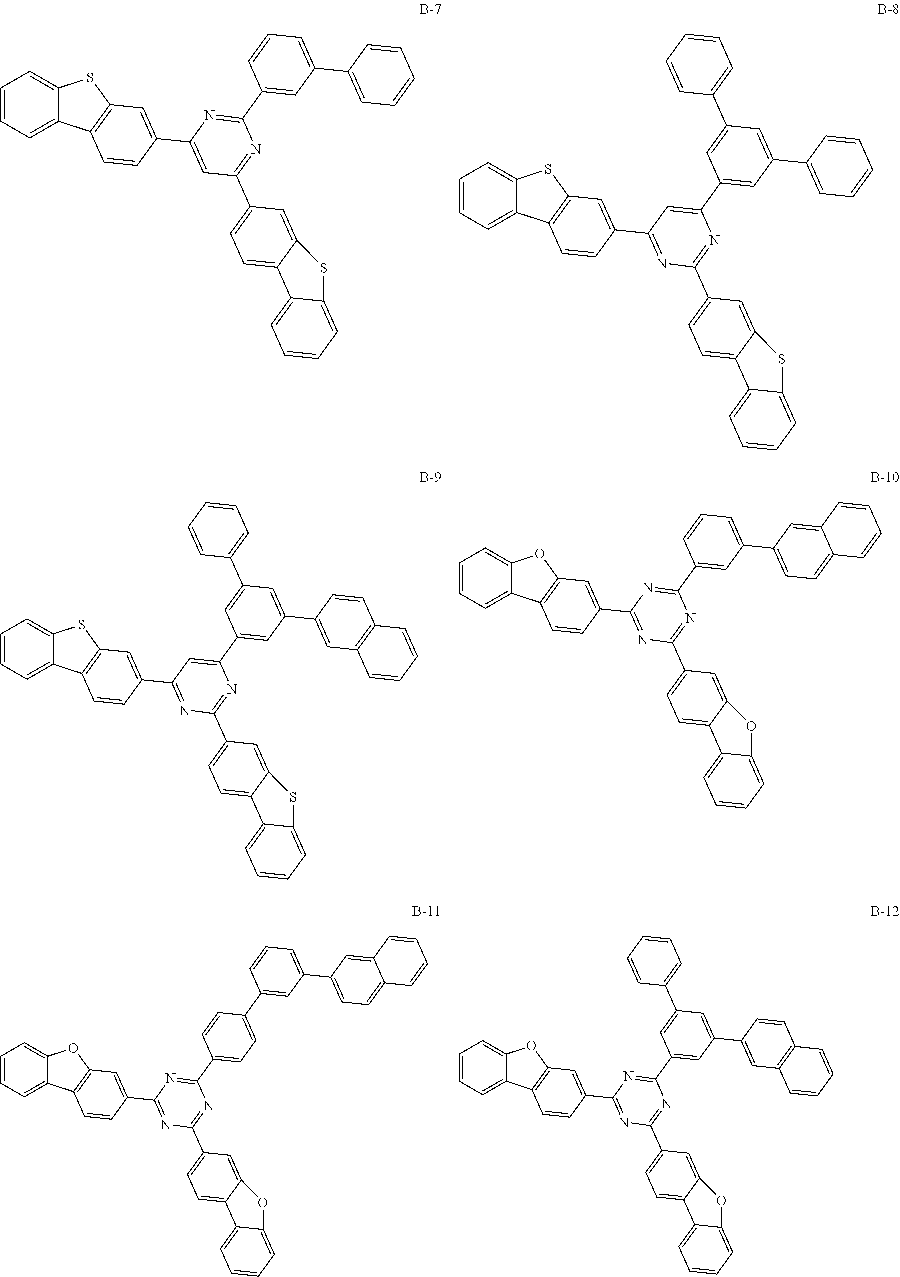

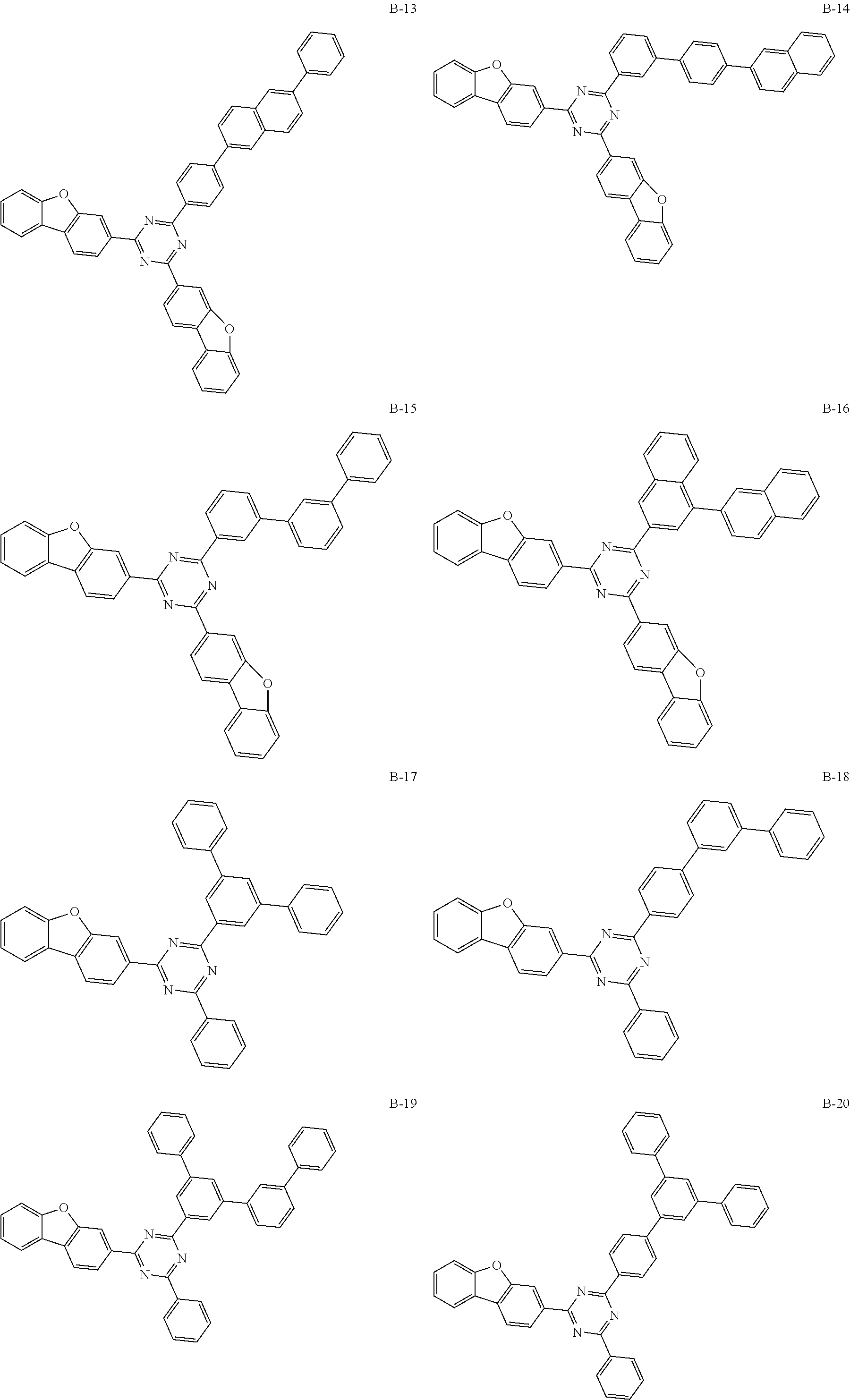

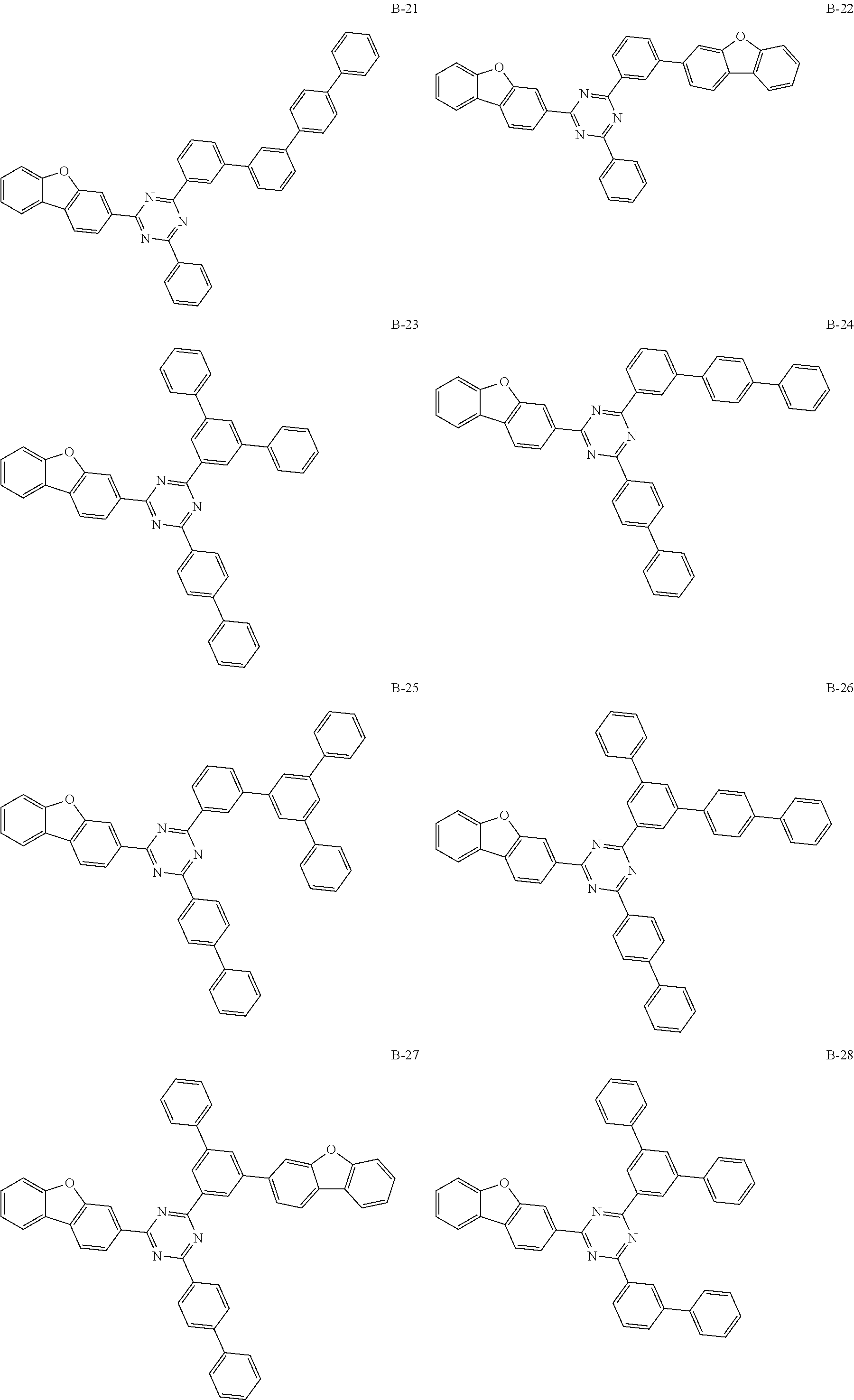

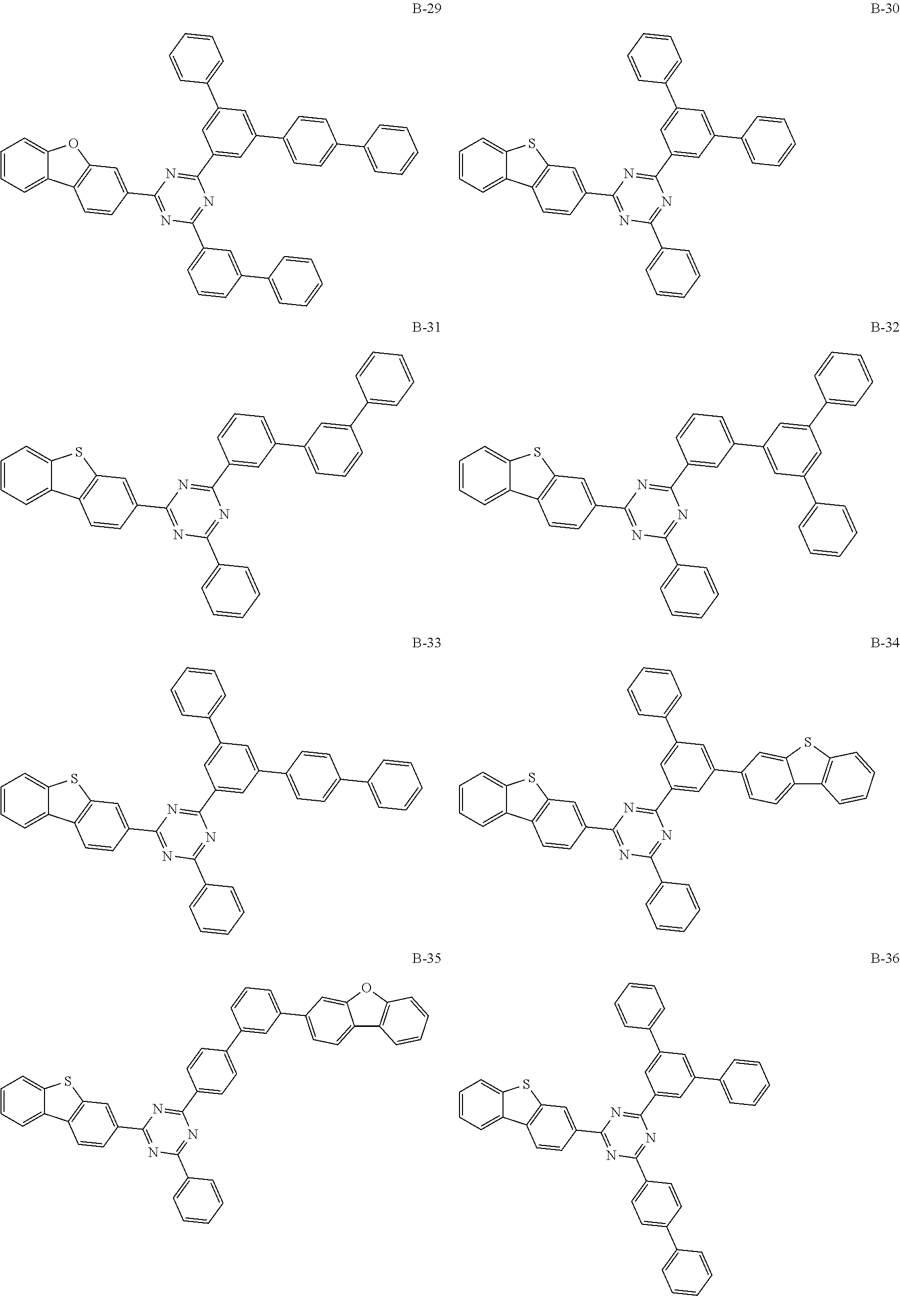

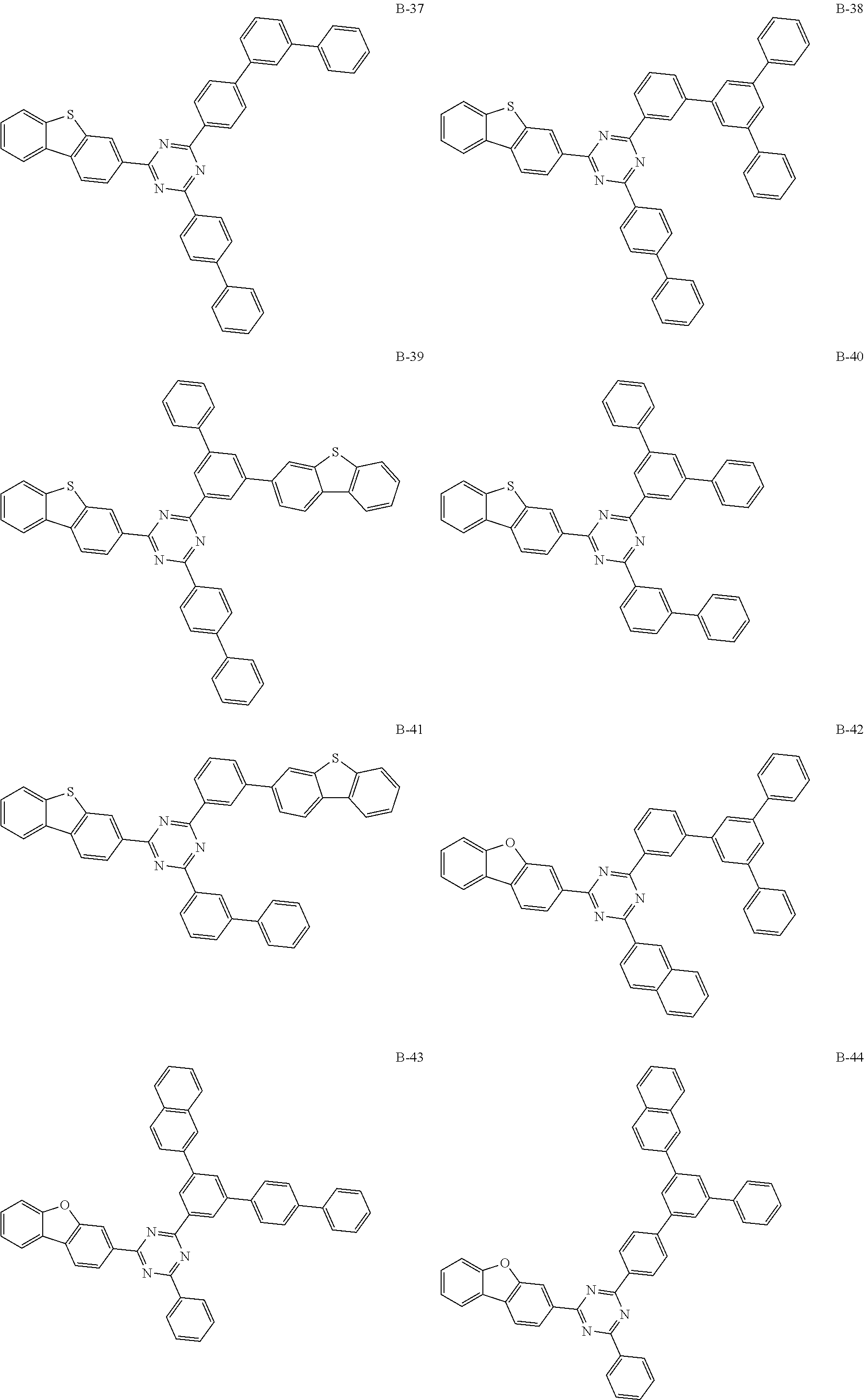

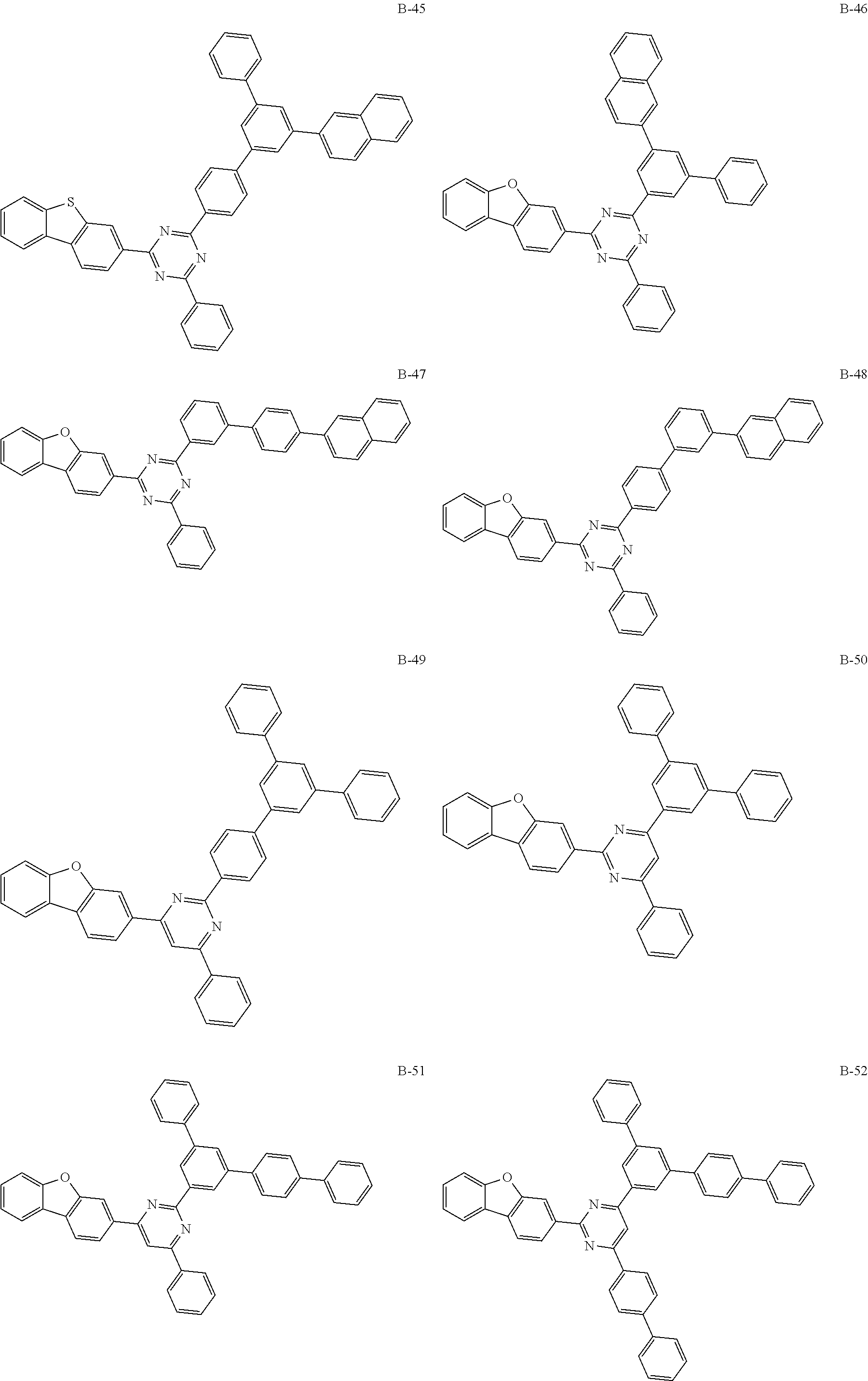

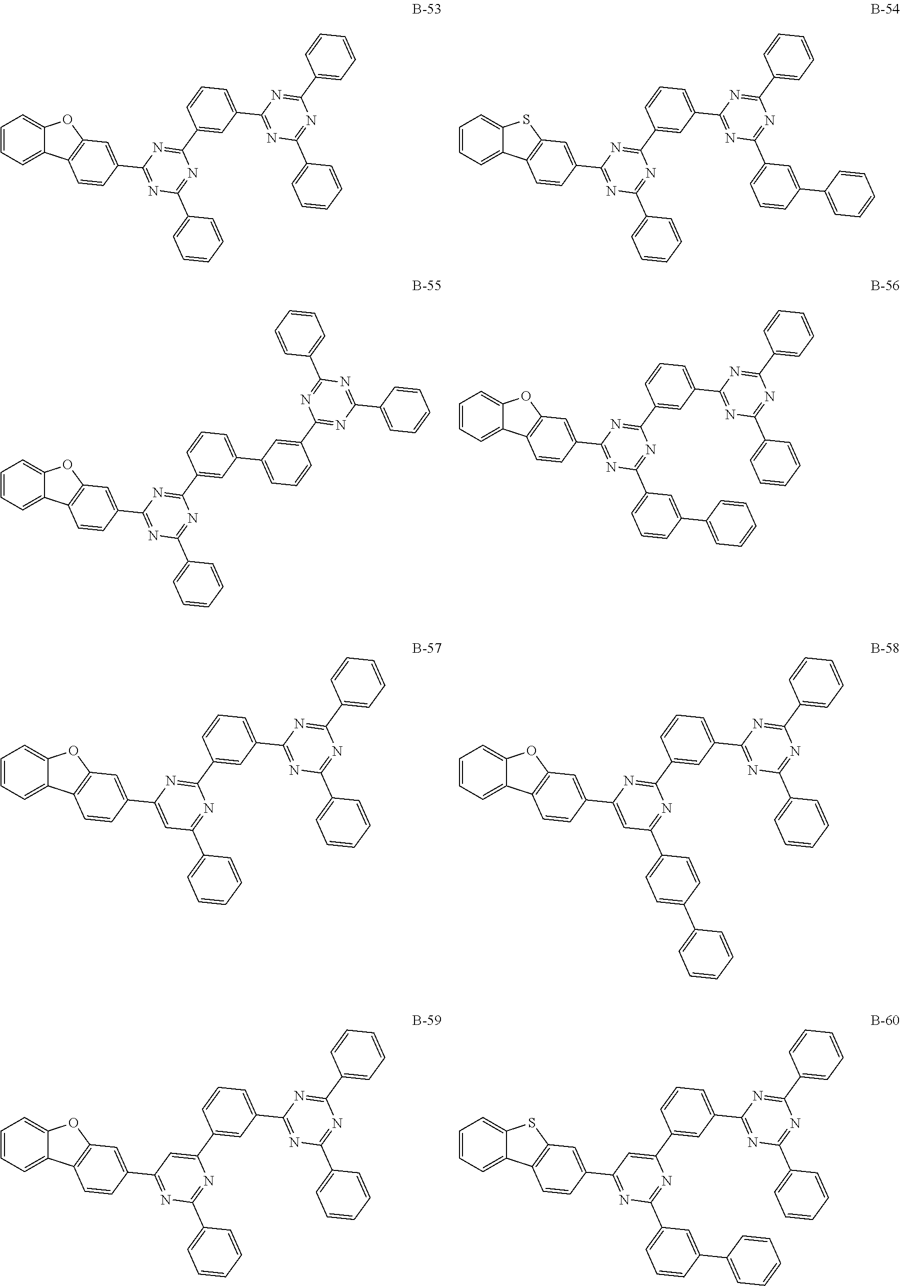

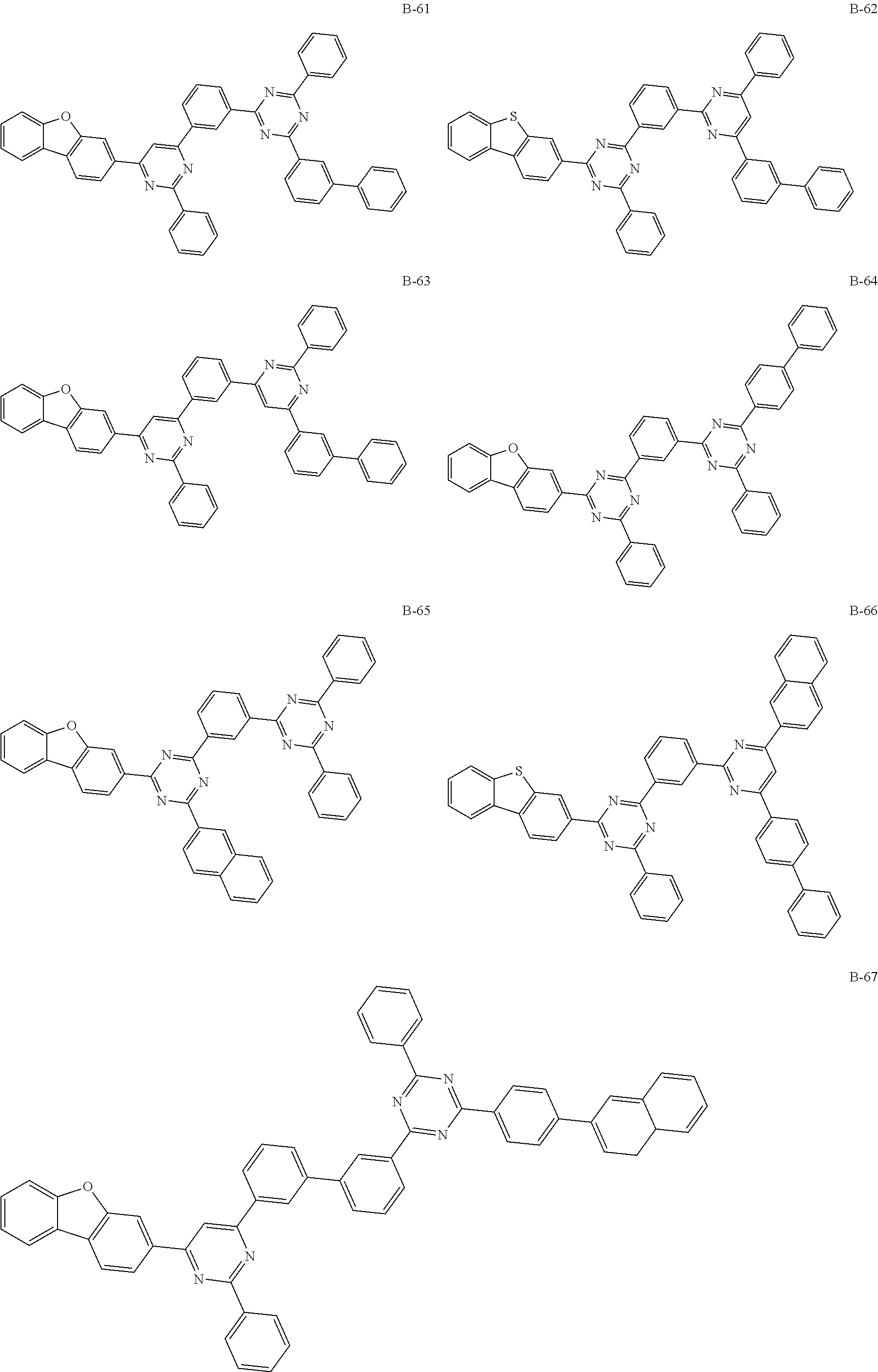

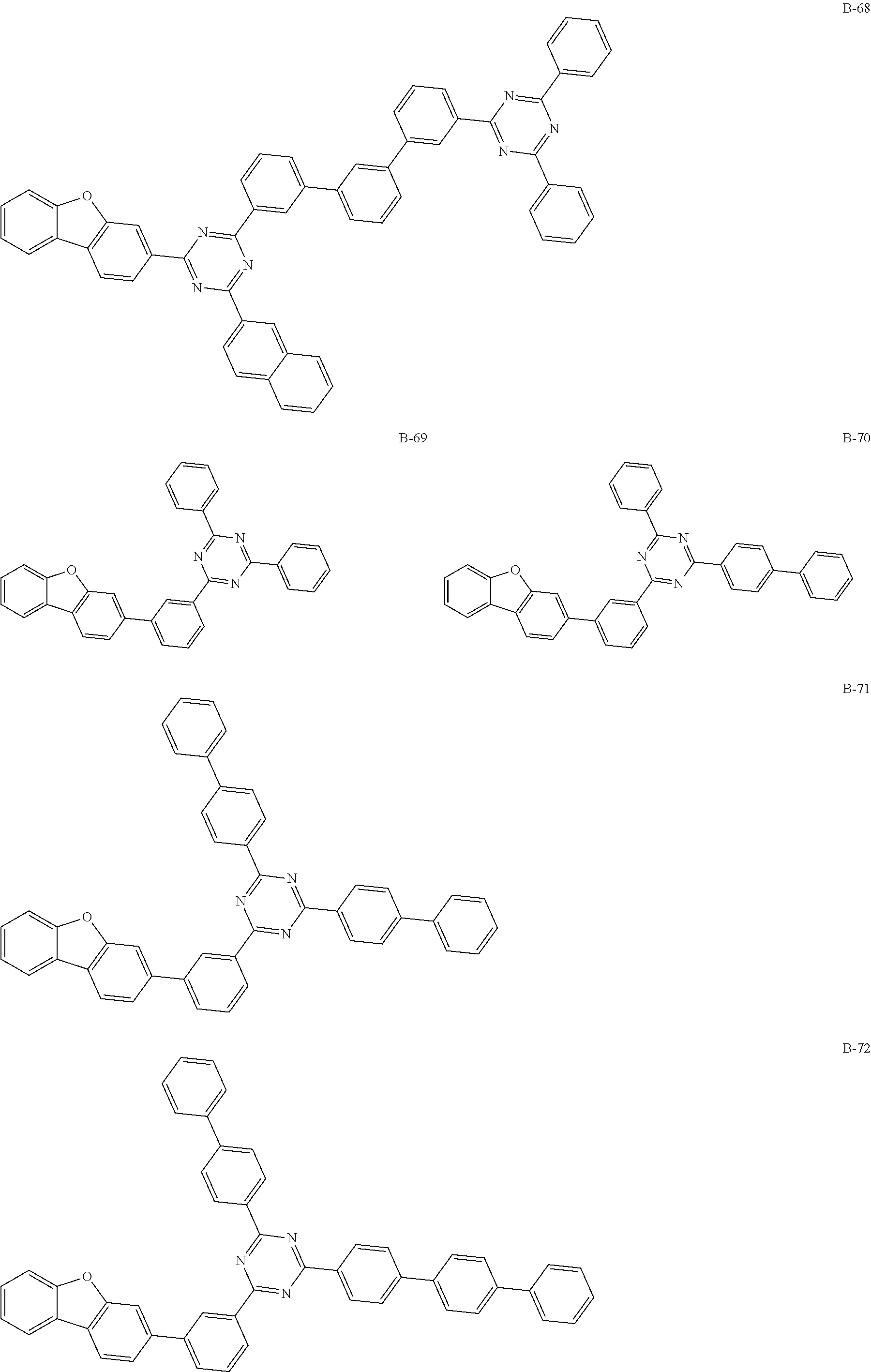 [SMILES: c1ccc(-c2nc(-c3ccccc3)nc(-c3cccc(-c4cccc(-c5cccc(-c6nc(-c7ccc8ccccc8c7)nc(-c7ccc8c(c7)oc7ccccc78)n6)c5)c4)c3)n2)cc1 ; c1ccc(-c2nc(-c3ccccc3)nc(-c3cccc(-c4ccc5c(c4)oc4ccccc45)c3)n2)cc1 ; c1ccc(-c2ccc(-c3nc(-c4ccccc4)nc(-c4cccc(-c5ccc6c(c5)oc5ccccc56)c4)n3)cc2)cc1 ; c1ccc(-c2ccc(-c3nc(-c4ccc(-c5ccccc5)cc4)nc(-c4cccc(-c5ccc6c(c5)oc5ccccc56)c4)n3)cc2)cc1 ; c1ccc(-c2ccc(-c3ccc(-c4nc(-c5ccc(-c6ccccc6)cc5)nc(-c5cccc(-c6ccc7c(c6)oc6ccccc67)c5)n4)cc3)cc2)cc1]

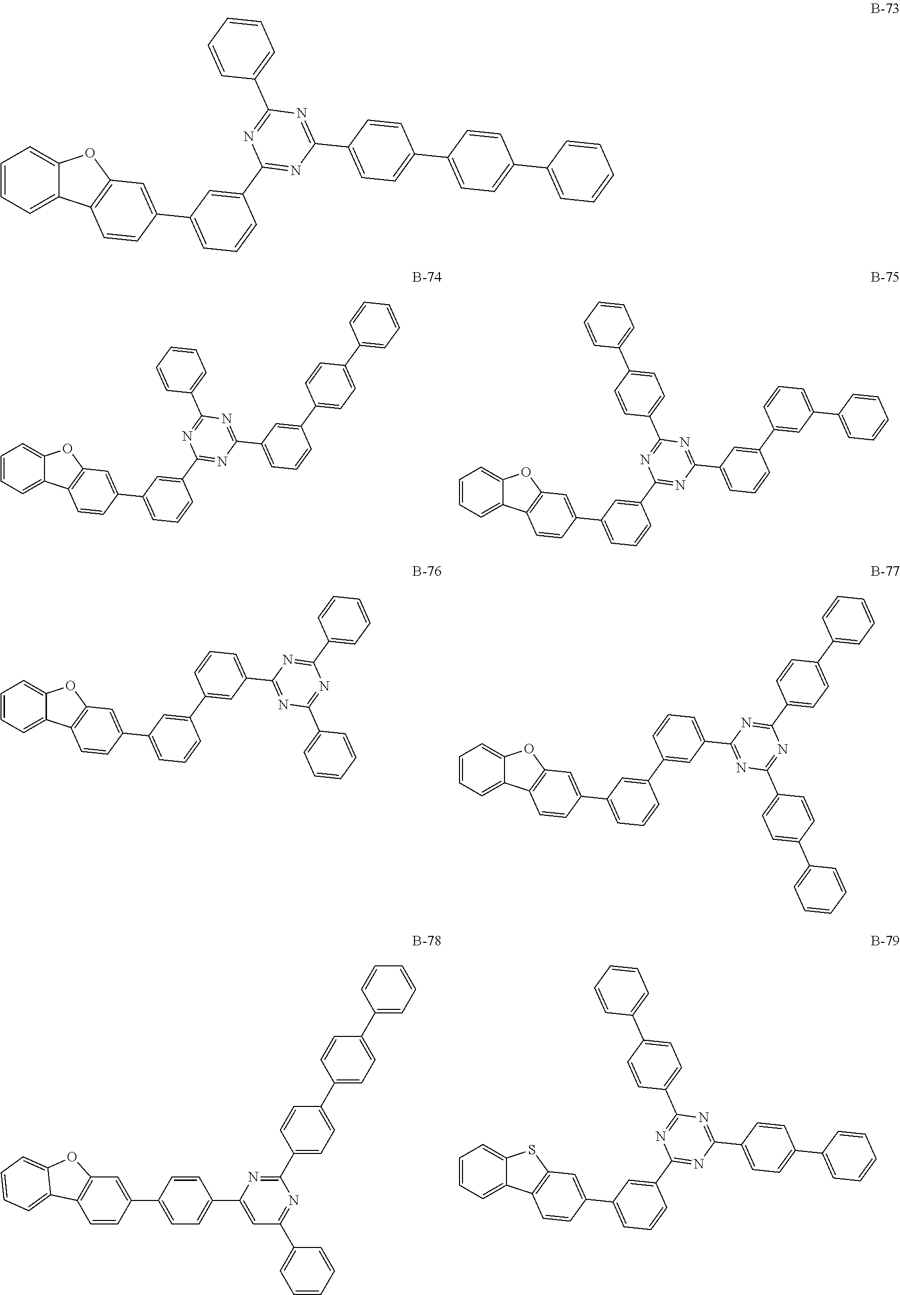

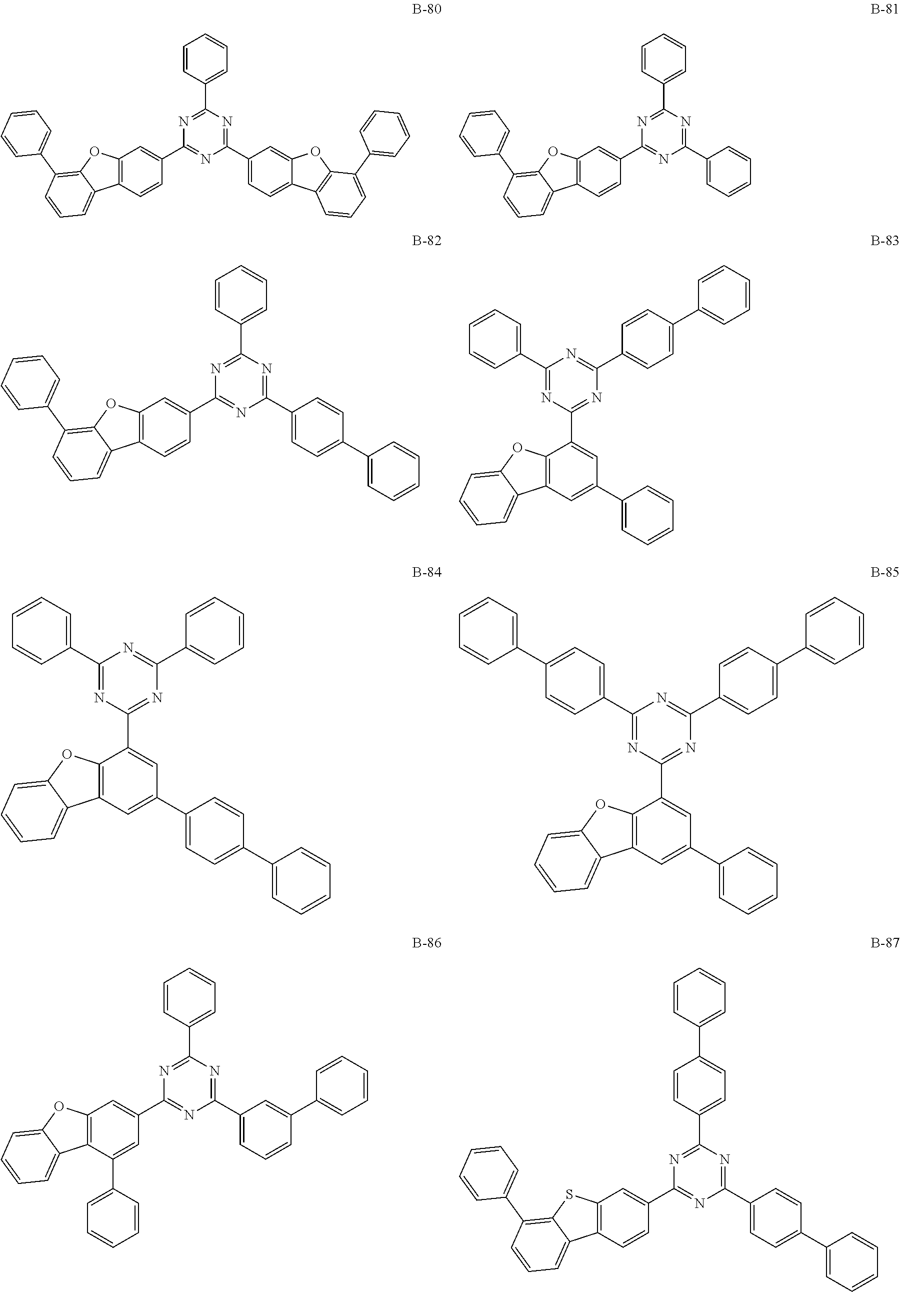

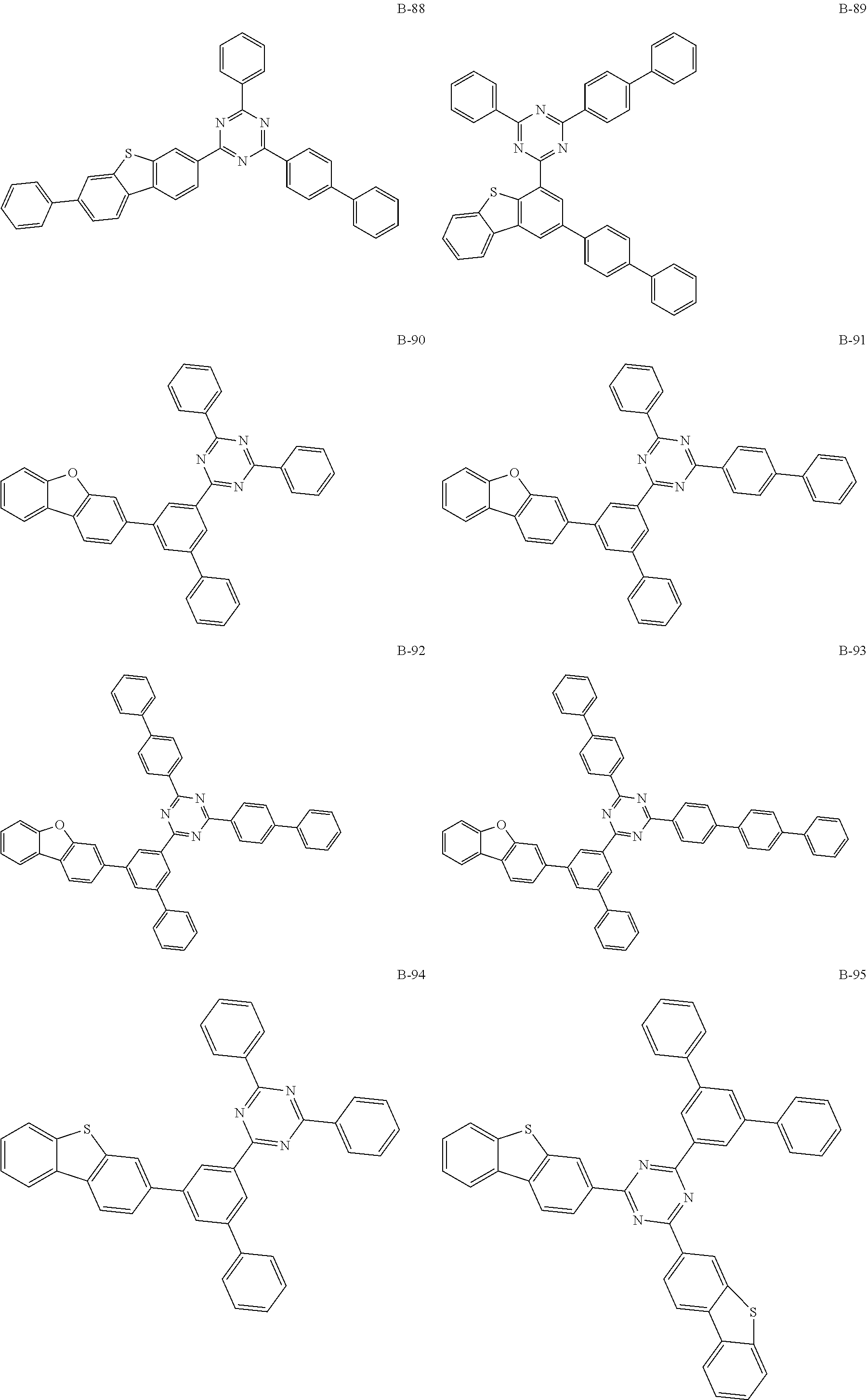

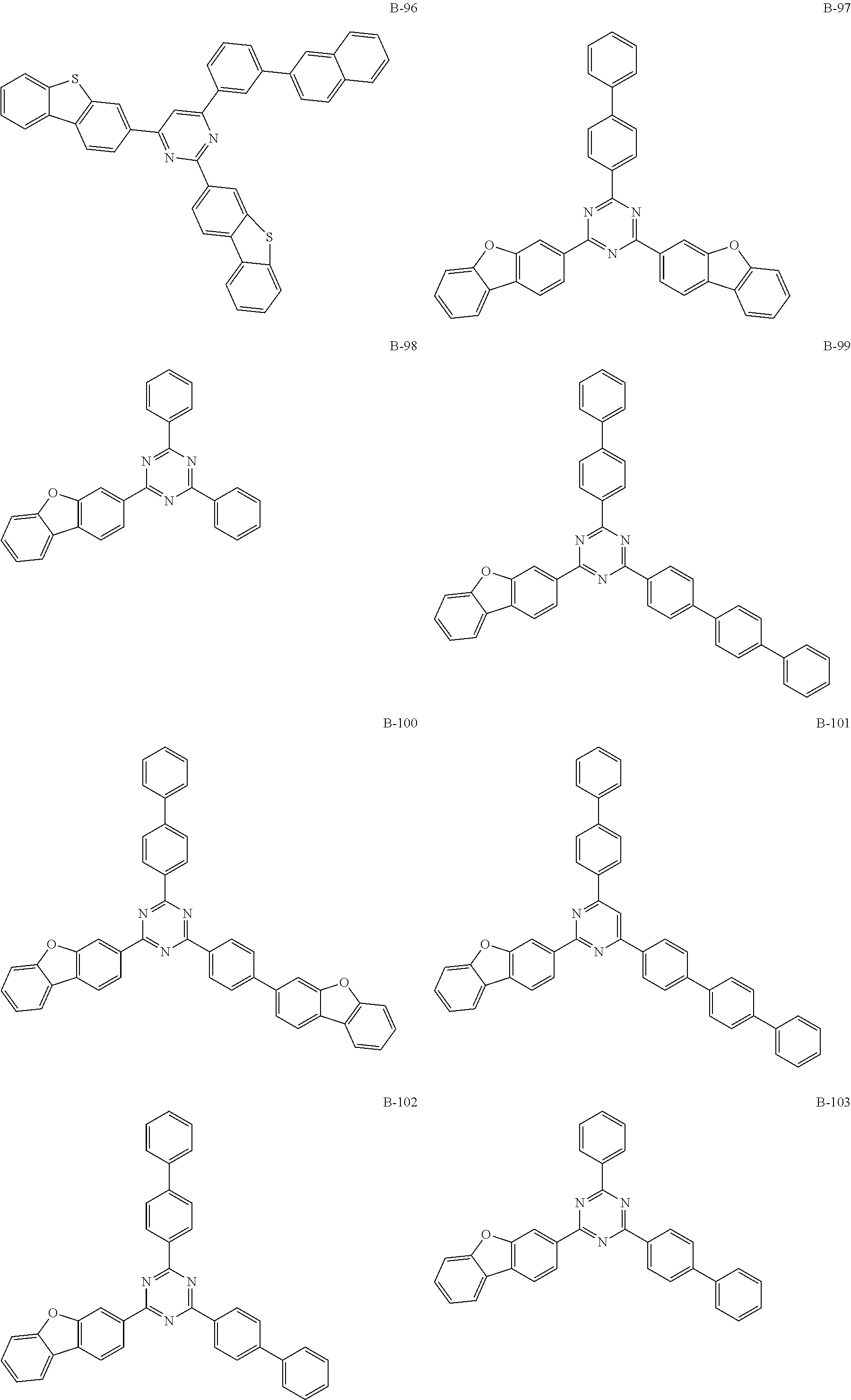

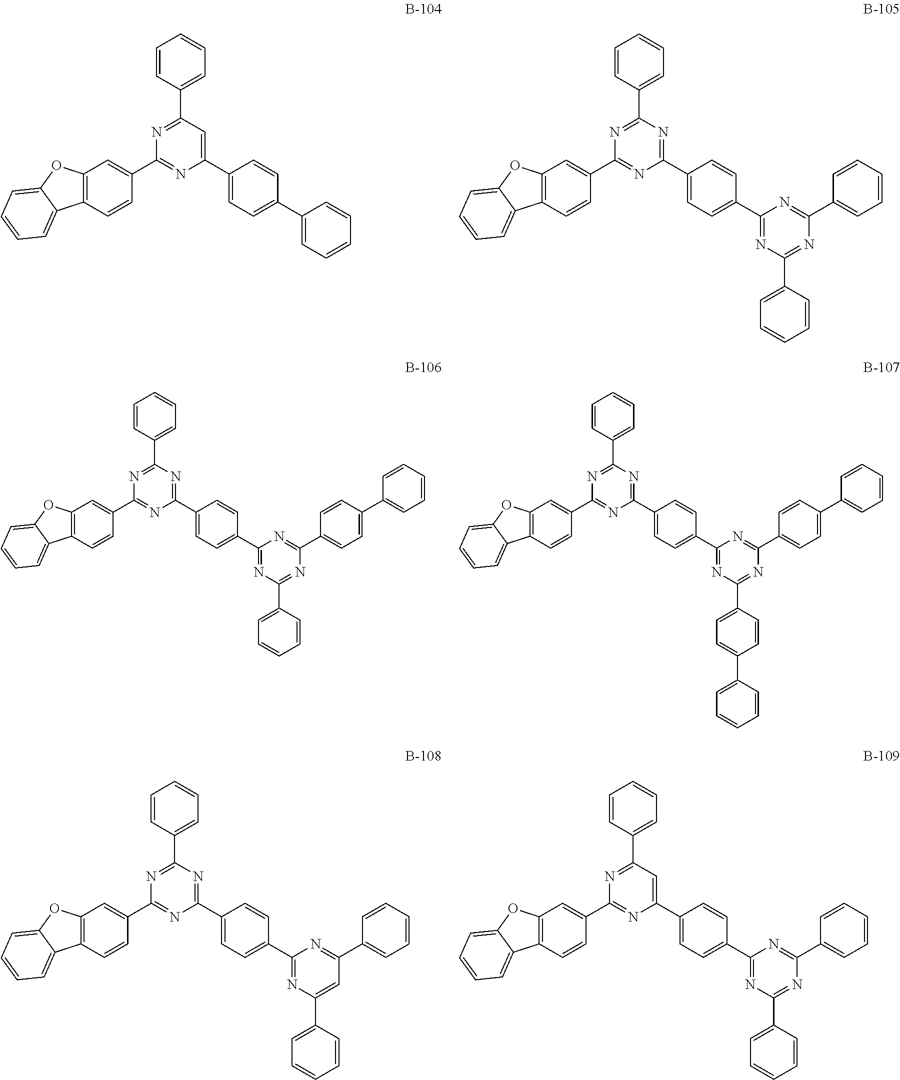

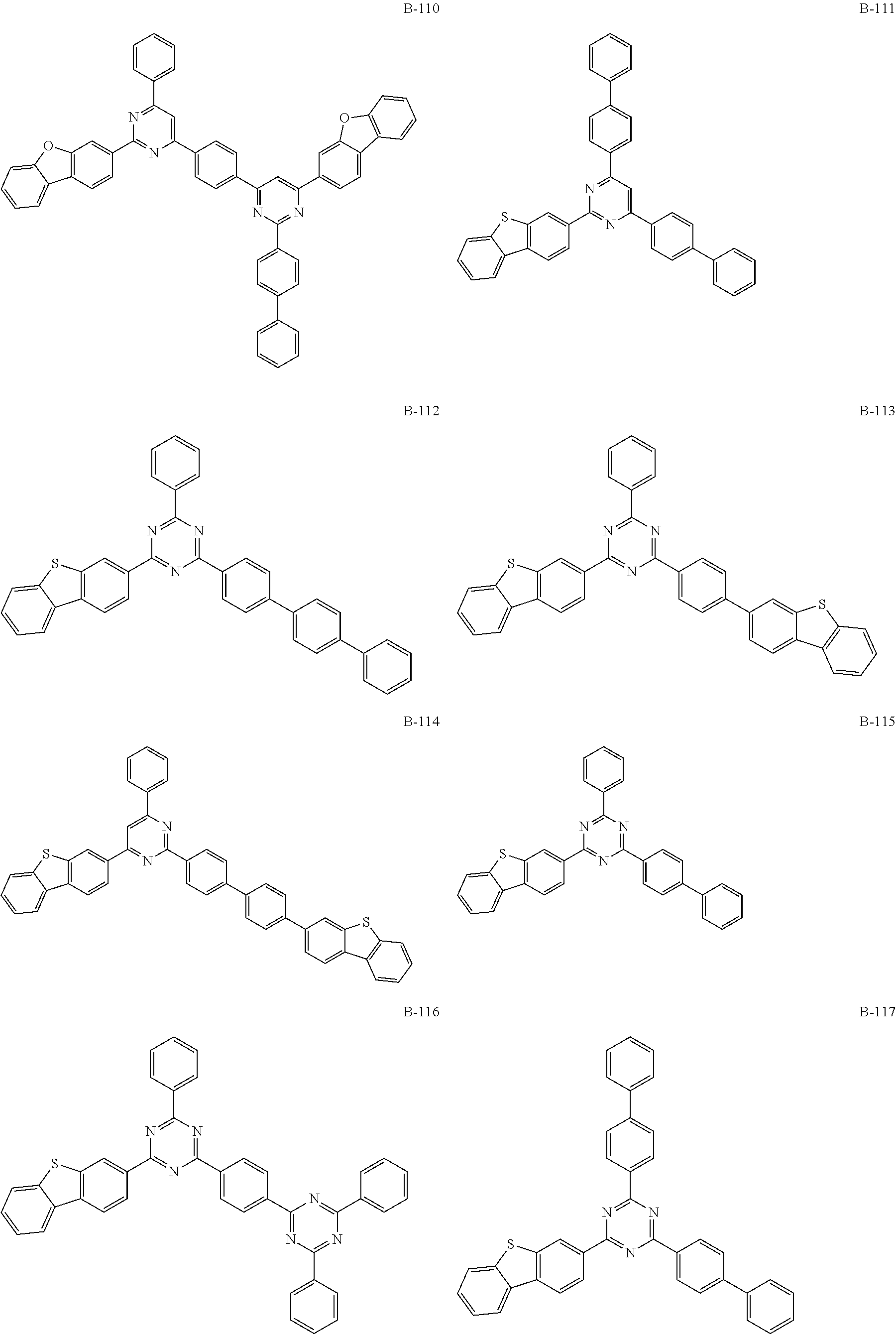

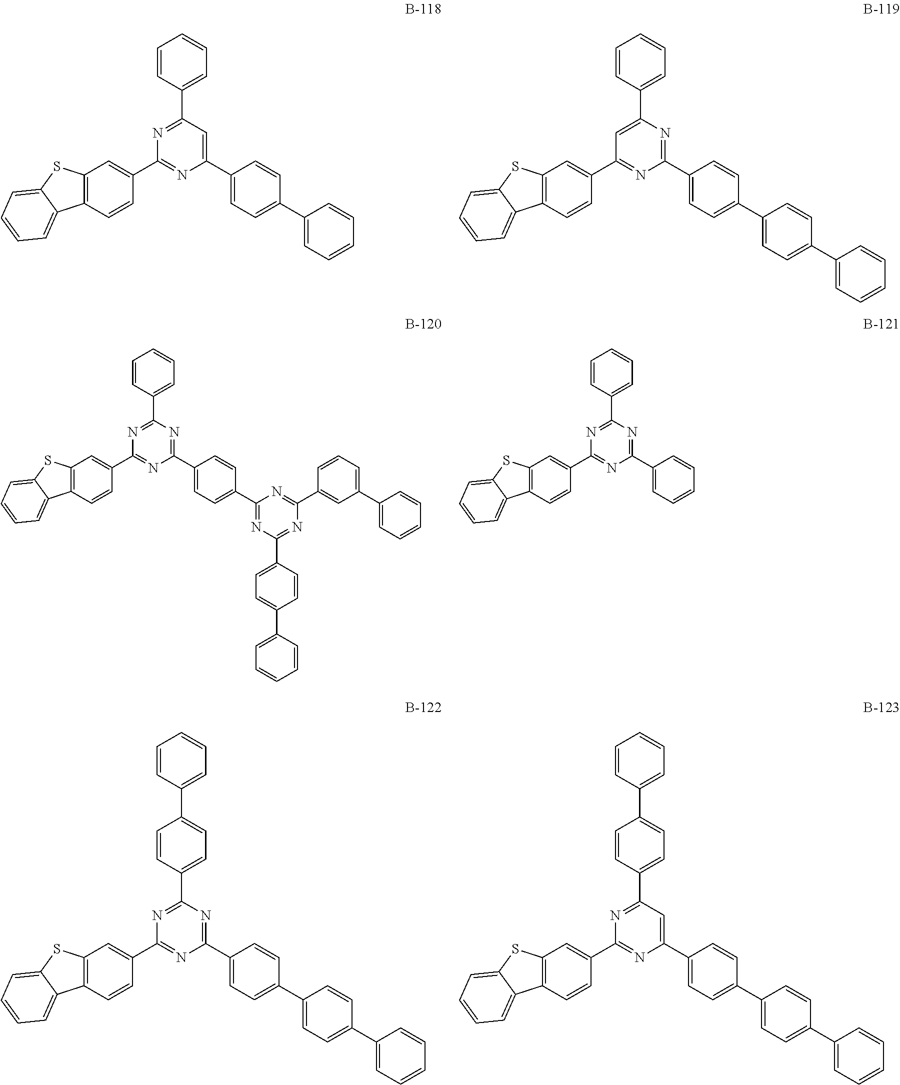

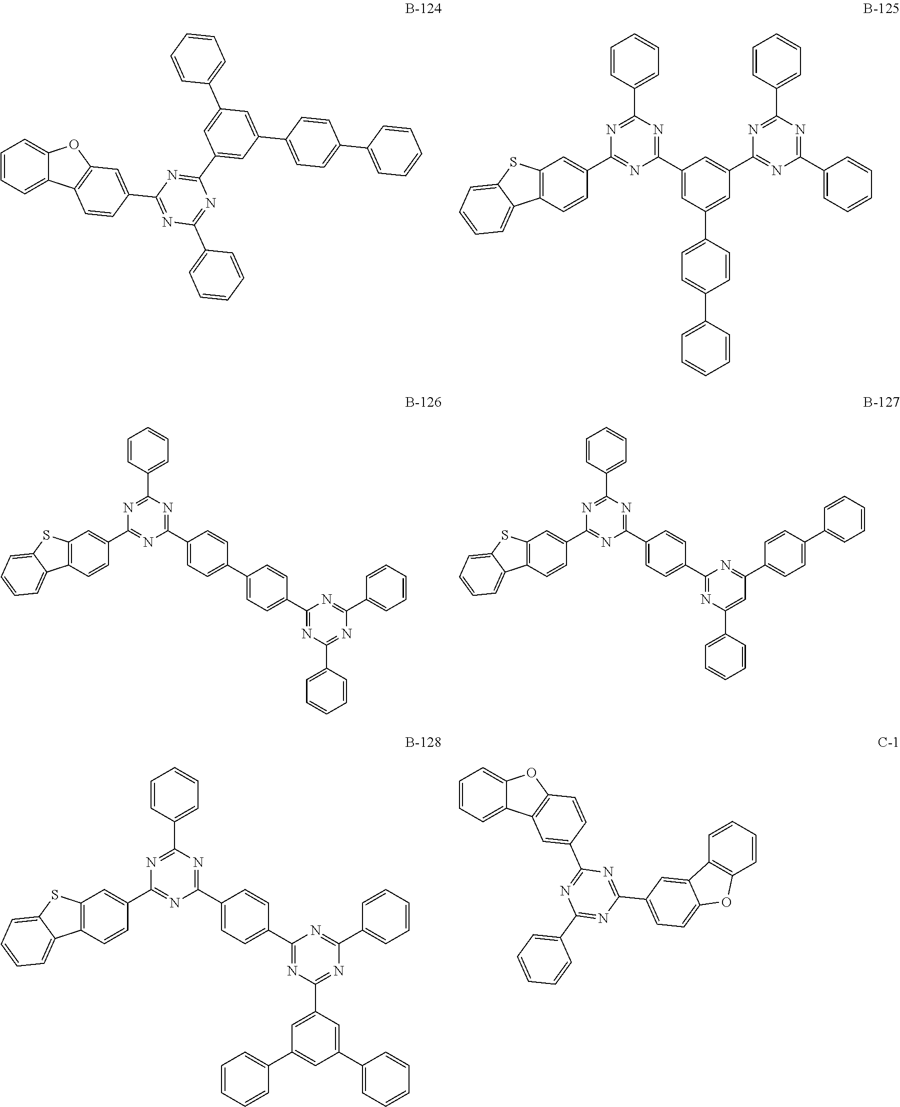

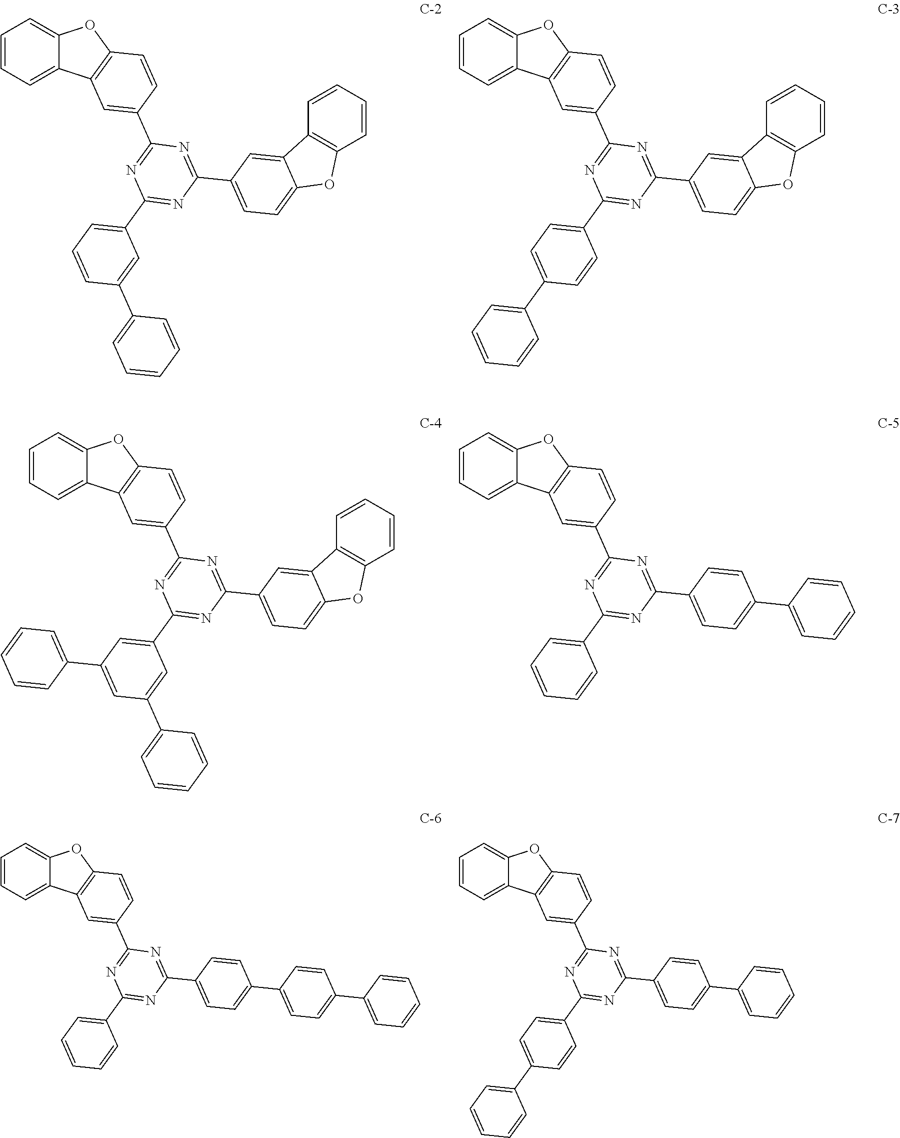 [SMILES: c1ccc(-c2cccc(-c3nc(-c4ccc5oc6ccccc6c5c4)nc(-c4ccc5oc6ccccc6c5c4)n3)c2)cc1 ; c1ccc(-c2ccc(-c3nc(-c4ccc5oc6ccccc6c5c4)nc(-c4ccc5oc6ccccc6c5c4)n3)cc2)cc1 ; c1ccc(-c2cc(-c3ccccc3)cc(-c3nc(-c4ccc5oc6ccccc6c5c4)nc(-c4ccc5oc6ccccc6c5c4)n3)c2)cc1 ; c1ccc(-c2ccc(-c3nc(-c4ccccc4)nc(-c4ccc5oc6ccccc6c5c4)n3)cc2)cc1 ; c1ccc(-c2ccc(-c3ccc(-c4nc(-c5ccccc5)nc(-c5ccc6oc7ccccc7c6c5)n4)cc3)cc2)cc1 ; c1ccc(-c2ccc(-c3nc(-c4ccc(-c5ccccc5)cc4)nc(-c4ccc5oc6ccccc6c5c4)n3)cc2)cc1]

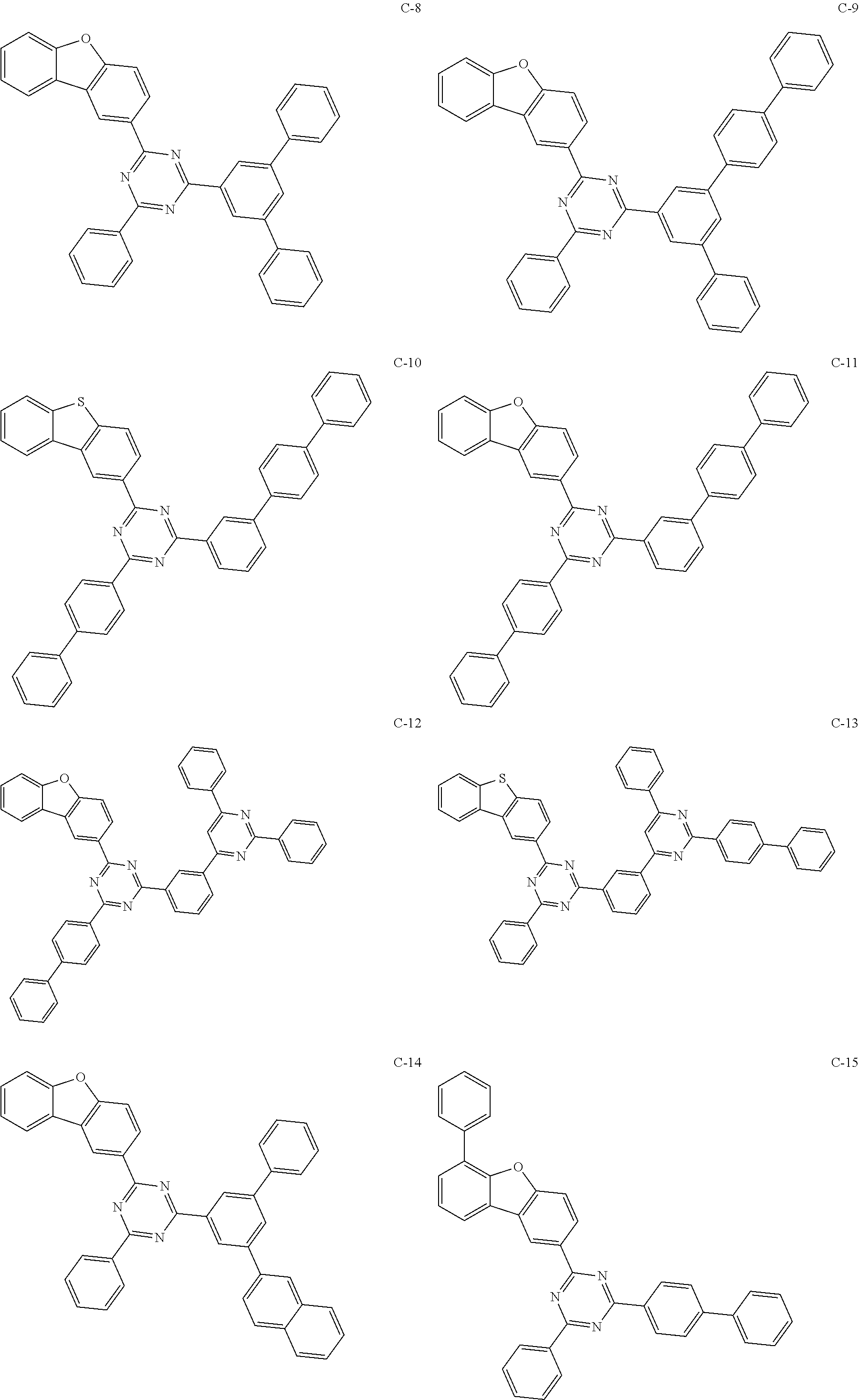

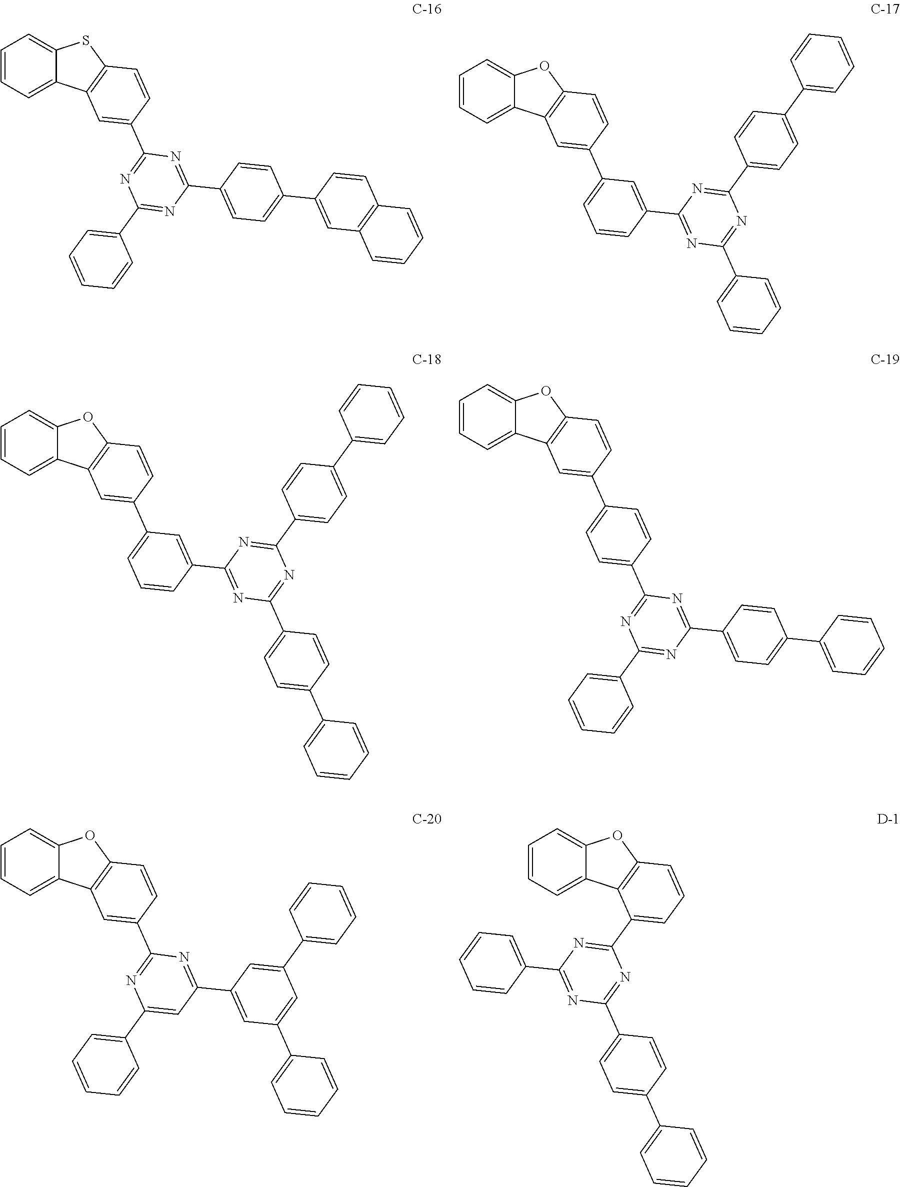 [SMILES: c1ccc(-c2nc(-c3ccc(-c4ccc5ccccc5c4)cc3)nc(-c3ccc4sc5ccccc5c4c3)n2)cc1 ; c1ccc(-c2ccc(-c3nc(-c4ccccc4)nc(-c4cccc(-c5ccc6oc7ccccc7c6c5)c4)n3)cc2)cc1 ; c1ccc(-c2ccc(-c3nc(-c4ccc(-c5ccccc5)cc4)nc(-c4cccc(-c5ccc6oc7ccccc7c6c5)c4)n3)cc2)cc1 ; c1ccc(-c2ccc(-c3nc(-c4ccccc4)nc(-c4ccc(-c5ccc6oc7ccccc7c6c5)cc4)n3)cc2)cc1 ; c1ccc(-c2cc(-c3ccccc3)cc(-c3cc(-c4ccccc4)nc(-c4ccc5oc6ccccc6c5c4)n3)c2)cc1 ; c1ccc(-c2ccc(-c3nc(-c4ccccc4)nc(-c4cccc5oc6ccccc6c45)n3)cc2)cc1]

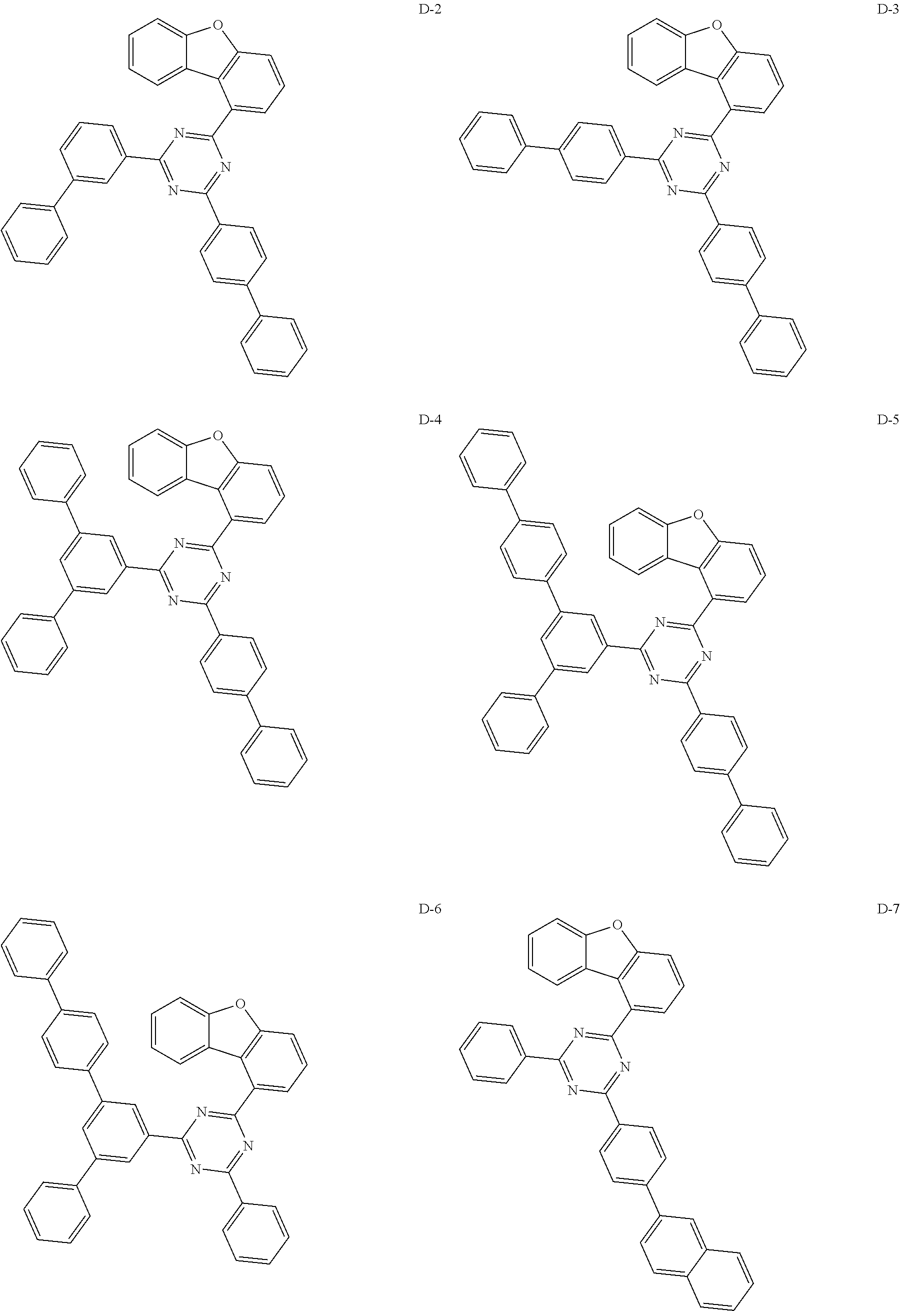 [SMILES: c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cc(-c4ccccc4)cc(-c4nc(-c5ccc(-c6ccccc6)cc5)nc(-c5cccc6oc7ccccc7c56)n4)c3)cc2)cc1 ; c1ccc(-c2ccc(-c3cc(-c4ccccc4)cc(-c4nc(-c5ccccc5)nc(-c5cccc6oc7ccccc7c56)n4)c3)cc2)cc1 ; c1ccc(-c2nc(-c3ccc(-c4ccc5ccccc5c4)cc3)nc(-c3cccc4oc5ccccc5c34)n2)cc1]

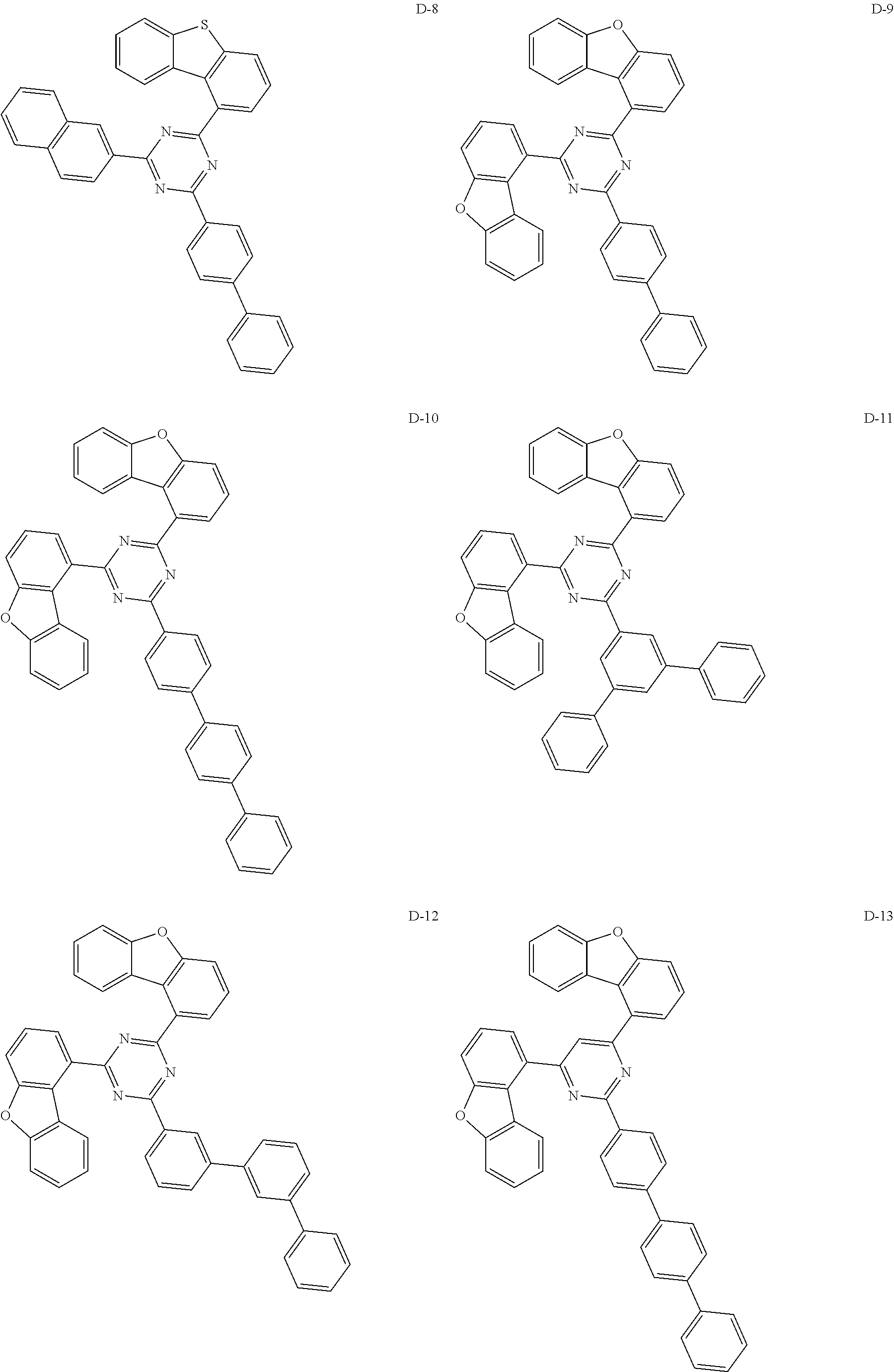 [SMILES: c1ccc(-c2ccc(-c3nc(-c4ccc5ccccc5c4)nc(-c4cccc5sc6ccccc6c45)n3)cc2)cc1 ; c1ccc(-c2ccc(-c3nc(-c4cccc5oc6ccccc6c45)nc(-c4cccc5oc6ccccc6c45)n3)cc2)cc1 ; c1ccc(-c2ccc(-c3ccc(-c4nc(-c5cccc6oc7ccccc7c56)nc(-c5cccc6oc7ccccc7c56)n4)cc3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1 ; c1ccc(-c2cccc(-c3cccc(-c4nc(-c5cccc6oc7ccccc7c56)nc(-c5cccc6oc7ccccc7c56)n4)c3)c2)cc1 ; c1ccc(-c2ccc(-c3ccc(-c4nc(-c5cccc6oc7ccccc7c56)cc(-c5cccc6oc7ccccc7c56)n4)cc3)cc2)cc1]

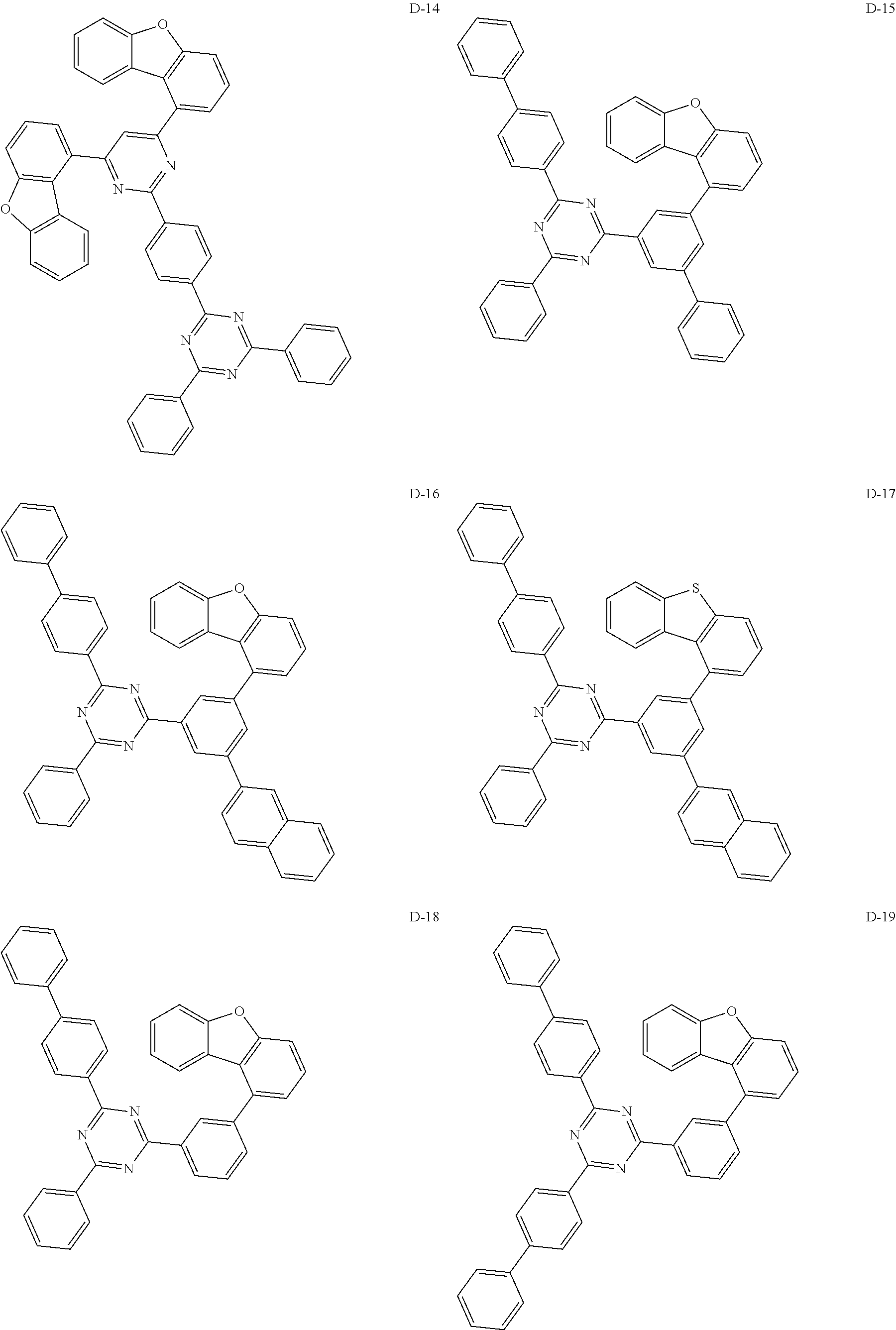

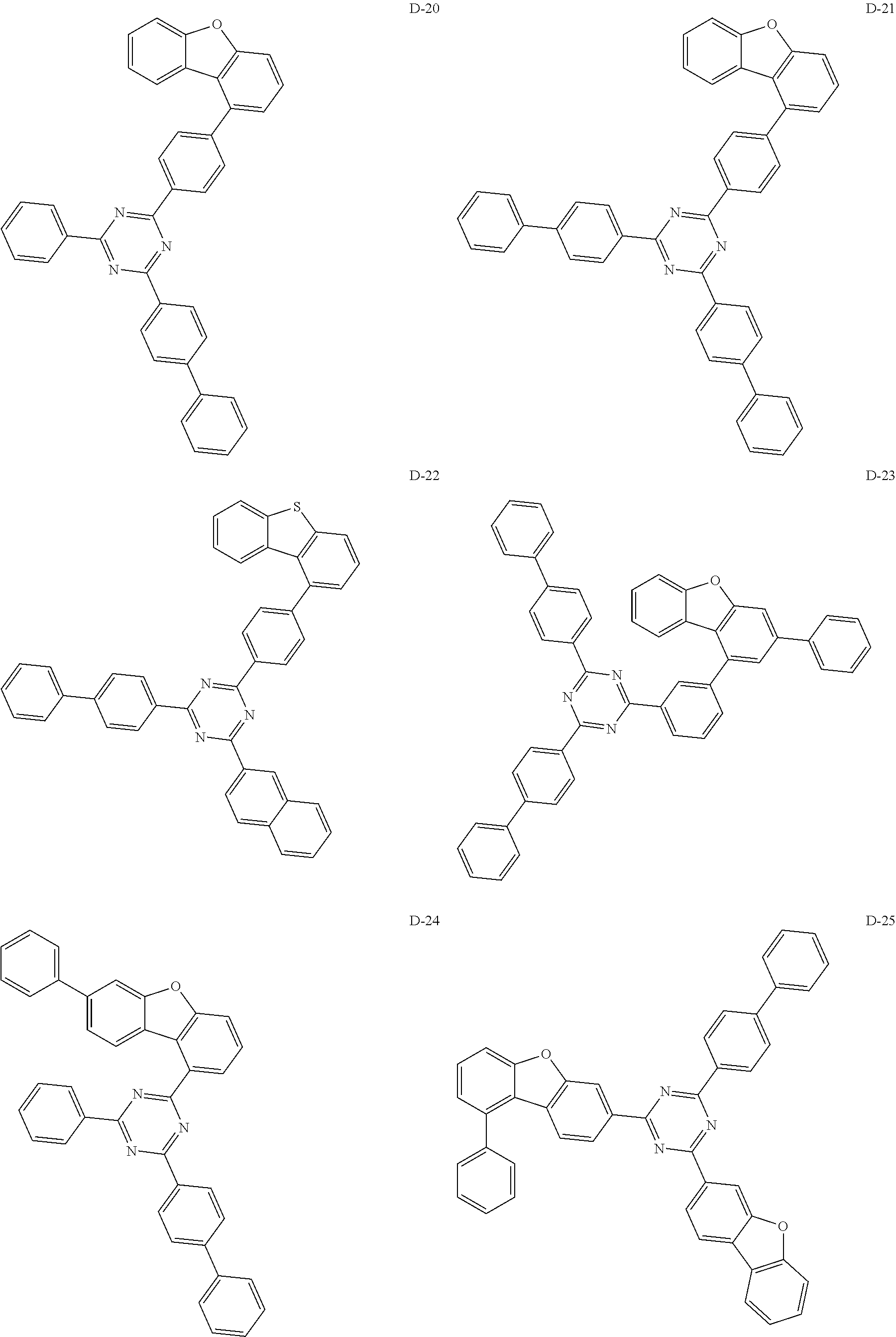

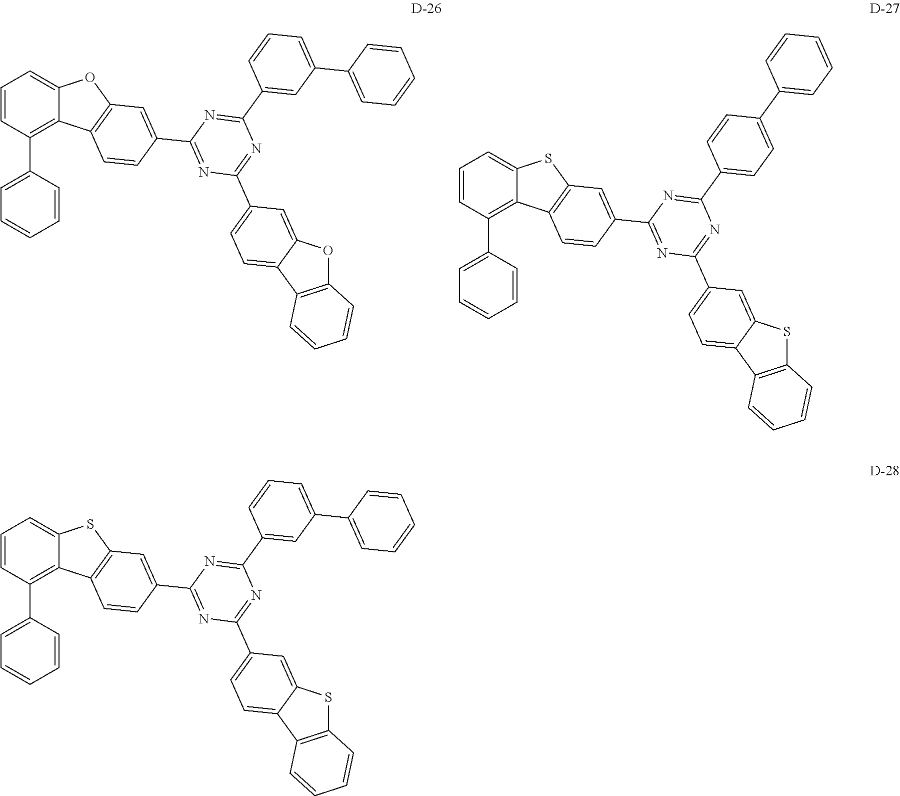 [SMILES: c1ccc(-c2cccc(-c3nc(-c4ccc5c(c4)oc4ccccc45)nc(-c4ccc5c(c4)oc4cccc(-c6ccccc6)c45)n3)c2)cc1 ; c1ccc(-c2ccc(-c3nc(-c4ccc5c(c4)sc4ccccc45)nc(-c4ccc5c(c4)sc4cccc(-c6ccccc6)c45)n3)cc2)cc1 ; c1ccc(-c2cccc(-c3nc(-c4ccc5c(c4)sc4ccccc45)nc(-c4ccc5c(c4)sc4cccc(-c6ccccc6)c45)n3)c2)cc1]

The second compound may be a compound having hole characteristics and may be included with the first compound to provide bipolar characteristics.

The second compound may be, e.g., represented by Chemical Formula 2 or 3.

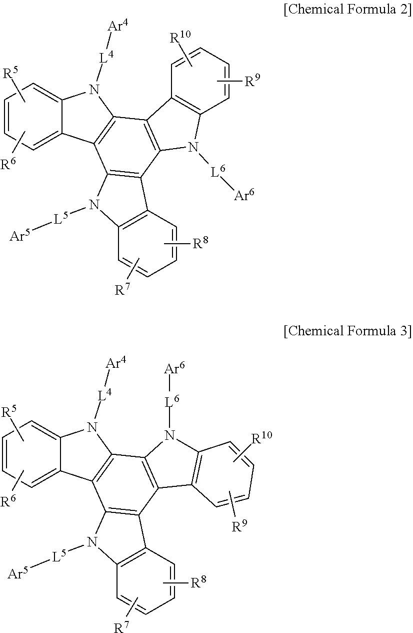

In Chemical Formulae 2 and 3,

Ar⁴ to Ar⁶ may independently be or include, e.g., hydrogen, deuterium, a substituted or unsubstituted C1 to C30 alkyl group, a substituted or unsubstituted C6 to C30 aryl group, a substituted or unsubstituted C3 to C30 heterocyclic group, or a combination thereof,

L⁴ to L⁶ may independently be or include, e.g., a single bond, a substituted or unsubstituted C6 to C20 arylene group, a substituted or unsubstituted divalent C3 to C20 heterocyclic group, or a combination thereof, and

R⁵ to R¹⁰ may independently be or include, e.g., hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof.

R⁵ and R⁶ may be separate or linked with each other to form a ring,

R⁷ and R⁸ may be separate or linked with each other to form a ring, and

R⁹ and R¹⁰ may be separate or linked with each other to form a ring.

The second compound may have good hole characteristics due to fused indolocarbazole structure. Good interface characteristics and hole and electron transport property may be achieved by including the first compound together with the second compound, and thus a device including the compounds may have a lowered driving voltage.

In addition, the second compound may have a relatively high glass transition temperature due to highly rigid planar structure, and crystallinity of the organic compound may be decreased and degradation thereof may be prevented during processes or driving to help thermal stability of the second compound. A device including the second compound may have improved life-span. For example, the second compound may have a glass transition temperature of about 50° C. to about 300° C.

In an implementation, Ar⁴ to Ar⁶ may independently be, e.g., a substituted or unsubstituted C6 to C30 aryl group or a substituted or unsubstituted C3 to C30 heterocyclic group.

In an implementation, Ar⁴ to Ar⁶ may independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a combination thereof.

In an implementation, L⁴ to L⁶ may independently be, e.g., a single bond or a substituted or unsubstituted C6 to C20 arylene group.

In an implementation, L⁴ to L⁶ may independently be, e.g., a single bond, a substituted or unsubstituted phenylene group, a substituted or unsubstituted biphenylene group, a substituted or unsubstituted terphenylene group, or a substituted or unsubstituted naphthylene group.

In an implementation, L⁴ to L⁶ may independently be, e.g., a single bond, a substituted or unsubstituted m-phenylene group, a substituted or unsubstituted p-phenylene group, a substituted or unsubstituted o-phenylene group, a substituted or unsubstituted m-biphenylene group, a substituted or unsubstituted p-biphenylene group, a substituted or unsubstituted o-biphenylene group, a substituted or unsubstituted m-terphenylene group, a substituted or unsubstituted p-terphenylene group, or a substituted or unsubstituted o-terphenylene group. Herein, “substituted” may for example refer to replacement of at least one hydrogen by deuterium, a C1 to C20 alkyl group, a C6 to C20 aryl group, a halogen, a cyano group, or a combination thereof.

In an implementation, the second compound may be, e.g., one of the compounds of Group 2.

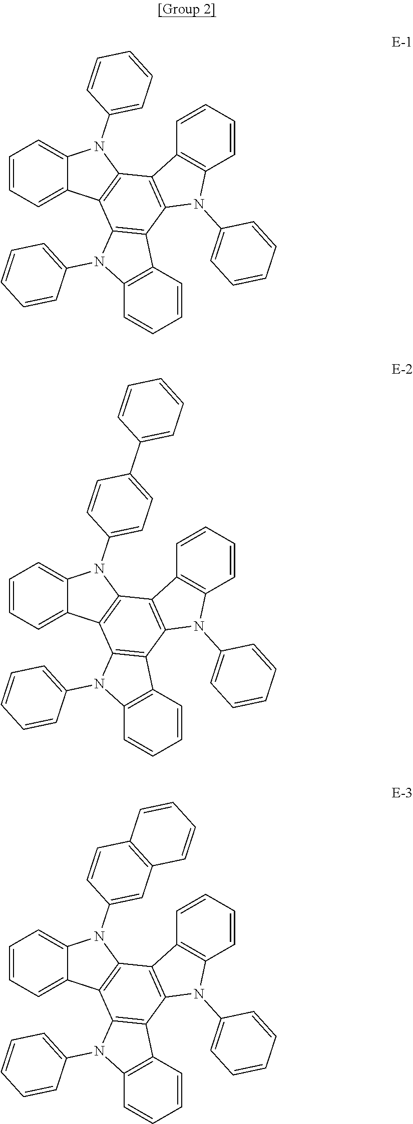

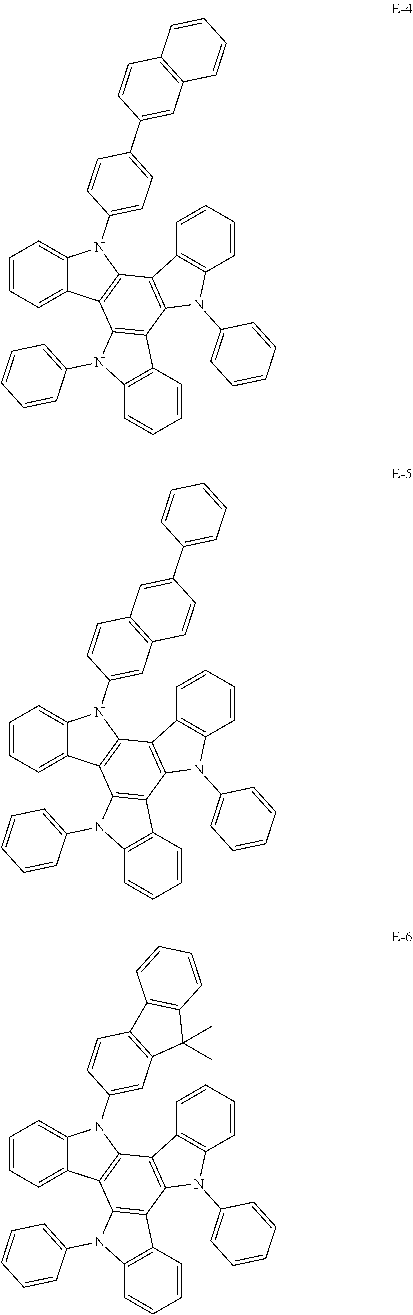 [SMILES: c1ccc(-n2c3ccccc3c3c2c2c4ccccc4n(-c4ccc(-c5ccc6ccccc6c5)cc4)c2c2c4ccccc4n(-c4ccccc4)c32)cc1 ; c1ccc(-c2ccc3cc(-n4c5ccccc5c5c6c(c7ccccc7n6-c6ccccc6)c6c(c7ccccc7n6-c6ccccc6)c54)ccc3c2)cc1 ; CC1(C)c2ccccc2-c2ccc(-n3c4ccccc4c4c5c(c6ccccc6n5-c5ccccc5)c5c(c6ccccc6n5-c5ccccc5)c43)cc21]

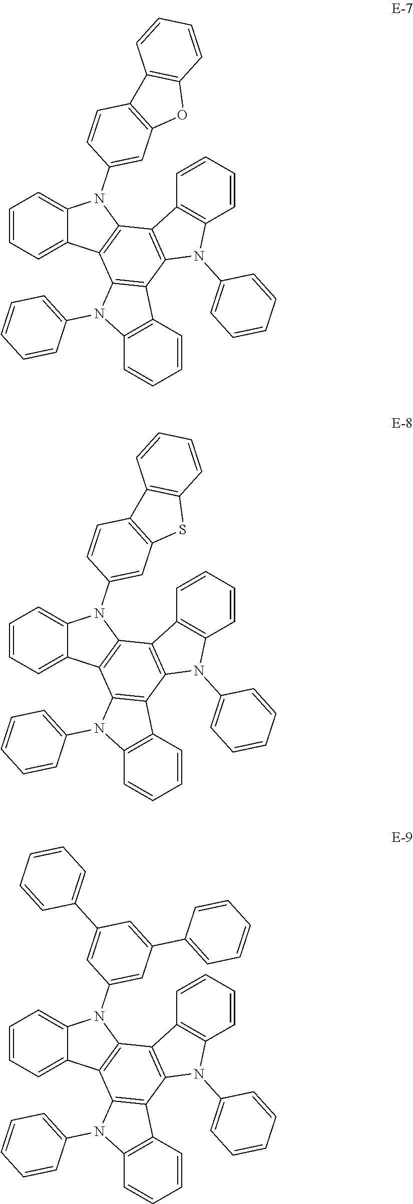 [SMILES: c1ccc(-n2c3ccccc3c3c2c2c4ccccc4n(-c4ccc5c(c4)oc4ccccc45)c2c2c4ccccc4n(-c4ccccc4)c32)cc1 ; c1ccc(-n2c3ccccc3c3c2c2c4ccccc4n(-c4ccc5c(c4)sc4ccccc45)c2c2c4ccccc4n(-c4ccccc4)c32)cc1 ; c1ccc(-c2cc(-c3ccccc3)cc(-n3c4ccccc4c4c5c(c6ccccc6n5-c5ccccc5)c5c(c6ccccc6n5-c5ccccc5)c43)c2)cc1]

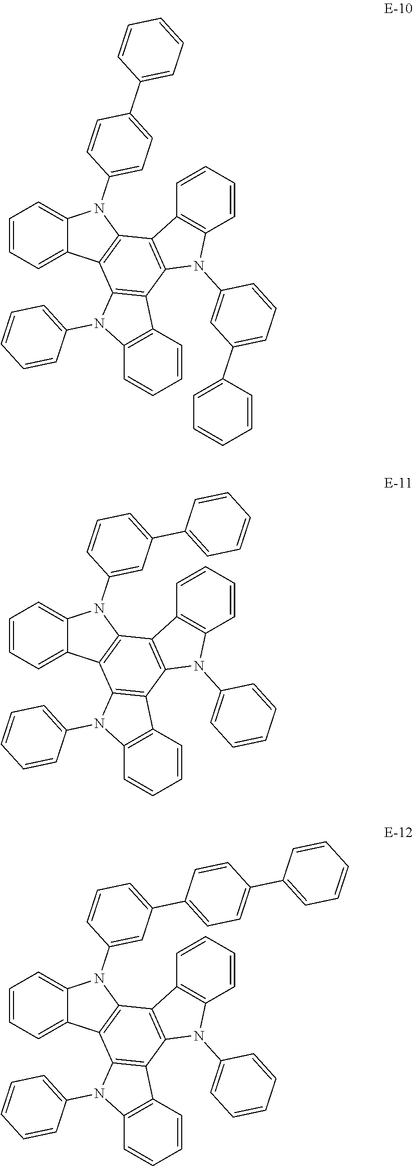 [SMILES: c1ccc(-c2ccc(-n3c4ccccc4c4c3c3c5ccccc5n(-c5cccc(-c6ccccc6)c5)c3c3c5ccccc5n(-c5ccccc5)c43)cc2)cc1 ; c1ccc(-c2cccc(-n3c4ccccc4c4c5c(c6ccccc6n5-c5ccccc5)c5c(c6ccccc6n5-c5ccccc5)c43)c2)cc1 ; c1ccc(-c2ccc(-c3cccc(-n4c5ccccc5c5c6c(c7ccccc7n6-c6ccccc6)c6c(c7ccccc7n6-c6ccccc6)c54)c3)cc2)cc1]

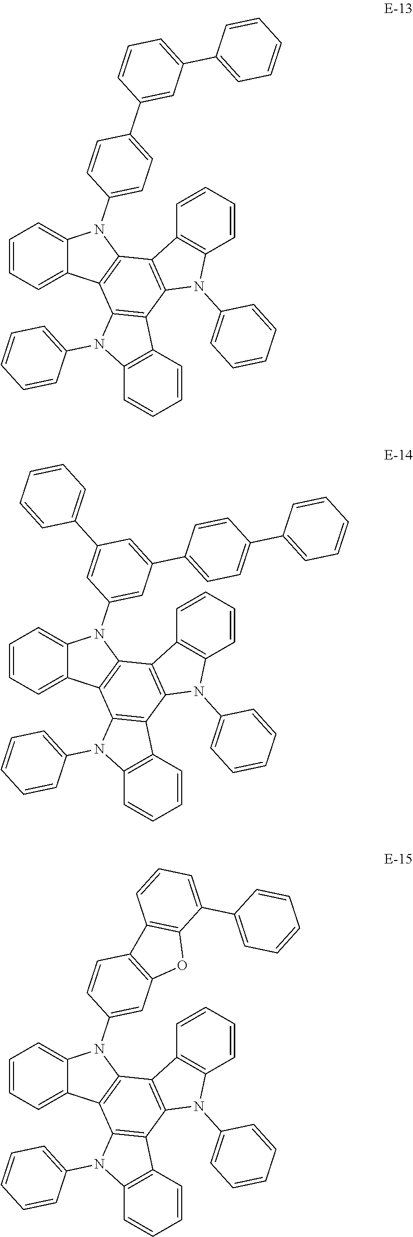 [SMILES: c1ccc(-c2cccc(-c3ccc(-n4c5ccccc5c5c6c(c7ccccc7n6-c6ccccc6)c6c(c7ccccc7n6-c6ccccc6)c54)cc3)c2)cc1 ; c1ccc(-c2ccc(-c3cc(-c4ccccc4)cc(-n4c5ccccc5c5c6c(c7ccccc7n6-c6ccccc6)c6c(c7ccccc7n6-c6ccccc6)c54)c3)cc2)cc1 ; c1ccc(-c2cccc3c2oc2cc(-n4c5ccccc5c5c6c(c7ccccc7n6-c6ccccc6)c6c(c7ccccc7n6-c6ccccc6)c54)ccc23)cc1]

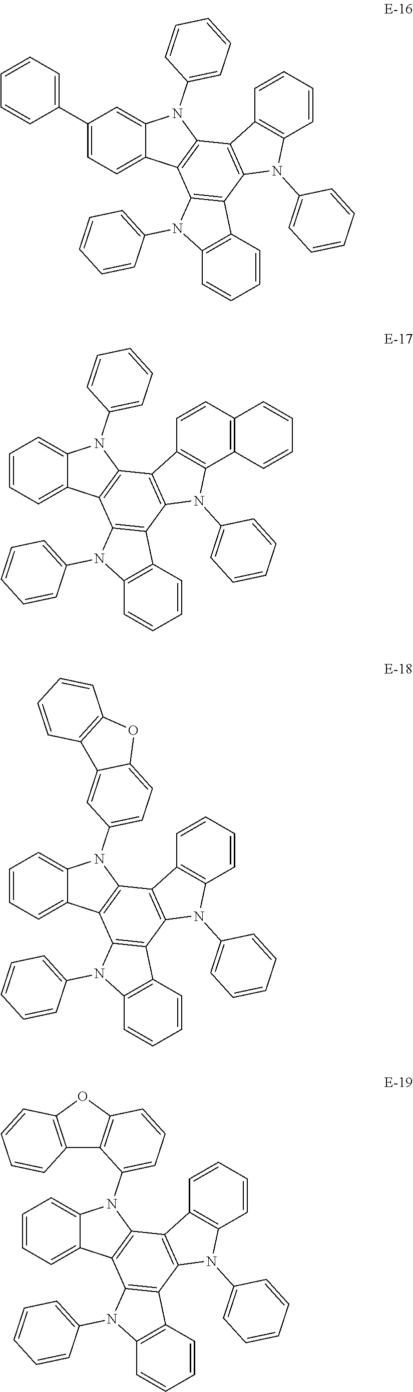 [SMILES: c1ccc(-c2ccc3c4c5c(c6ccccc6n5-c5ccccc5)c5c(c6ccccc6n5-c5ccccc5)c4n(-c4ccccc4)c3c2)cc1 ; c1ccc(-n2c3ccccc3c3c2c2c4ccc5ccccc5c4n(-c4ccccc4)c2c2c4ccccc4n(-c4ccccc4)c32)cc1 ; c1ccc(-n2c3ccccc3c3c2c2c4ccccc4n(-c4ccc5oc6ccccc6c5c4)c2c2c4ccccc4n(-c4ccccc4)c32)cc1 ; c1ccc(-n2c3ccccc3c3c2c2c4ccccc4n(-c4cccc5oc6ccccc6c45)c2c2c4ccccc4n(-c4ccccc4)c32)cc1]

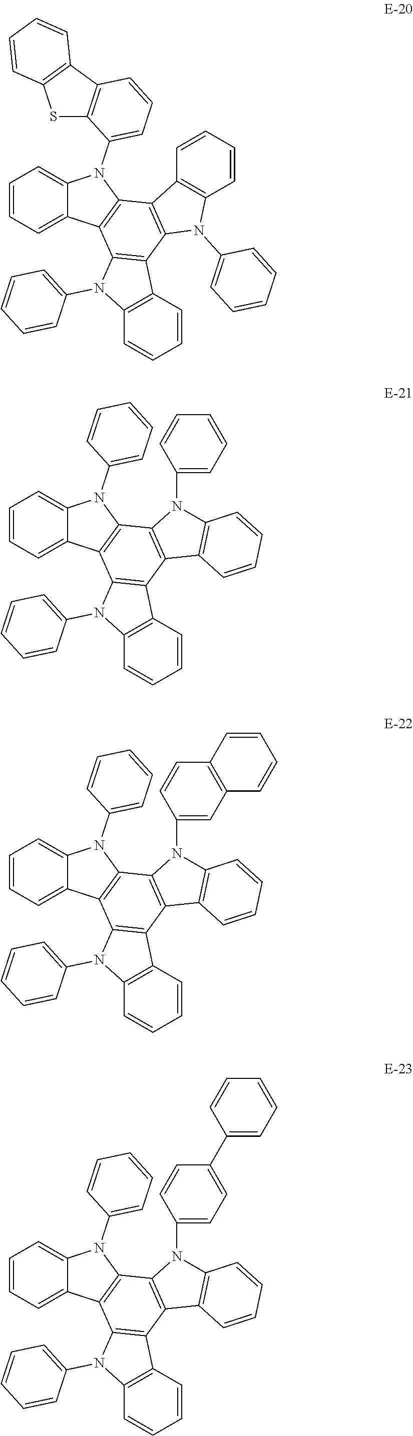 [SMILES: c1ccc(-n2c3ccccc3c3c2c2c4ccccc4n(-c4cccc5c4sc4ccccc45)c2c2c4ccccc4n(-c4ccccc4)c32)cc1 ; c1ccc(-n2c3ccccc3c3c4c5ccccc5n(-c5ccccc5)c4c4c(c5ccccc5n4-c4ccccc4)c32)cc1 ; c1ccc(-n2c3ccccc3c3c4c5ccccc5n(-c5ccc6ccccc6c5)c4c4c(c5ccccc5n4-c4ccccc4)c32)cc1 ; c1ccc(-c2ccc(-n3c4ccccc4c4c5c6ccccc6n(-c6ccccc6)c5c5c6ccccc6n(-c6ccccc6)c5c43)cc2)cc1]

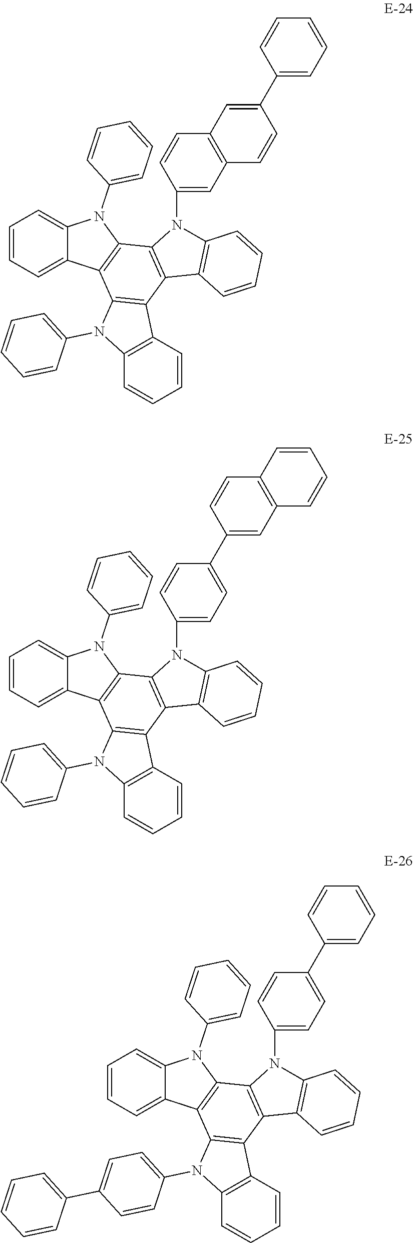 [SMILES: c1ccc(-c2ccc3cc(-n4c5ccccc5c5c6c7ccccc7n(-c7ccccc7)c6c6c7ccccc7n(-c7ccccc7)c6c54)ccc3c2)cc1 ; c1ccc(-n2c3ccccc3c3c4c5ccccc5n(-c5ccc(-c6ccc7ccccc7c6)cc5)c4c4c(c5ccccc5n4-c4ccccc4)c32)cc1 ; c1ccc(-c2ccc(-n3c4ccccc4c4c5c6ccccc6n(-c6ccc(-c7ccccc7)cc6)c5c5c(c6ccccc6n5-c5ccccc5)c43)cc2)cc1]

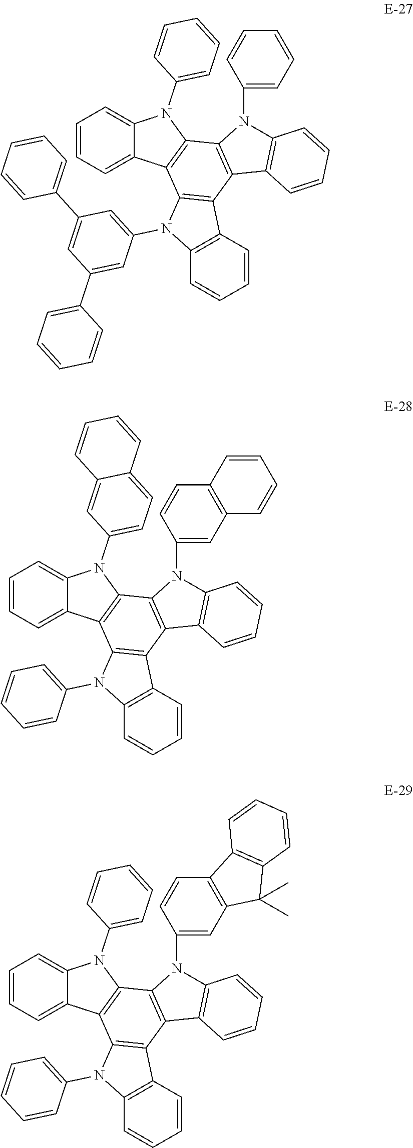 [SMILES: c1ccc(-c2cc(-c3ccccc3)cc(-n3c4ccccc4c4c5c6ccccc6n(-c6ccccc6)c5c5c(c6ccccc6n5-c5ccccc5)c43)c2)cc1 ; c1ccc(-n2c3ccccc3c3c4c5ccccc5n(-c5ccc6ccccc6c5)c4c4c(c5ccccc5n4-c4ccc5ccccc5c4)c32)cc1 ; CC1(C)c2ccccc2-c2ccc(-n3c4ccccc4c4c5c6ccccc6n(-c6ccccc6)c5c5c6ccccc6n(-c6ccccc6)c5c43)cc21]

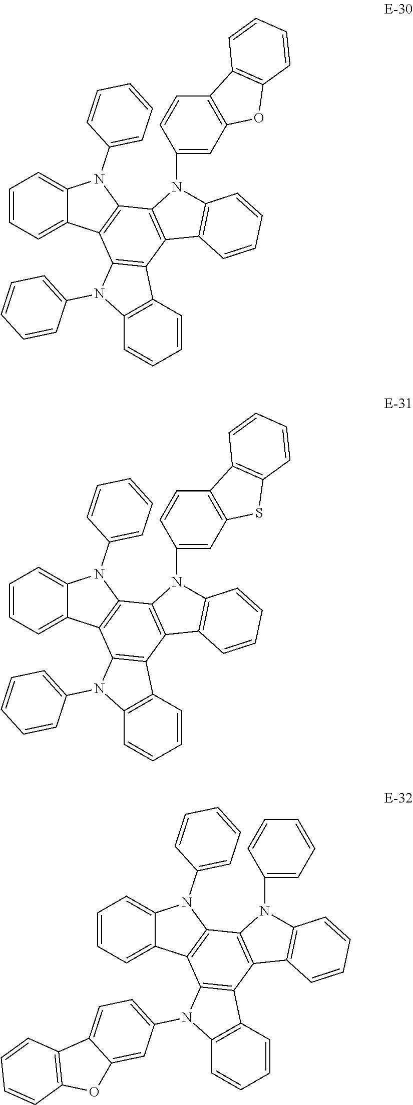 [SMILES: c1ccc(-n2c3ccccc3c3c4c5ccccc5n(-c5ccc6c(c5)oc5ccccc56)c4c4c(c5ccccc5n4-c4ccccc4)c32)cc1 ; c1ccc(-n2c3ccccc3c3c4c5ccccc5n(-c5ccc6c(c5)sc5ccccc56)c4c4c(c5ccccc5n4-c4ccccc4)c32)cc1 ; c1ccc(-n2c3ccccc3c3c4c5ccccc5n(-c5ccc6c(c5)oc5ccccc56)c4c4c5ccccc5n(-c5ccccc5)c4c32)cc1]

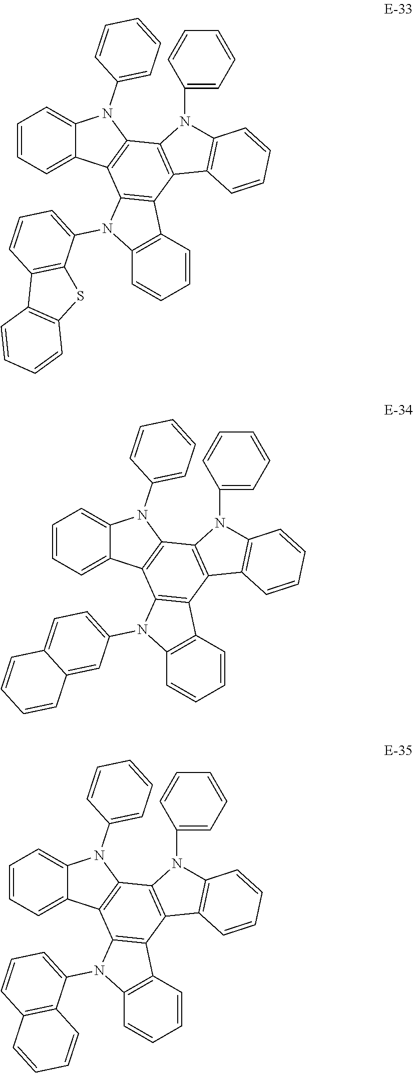 [SMILES: c1ccc(-n2c3ccccc3c3c4c5ccccc5n(-c5cccc6c5sc5ccccc56)c4c4c5ccccc5n(-c5ccccc5)c4c32)cc1 ; c1ccc(-n2c3ccccc3c3c4c5ccccc5n(-c5ccc6ccccc6c5)c4c4c5ccccc5n(-c5ccccc5)c4c32)cc1 ; c1ccc(-n2c3ccccc3c3c4c5ccccc5n(-c5cccc6ccccc56)c4c4c5ccccc5n(-c5ccccc5)c4c32)cc1]

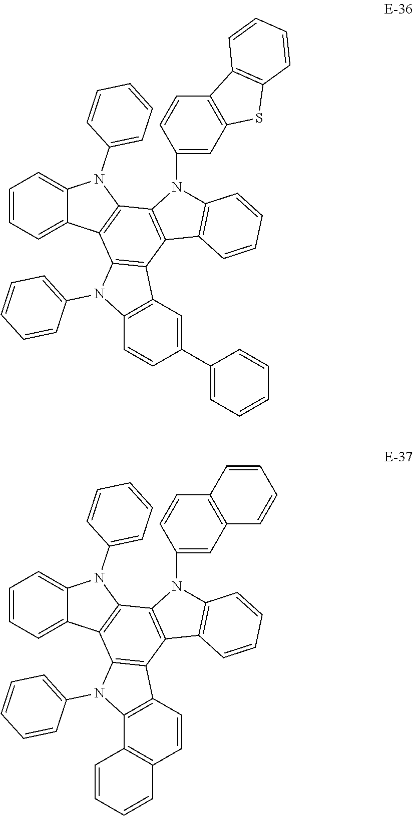 [SMILES: c1ccc(-c2ccc3c(c2)c2c4c5ccccc5n(-c5ccc6c(c5)sc5ccccc56)c4c4c(c5ccccc5n4-c4ccccc4)c2n3-c2ccccc2)cc1 ; c1ccc(-n2c3ccccc3c3c2c2c(c4ccccc4n2-c2ccc4ccccc4c2)c2c4ccc5ccccc5c4n(-c4ccccc4)c23)cc1]

The first compound and the second compound may be included, e.g., in a weight ratio of about 1:99 to about 99:1. Within the range, a desirable weight ratio may be adjusted using an electron transport property of the first compound and a hole transport property of the second compound to realize bipolar characteristics and thus to improve efficiency and a life-span. In an implementation, they may be, e.g., included in a weight ratio of about 10:90 to about 90:10, about 20:80 to about 80:20, about 30:70 to about 70:30, about 40:60 to about 60:40, or about 50:50.

In an implementation, the composition may further include at least one compound in addition to the first compound and the second compound.

The composition may further include a dopant. The dopant may be, e.g., a phosphorescent dopant. In an implementation, the dopant may be, e.g., a red, green, or blue phosphorescent dopant. In an implementation, the dopant may be, e.g., a red phosphorescent dopant.

The dopant is a material mixed with the first compound and the second compound in a small amount to cause light emission, e.g., a material such as a metal complex that emits light by multiple excitation into a triplet or more. The dopant may be, e.g., an inorganic, organic, or organic/inorganic compound, and one or more kinds thereof may be used.

The dopant may include a phosphorescent dopant. Examples of the phosphorescent dopant may include organometallic compounds including Ir, Pt, Os, Ti, Zr, Hf, Eu, Tb, Tm, Fe, Co, Ni, Ru, Rh, Pd, or a combination thereof. In an implementation, the phosphorescent dopant may be, e.g., a compound represented by Chemical Formula Z.

L₂MX [Chemical Formula Z]

In Chemical Formula Z, M may be a metal, and L and X may independently be a ligand to form a complex compound with M.

The M may be, e.g., Ir, Pt, Os, Ti, Zr, Hf, Eu, Tb, Tm, Fe, Co, Ni, Ru, Rh, Pd, or a combination thereof, and L and X may be, e.g., a bidendate ligand.

The composition may be formed by a dry film formation method such as chemical vapor deposition (CVD).

Hereinafter, an organic optoelectronic device including the composition is described.

The organic optoelectronic device may be a device to convert electrical energy into photoenergy and vice versa, and may be, e.g., an organic photoelectric device, an organic light emitting diode, an organic solar cell, or an organic photo conductor drum.

Herein, an organic light emitting diode as one example of an organic optoelectronic device is described referring to drawings.

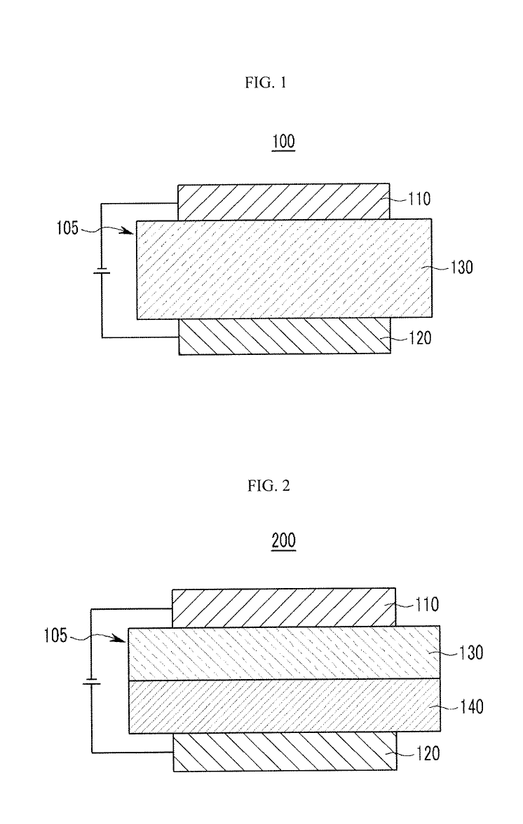

FIGS. 1 and 2 illustrate cross-sectional views showing organic light emitting diodes according to embodiments.

Referring to FIG. 1, an organic light emitting diode 100 according to an embodiment may include an anode 120 and a cathode 110 and facing each other and an organic layer 105 disposed between the anode 120 and the cathode 110.

The anode 120 may be made of a conductor having a high work function to help hole injection, and may be, e.g., a metal, a metal oxide and/or a conductive polymer. The anode 120 may be, for example a metal such as nickel, platinum, vanadium, chromium, copper, zinc, gold, and the like or an alloy thereof; metal oxide such as zinc oxide, indium oxide, indium tin oxide (ITO), indium zinc oxide (IZO), and the like; a combination of metal and oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline.

The cathode 110 may be made of a conductor having a low work function to help electron injection, and may be for example a metal, a metal oxide and/or a conductive polymer. The cathode 110 may be for example a metal such as magnesium, calcium, sodium, potassium, titanium, indium, yttrium, lithium, gadolinium, aluminum silver, tin, lead, cesium, barium, and the like or an alloy thereof; a multi-layer structure material such as LiF/Al, LiO₂/Al, LiF/Ca, LiF/Al and BaF₂/Ca.

The organic layer 105 may include a light emitting layer 130.

The light emitting layer 130 may include, e.g., the composition according to an embodiment.

Referring to FIG. 2, an organic light emitting diode 200 may further include, e.g., a hole auxiliary layer 140 in addition to the light emitting layer 130. The hole auxiliary layer 140 may further increase hole injection and/or hole mobility and may block electrons between the anode 120 and the light emitting layer 130. The hole auxiliary layer 140 may be for example a hole transport layer, a hole injection layer, and/or an electron blocking layer, and may include at least one layer.

In an implementation, an organic light emitting diode may further include an electron transport layer, an electron injection layer, a hole injection layer, and the like in the organic layer 105.

The organic light emitting diodes 100 and 200 may be manufactured by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Preparation of First Host

### SYNTHESIS EXAMPLE 1

### Synthesis of Intermediate 1

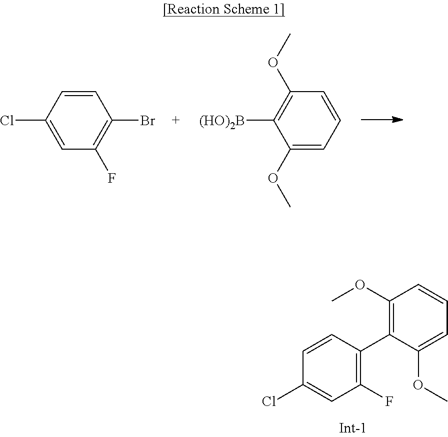

1-bromo-4-chloro-2-fluorobenzene (61 g, 291 mmol), 2,6-dimethoxyphenylboronic acid (50.4 g, 277 mmol), K₂CO₃ (60.4 g, 437 mmol), and Pd(PPh₃)₄ (10.1 g, 8.7 mmol) were added into a round-bottomed flask and dissolved in THF (500 ml) and distilled water (200 ml) and then stirred under reflux at 60° C. for 12 hours. When the reaction was completed, an aqueous layer was removed to obtain 38 g (51%) of an intermediate (Int-1) using a column chromatography (hexane:DCM (20%)).

### SYNTHESIS EXAMPLE 2

### Synthesis of Intermediate 2

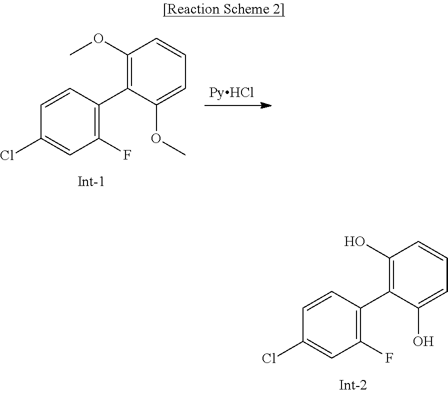 [SMILES: COc1cccc(OC)c1-c1ccc(Cl)cc1F ; Oc1cccc(O)c1-c1ccc(Cl)cc1F]

The intermediate (Int-1) (38 g, 142 mmol) and pyridine hydrochloride (165 g, 1425 mmol) were added into a round-bottomed flask and stirred under reflux at 200° C. for 24 hours. When the reaction was completed, it was cooled down at ambient temperature and then slowly poured into distilled water and stirred for 1 hour. The solid was filtered to provide 23 g (68%) of an intermediate (Int-2).

### SYNTHESIS EXAMPLE 3

### Synthesis of Intermediate 3

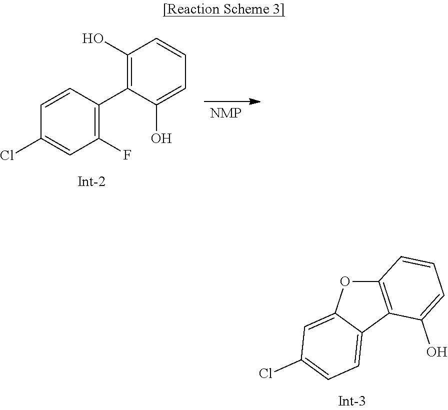 [SMILES: Oc1cccc(O)c1-c1ccc(Cl)cc1F ; Oc1cccc2oc3cc(Cl)ccc3c12]

The intermediate (Int-2) (23 g, 96 mmol) and K₂CO₃ (20 g, 144 mmol) were added into a round-bottomed flask and dissolved in NMP (100 ml) and then, stirred and refluxed at 180° C. for 12 hours. When the reaction was completed, the mixture was poured into an excess of distilled water. The solid was filtered and dissolved in ethyl acetate and then dried with MgSO₄, and an organic layer was removed under reduced pressure. Using a column chromatography (hexane:EA (30%)), 16 g (76%) of an intermediate (Int-3) was obtained.

### SYNTHESIS EXAMPLE 4

### Synthesis of Intermediate 4

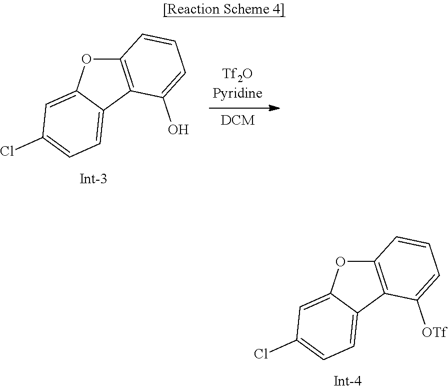 [SMILES: Oc1cccc2oc3cc(Cl)ccc3c12 ; O=S(=O)(Oc1cccc2oc3cc(Cl)ccc3c12)C(F)(F)F]

The intermediate (Int-3) (16 g, 73 mmol) and pyridine (12 ml, 146 mmol) were added into a round-bottomed flask and dissolved in DCM (200 ml). The temperature was cooled down to 0° C. and then trifluoromethane sulfonic anhydride (14.7 ml, 88 mmol) was slowly added in a dropwise fashion. After stirring for 6 hours, when the reaction is completed, an excess of distilled water was added and stirred for 30 minutes, and then it was extracted by DCM. The organic solvent was removed under reduced pressure, and it was vacuum-dried to provide 22.5 g (88%) of an intermediate (Int-4).

### SYNTHESIS EXAMPLE 5

### Synthesis of Intermediate 5

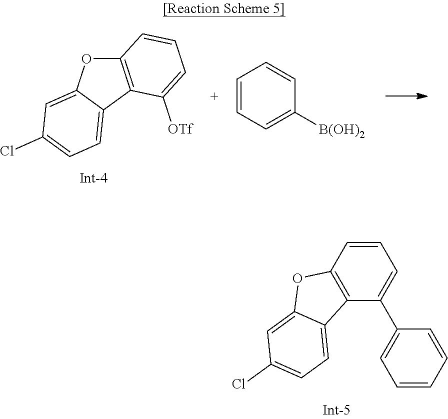 [SMILES: O=S(=O)(Oc1cccc2oc3cc(Cl)ccc3c12)C(F)(F)F ; OB(O)c1ccccc1 ; Clc1ccc2c(c1)oc1cccc(-c3ccccc3)c12]

14.4 g (81%) of an intermediate (Int-5) was obtained in accordance with the same procedure as in Synthesis Example 1, using the intermediate (Int-4) (22.5 g, 64 mmol) and phenyl boronic acid (7.8 g, 64 mmol), K₂CO₃ (13.3 g, 96 mmol), and Pd(PPh₃)₄ (3.7 g, 3.2 mmol).

### SYNTHESIS EXAMPLE 6

### Synthesis of Intermediate 6

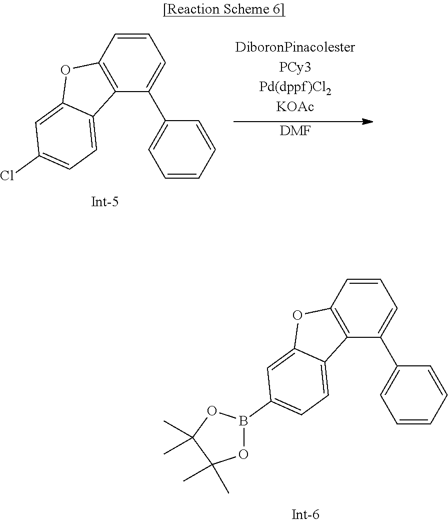

The intermediate (Int-5) (22.5 g, 80 mmol), bis(pinacolato)diboron (24.6 g, 97 mmol), Pd(dppf)Cl₂ (2 g, 2.4 mmol), tricyclohexylphosphine (3.9 g, 16 mmol), and potassium acetate (16 g, 161 mmol) were added to a round-bottomed flask and dissolved by DMF (320 ml). The mixture was stirred under reflux at 120° C. for 10 hours. When the reaction is completed, the mixture was poured into an excess of distilled water and stirred for 1 hour. The solid was filtered and dissolved in DCM. Moisture was removed using MgSO₄, and then an organic solvent was filtered using a silica gel pad and then removed under reduced pressure. The solid was recrystallized by EA and hexane to provide a 26.9 g (90%) of an intermediate (Int-6).

### SYNTHESIS EXAMPLE 7

### Synthesis of Intermediate 7

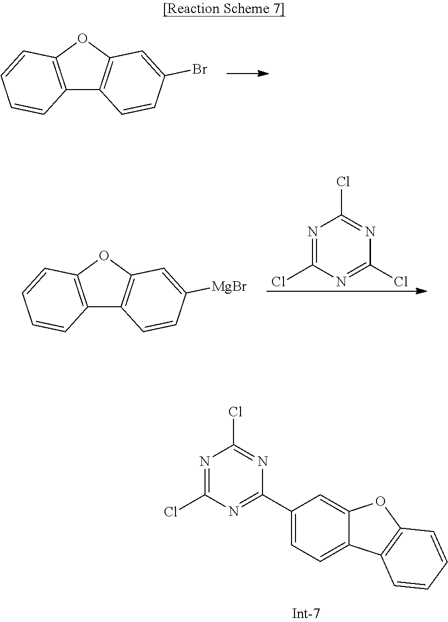

Mg (4.9 g, 202 mmol) was put in a round-bottomed flask under a nitrogen atmosphere and then heated with a heat gun. When cooled down to a moderate temperature, iodine (0.5 g, 2 mmol) and 30 ml of THF were subsequently added thereto, and the mixture was stirred. Subsequently, 3-bromodibenzofuran (50 g, 202 mmol) dissolved in 100 ml of THF was put in a dropping funnel and then, slowly added to the round-bottomed flask in a dropwise fashion. The mixed solution was stirred until it started to be transparent and generated heat and then, became completely transparent. In a new round-bottomed flask, cyanuric chloride (37.3 g, 202 mmol) was dissolved in 200 ml of THF, and the solution was put under an ice bath condition. Then, a Grignard reagent prepared in advance was slowly added thereto in a dropwise fashion. The obtained mixture was additionally stirred for about 1 hour, and distilled water was added thereto to complete a reaction. Subsequently, an organic layer was separated therefrom, and a solvent was removed under a reduced pressure. Then, a small amount of DCM was added thereto to dissolve a resultant therefrom, the solution was added to an excess of methanol in a dropwise fashion to precipitate a solid, and the solid was filtered to obtain 38 g (60%) of an intermediate (Int-7).

### SYNTHESIS EXAMPLE 8

### Synthesis of Intermediate 8

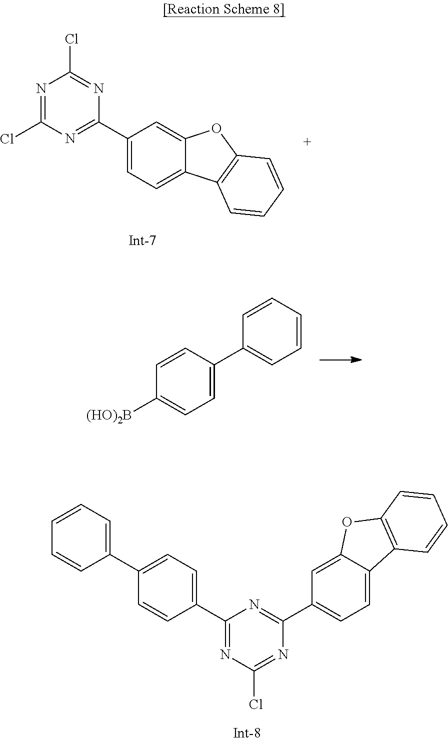 [SMILES: Clc1nc(Cl)nc(-c2ccc3c(c2)oc2ccccc23)n1 ; OB(O)c1ccc(-c2ccccc2)cc1 ; Clc1nc(-c2ccc(-c3ccccc3)cc2)nc(-c2ccc3c(c2)oc2ccccc23)n1]

The intermediate (Int-7) (19 g, 60 mmol), 4-biphenylboronic acid (11.9 g, 60 mmol), K₂CO₃ (12.5 g, 90 mmol), and Pd(PPh₃)₄ (3.5 g, 3 mmol) were added, under a nitrogen atmosphere, to a round-bottomed flask according to the same method as Synthesis Example 1 to synthesize 15.1 g (58%) of an intermediate (Int-8).

### SYNTHESIS EXAMPLE 9

### Synthesis of Compound D-25

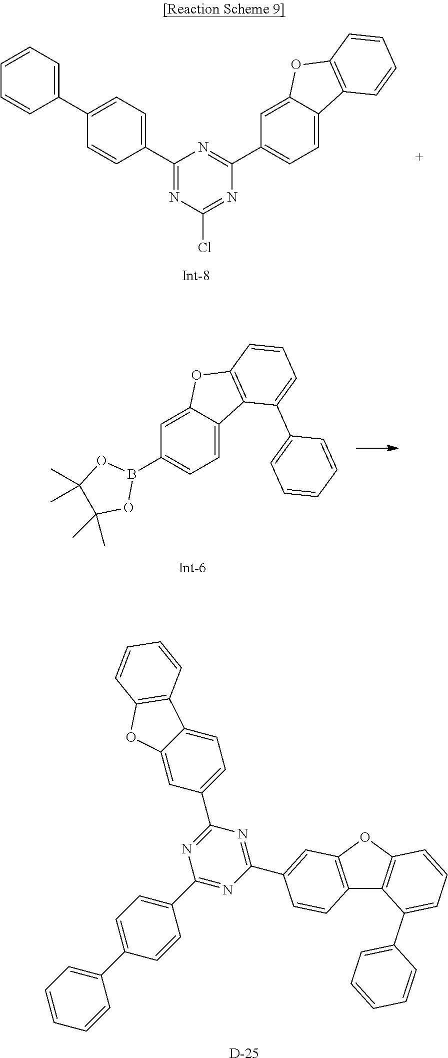

The intermediate (Int-8) (15 g, 35 mmol), the intermediate (Int-6) (12.8 g, 35 mmol), K₂CO₃ (7.2 g, 52 mmol), and Pd(PPh₃)₄ (2 g, 1.7 mmol) were added, under a nitrogen atmosphere, to a round-bottomed flask according to the same method as Synthesis Example 1 to synthesize 15.5 g (70%) of Compound D-25.

LC/MS calculated for: C₄₅H₂₇N₃O₂ Exact Mass: 641.21 found for: 642.64

Preparation of Second Host

### SYNTHESIS EXAMPLE 10

### Synthesis of Compound E-21

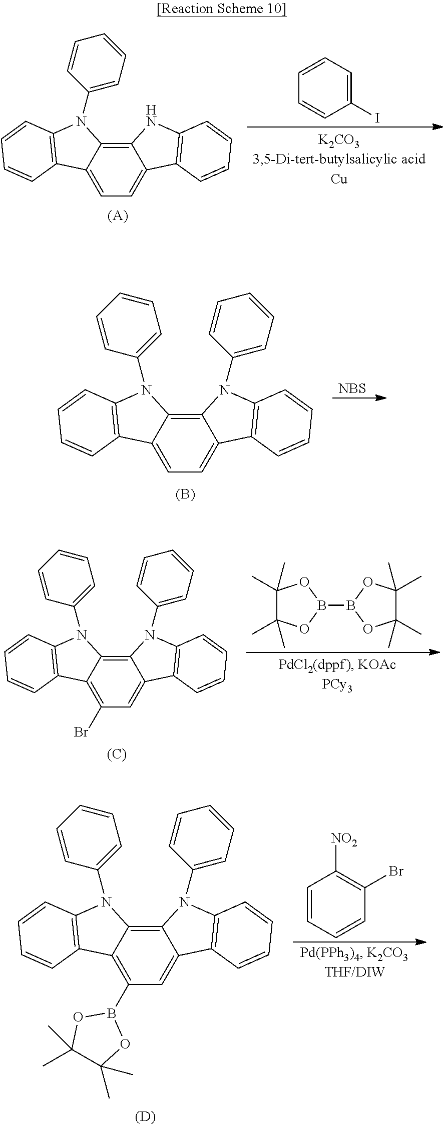

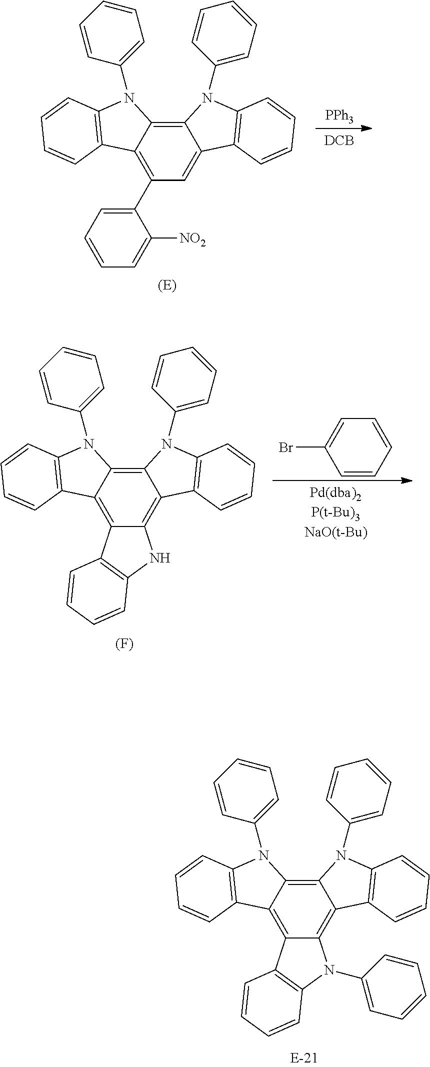

First Step; Synthesis of Intermediate Product (B)

100 g (0.301 mol) of a starting material (A), 122.75 g (0.602 mol) of iodobenzene, 3.82 g (0.06 mol) of Cu, 15.06 g (0.06 mol) of 3,5-di-tert-butylsalicylic acid, and 62.37 g (0.451 mol) of K₂CO₃ were put in a round-bottomed flask, 750 ml of dodecylbenzene was added thereto, and the mixture was refluxed and stirred under a nitrogen atmosphere for 48 hours. When a reaction was complete, an excess of methanol was added to precipitate a solid, and the solid was filtered. The solid was dissolved in 1,400 ml of chlorobenzene and filtered through silica gel to precipitate a white solid and obtain 107.3 g (yield: 87%) of an intermediate product (B).

Second Step; Synthesis of Intermediate Product (C)

107.3 g (0.263 mol) of the intermediate (B) was dissolved in 1,300 mL of dichloromethane, another solution prepared by dissolving 44.41 g (0.25 mol of N-bromosuccinimide in dimethyl formamide was slowly added thereto for 4 hours, while the fomer solution was stirred at 0° C. The reactants were stirred at ambient temperature for 2 hours and then, extracted with distilled water and dichloromethane. An organic layer therefrom was dried with potassium carbonate, filtered, and concentrated under a reduced pressure. A product therefrom was recrystallized with dichloromethane and n-hexane to obtain 122.7 g (yield: 96%) of an intermediate product (C) as a white solid.

Third Step; Synthesis of Intermediate Product (D)

122.7 g (0.252 mol) of the intermediate (C), 12.34 g (0.015 mol) of Pd(dppf)Cl₂, 83.11 g (0.327 mol) of bis(pinacolato)diboron, 98.15 g (0.755 mol) of potassium acetate, and 14.12 g (0.05 mol) of PCy₃ were dissolved in 1,260 ml of dimethyl formamide. The reactants were refluxed and stirred under a nitrogen atmosphere for 12 hours, and distilled water was added thereto to complete a reaction. The resultant was concentrated under a reduced pressure with dimethyl formamide and extracted three times with dichloromethane. An extraction solution was dried with magnesium sulfite and filtered, and a filtrate therefrom was concentrated under a reduced pressure. A product therefrom was purified with n-hexane/dichloromethane (9:1 volume ratio) through silica gel column chromatography to obtain 100 g (yield: 74%) of intermediate product (D) as a white solid.

Fourth Step; Synthesis of Intermediate Product (E)

67 g (0.125 mol) of the intermediate (D), 25.32 g (0.125 mol) of 1-bromo-2-nitrobenzene, 43.32 g (0.313 mol) of potassium carbonate, and 7.24 g (0.006 mmol) of tetrakis(triphenylphosphine)palladium were suspended in 600 ml of 1,4-dioxane and 200 ml of distilled water and then, suspended and stirred for 12 hours. When a reaction was complete, the resultant was concentrated under a reduced pressure to remove dioxane and then, extracted with dichloromethane and distilled water, and an organic layer therefrom was filtered with silica gel. After removing an organic solvent therefrom, the rest thereof was silica gel columned with n-hexane/dichloromethane (2:8 volume ratio) to obtain 40 g (yield: 60%) of an intermediate product (E).

Fifth Step; Synthesis of Intermediate Product (F)

18.6 g (0.035 mol) of the intermediate (E) and 36.85 g (0.14 mol) of triphenylphosphine were dissolved in 120 ml of dichlorobenzene, and the solution was stirred under a nitrogen atmosphere for 12 hours at 200° C. When a reaction was complete, the resultant was concentrated under a reduced pressure to remove dichlorobenzene, and a solid was extracted by adding an excess of n-hexane thereto and filtered. A product was dissolved in 500 ml of toluene and then, filtered with silica gel, and a filtrate therefrom was concentrated under a reduced pressure. A product therefrom was recrystallized with dichloromethane and n-hexane to obtain 14.5g (yield: 83%) of an intermediate product (F) as a light yellow solid.

Sixth Step; Synthesis of Compound E-21

7.5 g (0.015 mol) of the intermediate (F), 3.55 g (0.023 mol) of bromobenzene, and 2.17g (0.023 mol of NaO(t-Bu)₃ were dissolved in 70 ml of xylene. Subsequently, 0.52 g (0.001 mol) of Pd(dba)₂ and 1.83 g (0.005 mol) of P(t-Bu)₃ were sequentially added thereto, and the obtained mixture was refluxed and stirred under a nitrogen atmosphere for 12 hours. When a reaction was complete, an excess of methanol was added thereto to precipitate a solid. The solid was filtered, dissolved in toluene, and silica gel-filtered, and a filtrate was concentrated under a reduced pressure. A product therefrom was recrystallized with dichloromethane and n-hexane to obtain 7.9 g (yield: 91%) of Compound E-21 as a white solid.

### SYNTHESIS EXAMPLE 11

### Synthesis of Compound E-34

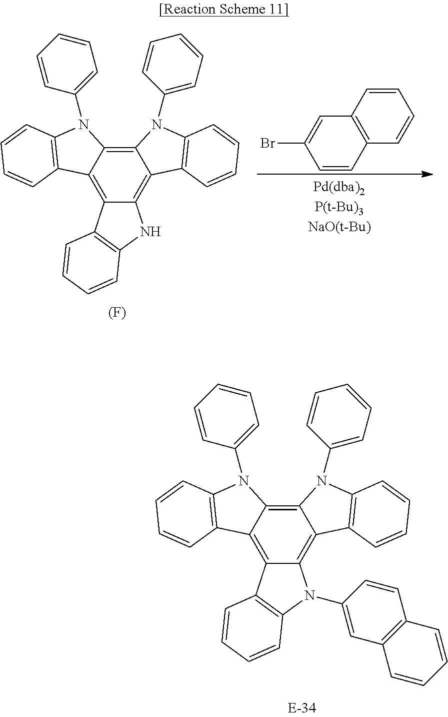

7.5 g (0.015 mol) of the intermediate (F), 4.68 g (0.023 mol) of 2-bromonaphthalene, and 2.17 g (0.023 mol) of NaO(t-Bu)₃ were dissolved in 70 ml of xylene. Then, 0.52 g (0.001 mol) of Pd(dba)₂ and 1.83 g (0.005 mol) of P(t-Bu)₃ were sequentially added thereto, and the obtained mixture was refluxed and stirred under a nitrogen atmosphere for 12 hours. When a reaction was complete, an excess of methanol was added thereto to precipitate a solid. The solid was filtered, dissolved in toluene, and silica gel-filtered, and a filtrate therefrom was concentrated under a reduced pressure. A product therefrom was recrystallized with dichloromethane and n-hexane to obtain 8.3 g (yield: 88%) of Compound E-34 as a white solid.

Manufacture of Organic Light Emitting Diode

### EXAMPLE 1

A glass substrate coated with ITO (indium tin oxide) as a 1,500 Å-thick thin film was washed with distilled water. After washing with the distilled water, the glass substrate was ultrasonic wave-washed with a solvent such as isopropyl alcohol, acetone, methanol, and the like and dried and then, moved to a plasma cleaner, cleaned by using oxygen plasma for 10 minutes, and moved to a vacuum depositor. This obtained ITO transparent electrode was used as an anode, Compound A was vacuum-deposited on the ITO substrate to form a 700 Å-thick hole injection layer, Compound B was deposited to be 50 Å thick on the injection layer, and Compound C was deposited to be 700 Å thick to form a hole transport layer. On the hole transport layer, a 400 A-thick hole transport auxiliary layer was formed by vacuum-depositing Compound C-1. On the hole transport auxiliary layer, a 400 Å-thick light emitting layer was formed by vacuum-depositing Compound D-25 of Synthesis Example 9 and Compound E-21 of Synthesis Example 10 simultaneously as a host and doping 2 wt % of [Ir(piq)₂acac] as a dopant. Herein, Compound D-25 and Compound E-21 were used in a weight ratio of 5:5, and their weight ratios are separately in the following examples. Subsequently, on the light emitting layer, a 300 Å-thick electron transport layer was formed by simultaneously vacuum-depositing Compound D and Liq in a ratio of 1:1, and on the electron transport layer, Liq and Al were sequentially vacuum-deposited to be 15 Å thick and 1,200 Å thick, manufacturing an organic light emitting diode.

The organic light emitting diode included a five-layered organic thin layer, and specifically the following structure.

ITO/Compound A (700 Å)/Compound B (50 Å)/Compound C (700 Å)/Compound C-1 (400 A)/EML [Compound D-25: E-21: [Ir(piq)₂acac] (2 wt %)] 400 Å/Compound D: Liq (300 Å)/Liq (15 Å)/Al (1,200 Å).

Compound A: N4,N4′-diphenyl-N4,N4′-bis(9-phenyl-9H-carbazol-3-yl)biphenyl-4,4′-diamine

Compound B: 1,4,5,8,9,11-hexaazatriphenylene-hexacarbonitrile (HAT-CN)

Compound C: N-(biphenyl-4-yl)-9,9-dimethyl-N-(4-(9-phenyl-9H-carbazol-3-yl)phenyl)-9H-fluoren-2-amine

Compound C-1: N,N-di([1,1′-biphenyl]-4-yl)-7,7-dimethyl-7H-fluoreno[4,3-b]benzofuran-10-amine

Compound D: 8-(4-(4,6-di(naphthalen-2-yl)-1,3,5-triazin-2-yl)phenyl)quinolone

### EXAMPLE 2

An organic light emitting diode was manufactured according to the same method as Example 1 except for depositing Compound D-25 and Compound E-34 according to Synthesis Example 11 in a weight ratio of 5:5 instead of Compound D-25 and Compound E-21 in a weight ratio of 5:5.

### COMPARATIVE EXAMPLE 1

An organic light emitting diode was manufactured according to the same method as Example 1 except for depositing Compound D-25 and Compound R-1 in a weight ratio of 5:5 instead of Compound D-25 and Compound E-21 in a weight ratio of 5:5.

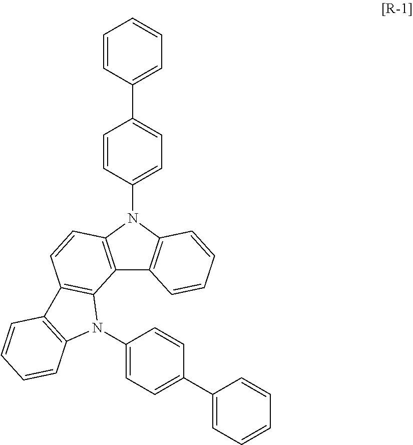 [SMILES: c1ccc(-c2ccc(-n3c4ccccc4c4c3ccc3c5ccccc5n(-c5ccc(-c6ccccc6)cc5)c34)cc2)cc1]

### COMPARATIVE EXAMPLE 2

An organic light emitting diode was manufactured according to the same method as Example 1 except for depositing compound D-25 and Compound R-2 in a weight ratio of 5:5 instead of Compound D-25 and Compound E-21 in a weight ratio of 5:5.

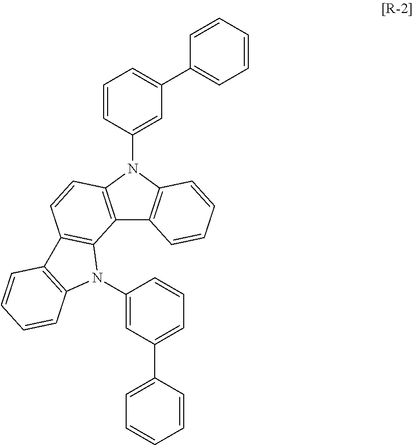 [SMILES: c1ccc(-c2cccc(-n3c4ccccc4c4c3ccc3c5ccccc5n(-c5cccc(-c6ccccc6)c5)c34)c2)cc1]

Evaluation

Power efficiency of the organic light emitting diodes according to Examples 1 and 2 and Comparative Examples 1 and 2 was evaluated.

Specific measurement methods are as follows, and the results are shown in Table 1.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltages of the organic light emitting diodes were increased from 0 V to 10 V.

(3) Measurement of Power Efficiency

Power efficiency (cd/A) at the same current density (10 mA/cm²) were calculated by using the luminance, current density, and voltages (V) from the items (1) and (2).

TABLE 1

| | | | Ratio of First | | |
|---|---|---|---|---|---|
| | First | Second | host:Second | | Power efficiency |
| | host | host | host (wt:wt) | Color | (cd/A) |
| Example 1 | D-25 | E-21 | 5:5 | red | 19.1 |
| Example 2 | D-25 | E-34 | 5:5 | red | 18.6 |
| Comparative | D-25 | R-1 | 5:5 | red | 14.2 |
| Example 1 | | | | | |
| Comparative | D-25 | R-2 | 5:5 | red | 14.1 |
| Example 2 | | | | | |

Referring to Table 1, the organic light emitting diodes according to Examples 1 and 2 exhibited high power efficiency, compared with the organic light emitting diodes according to Comparative Examples 1 and 2.

One or more embodiments may provide a composition for an organic optoelectronic device capable of realizing an organic optoelectronic device having high efficiency and a long life-span.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A composition, comprising:
a first compound represented by Chemical Formula 1, and
a second compound represented by Chemical Formula 2 or Chemical Formula 3: 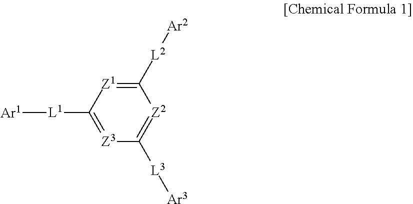
wherein, in Chemical Formula 1,
Z¹ to Z³ are independently N or CR^{a}, in which R^{a} is hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof,
at least two of Z¹ to Z³ are N,
L¹ to L³ are independently a single bond, a substituted or unsubstituted C6 to C20 arylene group, a substituted or unsubstituted divalent C3 to C20 heterocyclic group, or a combination thereof,
Ar¹ to Ar³ are independently a substituted or unsubstituted C6 to C30 aryl group, a substituted or unsubstituted C3 to C30 heterocyclic group, or a combination thereof, and
at least one of Ar¹ to Ar³ is a group represented by Chemical Formula A, 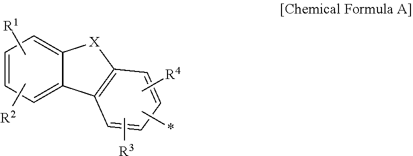
wherein, in Chemical Formula A,
X is O or S,
R¹ to R⁴ are independently hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof,
R¹ and R² are separate or are linked with each other to form a ring,
R³ and R⁴ are separate or are linked with each other to form a ring, and
* is a linking point with Chemical Formula 1, 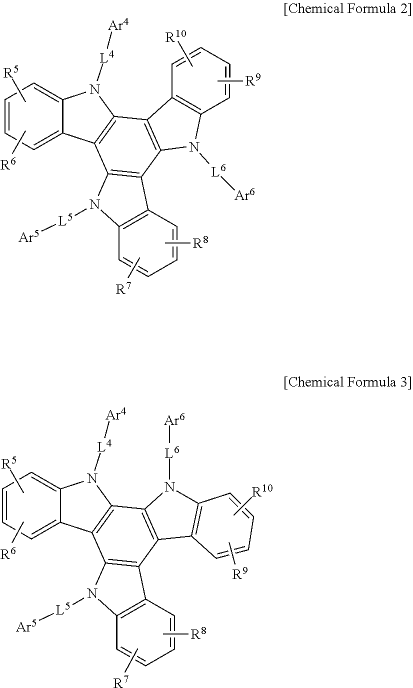
wherein, in Chemical Formula 2 and Chemical Formula 3,
Ar⁴ to Ar⁶ are independently hydrogen, deuterium, a substituted or unsubstituted C1 to C30 alkyl group, a substituted or unsubstituted C6 to C30 aryl group, a substituted or unsubstituted C3 to C30 heterocyclic group, or a combination thereof,
L⁴ to L⁶ are independently a single bond, a substituted or unsubstituted C6 to C20 arylene group, a substituted or unsubstituted divalent C3 to C20 heterocyclic group, or a combination thereof,
R⁵ to R¹⁰ are independently hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof,
R⁵ and R⁶ are separate or are linked with each other to form a ring,
R⁷ and R⁸ are separate or are linked with each other to form a ring, and
R⁹ and R¹⁰ are separate or are linked with each other to form a ring.

2. The composition as claimed in claim 1, wherein the first compound is represented by one of Chemical Formulae 1A to 1C: 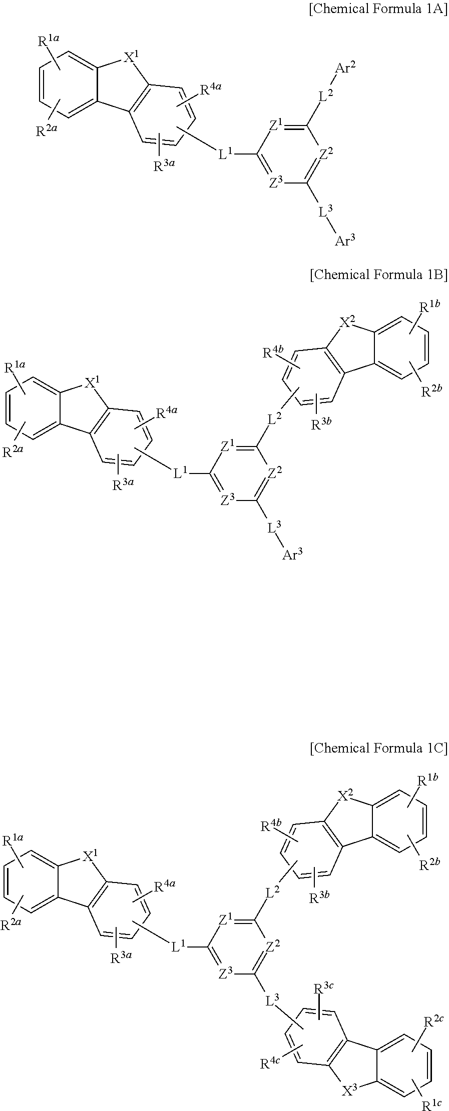
wherein, in Chemical Formulae 1A to 1C,
Z¹ to Z³ are independently N or CR^{a}, in which R^{a} is hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof,
at least two of Z¹ to Z³ are N,
L¹ to L³ are independently a single bond, a substituted or unsubstituted C6 to C20 arylene group, a substituted or unsubstituted divalent C3 to C20 heterocyclic group, or a combination thereof,
Ar² and Ar³ are independently a substituted or unsubstituted C6 to C30 aryl group, a substituted or unsubstituted C3 to C30 heterocyclic group, or a combination thereof,
X¹ to X³ are independently O or S, and
R^{1a} to R^{4a}, R^{1b} to R^{4b}, and R^{1c} to R^{4c} are independently hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof.

3. The composition as claimed in claim 2, wherein Ar² and Ar³ are independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted pyridinyl group, a substituted or unsubstituted pyrimidinyl group, a substituted or unsubstituted triazinyl group, or a combination thereof.

4. The composition as claimed in claim 1, wherein the group represented by Chemical Formula A is a group represented by one of Chemical Formulae A-1 to A-4: 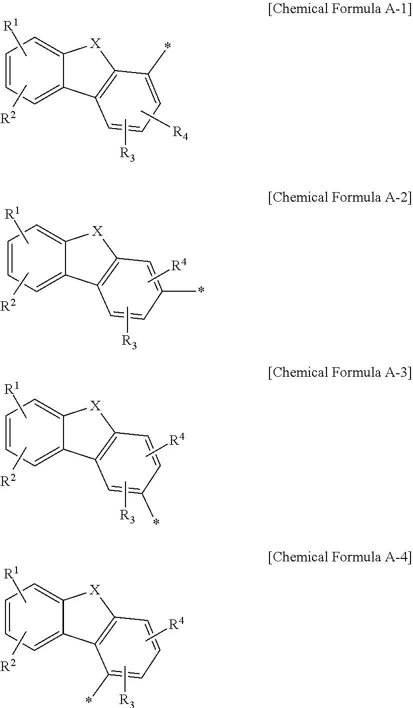
wherein, in Chemical Formulae A-1 to A-4,
X is O or S,
R¹ to R⁴ are independently hydrogen, deuterium, a substituted or unsubstituted C1 to C30 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof,
R¹ and R² are separate or are linked with each other to form a ring,
R³ and R⁴ are separate or are linked with each other to form a ring, and
* is a linking point with Chemical Formula 1.

5. The composition as claimed in claim 1, wherein A¹ to Ar³ are independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted pyridinyl group, a substituted or unsubstituted pyrimidinyl group, a substituted or unsubstituted triazinyl group, a group represented by Chemical Formula A, or a combination thereof.

6. The composition as claimed in claim 1, wherein the first compound is one of the compounds listed in Group 1:

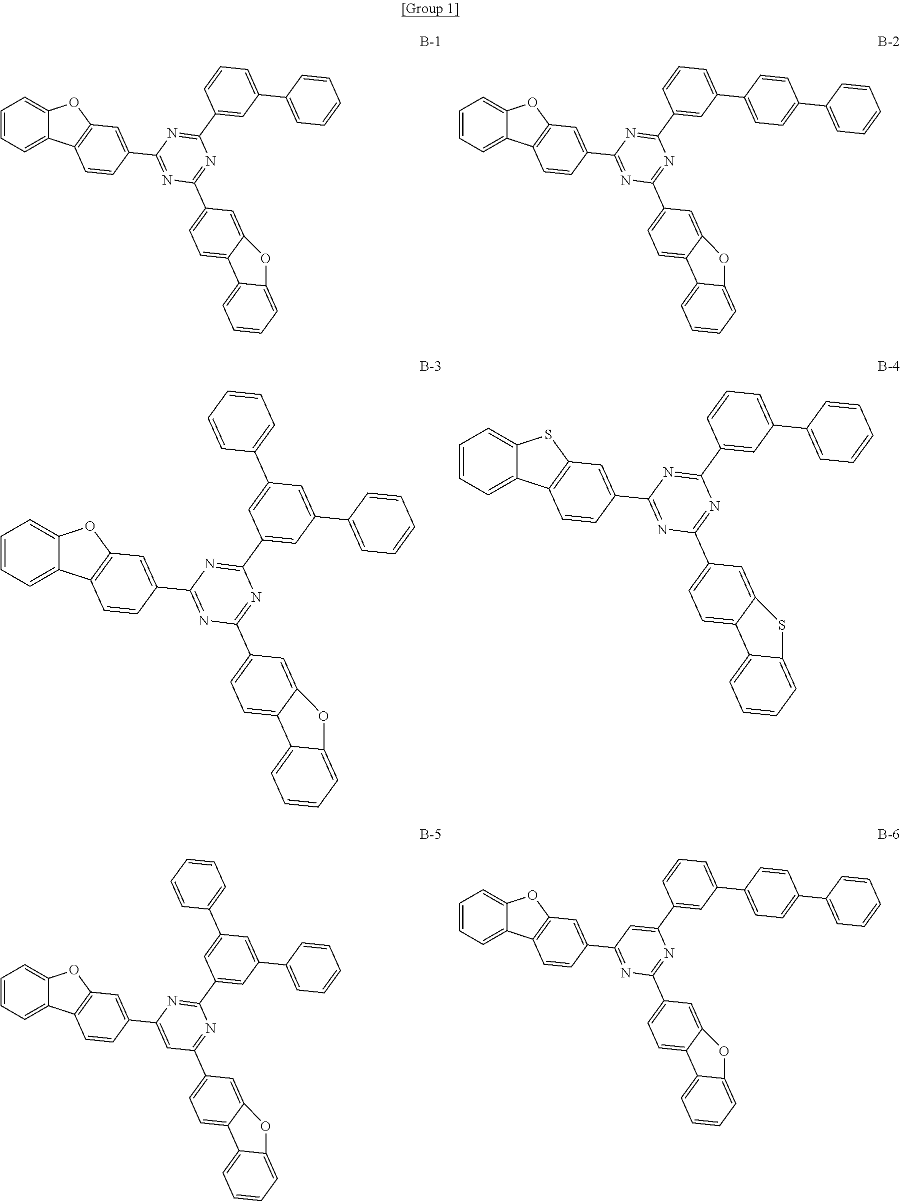

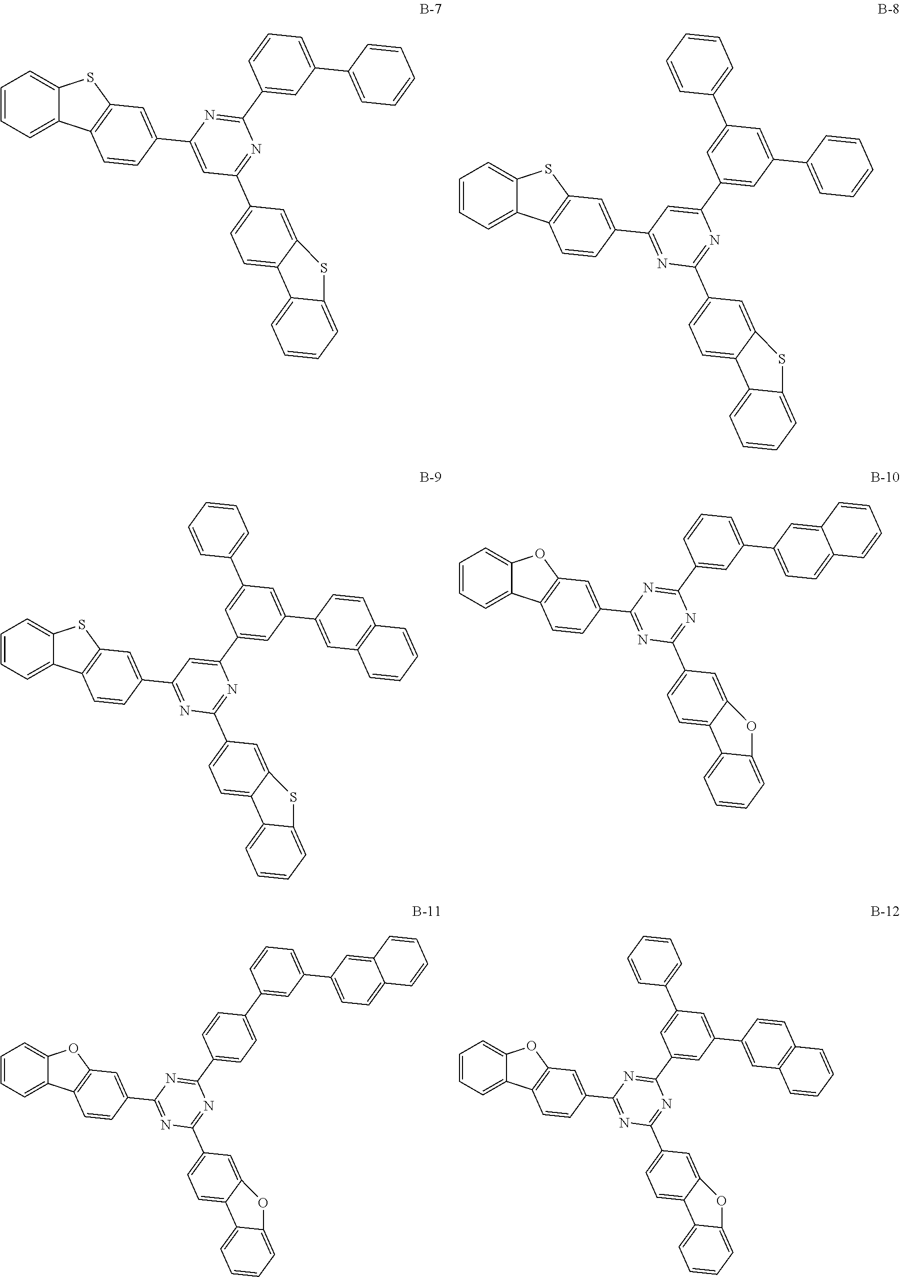

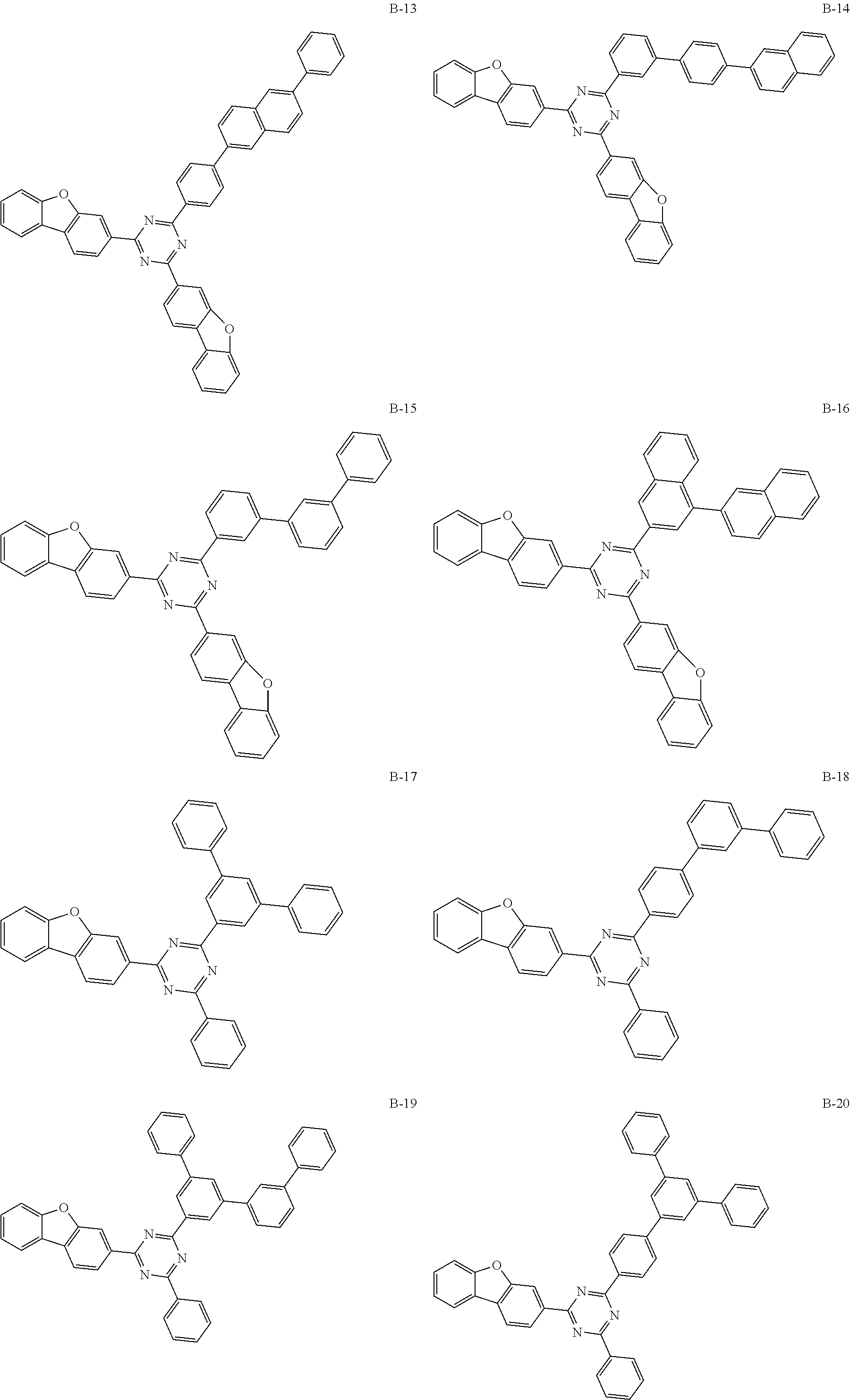

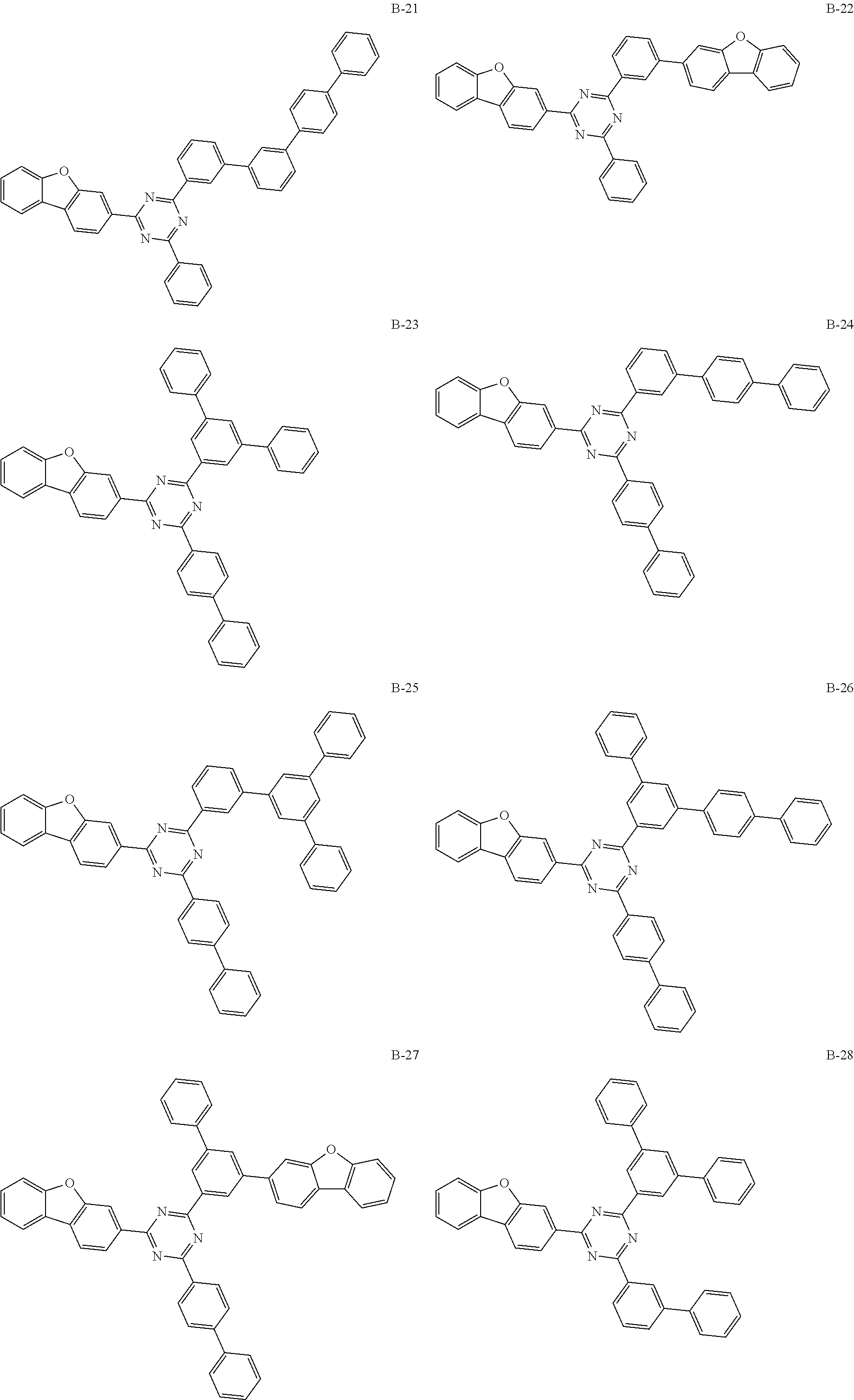

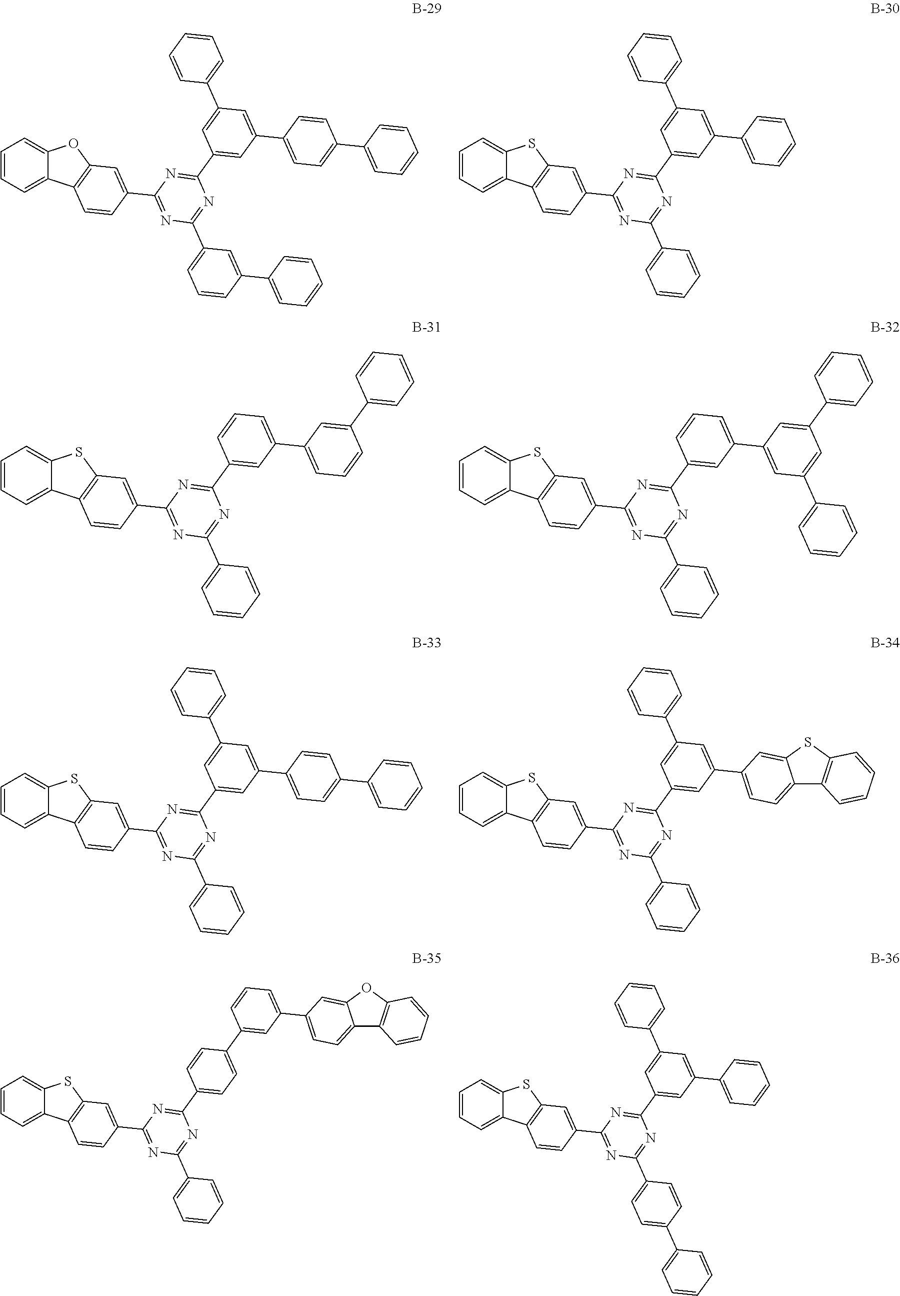

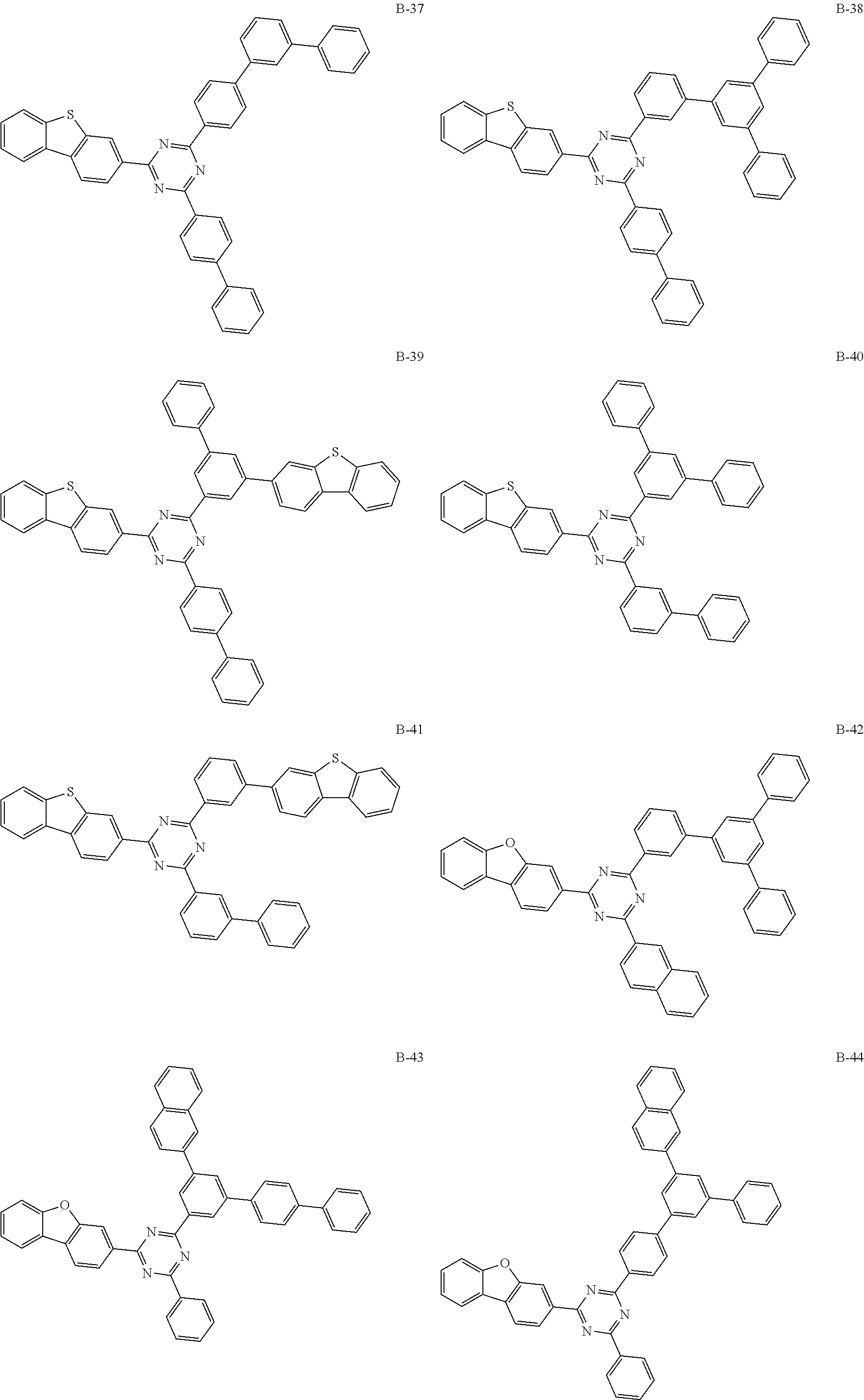

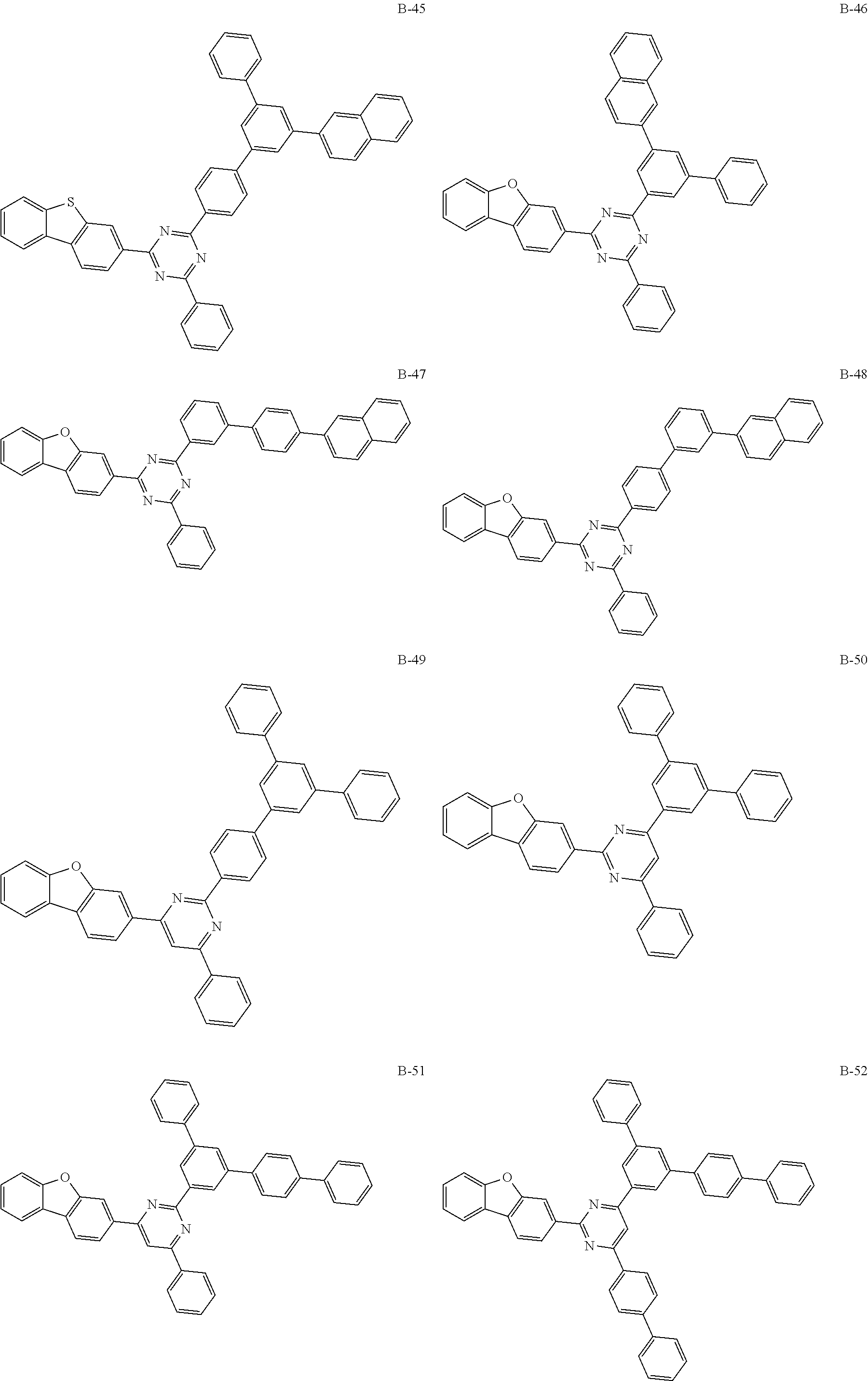

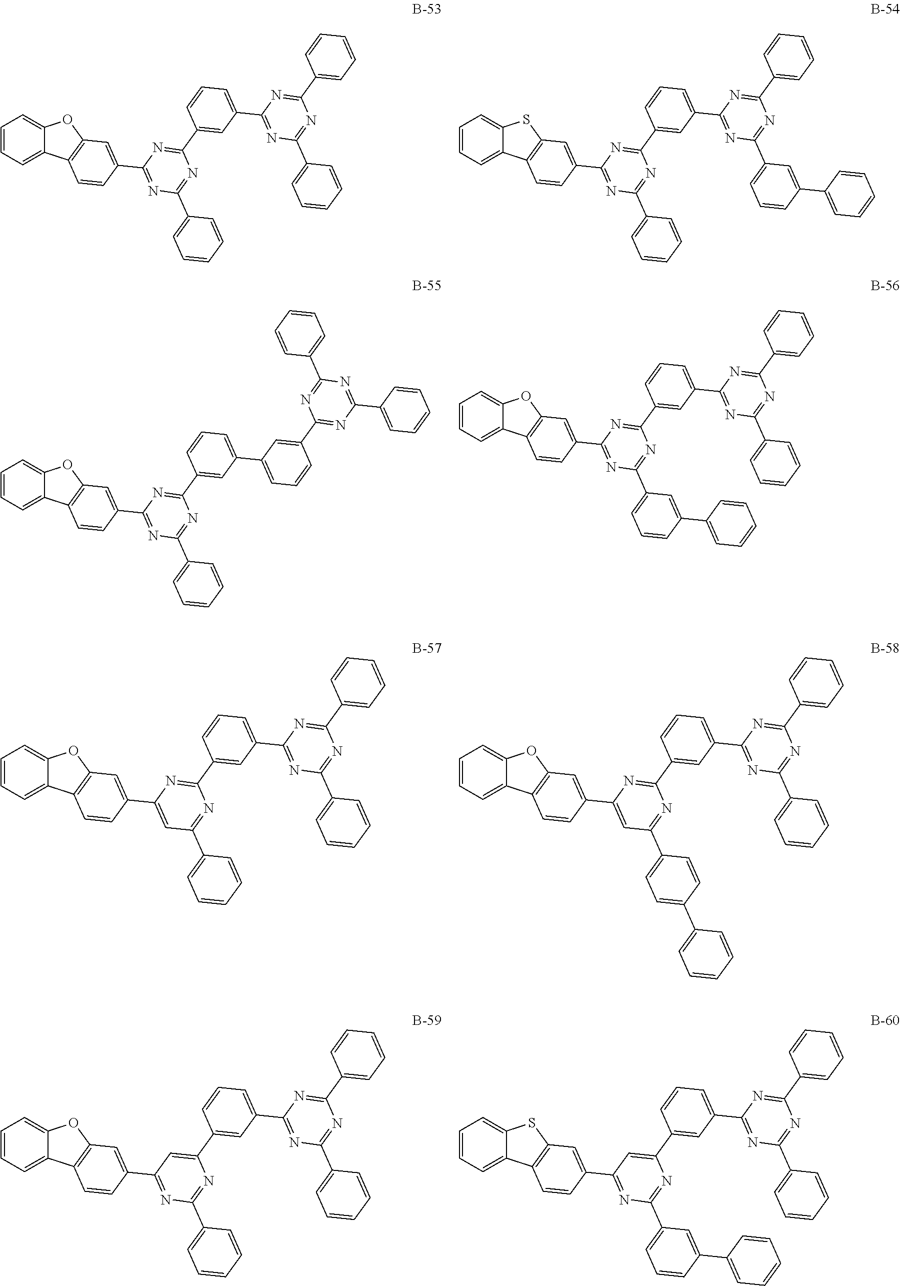

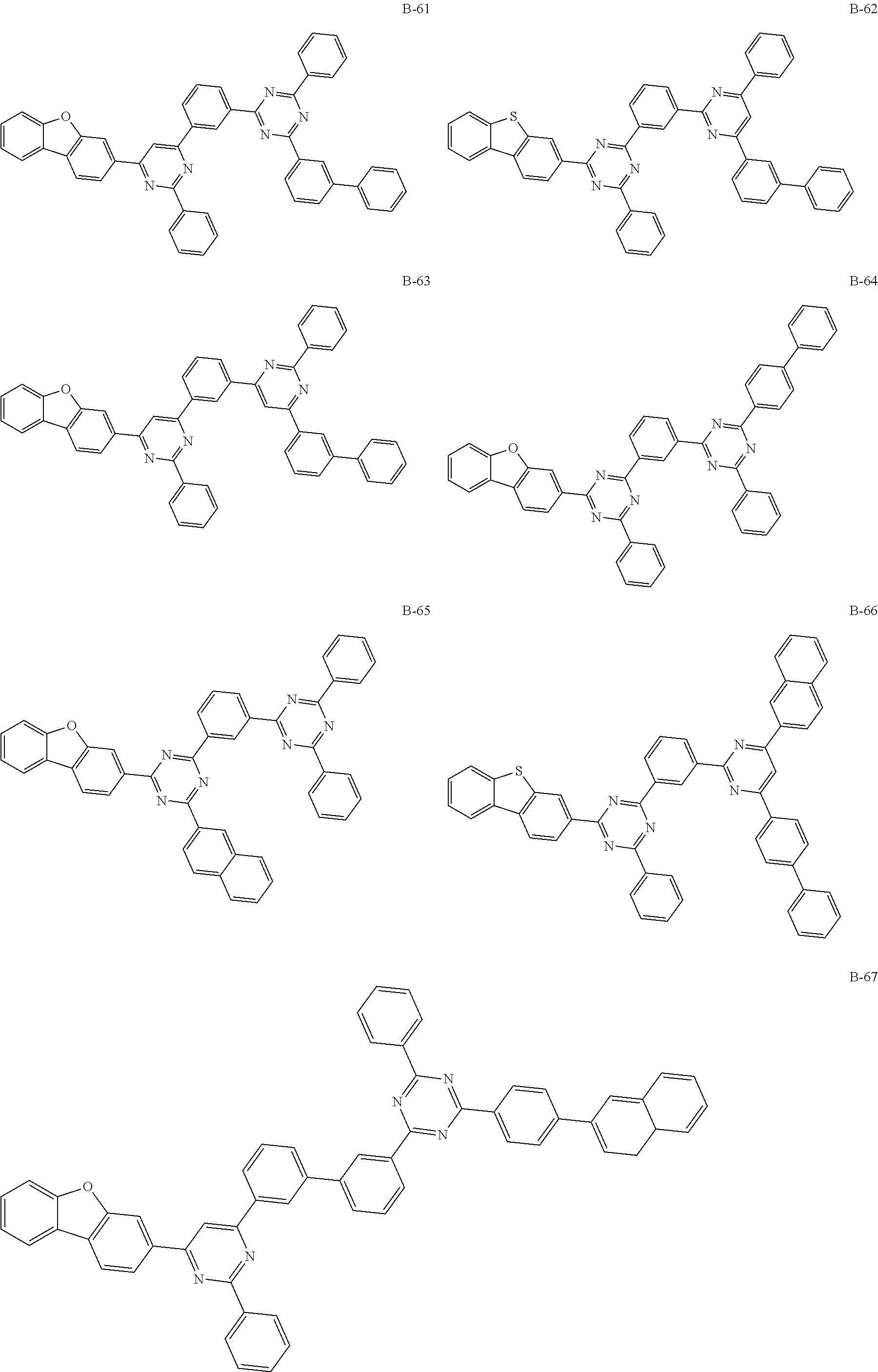

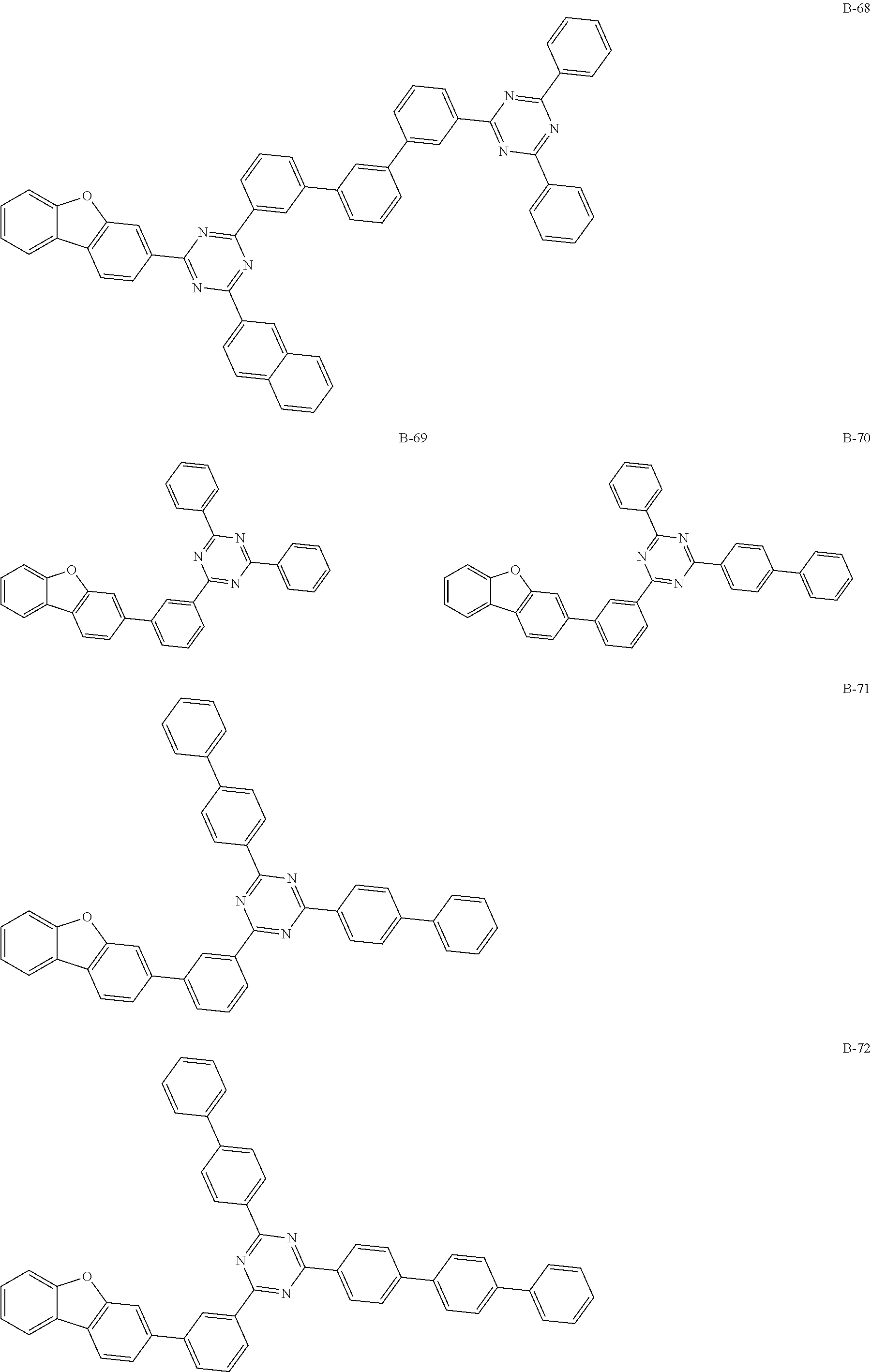 [SMILES: c1ccc(-c2nc(-c3ccccc3)nc(-c3cccc(-c4cccc(-c5cccc(-c6nc(-c7ccc8ccccc8c7)nc(-c7ccc8c(c7)oc7ccccc78)n6)c5)c4)c3)n2)cc1 ; c1ccc(-c2nc(-c3ccccc3)nc(-c3cccc(-c4ccc5c(c4)oc4ccccc45)c3)n2)cc1 ; c1ccc(-c2ccc(-c3nc(-c4ccccc4)nc(-c4cccc(-c5ccc6c(c5)oc5ccccc56)c4)n3)cc2)cc1 ; c1ccc(-c2ccc(-c3nc(-c4ccc(-c5ccccc5)cc4)nc(-c4cccc(-c5ccc6c(c5)oc5ccccc56)c4)n3)cc2)cc1 ; c1ccc(-c2ccc(-c3ccc(-c4nc(-c5ccc(-c6ccccc6)cc5)nc(-c5cccc(-c6ccc7c(c6)oc6ccccc67)c5)n4)cc3)cc2)cc1]

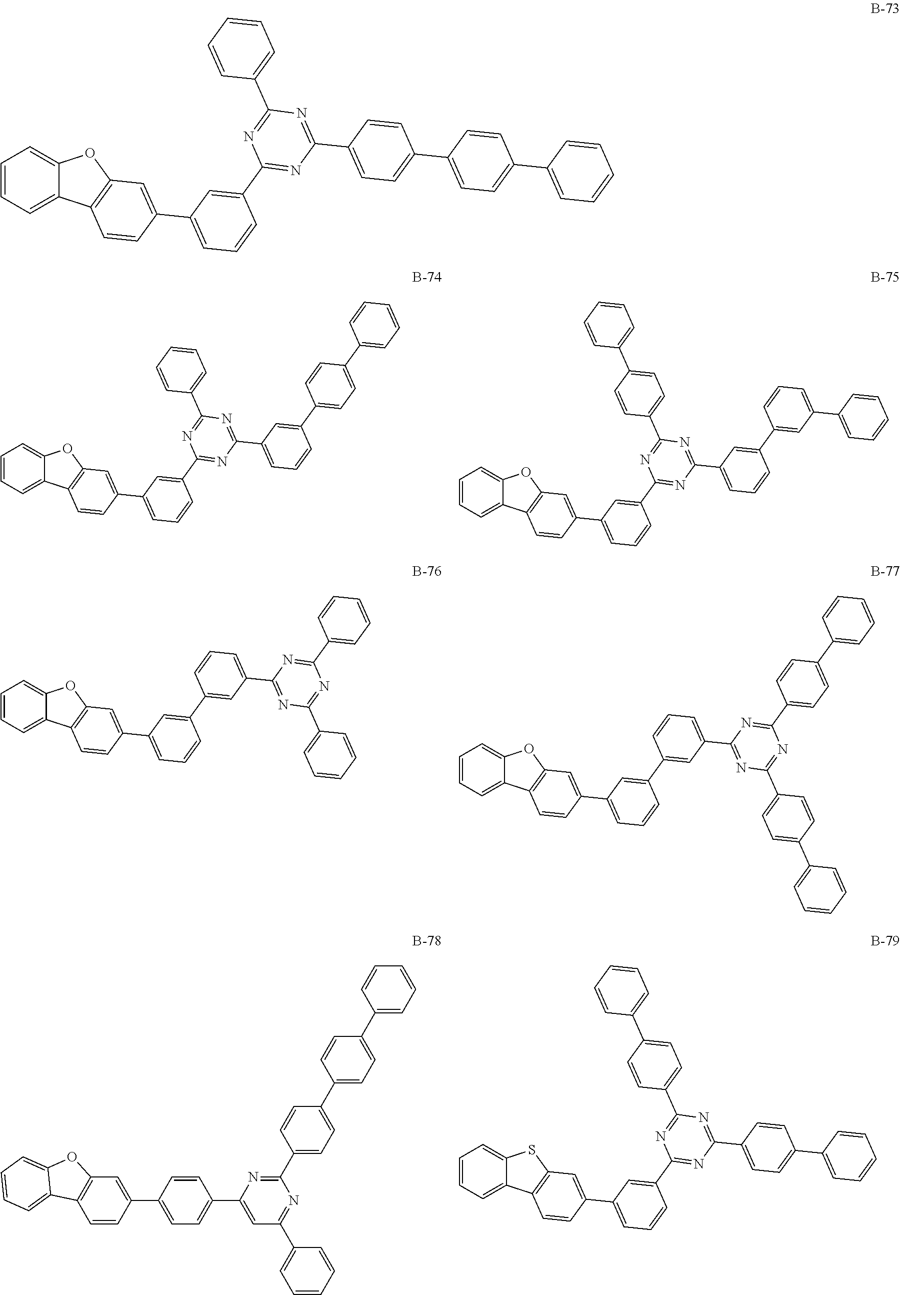

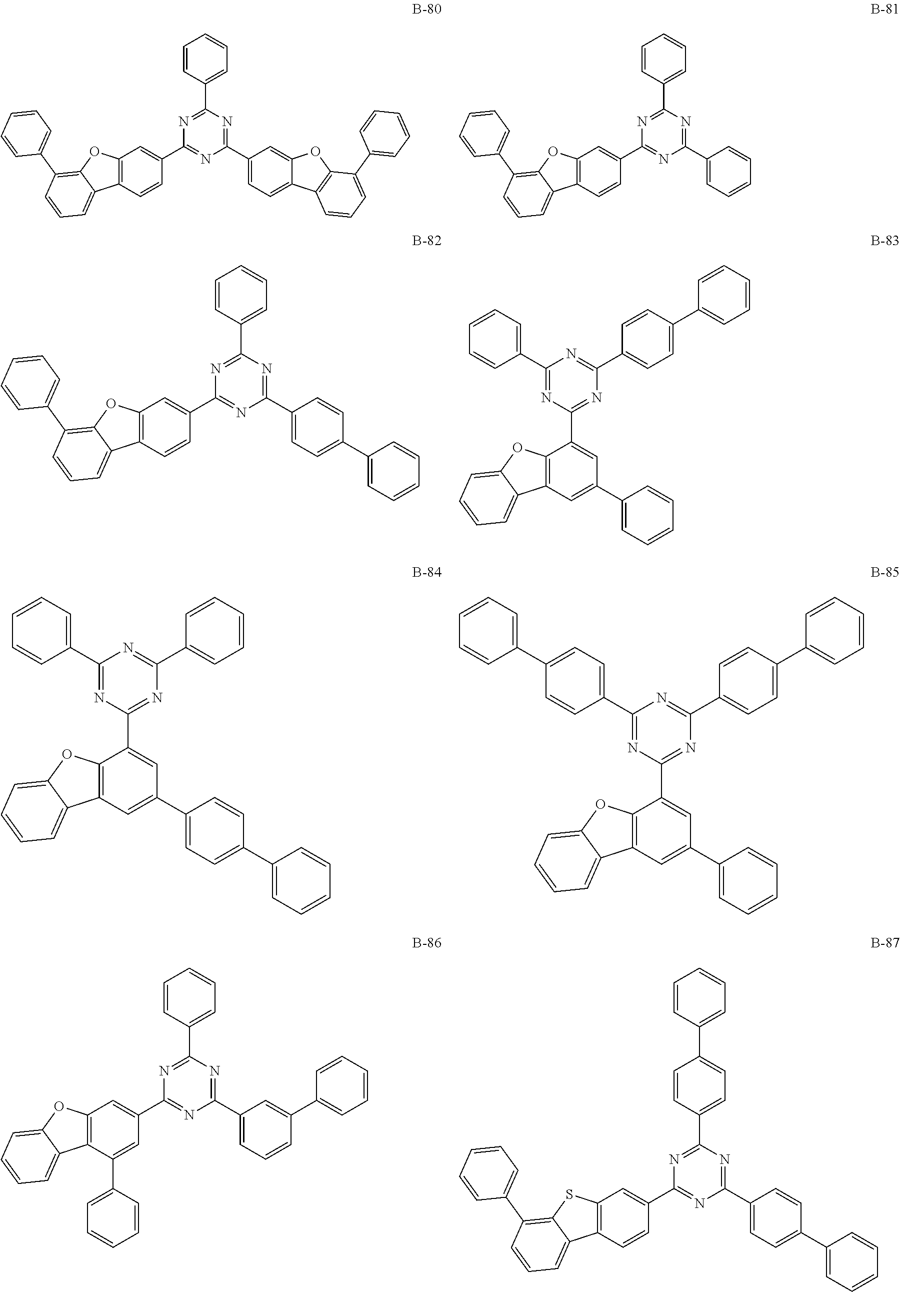

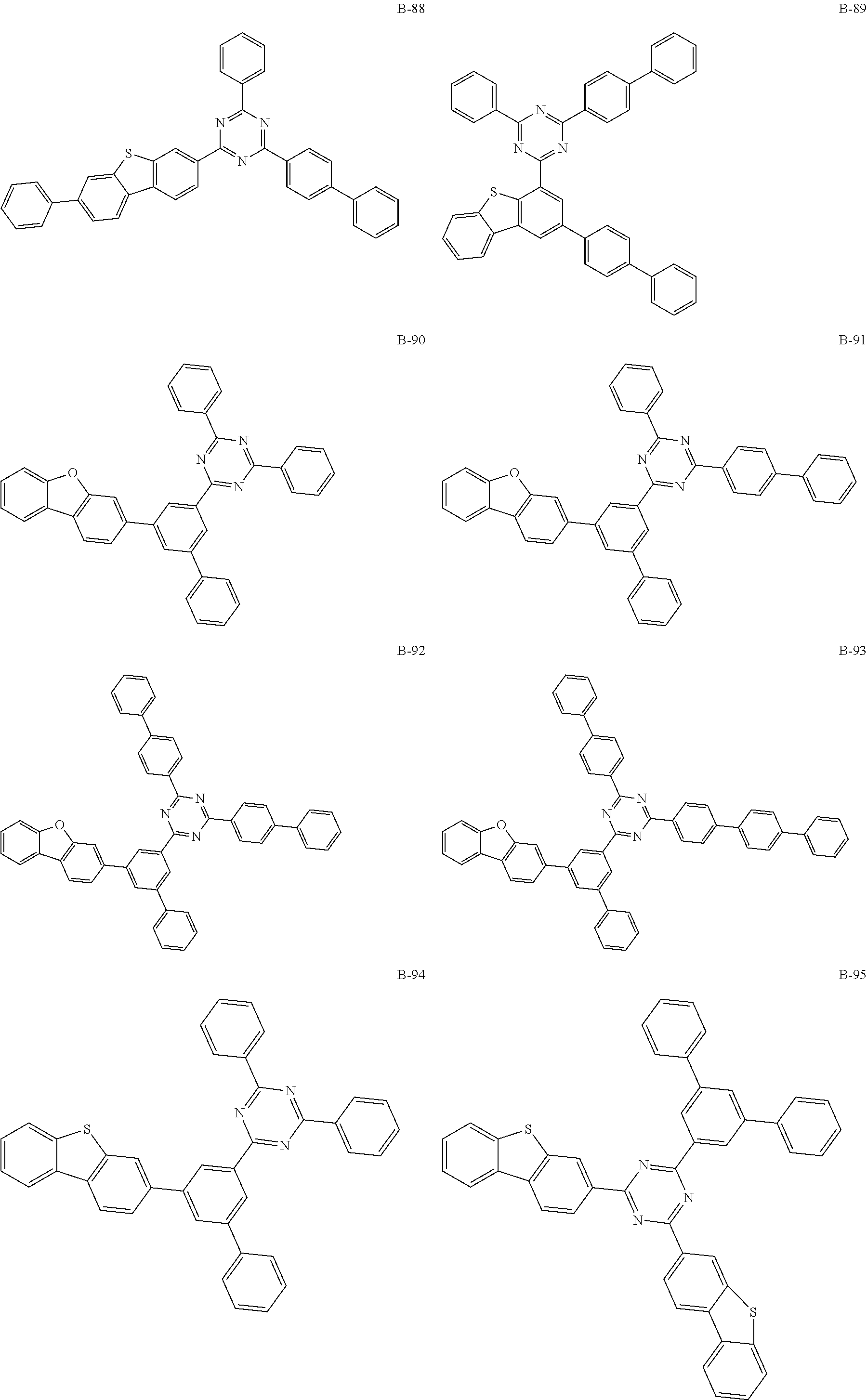

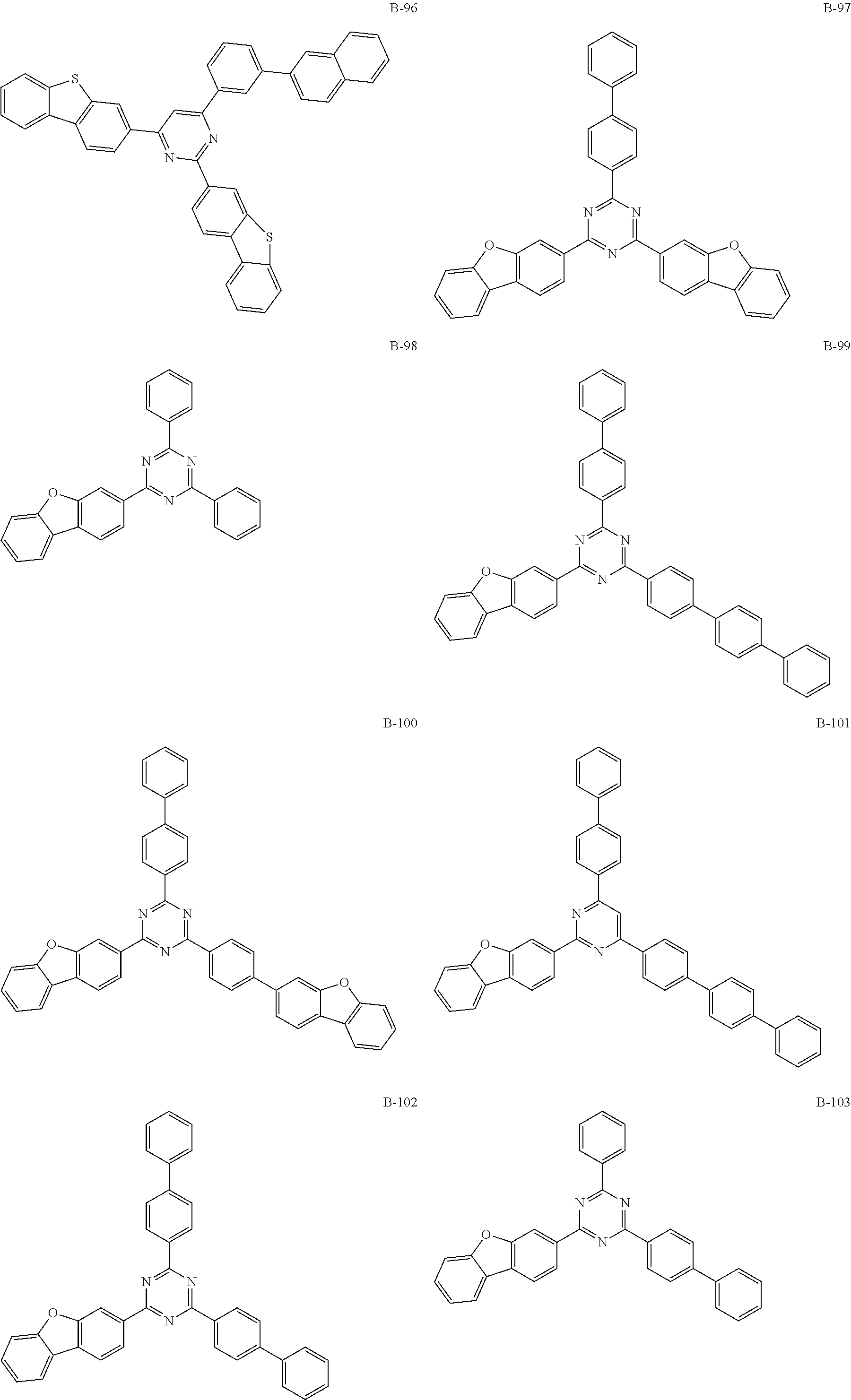

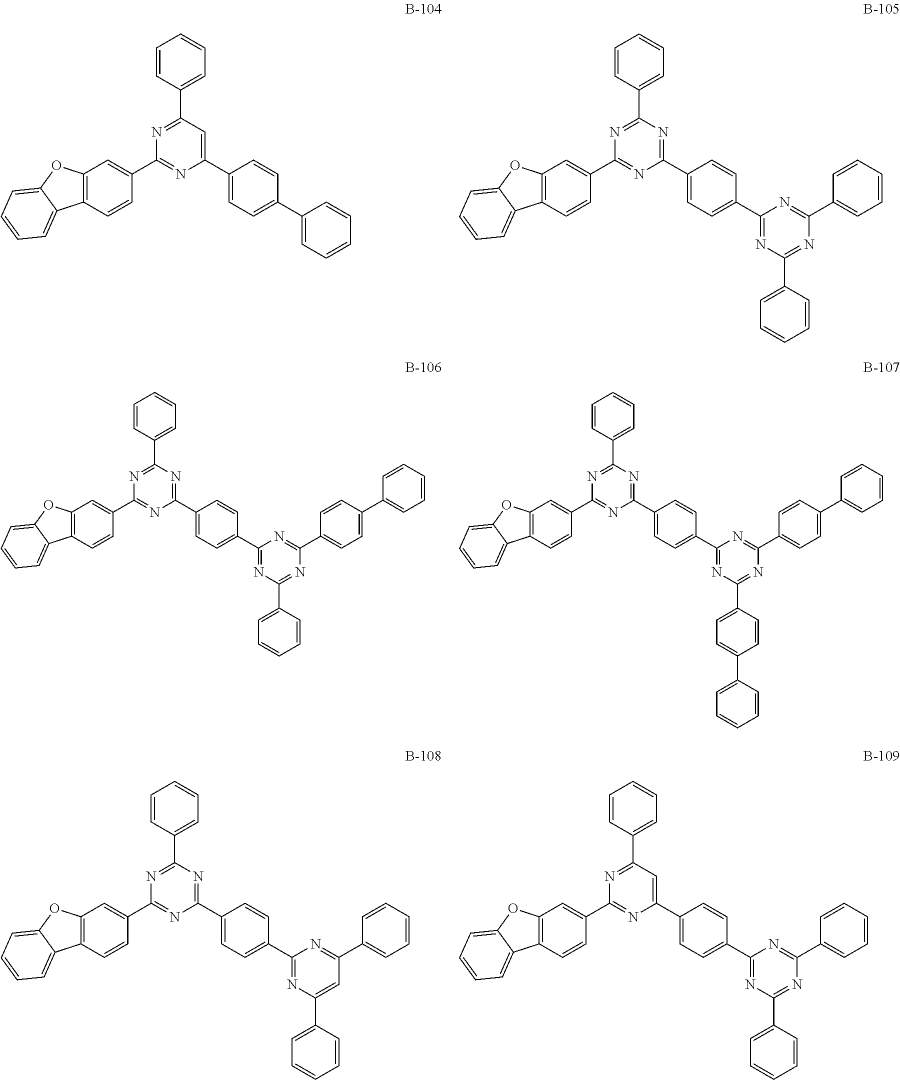

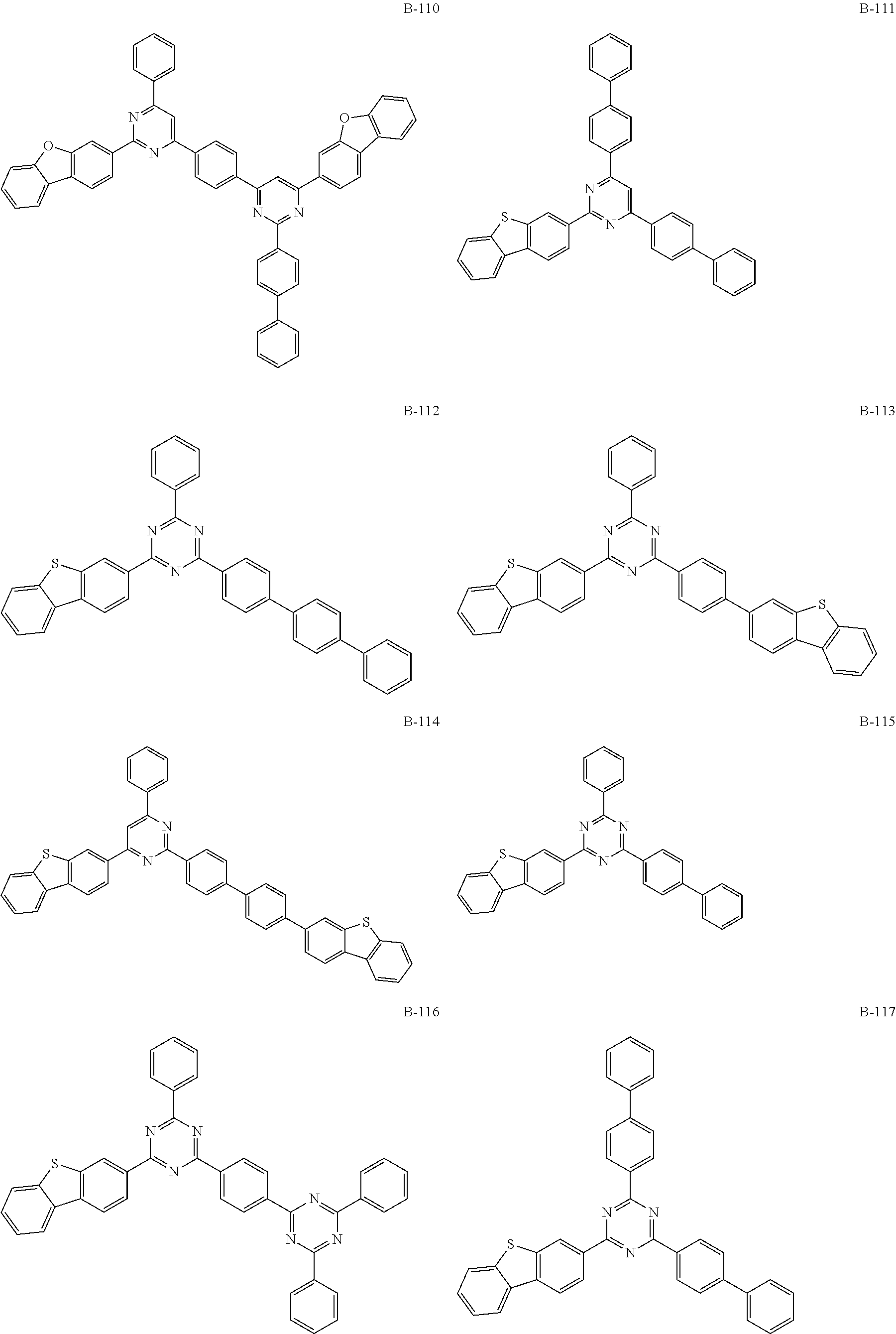

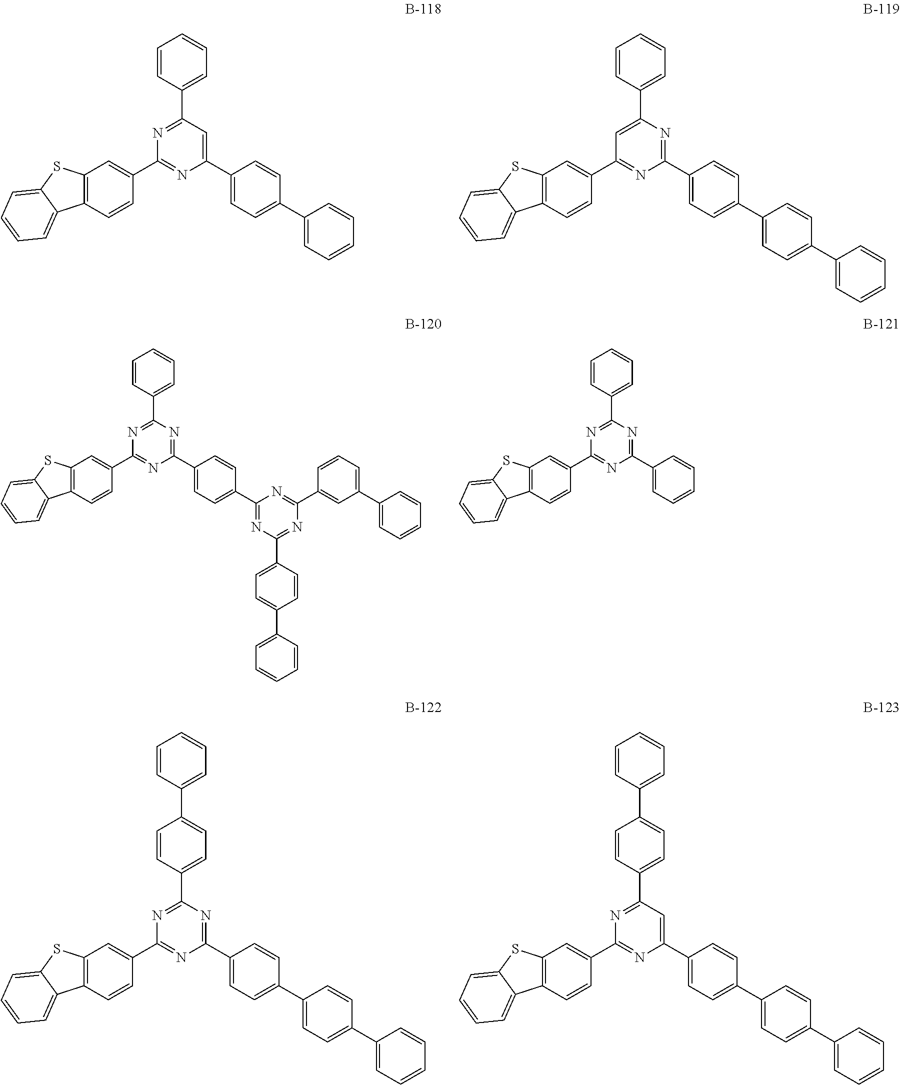

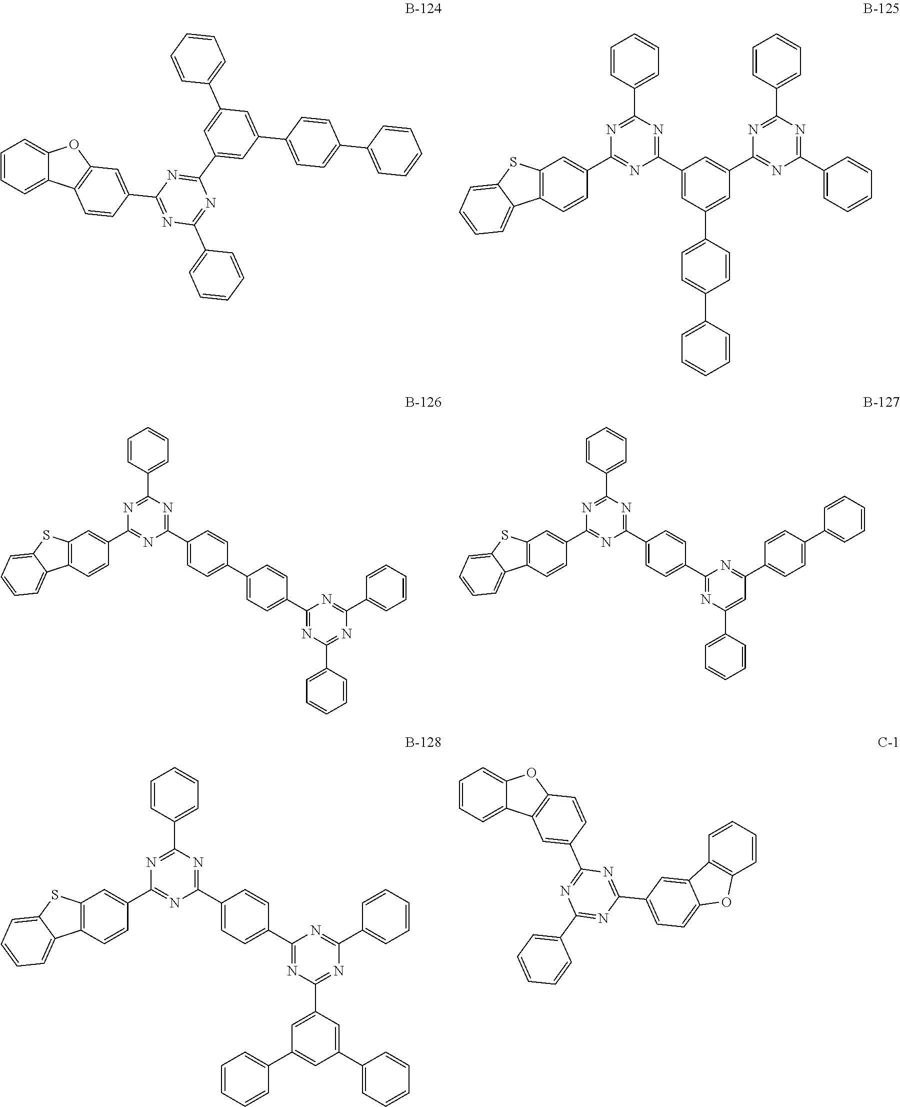

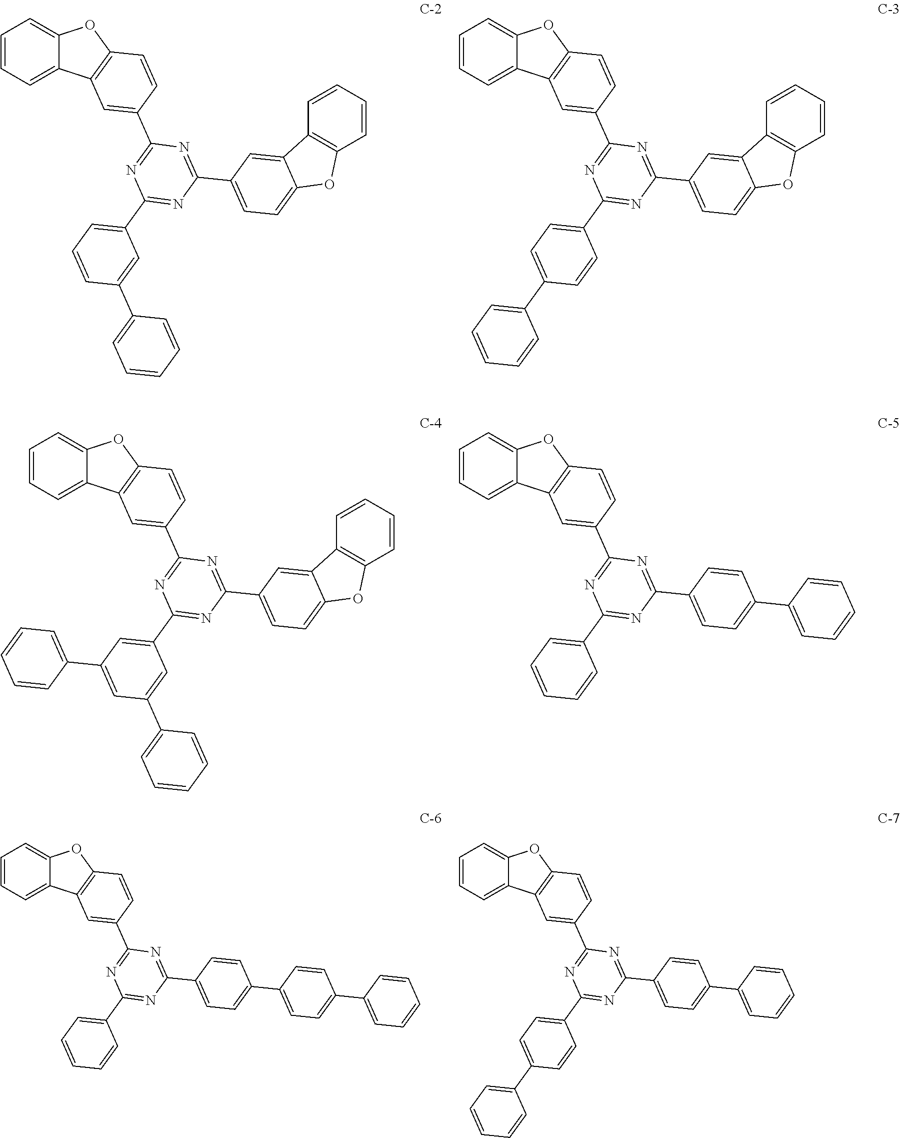 [SMILES: c1ccc(-c2cccc(-c3nc(-c4ccc5oc6ccccc6c5c4)nc(-c4ccc5oc6ccccc6c5c4)n3)c2)cc1 ; c1ccc(-c2ccc(-c3nc(-c4ccc5oc6ccccc6c5c4)nc(-c4ccc5oc6ccccc6c5c4)n3)cc2)cc1 ; c1ccc(-c2cc(-c3ccccc3)cc(-c3nc(-c4ccc5oc6ccccc6c5c4)nc(-c4ccc5oc6ccccc6c5c4)n3)c2)cc1 ; c1ccc(-c2ccc(-c3nc(-c4ccccc4)nc(-c4ccc5oc6ccccc6c5c4)n3)cc2)cc1 ; c1ccc(-c2ccc(-c3ccc(-c4nc(-c5ccccc5)nc(-c5ccc6oc7ccccc7c6c5)n4)cc3)cc2)cc1 ; c1ccc(-c2ccc(-c3nc(-c4ccc(-c5ccccc5)cc4)nc(-c4ccc5oc6ccccc6c5c4)n3)cc2)cc1]

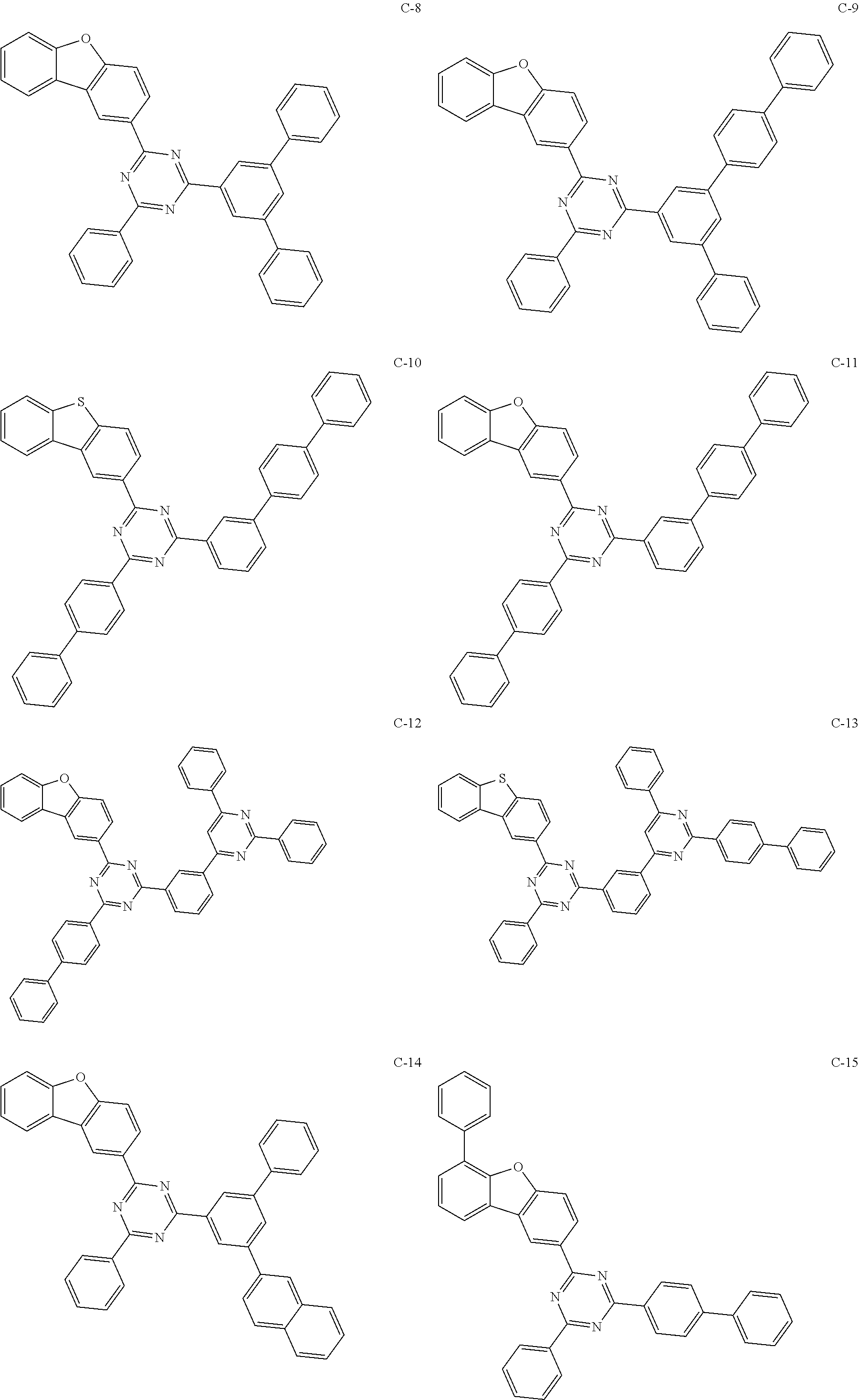

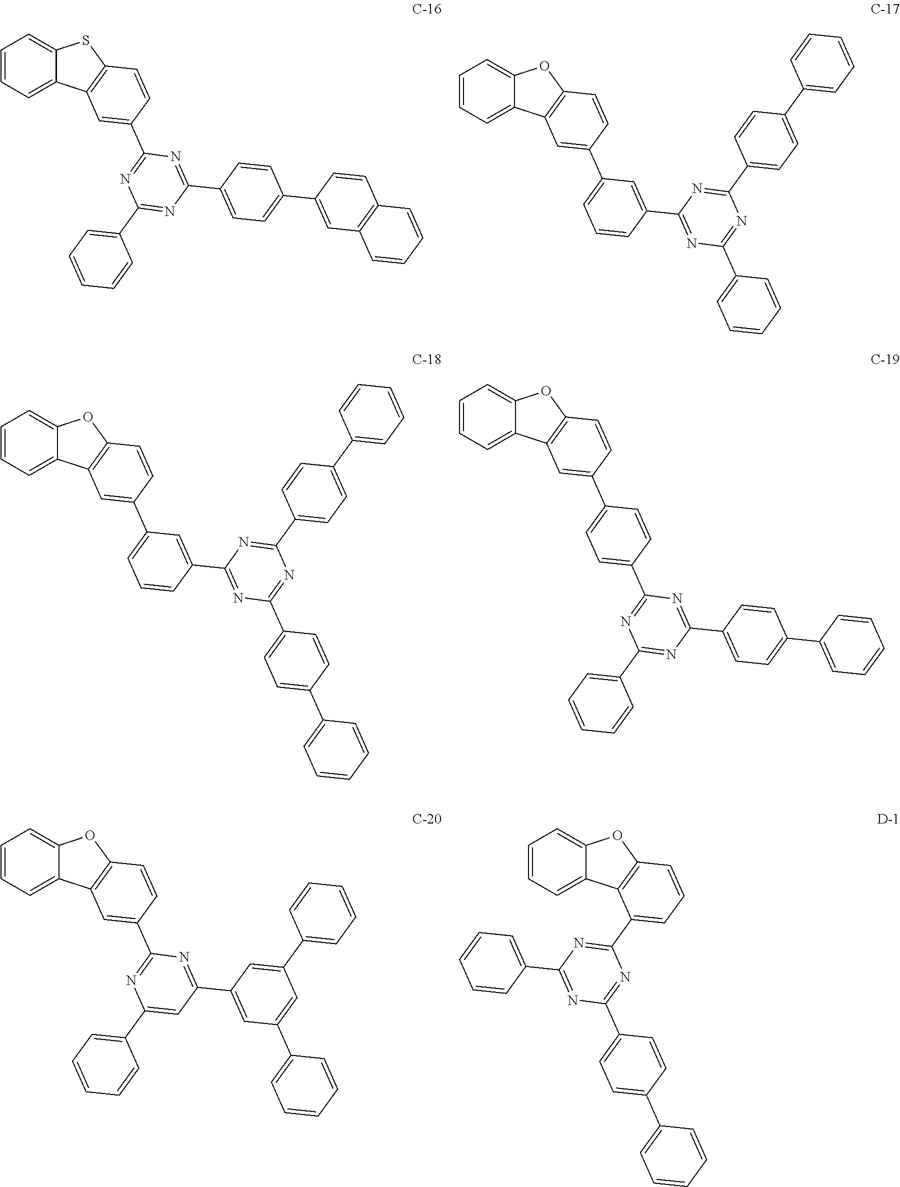 [SMILES: c1ccc(-c2nc(-c3ccc(-c4ccc5ccccc5c4)cc3)nc(-c3ccc4sc5ccccc5c4c3)n2)cc1 ; c1ccc(-c2ccc(-c3nc(-c4ccccc4)nc(-c4cccc(-c5ccc6oc7ccccc7c6c5)c4)n3)cc2)cc1 ; c1ccc(-c2ccc(-c3nc(-c4ccc(-c5ccccc5)cc4)nc(-c4cccc(-c5ccc6oc7ccccc7c6c5)c4)n3)cc2)cc1 ; c1ccc(-c2ccc(-c3nc(-c4ccccc4)nc(-c4ccc(-c5ccc6oc7ccccc7c6c5)cc4)n3)cc2)cc1 ; c1ccc(-c2cc(-c3ccccc3)cc(-c3cc(-c4ccccc4)nc(-c4ccc5oc6ccccc6c5c4)n3)c2)cc1 ; c1ccc(-c2ccc(-c3nc(-c4ccccc4)nc(-c4cccc5oc6ccccc6c45)n3)cc2)cc1]

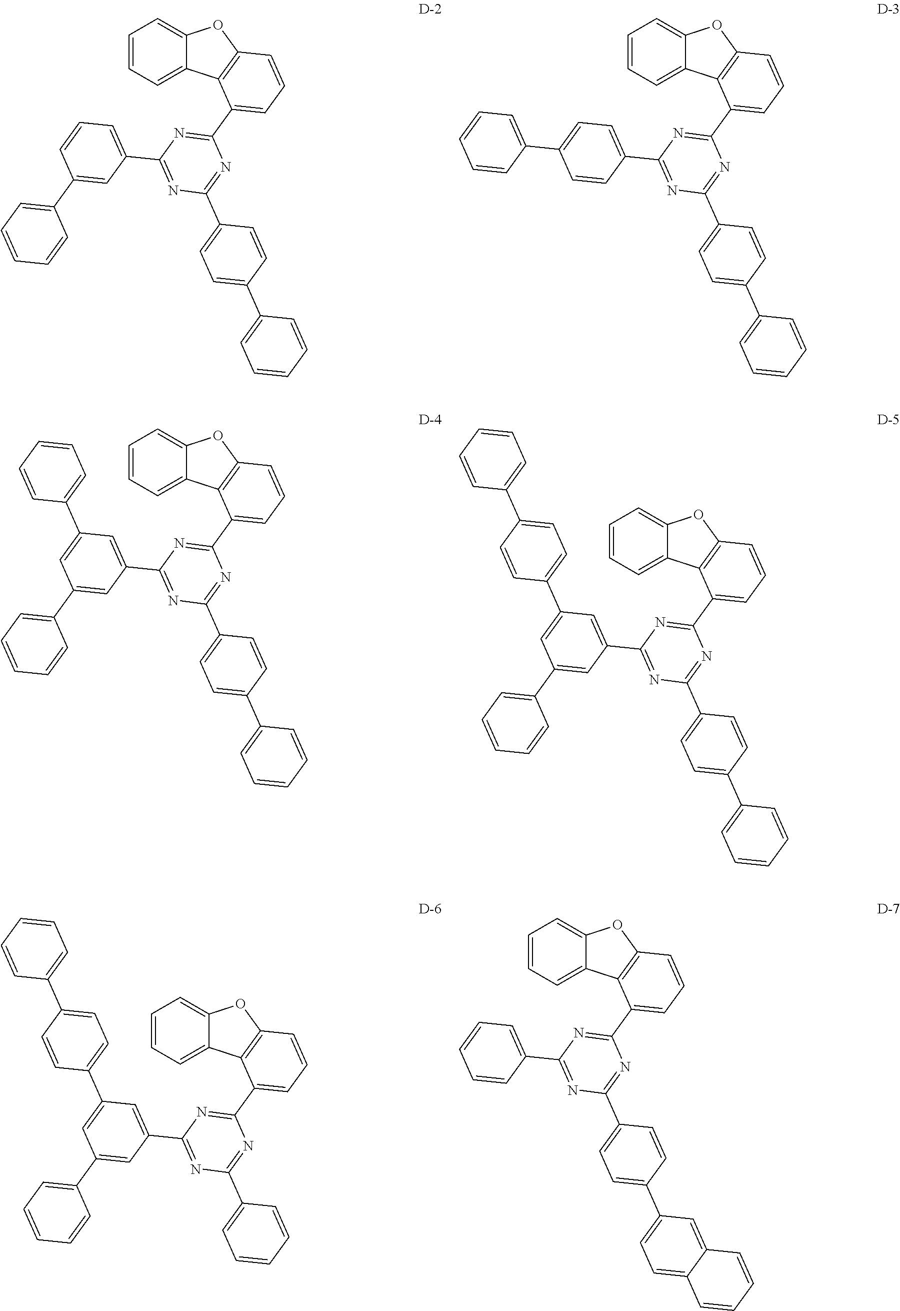 [SMILES: c1ccc(-c2ccc(-c3nc(-c4cccc(-c5ccccc5)c4)nc(-c4cccc5oc6ccccc6c45)n3)cc2)cc1 ; c1ccc(-c2ccc(-c3nc(-c4ccc(-c5ccccc5)cc4)nc(-c4cccc5oc6ccccc6c45)n3)cc2)cc1 ; c1ccc(-c2ccc(-c3nc(-c4cc(-c5ccccc5)cc(-c5ccccc5)c4)nc(-c4cccc5oc6ccccc6c45)n3)cc2)cc1 ; c1ccc(-c2ccc(-c3cc(-c4ccccc4)cc(-c4nc(-c5ccc(-c6ccccc6)cc5)nc(-c5cccc6oc7ccccc7c56)n4)c3)cc2)cc1 ; c1ccc(-c2ccc(-c3cc(-c4ccccc4)cc(-c4nc(-c5ccccc5)nc(-c5cccc6oc7ccccc7c56)n4)c3)cc2)cc1 ; c1ccc(-c2nc(-c3ccc(-c4ccc5ccccc5c4)cc3)nc(-c3cccc4oc5ccccc5c34)n2)cc1]

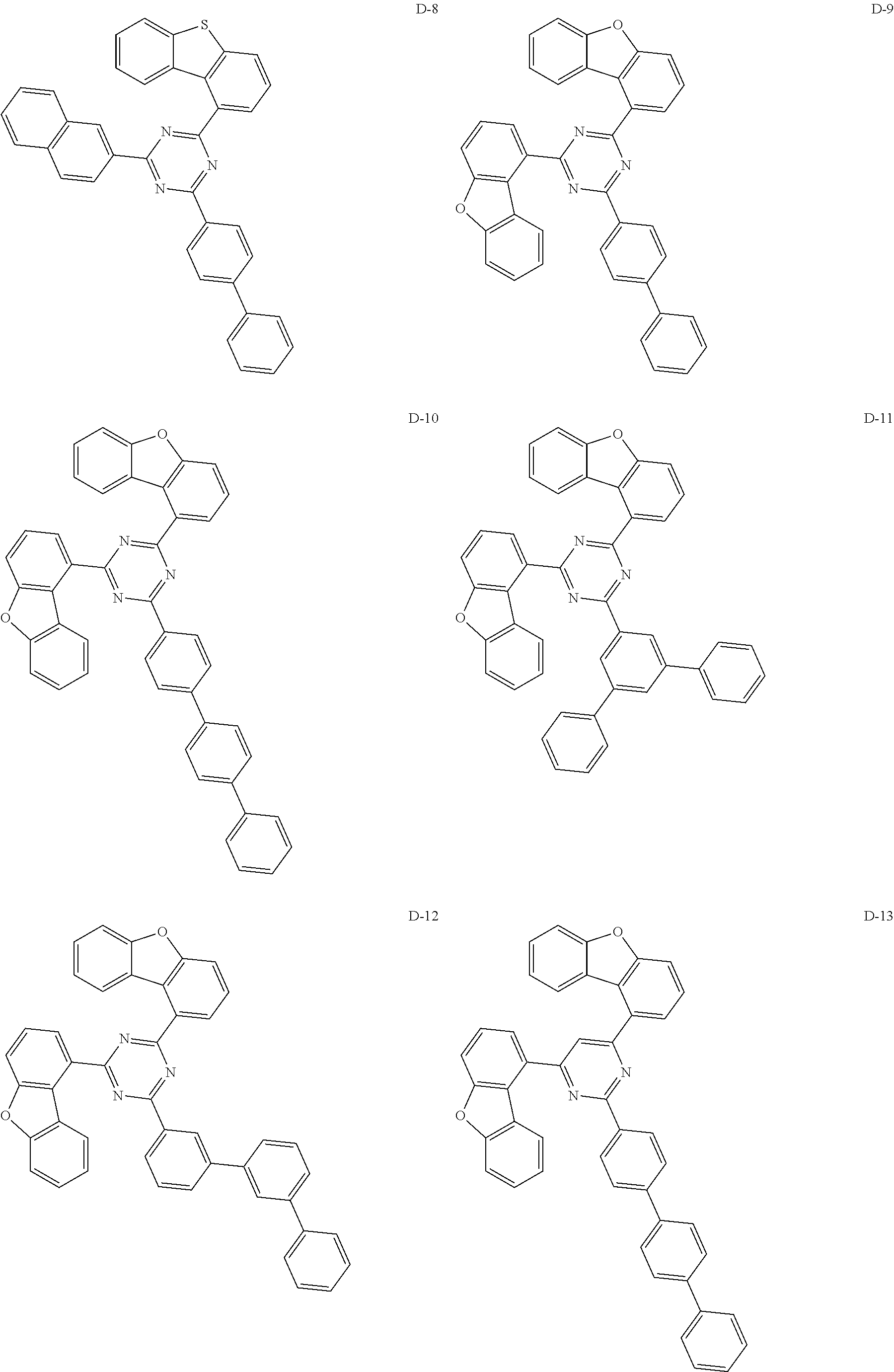 [SMILES: c1ccc(-c2ccc(-c3nc(-c4ccc5ccccc5c4)nc(-c4cccc5sc6ccccc6c45)n3)cc2)cc1 ; c1ccc(-c2ccc(-c3nc(-c4cccc5oc6ccccc6c45)nc(-c4cccc5oc6ccccc6c45)n3)cc2)cc1 ; c1ccc(-c2ccc(-c3ccc(-c4nc(-c5cccc6oc7ccccc7c56)nc(-c5cccc6oc7ccccc7c56)n4)cc3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1 ; c1ccc(-c2cccc(-c3cccc(-c4nc(-c5cccc6oc7ccccc7c56)nc(-c5cccc6oc7ccccc7c56)n4)c3)c2)cc1 ; c1ccc(-c2ccc(-c3ccc(-c4nc(-c5cccc6oc7ccccc7c56)cc(-c5cccc6oc7ccccc7c56)n4)cc3)cc2)cc1]

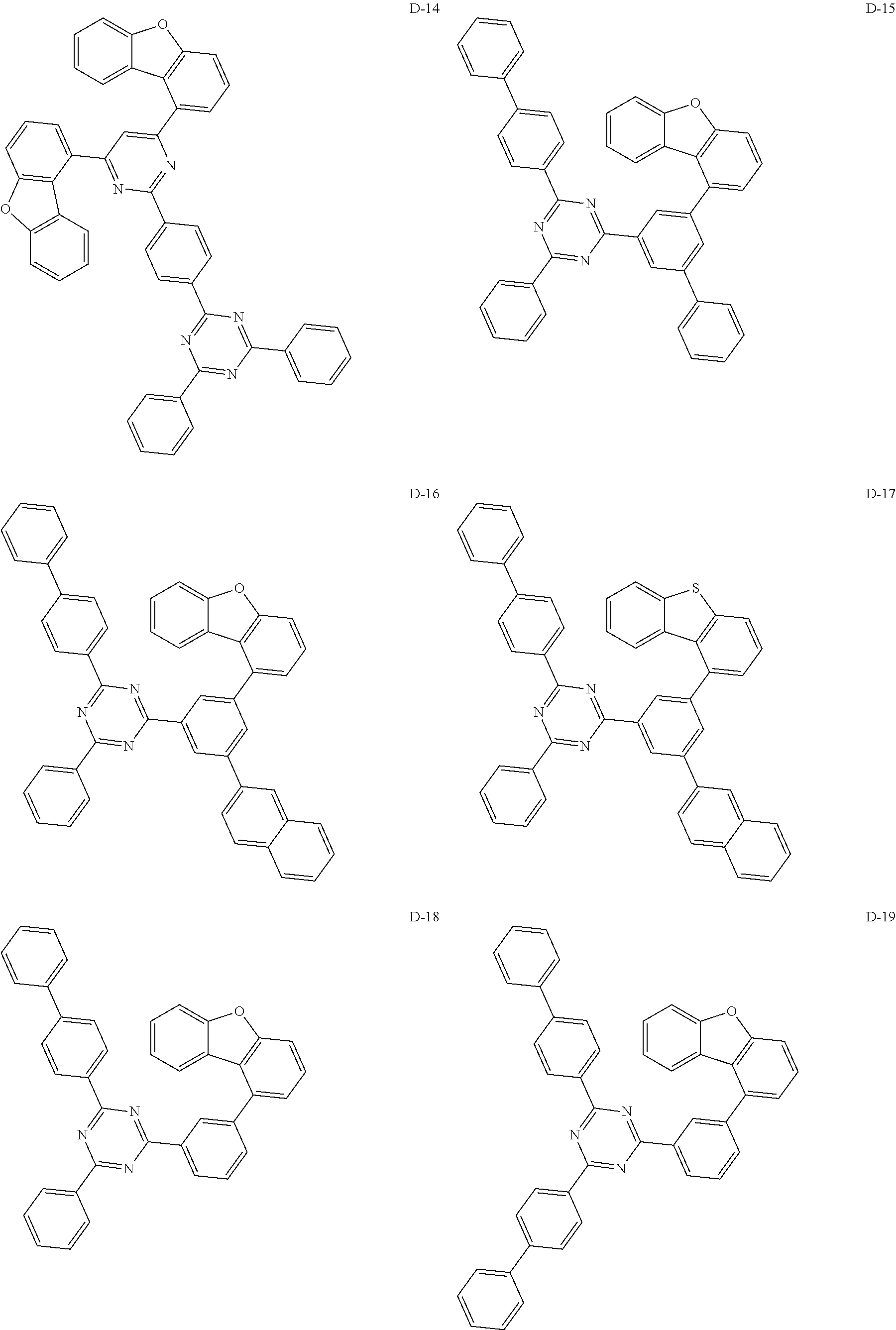

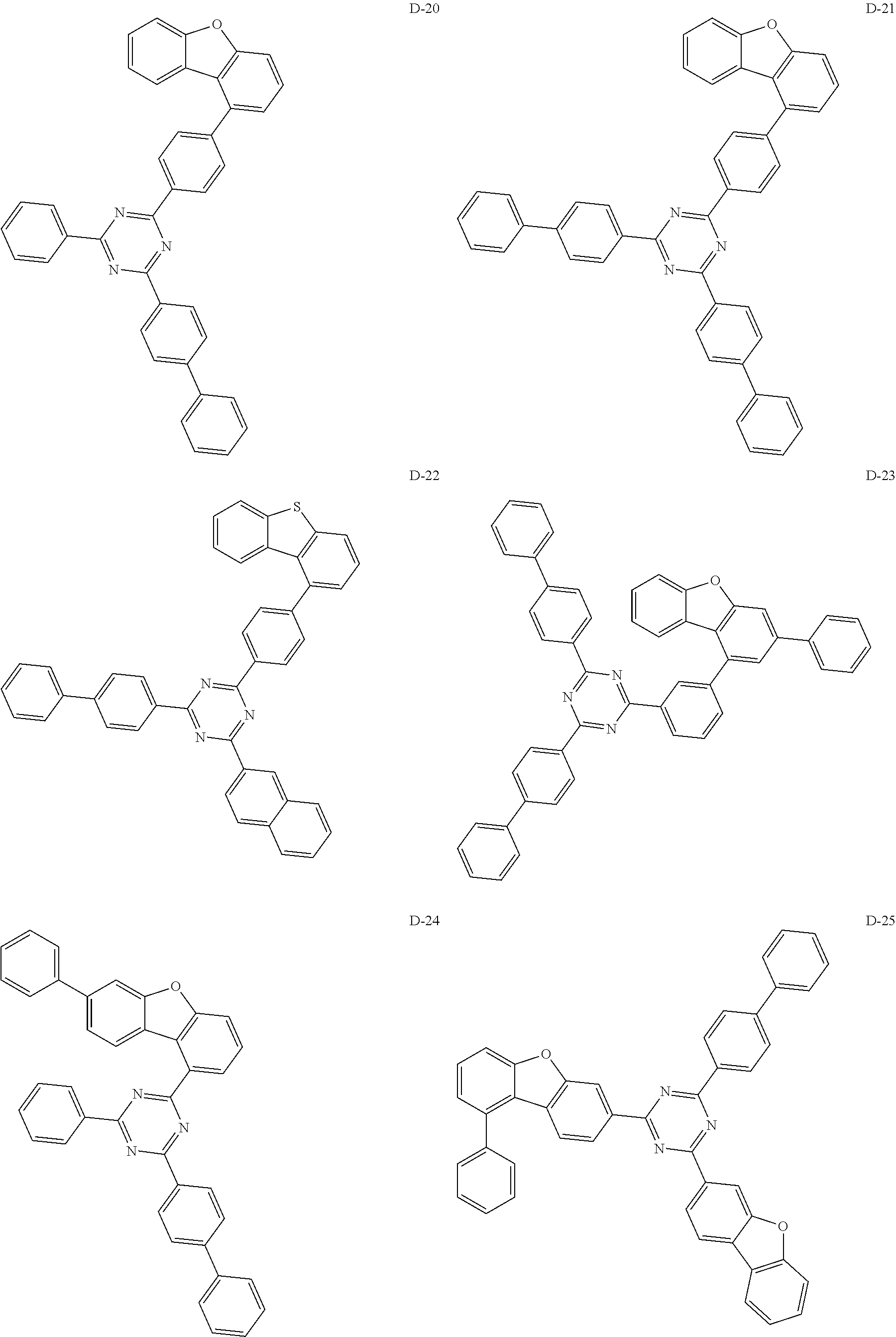

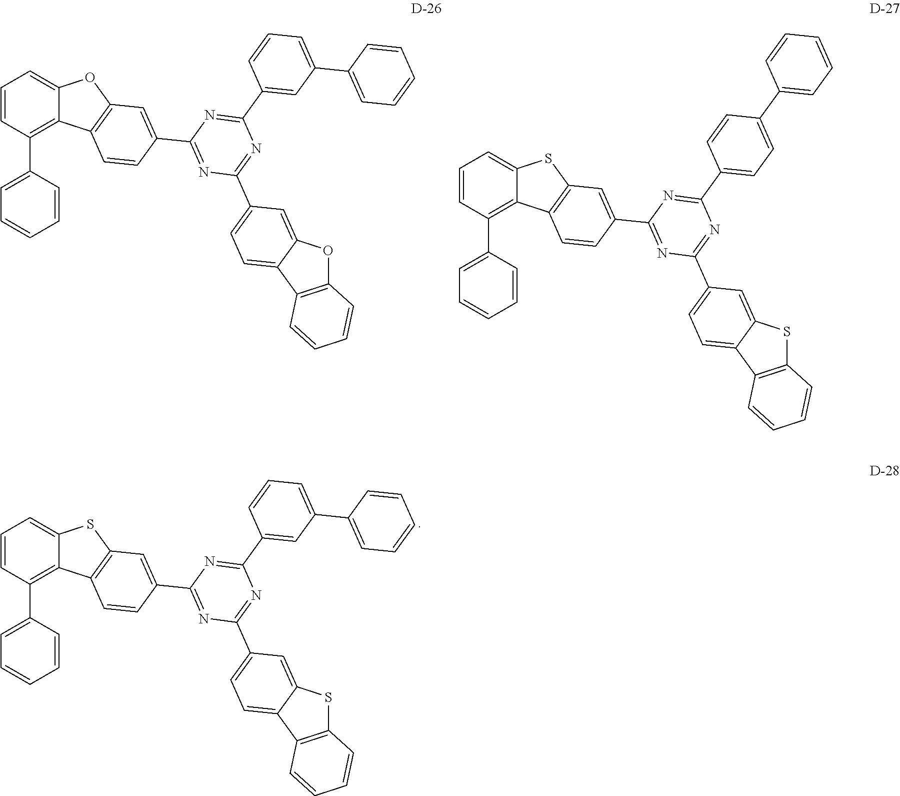 [SMILES: c1ccc(-c2cccc(-c3nc(-c4ccc5c(c4)oc4ccccc45)nc(-c4ccc5c(c4)oc4cccc(-c6ccccc6)c45)n3)c2)cc1 ; c1ccc(-c2ccc(-c3nc(-c4ccc5c(c4)sc4ccccc45)nc(-c4ccc5c(c4)sc4cccc(-c6ccccc6)c45)n3)cc2)cc1 ; c1ccc(-c2cccc(-c3nc(-c4ccc5c(c4)sc4ccccc45)nc(-c4ccc5c(c4)sc4cccc(-c6ccccc6)c45)n3)c2)cc1]

7. The composition as claimed in claim 1, wherein Ar⁴ to Ar⁶ of Chemical Formula 2 and Chemical Formula 3 are independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylenyl group, a substituted or unsubstituted fluorenyl group, a substituted or unsubstituted dibenzofuranyl group, a substituted or unsubstituted dibenzothiophenyl group, a substituted or unsubstituted carbazolyl group, or a combination thereof.

8. The composition as claimed in claim 1, wherein the second compound is one of the compounds listed in Group 2:

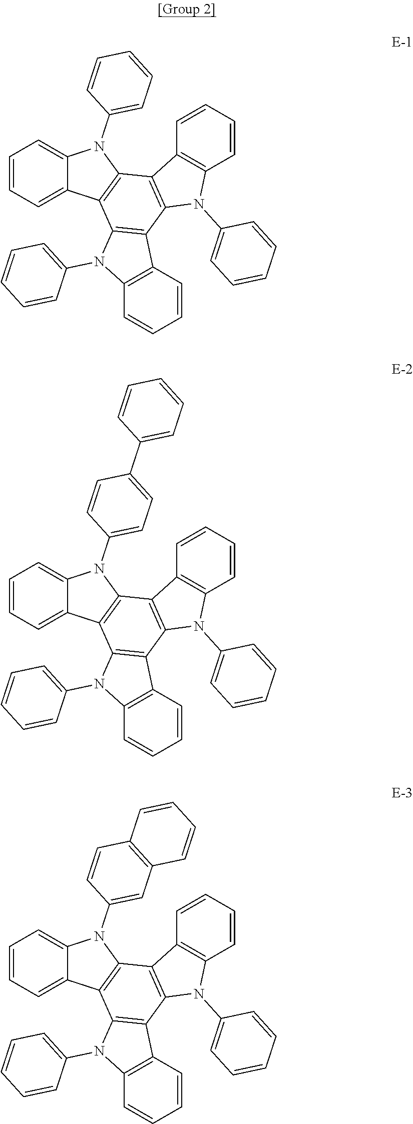

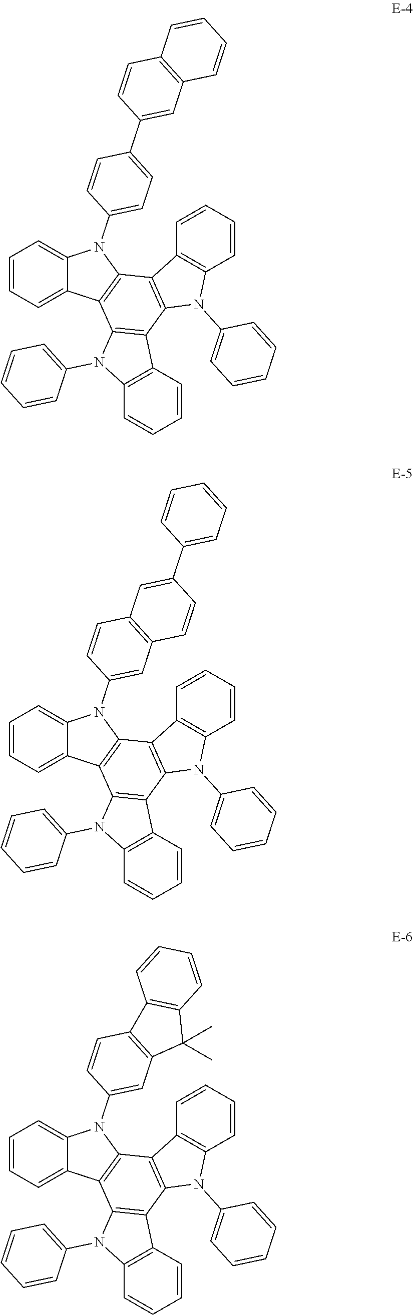 [SMILES: c1ccc(-n2c3ccccc3c3c2c2c4ccccc4n(-c4ccc(-c5ccc6ccccc6c5)cc4)c2c2c4ccccc4n(-c4ccccc4)c32)cc1 ; c1ccc(-c2ccc3cc(-n4c5ccccc5c5c6c(c7ccccc7n6-c6ccccc6)c6c(c7ccccc7n6-c6ccccc6)c54)ccc3c2)cc1 ; CC1(C)c2ccccc2-c2ccc(-n3c4ccccc4c4c5c(c6ccccc6n5-c5ccccc5)c5c(c6ccccc6n5-c5ccccc5)c43)cc21]

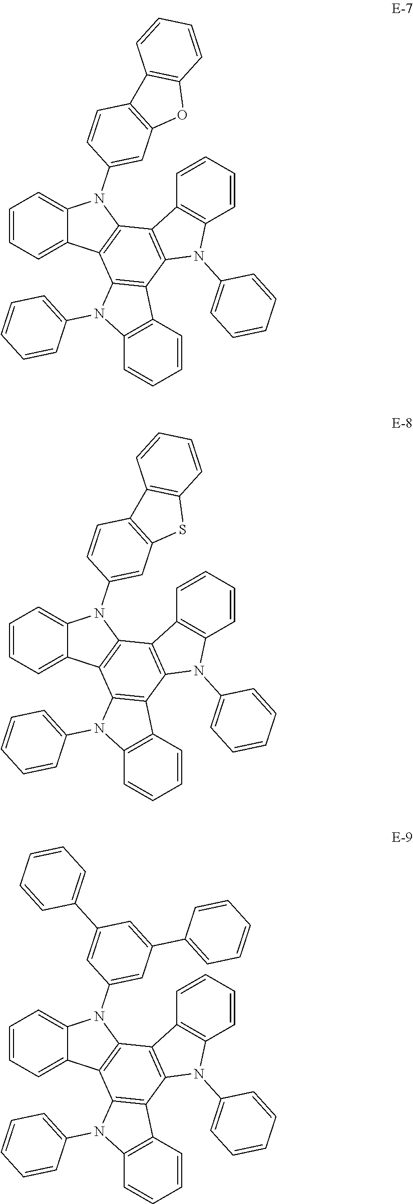 [SMILES: c1ccc(-n2c3ccccc3c3c2c2c4ccccc4n(-c4ccc5c(c4)oc4ccccc45)c2c2c4ccccc4n(-c4ccccc4)c32)cc1 ; c1ccc(-n2c3ccccc3c3c2c2c4ccccc4n(-c4ccc5c(c4)sc4ccccc45)c2c2c4ccccc4n(-c4ccccc4)c32)cc1 ; c1ccc(-c2cc(-c3ccccc3)cc(-n3c4ccccc4c4c5c(c6ccccc6n5-c5ccccc5)c5c(c6ccccc6n5-c5ccccc5)c43)c2)cc1]

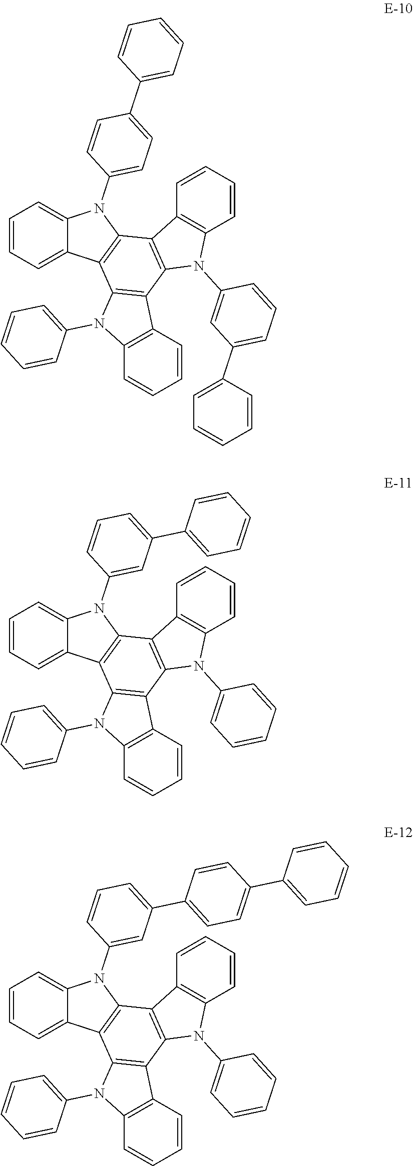 [SMILES: c1ccc(-c2ccc(-n3c4ccccc4c4c3c3c5ccccc5n(-c5cccc(-c6ccccc6)c5)c3c3c5ccccc5n(-c5ccccc5)c43)cc2)cc1 ; c1ccc(-c2cccc(-n3c4ccccc4c4c5c(c6ccccc6n5-c5ccccc5)c5c(c6ccccc6n5-c5ccccc5)c43)c2)cc1 ; c1ccc(-c2ccc(-c3cccc(-n4c5ccccc5c5c6c(c7ccccc7n6-c6ccccc6)c6c(c7ccccc7n6-c6ccccc6)c54)c3)cc2)cc1]

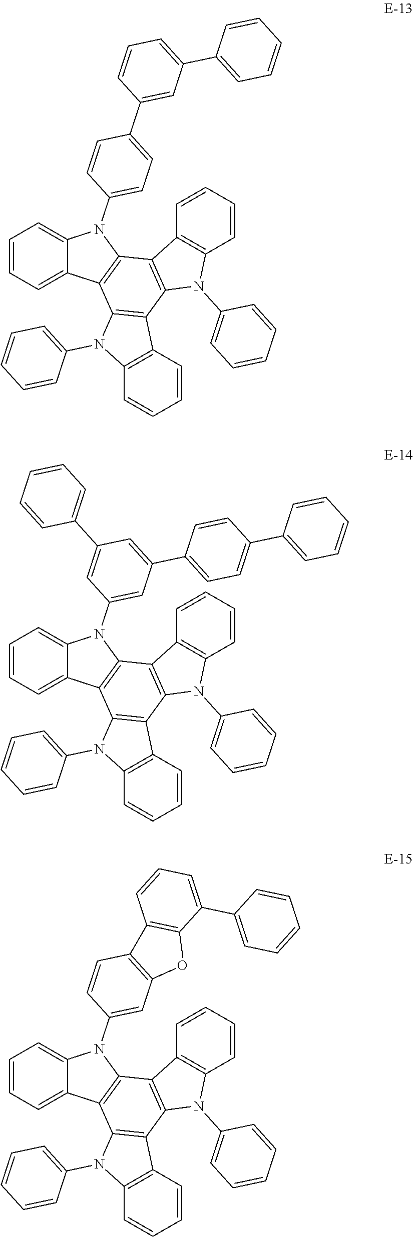 [SMILES: c1ccc(-c2cccc(-c3ccc(-n4c5ccccc5c5c6c(c7ccccc7n6-c6ccccc6)c6c(c7ccccc7n6-c6ccccc6)c54)cc3)c2)cc1 ; c1ccc(-c2ccc(-c3cc(-c4ccccc4)cc(-n4c5ccccc5c5c6c(c7ccccc7n6-c6ccccc6)c6c(c7ccccc7n6-c6ccccc6)c54)c3)cc2)cc1 ; c1ccc(-c2cccc3c2oc2cc(-n4c5ccccc5c5c6c(c7ccccc7n6-c6ccccc6)c6c(c7ccccc7n6-c6ccccc6)c54)ccc23)cc1]

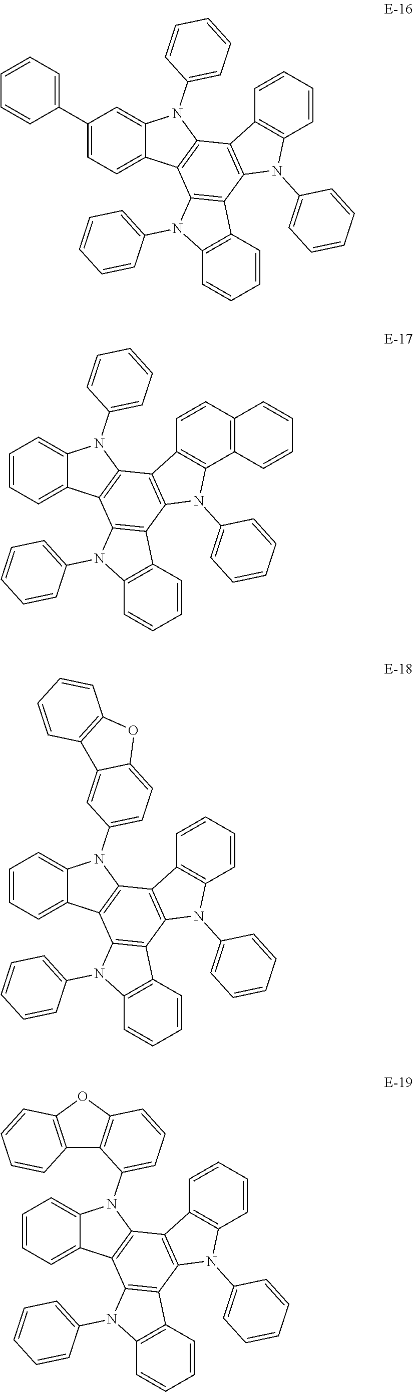 [SMILES: c1ccc(-c2ccc3c4c5c(c6ccccc6n5-c5ccccc5)c5c(c6ccccc6n5-c5ccccc5)c4n(-c4ccccc4)c3c2)cc1 ; c1ccc(-n2c3ccccc3c3c2c2c4ccc5ccccc5c4n(-c4ccccc4)c2c2c4ccccc4n(-c4ccccc4)c32)cc1 ; c1ccc(-n2c3ccccc3c3c2c2c4ccccc4n(-c4ccc5oc6ccccc6c5c4)c2c2c4ccccc4n(-c4ccccc4)c32)cc1 ; c1ccc(-n2c3ccccc3c3c2c2c4ccccc4n(-c4cccc5oc6ccccc6c45)c2c2c4ccccc4n(-c4ccccc4)c32)cc1]

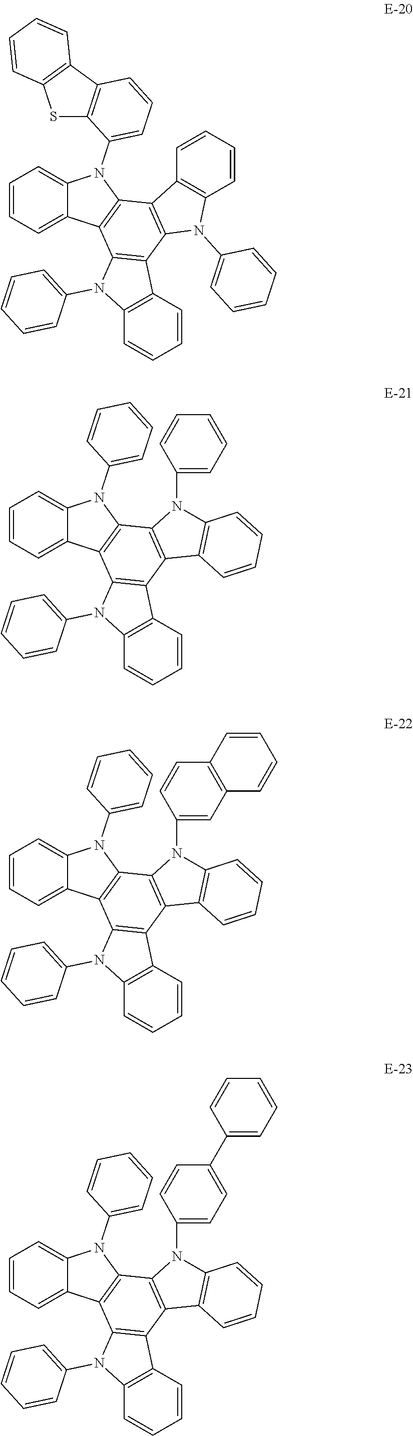 [SMILES: c1ccc(-n2c3ccccc3c3c2c2c4ccccc4n(-c4cccc5c4sc4ccccc45)c2c2c4ccccc4n(-c4ccccc4)c32)cc1 ; c1ccc(-n2c3ccccc3c3c4c5ccccc5n(-c5ccccc5)c4c4c(c5ccccc5n4-c4ccccc4)c32)cc1 ; c1ccc(-n2c3ccccc3c3c4c5ccccc5n(-c5ccc6ccccc6c5)c4c4c(c5ccccc5n4-c4ccccc4)c32)cc1 ; c1ccc(-c2ccc(-n3c4ccccc4c4c5c6ccccc6n(-c6ccccc6)c5c5c6ccccc6n(-c6ccccc6)c5c43)cc2)cc1]

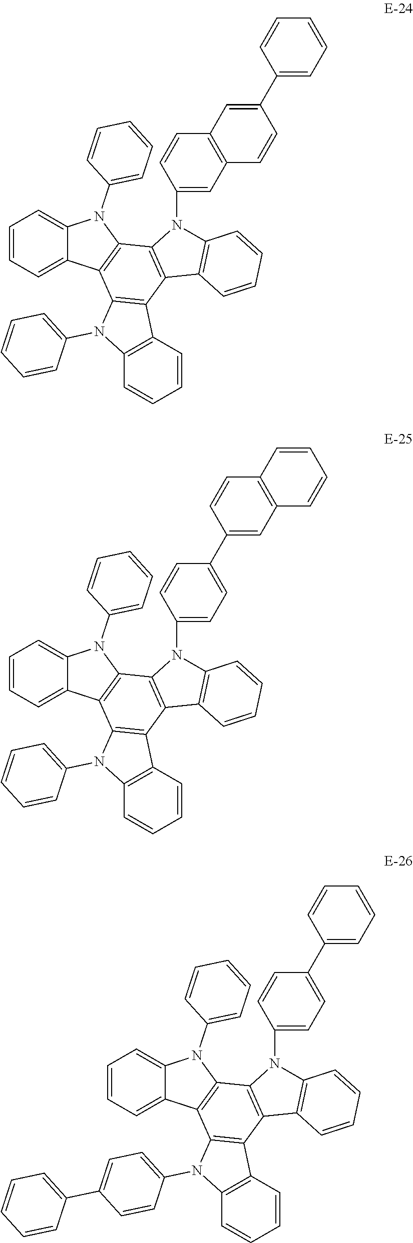 [SMILES: c1ccc(-c2ccc3cc(-n4c5ccccc5c5c6c7ccccc7n(-c7ccccc7)c6c6c7ccccc7n(-c7ccccc7)c6c54)ccc3c2)cc1 ; c1ccc(-n2c3ccccc3c3c4c5ccccc5n(-c5ccc(-c6ccc7ccccc7c6)cc5)c4c4c(c5ccccc5n4-c4ccccc4)c32)cc1 ; c1ccc(-c2ccc(-n3c4ccccc4c4c5c6ccccc6n(-c6ccc(-c7ccccc7)cc6)c5c5c(c6ccccc6n5-c5ccccc5)c43)cc2)cc1]

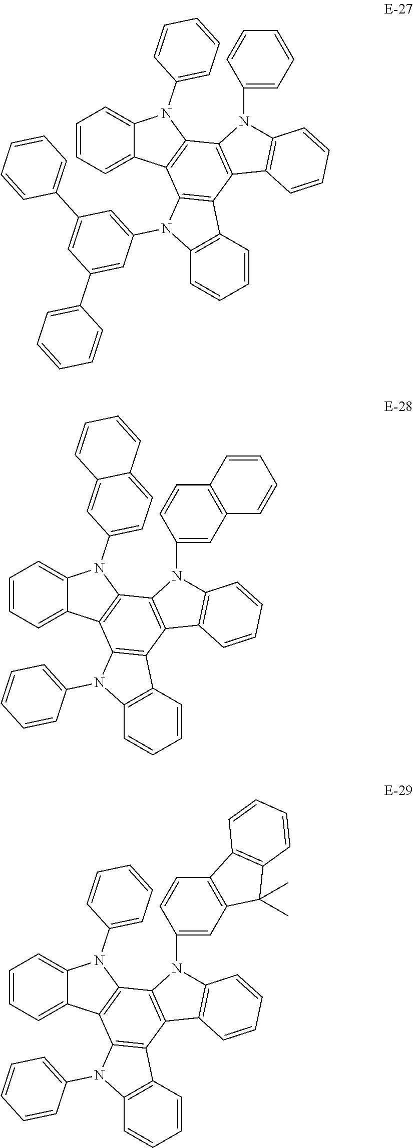 [SMILES: c1ccc(-c2cc(-c3ccccc3)cc(-n3c4ccccc4c4c5c6ccccc6n(-c6ccccc6)c5c5c(c6ccccc6n5-c5ccccc5)c43)c2)cc1 ; c1ccc(-n2c3ccccc3c3c4c5ccccc5n(-c5ccc6ccccc6c5)c4c4c(c5ccccc5n4-c4ccc5ccccc5c4)c32)cc1 ; CC1(C)c2ccccc2-c2ccc(-n3c4ccccc4c4c5c6ccccc6n(-c6ccccc6)c5c5c6ccccc6n(-c6ccccc6)c5c43)cc21]

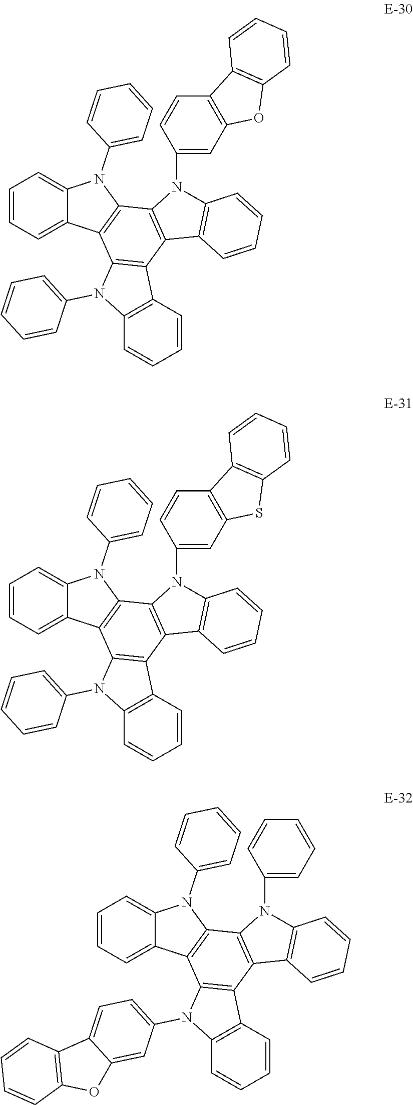 [SMILES: c1ccc(-n2c3ccccc3c3c4c5ccccc5n(-c5ccc6c(c5)oc5ccccc56)c4c4c(c5ccccc5n4-c4ccccc4)c32)cc1 ; c1ccc(-n2c3ccccc3c3c4c5ccccc5n(-c5ccc6c(c5)sc5ccccc56)c4c4c(c5ccccc5n4-c4ccccc4)c32)cc1 ; c1ccc(-n2c3ccccc3c3c4c5ccccc5n(-c5ccc6c(c5)oc5ccccc56)c4c4c5ccccc5n(-c5ccccc5)c4c32)cc1]

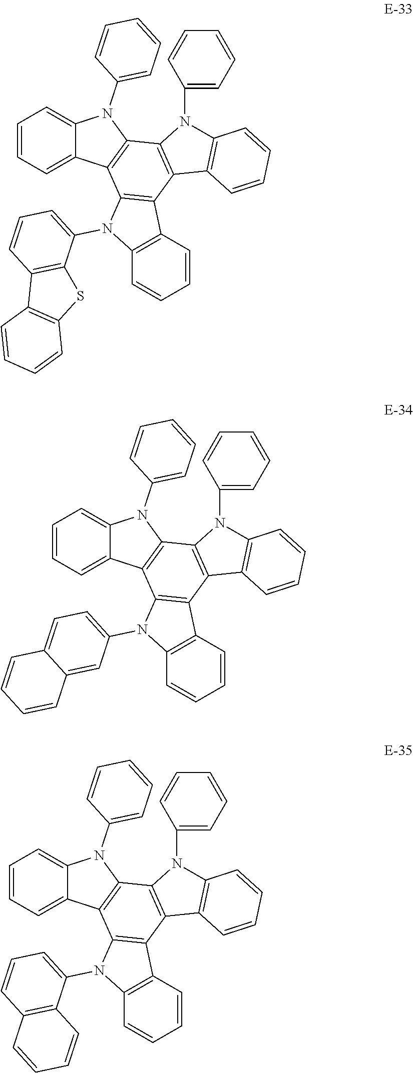 [SMILES: c1ccc(-n2c3ccccc3c3c4c5ccccc5n(-c5cccc6c5sc5ccccc56)c4c4c5ccccc5n(-c5ccccc5)c4c32)cc1 ; c1ccc(-n2c3ccccc3c3c4c5ccccc5n(-c5ccc6ccccc6c5)c4c4c5ccccc5n(-c5ccccc5)c4c32)cc1 ; c1ccc(-n2c3ccccc3c3c4c5ccccc5n(-c5cccc6ccccc56)c4c4c5ccccc5n(-c5ccccc5)c4c32)cc1]

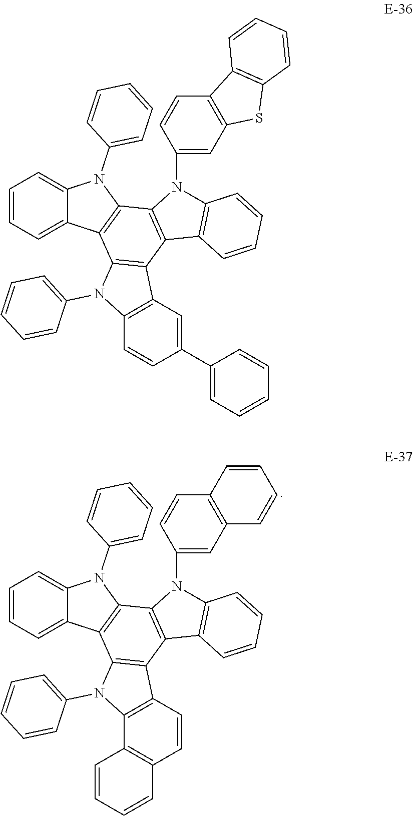 [SMILES: c1ccc(-c2ccc3c(c2)c2c4c5ccccc5n(-c5ccc6c(c5)sc5ccccc56)c4c4c(c5ccccc5n4-c4ccccc4)c2n3-c2ccccc2)cc1 ; c1ccc(-n2c3ccccc3c3c2c2c(c4ccccc4n2-c2ccc4ccccc4c2)c2c4ccc5ccccc5c4n(-c4ccccc4)c23)cc1]

9. The composition as claimed in claim 1, further comprising a dopant.

10. An organic optoelectronic device, comprising:
an anode and a cathode facing each other, and
at least one organic layer between the anode and the cathode,
wherein the organic layer includes the composition as claimed in claim 1.

11. The organic optoelectronic device as claimed in claim 10, wherein:
the organic layer includes a light emitting layer, and
the light emitting layer includes the composition.

12. The organic optoelectronic device as claimed in claim 11, wherein the first compound and the second compound are a phosphorescent host of the light emitting layer.

13. The organic optoelectronic device as claimed in claim 11, wherein the composition is a red light emitting composition.

14. The organic optoelectronic device as claimed in claim 11, wherein the composition is a green light emitting composition.

15. A display device comprising the organic optoelectronic device as claimed in claim 10.
